(19) United States
(12) Patent Application Publication
(10) Pub. No.: US 2005/0209284 A1
(43) Pub. Date: Sep. 22, 2005

(54) Tec kinase inhibitors

(75) Inventors: Joerg Martin Bentzien, White Plains, NY (US); Brian Nicholas Cook, Danbury, CT (US); Xiang Li, New Milford, CT (US); Ho Yin Lo, Bethel, CT (US); Chuk Chui Man, Ridgefield, CT (US); Ingo Andreas Mugge, New Haven, CT (US); Peter Allen Nemoto, Southbury, CT (US); Steven S. Pullen, Danbury, CT (US); Doris Edith Riether, New York, NY (US); Gregory Paul Roth, Woodstock, CT (US); Fariba Soleymanzadeh, Brewster, NY (US); Hidenori Takahashi, LaGrangeville, NY (US); Ji Wang, Danbury, CT (US); Andre White, Newtown, CT (US); Renee Zindell, New Milford, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 11/055,798

(22) Filed: Feb. 9, 2005

Related U.S. Application Data
(60) Provisional application No. 60/544,218, filed on Feb. 12, 2004.

(51) Int. Cl. A61K 31/4439; A61K 31/427; A61K 31/422; C07D 417/02; C07D 413/02; C07D 43/02; A61K 31/4184

(52) U.S. Cl. 514/338; 514/365; 514/374; 514/394; 546/273.4; 548/181; 546/215; 548/305.1; 548/307.4

(57) ABSTRACT

Disclosed are compounds of formula(I): 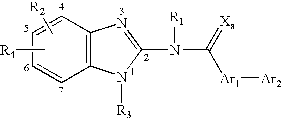
wherein Ar₁, Ar₂, R₁, R₂, R₃, R₄ and Xₐ are defined herein. The compounds of the invention inhibit Itk kinase and are therefore useful for treating diseases and pathological conditions involving inflammation, immunological disorders and allergic disorders. Also disclosed are processes for preparing these compounds and to pharmaceutical compositions comprising these compounds.

### APPLICATION DATA

This application claims benefit to U.S. provisional application No. 60/544,218 filed Feb. 12, 2004.

### TECHNICAL FIELD OF THE INVENTION

This invention relates to substituted benzimidazole compounds of formula(I): 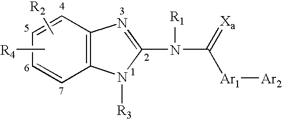
wherein Ar₁, Ar₂, R₁, R₂, R₃, R₄ and Xₐ are defined herein below. The compounds of the invention inhibit Itk kinase and are therefore useful for treating diseases and pathological conditions involving inflammation, immunological disorders and allergic disorders. This invention also relates to processes for preparing these compounds and to pharmaceutical compositions comprising these compounds.

### BACKGROUND OF THE INVENTION

Protein kinases play a critical role in mediating signaling events leading to cellular responses such as activation, growth and differentiation, in response to extracellular signals. Protein kinases transmit their signal by phosphorylating specific residues in a target protein. Protein kinases that specifically phosphorylate tyrosine residues are referred to as protein tyrosine kinases. Protein tyrosine kinases can be divided into two general groups: receptor such as epidermal growth factor (EGF) receptor (S. Iwashita and M. Kobayashi, 1992, Cellular Signalling, 4, 123-132) and cytosolic non-receptor (C. Chan et al., 1994, Ann. Rev. Immunol., 12, 555-592).

Interleukin-2-inducible T cell kinase (Itk), also referred to as T cell-specific kinase (Tsk) and expressed mainly in T-lymphocytes (EMT), is a member of the Tec family of protein tyrosine kinases that also includes Txk, Tec, Btk, and Bmx. Tec family members are characterized by the presence of a pleckstrin-homology domain (PH), a proline rich Tec homology domain (TH) and Src homology SH3, SH2 and SH1 kinase domains positioned from the N-terminus to the C-terminus respectively (S. Gibson et al., 1993, Blood, 82, 1561-1572; J. D. Siliciano et al., 1992, Proc. Nat. Acad. Sci., 89, 11194-11198; N. Yamada et al., 1993 Biochem. and Biophys Res. Comm., 192, 231-240).

Itk is expressed in T cells, mast cells and natural killer cells. It is activated in T cells upon stimulation of the T cell receptor (TCR), and in mast cells upon activation of the high affinity IgE receptor. Following receptor stimulation in T cells, Lck, a src tyrosine kinase family member, phosphorylates Y511 in the kinase domain activation loop of Itk (S. D. Heyeck et al., 1997, J. Biol. Chem, 272, 25401-25408). Activated Itk, together with Zap-70 is required for phosphorylation and activation of PLC-γ (S. C. Bunnell et al., 2000, J. Biol. Chem., 275, 2219-2230). PLC-γ catalyzes the formation of inositol 1,4,5-triphosphate and diacylglycerol, leading to calcium mobilization and PKC activation, respectively. These events activate numerous downstream pathways and lead ultimately to degranulation (mast cells) and cytokine gene expression (T cells) (Y. Kawakami et al., 1999, J. Leukocyte Biol., 65, 286-290).

The role of Itk in T cell activation has been confirmed in Itk knockout mice. CD4⁺T cells from Itk knockout mice have a diminished proliferative response in a mixed lymphocyte reaction or upon Con A or anti-CD3 stimulation. (X. C. Liao and D. R. Littman, 1995, Immunity, 3, 757-769). Also, T cells from Itk knockout mice produced little IL-2 upon TCR stimulation resulting in reduced proliferation of these cells. In another study, Itk deficient CD4⁺ T cells produced reduced levels of cytokines including IL-4, IL-5 and IL-13 upon stimulation of the TCR, even after priming with inducing conditions. (D. J. Fowell, 1999, Immunity, 11, 399-409).

The role of Itk in PLC-γ activation and in calcium mobilization was also confirmed in the T cells of these knockout mice, which had severely impaired IP₃ generation and no extracellular calcium influx upon TCR stimulation (K. Liu et al., 1998, J. Exp. Med. 187, 1721-1727). The studies described above support a key role for Itk in activation of T cells and mast cells. Thus an inhibitor of Itk would be of therapeutic benefit in diseases mediated by inappropriate activation of these cells.

It has been well established that T cells play an important role in regulating the immune response (Powrie and Coffman, 1993, Immunology Today, 14, 270-274). Indeed, activation of T cells is often the initiating event in immunological disorders. Following activation of the TCR, there is an influx of calcium that is required for T cell activation. Upon activation, T cells produce cytokines, including IL-2, 4, 5, 9, 10, and 13 leading to T cell proliferation, differentiation, and effector function. Clinical studies with inhibitors of IL-2 have shown that interference with T cell activation and proliferation effectively suppresses immune response in vivo (Waldmann, 1993, Immunology Today, 14, 264-270). Accordingly, agents that inhibit T lymphocyte activation and subsequent cytokine production, are therapeutically useful for selectively suppressing the immune response in a patient in need of such immunosuppression.

Mast cells play a critical roll in asthma and allergic disorders by releasing pro-inflammatory mediators and cytokines. Antigen-mediated aggregation of FcεRI, the high-affinity receptor for IgE results in activation of mast cells (D. B. Corry et al., 1999, Nature, 402, B18-23). This triggers a series of signaling events resulting in the release of mediators, including histamine, proteases, leukotrienes and cytokines (J. R. Gordon et al., 1990, Immunology Today, 11, 458-464.) These mediators cause increased vascular permeability, mucus production, bronchoconstriction, tissue degradation and inflammation thus playing key roles in the etiology and symptoms of asthma and allergic disorders.

Recent published data using Itk knockout mice suggests that in the absence of Itk function, increased numbers of memory T cells are generated (A. T. Miller et al., 2002 The Journal of Immunology, 168, 2163-2172). One strategy to improve vaccination methods is to increase the number of memory T cells generated (S. M. Kaech et al., Nature Reviews Immunology, 2, 251-262).

All patent and literature documents cited in this application are incorporated by reference in their entirety.

### SUMMARY OF THE INVENTION

It is therefore an object of the invention to provide a compound of the formula (I): 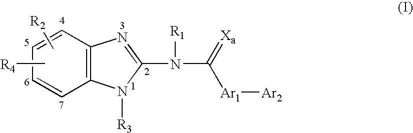
wherein Ar₁, Ar₂, R₁, R₂, R₃, R₄ and Xₐ are defined herein below.

It is another object of the invention to provide a method of inhibiting the Tec kinase family, including Itk kinase, and methods of treating diseases or conditions related to such kinase activity activity, by administering to a patient in need thereof a therapeutically effective amount of a compound of the formula (I).

It is yet another object of the invention to provide pharmaceutical compositions and processes of making compounds of the formula (I) as described herein below.

### DETAILED DESCRIPTION OF THE PREFERRED EMBODIMENTS

In it's broadest generic embodiment, the invention provides for a compound of the formula (I): 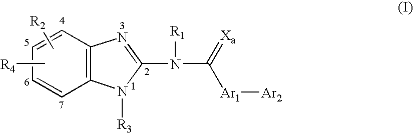
wherein:
R₁ is hydrogen or alkyl;
R₂, covalently attached at the indicated 4-, 5-, 6- or 7-position of the formula (I), is chosen from hydrogen, alkyl, alkoxy and halogen;
Ar₁ is chosen from carbocycle and heteroaryl each optionally substituted with one or more amine, alkyl, alkoxy or halogen;
Ar₂ is chosen from carbocycle, heterocycle and heteroaryl each optionally substituted with one or more Rₐ;
R₃ is C₁₋₁₀ alkyl chain branched or unbranched optionally substituted with one or more R_{b},
   or R₃ is the group:
      —(CH₂)ₙ-L-R₆, wherein L is chosen from a bond, —NH—C(O)—, —O—C(O)—, —C(O)— and —S(O)ₘ— wherein m is 0, 1 or 2, and wherein said group is optionally substituted by one or more R_{b};
   wherein R₆ is independently chosen from hydrogen, hydroxy, alkyl, alkoxy, alkylthio, arylC₀₋₅ alkyl, aryloxyC₀₋₅ alkyl, heteroarylC₀₋₅ alkyl, cycloalkylC₀₋₅ alkyl, heterocyclylC₀₋₅ alkyl and amino said amino is optionally mono-or di-substituted by acyl, alkyl, alkoxycarbonyl, cycloalkylC₀₋₅ alkyl, arylC₀₋₅ alkyl, heteroarylC₀₋₅ alkyl or heterocyclylC₀₋₅ alkyl;
   n is 1-10;
R₄ is a group chosen from: 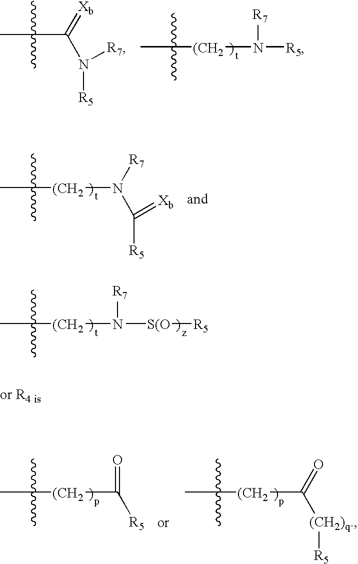
wherein if p or q>0 then a hydrogen atom from their respective —(CH₂)— group(s) may be replaced with a C₁₋₁₀ alkyl wherein one or more —CH₂— groups of said alkyl are optionally replaced by a heteroatom group chosen from O, S and NH,
wherein R₄ is covalently attached at the indicated 5- or 6-position of the formula (I), p, q, t and z are each independently chosen from 0,1 or 2;
R₅ is chosen from arylC₀₋₅ alkyl, alkyl, heteroarylC₀₋₅ alkyl, cycloalkylC₀₋₅ alkyl and heterocyclylC₀₋₅ alkyl, each R₅ optionally substituted with one or more R_{c};
R₇ is hydrogen, alkenyl or alkyl;
or R₅ and R₇ together with the nitrogen atom to which they are attached form:
a 4-7-membered monocyclic ring or
an 8-14-membered bicyclic ring,
wherein each monocyclic or bicyclic ring optionally contains an additional 1 to 3 heteroatoms chosen from N, O and S and each ring is aromatic or nonaromatic, and wherein each monocyclic or bicyclic ring is optionally substituted by one or more R_{c};
each Rₐ, R_{b} or R_{c} are independently chosen from hydrogen, alkyl, alkenyl, alkynyl, cycloalkyl, cycloalkenyl, aryl, arylalkyl, aryloxy, alkoxy, alkylthio, acyl, alkoxycarbonyl, acyloxy, acylamino, sulphonylamino, aminosulfonyl, alkylsulfonyl, carboxy, carboxamide, oxo, hydroxy, halogen, trifluoromethyl, nitro, nitrile and amino optionally mono-or-di-substituted by alkyl, acyl or alkoxycarbonyl, wherein any of the above Rₐ, R_{b} or R_{c} are optionally halogenated where possible; and
Xₐ and X_{b} are oxygen or sulfur;
   or the pharmaceutically acceptable salts, esters, acids, isomers or tautomers thereof.

In another embodiment, there is provided a compound of the formula (I) as described immediately above and wherein:
R₁ is hydrogen;
R₂ is chosen from hydrogen, C₁₋₃ alkyl, C₁₋₃ alkoxy and halogen;
Ar₁ is phenyl or heteroaryl chosen from thienyl, furanyl, isoxazolyl, oxazolyl, thiazolyl, oxadiazolyl, thiadiazolyl, tetrazolyl, pyrazolyl, pyrrolyl, imidazolyl, pyridinyl, pyrimidinyl, pyrazinyl, pyridazinyl and pyranyl;
Ar₂ is chosen from C₃₋₈ cycloalkyl, C₄₋₈ cycloalkenyl, phenyl, naphthyl and heteroaryl chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothiophenyl, benzodioxolyl, quinolinyl, quinazolinyl and indazolyl each is optionally substituted with one or more Rₐ;
R₃ is C₁₋₁₀ alkyl chain branched or unbranched optionally substituted with one or more R_{b},
   or R₃ is:
—(CH₂)ₙ-L-R₆, wherein L is chosen from a bond, —O—C(O)—, —C(O)— and —S(O)ₘ— wherein m is 0, 1 or 2, and wherein said group is optionally substituted by one or more R_{b};
wherein R₆ is independently chosen from hydrogen, hydroxy, C₁₋₅ alkyl, C₁₋₅ alkoxy, C₁₋₅ alkylthio, phenyl, naphthyl, benzyl, phenethyl, heteroarylC₀₋₅ alkyl, C₃₋₇ cycloalkyl₀₋₅ alkyl, heterocyclylC₀₋₅ alkyl and amino said amino is optionally mono- or di-substituted by C₁₋₅ acyl, C₁₋₅ alkyl, C₁₋₅ alkoxycarbonyl, arylC₀₋₅ alkyl, heteroarylC₀₋₅ alkyl or heterocyclylC₀₋₅ alkyl; and wherein each recited heteroaryl in this paragraph is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl and wherein each recited heterocyclyl in this paragraph is chosen from pyrrolidinyl, morpholinyl, thiomorpholinyl, dioxalanyl, piperidinyl and piperazinyl;
R₄ is a group chosen from: 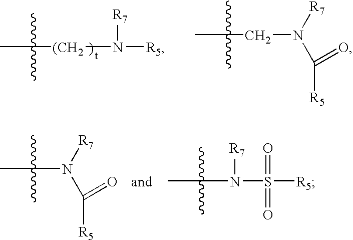
R₅ is chosen from phenyl, naphthyl, benzyl, phenethyl, C₁₋₅ alkyl, heteroarylC₀₋₅ alkyl wherein the heteroaryl is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl, C₃₋₇ cycloalkylC₀₋₅ alkyl and heterocyclylC₀₋₅ alkyl wherein the heterocyclyl is chosen from aziridinyl, pyrrolidinyl, morpholinyl, thiomorpholinyl, tetrahydrofuranyl, dioxalanyl, piperidinyl and piperazinyl, each R₅ is optionally substituted with one or more R_{c};
each Rₐ, R_{b} or R_{c} are independently chosen from hydrogen, C₁₋₅ alkyl, C₂₋₅ alkenyl, C₂₋₅ alkynyl, C₃₋₈ cycloalkyl, C₄₋₈ cycloalkenyl, phenyl, benzyl, phenoxy, C₁₋₅ alkoxy, C₁₋₅ alkylthio, C₁₋₅ acyl, C₁₋₅ alkoxycarbonyl, C₁₋₅ acyloxy, C₁₋₅ acylamino, C₁₋₅ sulphonylamino, aminosulfonyl, C₁₋₅ alkylsulfonyl, carboxy, carboxamide, oxo, hydroxy, halogen, trifluoromethyl, nitro, nitrile and amino optionally mono-or-di-substituted by C₁₋₅ alkyl, C₁₋₅ acyl or C₁₋₅ alkoxycarbonyl, wherein any of the above Rₐ, R_{b} or R_{c} are optionally halogenated where possible;
R₇ is hydrogen, C₃₋₁₀ alkenyl or C₁₋₅ alkyl; and
Xₐ is oxygen.

In yet another embodiment, there is provided a compound of the formula (I) as described immediately above and wherein:
R₂ is chosen from hydrogen and C₁₋₃ alkyl;
Ar₁ is chosen from phenyl, thienyl, furanyl, isoxazolyl, oxazolyl, imidazolyl, oxadiazolyl, thiadiazolyl, pyrazolyl and pyridinyl;
Ar₂ is chosen from C₄₋₈ cycloalkenyl, C₄₋₈ cycloalkyl, phenyl, naphthyl, benzothiophenyl, benzodioxolyl, quinolinyl, indolyl, thiazolyl, thienyl, furanyl, isoxazolyl, oxazolyl, imidazolyl, thiadiazolyl, pyrazolyl, pyrazinyl and pyridinyl each is optionally substituted with one or more Rₐ;
R₆ is independently chosen from hydroxy, C₁₋₅ alkyl, C₁₋₅ alkoxy, phenyl, benzyl, phenethyl, heteroarylC₀₋₅ alkyl, heterocyclylC₀₋₅ alkyl, C₃₋₇ cycloalkyl and amino said amino is optionally mono-or di-substituted by C₁₋₅ acyl, C₁₋₅ alkyl, C₁₋₅ alkoxycarbonyl, arylC₀₋₅ alkyl or heteroarylC₀₋₅ alkyl;
   and wherein each recited heteroaryl in this paragraph is chosen from thienyl, furanyl, isoxazolyl, oxazolyl, thiazolyl, thiadiazolyl, tetrazolyl, pyrazolyl, pyrrolyl and imidazolyl, each optionally substituted by R_{b};
n is 1-6;
R₅ is chosen from phenyl, naphthyl, benzyl, phenethyl, C₁₋₅ alkyl, heteroarylC₀₋₅ alkyl wherein the heteroaryl in this paragraph is chosen from thienyl, furanyl, imidazolyl and pyridinyl, C₃₋₇ cycloalkylC₀₋₅ alkyl and heterocyclylC₀₋₅ alkyl wherein the heterocyclyl is chosen from aziridinyl, pyrrolidinyl, tetrahydrofuranyl, tetrahydropyridinyl, morpholinyl, thiomorpholinyl, piperidinyl and piperazinyl, each R₅ is optionally substituted with one or more R_{c};
R₇ is hydrogen, propenyl or C₁₋₃ alkyl.

In yet still another embodiment, there is provided a compound of the formula (I) as described immediately above and wherein:
R₂ is chosen from hydrogen and methyl;
Ar₂ is chosen from cyclopentyl, cyclohexyl, cycloheptyl, cyclopentenyl, cyclohexenyl, cycloheptenyl, phenyl, naphthyl, benzothiophenyl, benzodioxolyl, quinolinyl, indolyl, thiazolyl, thienyl, furanyl, isoxazolyl, oxazolyl, imidazolyl, thiadiazolyl, oxadiazolyl, pyrazinyl and pyridinyl each is optionally substituted with one or more Rₐ;
R₃ is:
—(CH₂)ₙ—C(O)—R₆ or
—(CH₂)ₙ—R₆;
   wherein R₆ is independently chosen from hydroxy, C₁₋₅ alkyl, C₁₋₅ alkoxy, phenyl, morpholinylC₀₋₅ alkyl, piperazinylC₀₋₅ alkyl, imidazolylC₀₋₅ alkyl, pyrrolidinylC₀₋₅ alkyl, pyrrolidinonylC₀₋₅ alkyl, thienylC₀₋₅ alkyl, C₃₋₇ cycloalkyl and amino said amino is optionally mono-or di-substituted by C₁₋₅ alkyl or C₁₋₅ alkoxycarbonyl;
R₅ is chosen from phenyl, furanyl, benzyl, phenethyl, C₁₋₃ alkyl and C₃₋₇ cycloalkylC₀₋₅ alkyl each optionally substituted with one or more R_{c};
each Rₐ, R_{b} or R_{c} are independently chosen from C₁₋₅ alkyl, C₂₋₅ alkenyl, C₃₋₈ cycloalkyl, C₄₋₈ cycloalkenyl, phenyl, C₁₋₅ alkoxy, C₁₋₅ alkylthio, amino optionally mono-or-di-substituted by C₁₋₅ alkyl, C₁₋₅ alkoxycarbonyl, carboxamide, hydroxy, halogen, trifluoromethyl, nitro and nitrile, wherein any of the above Rₐ, R_{b} or R_{c} are optionally halogenated where possible;
R₇ is C₁₋₃ alkyl.

In a further embodiment, there is provided a compound of the formula (I) as described immediately above and wherein:
R₂ is hydrogen;
Ar₁ is chosen from phenyl, thienyl, furanyl, isoxazolyl and pyridinyl;
Ar₂ is chosen from cycloheptyl, cycloheptenyl, phenyl, naphthyl, benzothiophenyl, benzodioxolyl, quinolinyl, indolyl, thiazolyl, thienyl, furanyl, isoxazolyl, oxazolyl, thiadiazolyl, pyrazinyl and pyridinyl each is optionally substituted with one or more Rₐ;
R₅ is chosen from methyl, CF₃, cyclopentyl, phenyl and cyclohexyl each optionally substituted with one or more R_{c}; and
n is 2-5.

In yet another embodiment, there is provided a compound of the formula (I) as described immediately above and wherein:
Ar₁ is chosen from phenyl, thien-2-yl, isoxazol-5-yl and pyridin-3-yl;
Rₐ is chosen from phenyl, C₆₋₈ cycloalkyl, C₆₋₈ cycloalkenyl, C₁₋₃ alkoxy, C₁₋₄ alkyl, C₂₋₃ alkenyl, C₁₋₃ alkylthio, amino, carboxamide, fluoro, chloro, nitro and nitrile;
R₄ is chosen from: 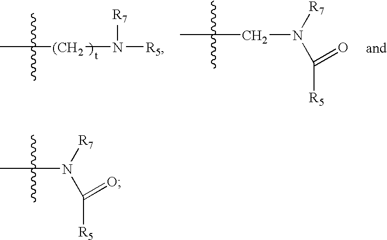
R₆ is independently chosen from hydroxy, methyl, ethyl, C₁₋₃ alkoxy, phenyl, morpholinyl, piperazinyl, imidazolyl, pyrrolidinyl, pyrrolidinonyl, thienylC₀₋₅ alkyl, C₃₋₇ cycloalkyl and amino said amino is optionally mono-or di-substituted by C₁₋₅ alkyl or C₁₋₅ alkoxycarbonyl; and
each R_{b} or R_{c} are independently chosen from C₁₋₃ alkoxy, amino optionally mono-or-di-substituted by C₁₋₃ alkyl, carboxamide, hydroxy, fluoro, chloro, bromo, trifluoromethyl, nitro and nitrine.

In any of the aforementioned embodiments, there are provided compounds of the formula (I) wherein:
R₄ is covalently attached at the indicated 5-position of the formula (I) or in another embodiment R₄ is covalently attached at the indicated 6-position of the formula (I).

In another embodiment, there is provided a compound in accordance with the broadest generic embodiment above: 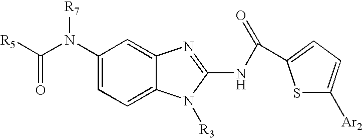
and wherein:
Ar₂ is chosen from cycloheptyl, cycloheptenyl, phenyl, naphthyl, benzothiophenyl, benzodioxolyl, quinolinyl, indolyl, thiazolyl, thienyl, furanyl, isoxazolyl, oxazolyl, thiadiazolyl, pyrazinyl and pyridinyl each is optionally substituted with one or more Rₐ chosen from phenyl, C₆₋₈ cycloalkyl, C₆₋₈ cycloalkenyl, C₁₋₃ alkoxy, C₁₋₄ alkyl, C₂₋₃ alkenyl, C₁₋₃ alkylthio, amino, carboxamide, fluoro, chloro, nitro and nitrile;
R₃ is:
—(CH₂)ₙ—C(O)—R₆ or
—(CH₂)ₙ—R₆;
R₆ is independently chosen from hydroxy, methyl, ethyl, C₁₋₃ alkoxy, phenyl, morpholinyl, piperazinyl, imidazolyl, pyrrolidinyl, pyrrolidinonyl, thienylC₀₋₅ alkyl, C₃₋₇ cycloalkyl and amino said amino is optionally mono-or di-substituted by C₁₋₅ alkyl or C₁₋₅ alkoxycarbonyl;
R₅ is chosen from methyl, CF₃, cyclopentyl, phenyl and cyclohexyl each optionally substituted with one or more R_{c} chosen from C₁₋₃ alkoxy, amino optionally mono-or-di-substituted by C₁₋₃ alkyl, carboxamide, hydroxy, fluoro, chloro, bromo, trifluoromethyl, nitro and nitrile and
R₇ is C₁₋₃ alkyl.

In another embodiment, there is provided a compound in accordance with the embodiment immediately above: 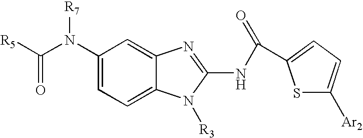
and wherein:
Ar₂ is chosen from phenyl, indolyl, thiazolyl, thienyl, furanyl, isoxazolyl, oxazolyl, thiadiazolyl, pyrazinyl and pyridinyl each is optionally substituted with one or more Rₐ chosen from phenyl, C₆₋₈ cycloalkyl, C₆₋₈ cycloalkenyl, C₁₋₃ alkoxy, C₁₋₄ alkyl, C₂₋₃ alkenyl, C₁₋₃ alkylthio, amino, carboxamide, fluoro, chloro, nitro and nitrile.

In another embodiment, there is provided a compound in accordance with the embodiment immediately above: 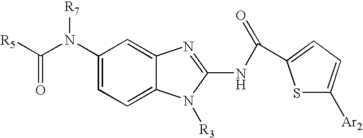
and wherein:
Ar₂ is chosen from 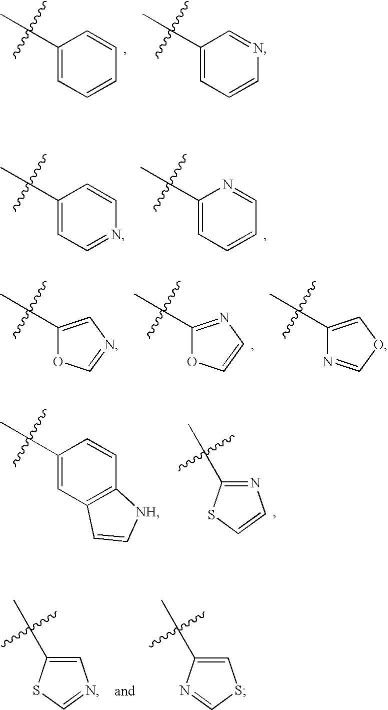
   each is optionally substituted with one Rₐ chosen from phenyl, C₁₋₃ alkoxy, C₁₋₄ alkyl, C₂₋₃ alkenyl, C₁₋₃ alkylthio, amino, carboxamide, fluoro, chloro, nitro and nitrile;
   with the proviso that if Ar₂ is: 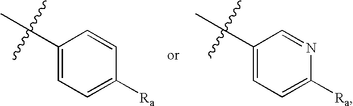
   then Rₐ must be —NO₂, —NH₂ or —CN.

In another embodiment there is provided representative compounds of the invention which can be made in accordance with the general schemes and working examples presented below:

TABLE I

| | |
|---|---|
| 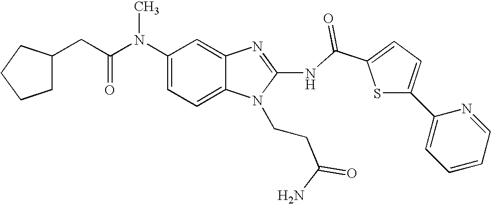 | 5-Pyridin-2-yl-thiophene-2-carboxylic acid {1- (2-carbamoyl-ethyl)-5-[(2-cyclopentyl-acetyl)- methyl-amino]-1H-benzoimidazol-2-yl}-amide |
| 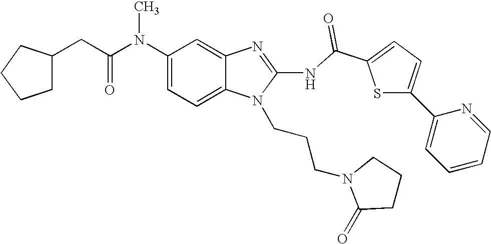 | 5-Pyridin-2-yl-thiophene-2-carboxylic acid {5- [(2-cyclopentyl-acetyl)-methyl-amino]-1-[3-(2- oxo-pyrrolidin-1-yl)-propyl]-1H- benzoimidazol-2-yl}-amide |
| 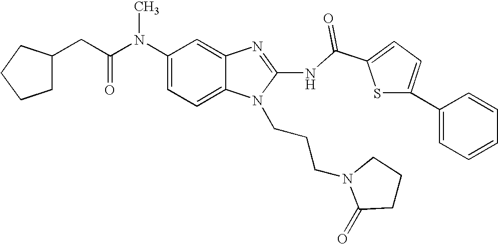 | 5-Phenyl-thiophene-2-carboxylic acid {5-[(2- cyclopentyl-acetyl)-methyl-amino]-1-[3-(2-oxo- pyrrolidin-1-yl)-propyl]-1H-benzoimidazol- 2-yl}-amide |
| 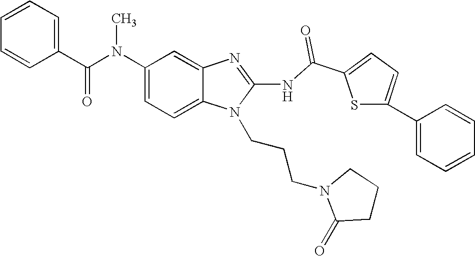 | 5-Phenyl-thiophene-2-carboxylic acid {5- (benzoyl-methyl-amino)-1-[3-(2-oxo- pyrrolidin-1-yl)-propyl]-1H-benzoimidazol- 2-yl}-amide |
| 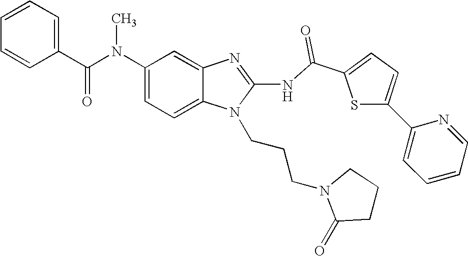 | 5-Pyridin-2-yl-thiophene-2-carboxylic acid {5- (benzoyl-methyl-amino)-1-[3-(2-oxo- pyrrolidin-1-yl)-propyl]-1H- benzoimidazol-2-yl}-amide |
| 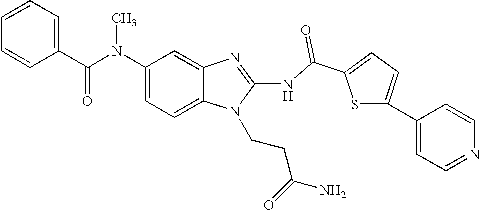 | 5-Pyridin-4-yl-thiophene-2-carboxylic acid [5- (benzoyl-methyl-amino)-1-(2-carbamoyl-ethyl)- 1H-benzoimidazol-2-yl]-amide |
| 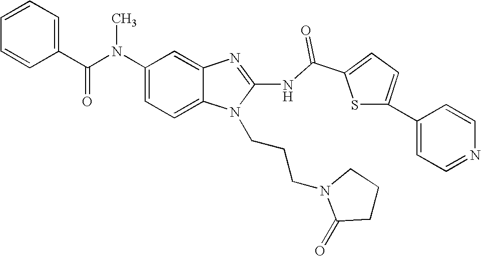 | 5-Pyridin-4-yl-thiophene-2-carboxylic acid {5- (benzoyl-methyl-amino)-1-[3-(2-oxo- pyrrolidin-1-yl)-propyl]-1H- benzoimidazol-2-yl}-amide |
| 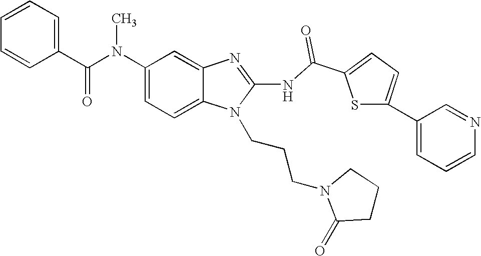 | 5-Pyridin-3-yl-thiophene-2-carboxylic acid {5- (benzoyl-methyl-amino)-1-[3-(2-oxo- pyrrolidin-1-yl)-propyl]-1H- benzoimidazol-2-yl}-amide |
| 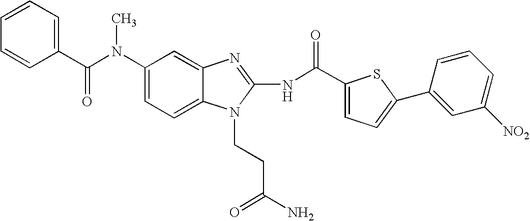 | 5-(3-Nitro-phenyl)-thiophene-2-carboxylic acid [5-(benzoyl-methyl-amino)-1-(2-carbamoyl- ethyl)-1H-benzoimidazol-2-yl]-amide |
| 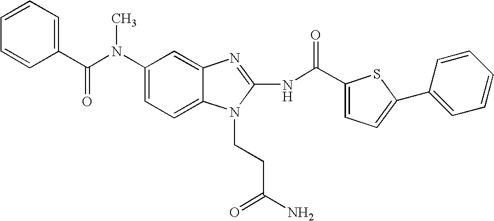 | 5-Phenyl-thiophene-2-carboxylic acid [5- (benzoyl-methyl-amino)-1-(2-carbamoyl-ethyl)- 1H-benzoimidazol-2-yl]-amide |
| 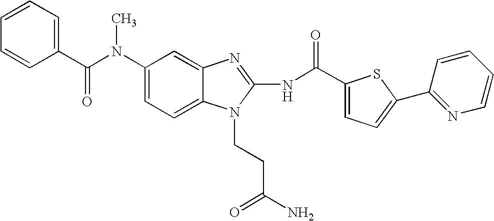 | 5-Pyridin-2-yl-thiophene-2-carboxylic acid [5- (benzoyl-methyl-amino)-1-(2-carbamoyl-ethyl)- 1H-benzoimidazol-2-yl]-amide |
| 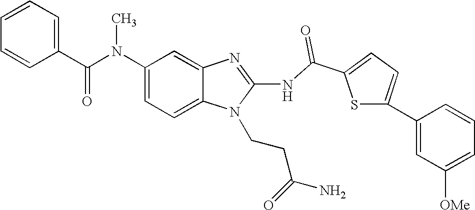 | 5-(3-Methoxy-phenyl)-thiophene-2-carboxylic acid [5-(benzoyl-methyl-amino)-1-(2- carbamoyl-ethyl)-1H-benzoimidazol- 2-yl]-amide |
| 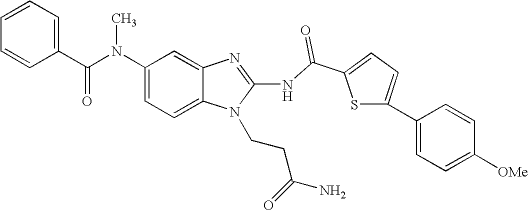 | 5-(4-Methoxy-phenyl)-thiophene-2-carboxylic acid [5-(benzoyl-methyl-amino)-1-(2- carbamoyl-ethyl)-1H-benzoimidazol-2-yl]- amide |
| 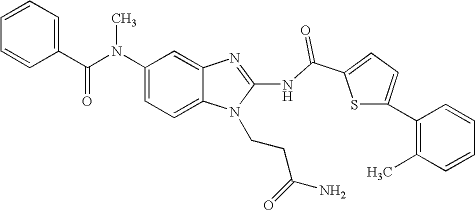 | 5-o-Tolyl-thiophene-2-carboxylic acid [5- (benzoyl-methyl-amino)-1-(2-carbamoyl- ethyl)-1H-benzoimidazol- 2-yl]-amide |
| 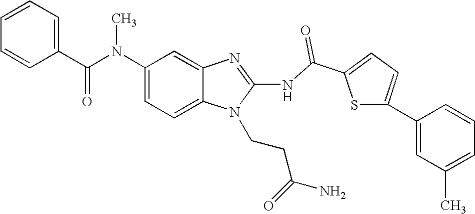 | 5-m-Tolyl-thiophene-2-carboxylic acid [5- (benzoyl-methyl-amino)-1-(2- carbamoyl-ethyl)-1H- benzoimidazol-2-yl]-amide |
| 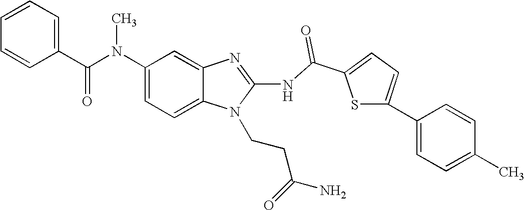 | 5-p-Tolyl-thiophene-2-carboxylic acid [5- (benzoyl-methyl-amino)-1-(2-carbamoyl-ethyl)- 1H-benzoimidazol-2-yl]-amide |
| 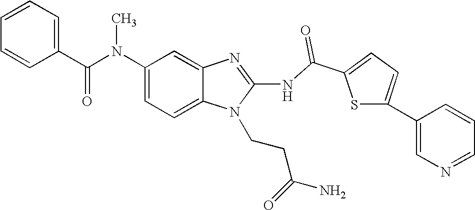 | 5-Pyridin-3-yl-thiophene-2-carboxylic acid [5- (benzoyl-methyl-amino)-1-(2-carbamoyl-ethyl)- 1H-benzoimidazol-2-yl]-amide |
| 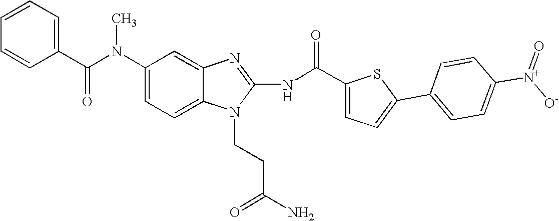 | 5-(4-Nitro-phenyl)-thiophene-2-carboxylic acid [5-(benzoyl-methyl-amino)-1-(2-carbamoyl- ethyl)-1H-benzoimidazol-2-yl]-amide |
| 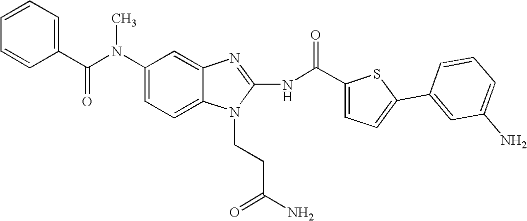 | 5-(3-Amino-phenyl)-thiophene-2-carboxylic acid [5-(benzoyl-methyl-amino)-1-(2- carbamoyl-ethyl)-1H-benzoimidazol-2-yl]- amide |
| 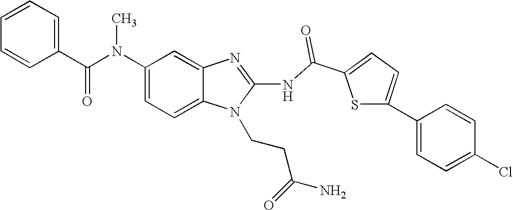 | 5-(4-Chloro-phenyl)-thiophene-2-carboxylic acid [5-(benzoyl-methyl-amino)-1-(2- carbamoyl-ethyl)-1H-benzoimidazol-2-yl]- amide |
| 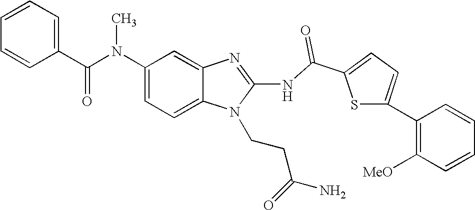 | 5-(2-Methoxy-phenyl)-thiophene-2-carboxylic acid [5-(benzoyl-methyl-amino)-1-(2- carbamoyl-ethyl)-1H-benzoimidazol-2-yl]- amide |
| 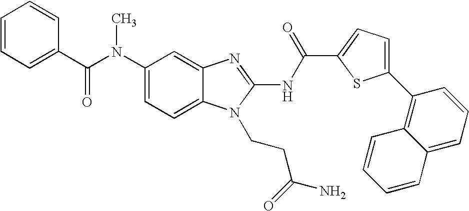 | 5-Naphthalen-1-yl-thiophene-2-carboxylic acid [5-(benzoyl-methyl-amino)-1-(2-carbamoyl- ethyl)-1H-benzoimidazol-2-yl]-amide |
| 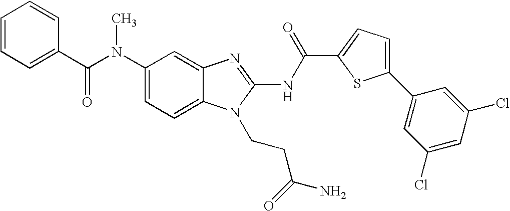 | 5-(3,5-Dichloro-phenyl)-thiophene-2- carboxylic acid [5-(benzoyl-methyl-amino)-1- (2-carbamoyl-ethyl)-1H-benzoimidazol-2-yl]- amide |
| 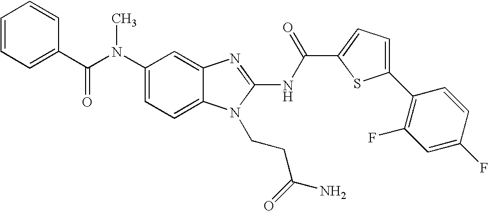 | 5-(2,4-Difluoro-phenyl)-thiophene-2-carboxylic acid [5-(benzoyl-methyl-amino)-1-(2- carbamoyl-ethyl)-1H-benzoimidazol-2-yl]- amide |
| 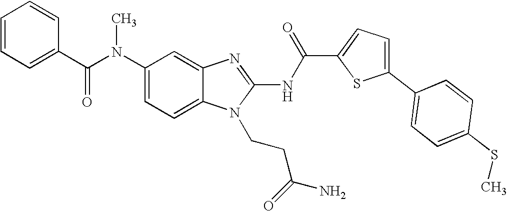 | 5-(4-Methylsulfanyl-phenyl)-thiophene-2- carboxylic acid [5-(benzoyl-methyl-amino)-1- (2-carbamoyl-ethyl)-1H-benzoimidazol-2-yl]- amide |
| 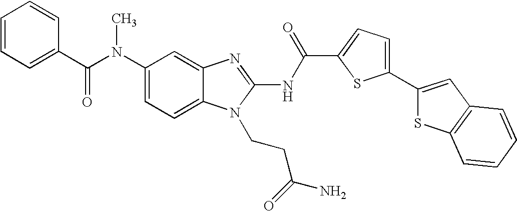 | 5-Benzo[b]thiophen-2-yl-thiophene-2- carboxylic acid [5-(benzoyl-methyl-amino)-1- (2-carbamoyl-ethyl)-1H-benzoimidazol-2-yl]- amide |
| 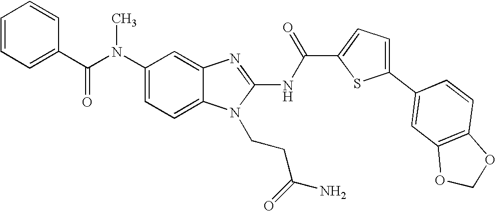 | 5-Benzo[1,3]dioxol-5-yl-thiophene-2- carboxylic acid [5-(benzoyl-methyl-amino)-1- (2-carbamoyl-ethyl)-1H-benzoimidazol-2-yl]- amide |
| 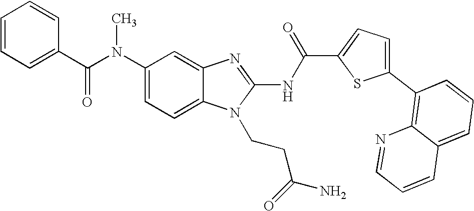 | 5-Quinolin-8-yl-thiophene-2-carboxylic acid [5- (benzoyl-methyl-amino)-1-(2-carbamoyl-ethyl)- 1H-benzoimidazol-2-yl]-amide |
| 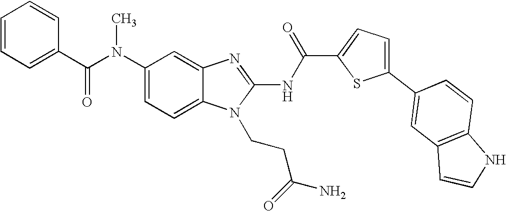 | 5-(1H-Indol-5-yl)-thiophene-2-carboxylic acid [5-(benzoyl-methyl-amino)-1-(2-carbamoyl- ethyl)-1H-benzoimidazol-2-yl]-amide |
| 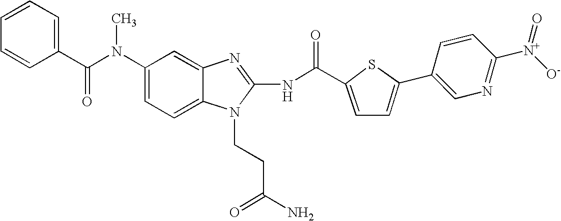 | 5-(6-Nitro-pyridin-3-yl)-thiophene-2-carboxylic acid [5-(benzoyl-methyl-amino)-1-(2- carbamoyl-ethyl)-1H-benzoimidazol- 2-yl]-amide |
| 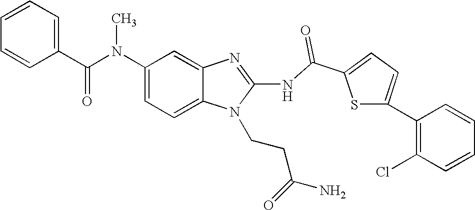 | 5-(2-Chloro-phenyl)-thiophene-2-carboxylic acid [5-(benzoyl-methyl-amino)-1-(2- carbamoyl-ethyl)-1H-benzoimidazol-2-yl]- amide |
| 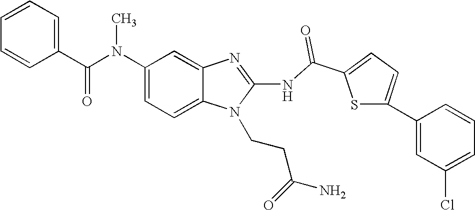 | 5-(3-Chloro-phenyl)-thiophene-2-carboxylic acid [5-(benzoyl-methyl-amino)-1-(2- carbamoyl-ethyl)-1H-benzoimidazol-2-yl]- amide |
| 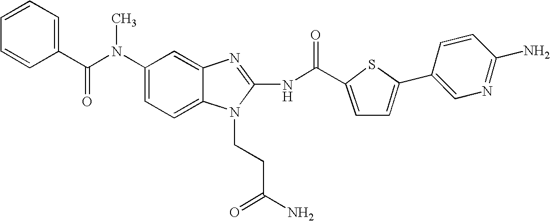 | 5-(6-Amino-pyridin-3-yl)-thiophene-2- carboxylic acid [5-(benzoyl-methyl-amino)-1- (2-carbamoyl-ethyl)-1H-benzoimidazol-2-yl]- amide |
| 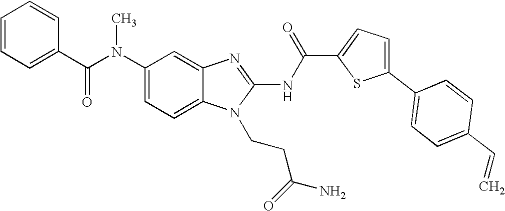 | 5-(4-Vinyl-phenyl)-thiophene-2-carboxylic acid [5-(benzoyl-methyl-amino)-1-(2-carbamoyl- ethyl)-1H-benzoimidazol-2-yl]-amide |
| 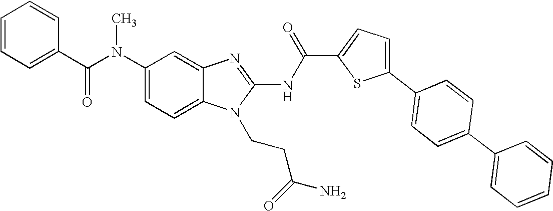 | 5-Biphenyl-4-yl-thiophene-2-carboxylic acid [5-(benzoyl-methyl-amino)-1-(2-carbamoyl- ethyl)-1H-benzoimidazol-2-yl]-amide |
| 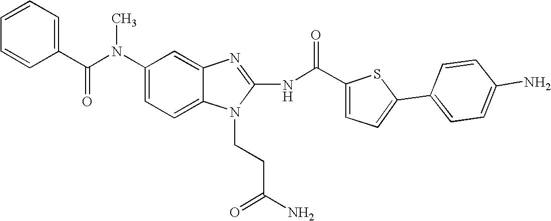 | 5-(4-Amino-phenyl)-thiophene-2-carboxylic acid [5-(benzoyl-methyl-amino)-1-(2- carbamoyl-ethyl)-1H-benzoimidazol-2-yl]- amide |
| 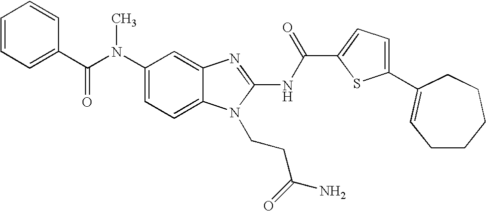 | 5-Cyclohept-1-enyl-thiophene-2-carboxylic acid [5-(benzoyl-methyl-amino)-1-(2- carbamoyl-ethyl)-1H-benzoimidazol-2-yl]- amide |
| 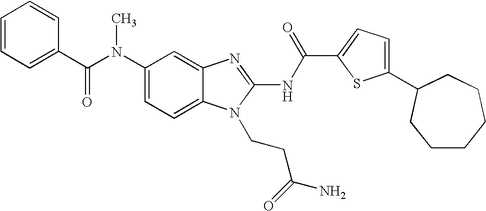 | 5-Cycloheptyl-thiophene-2-carboxylic acid [5- (benzoyl-methyl-amino)-1-(2 -carbamoyl-ethyl)- 1H-benzoimidazol-2-yl]-amide |
| 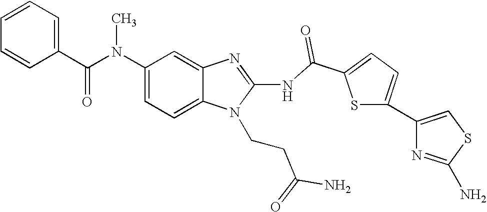 | 5-(2-Amino-thiazol-4-yl)-thiophene-2- carboxylic acid [5-(benzoyl-methyL-amino)-1- (2-carbamoyl-ethyl)-1H-benzoimidazol-2-yl]- amide |
| 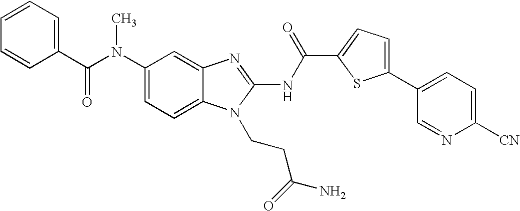 | 5-(6-Cyano-pyridin-3-yl)-thiophene-2- carboxylic acid [5-(benzoyl-methyl-amino)-1- (2-carbamoyl-ethyl)-1H-benzoimidazol-2-yl]- amide |
| 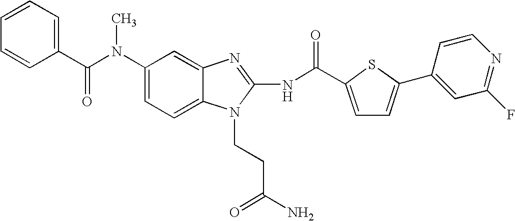 | 5-(2-Fluoro-pyridin-4-yl)-thiophene-2- carboxylic acid [5-(benzoyl-methyl-amino)-1- (2-carbamoyl-ethyl)-1H-benzoimidazol-2-yl]- amide |
| 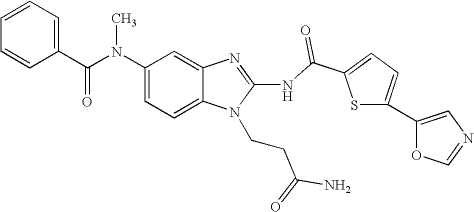 | 5-Oxazol-5-yl-thiophene-2-carboxylic acid [5- (benzoyl-methyl-amino)-1-(2-carbamoyl-ethyl)- 1H-benzoimidazol-2-yl]-amide |
| 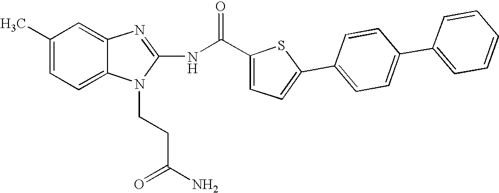 | 5-Biphenyl-4-yl-thiophene-2-carboxylic acid [1-(2-carbamoyl-ethyl)-5-methyl-1H- benzoimidazol-2-yl]-amide |
| 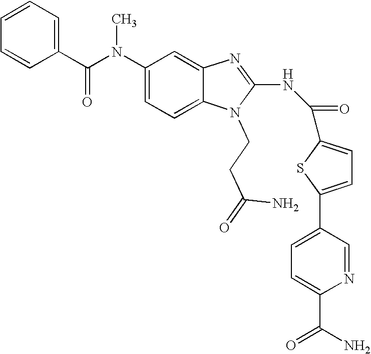 | 5-{5-[5-(Benzoyl-methyl-amino)-1-(2- carbamoyl-ethyl)-1H-benzoimidazol-2- ylcarbamoyl]-thiophen-2-yl}-pyridine-2- carboxylic acid amide |
| 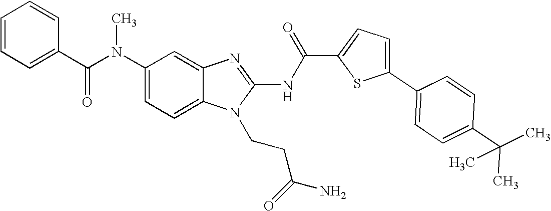 | 5-(4-tert-Butyl-phenyl)-thiophene-2-carboxylic acid [5-(benzoyl-methyl-amino)-1-(2- carbamoyl-ethyl)-1H-benzoimidazol-2-yl]- amide |
| 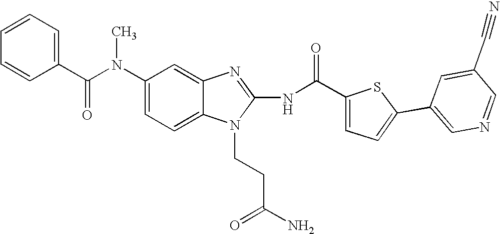 | 5-(5-Cyano-pyridin-3-yl)-thiophene-2- carboxylic acid [5-(benzoyl-methyl-amino)-1- (2-carbamoyl-ethyl)-1H-benzoimidazol-2-yl]- amide |
| 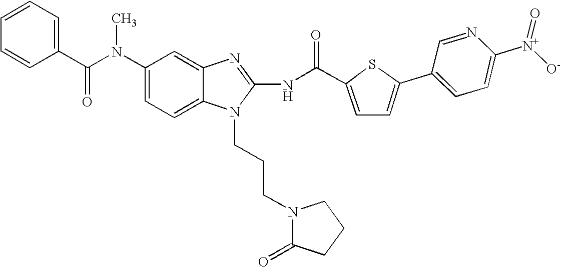 | 5-(6-Nitro-pyridin-3-yl)-thiophene-2-carboxylic acid {5-(benzoyl-methyl-amino)-1-[3-(2-oxo- pyrrolidin-1-yl)-propyl]-1H-benzoimidazol-2- yl}-amide |
| 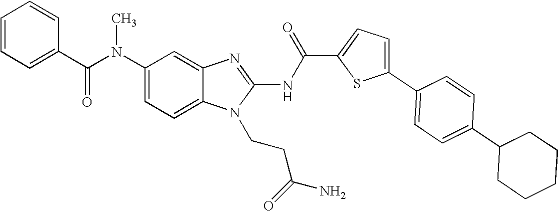 | 5-(4-Cyclohexyl-phenyl)-thiophene-2- carboxylic acid [5-(benzoyl-methyl-amino)-1- (2-carbamoyl-ethyl)-1H-benzoimidazol-2-yl]- amide |
| 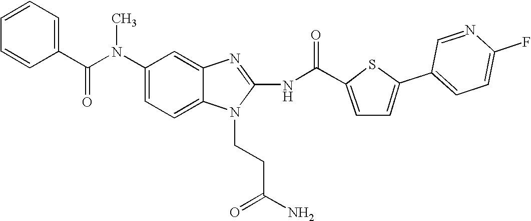 | 6-Fluoro-pyridin-3-yl)-thiophene-2- carboxylic acid [5-(benzoyl-methyl-amino)-1- (2-carbamoyl-ethyl)-1H-benzoimidazol-2-yl]- amide |
| 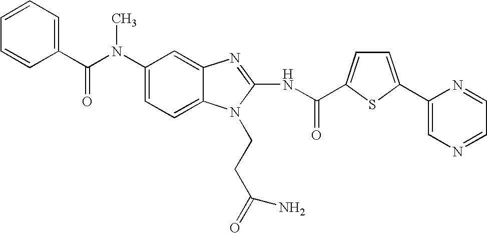 | 5-Pyrazin-2-yl-thiophene-2-carboxylic acid [5- (benzoyl-methyl-amino)-1-(2-carbamoyl-ethyl)- 1H-benzoimidazol-2-yl]-amide |
| 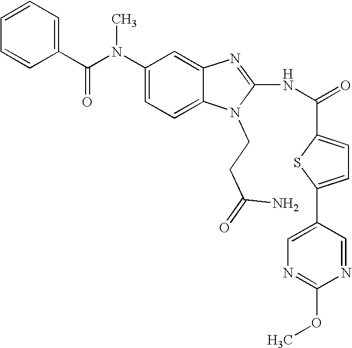 | 5-(2-Methoxy-pyrimidin-5-yl)-thiophene-2- carboxylic acid [5-(benzoyl-methyl-amino)-1- (2-carbamoyl-ethyl)-1H-benzoimidazol-2-yl]- amide |
| 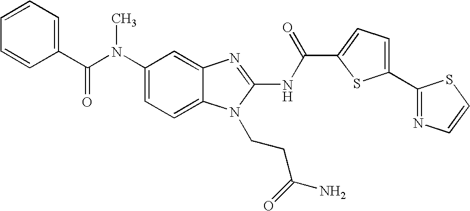 | 5-Thiazol-2-yl-thiophene-2-carboxylic acid [5- (benzoyl-methyl-amino)-1-(2-carbamoyl-ethyl)- 1H-benzoimidazol-2-yl]-amide |
| 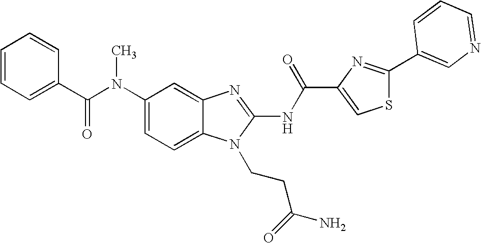 | 2-Pyridin-3-yl-thiazole-4-carboxylic [5- (benzoyl-methyl-amino)-1-(2-carbamoyl-ethyl)- 1H-benzoimidazol-2-yl]-amide |
| 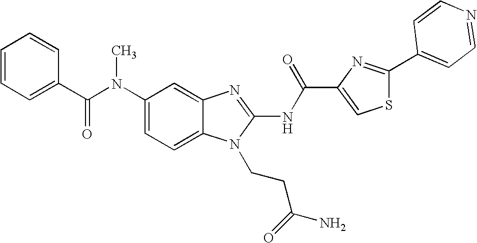 | 2-Pyridin-4-yl-thiazole-4-carboxylic acid [5- (benzoyl-methyl-amino)-1-(2-carbamoyl-ethyl)- 1H-benzoimidazol-2-yl]-amide |
| 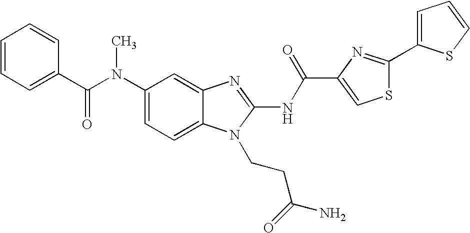 | 2-Thiophen-2-yl-thiazole-4-carboxylic acid [5- (benzoyl-methyl-amino)-1-(2-carbamoyl-ethyl)- 1H-benzoimidazol-2-yl]-amide |
| 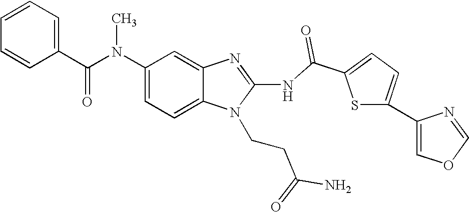 | 5-Oxazol-4-yl-thiophene-2-carboxylic acid [5- (benzoyl-methyl-amino)-1-(2-carbamoyl-ethyl)- 1H-benzoimidazol-2-yl]-amide |
| 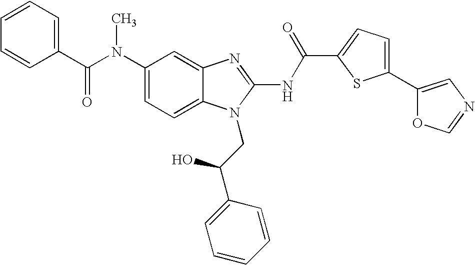 | 5-Oxazol-5-yl-thiophene-2-carboxylic acid [5- (benzoyl-methyl-amino)-1-(2R-hydroxyl-2- phenyl-ethyl)-1H-benzoimidazol-2-yl]-amide |
| 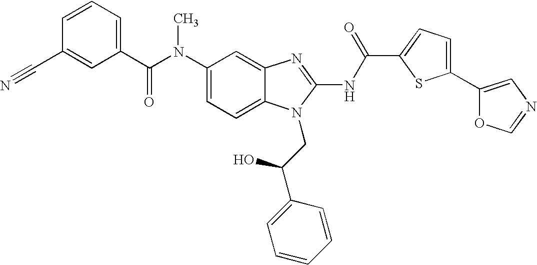 | 5-Oxazol-5-yl-thiophene-2-carboxylic acid [5- (3-cyanobenzoyl-methyl-amino)-1-(2R- hydroxyl-2-phenyl-ethyl)-1H- benzoimidazol-2-yl]-amide |
| 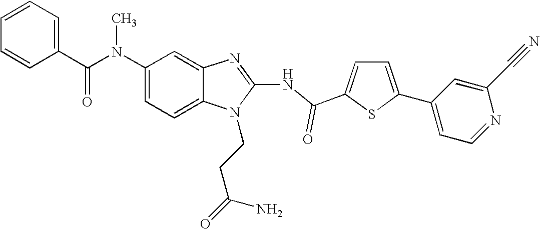 | 5-(2-Cyano-pyridin-4-yl)-thiophene-2- carboxylic acid [5-(benzoyl-methyl-amino)-1- (2-carbamoyl-ethyl)-1H-benzoimidazol- 2-yl]-amide |
| 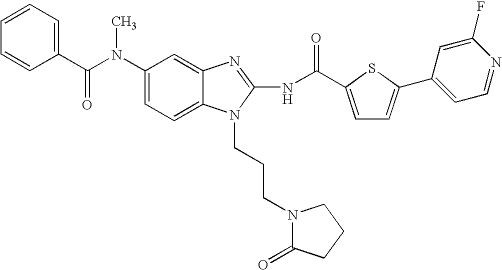 | 5-(2-Fluoro-pyridin-4-yl)-thiophene-2- carboxylic acid {5-(benzoyl-methyl-amino)-1- [3-(2-oxo-pyrrolidin-1-yI)-propyl]-1H- benzoimidazol-2-yl }-amide |
| 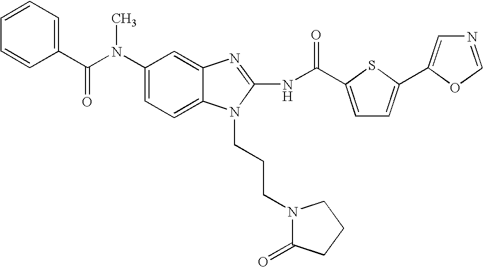 | 5-Oxazol-5-yl-thiophene-2-carboxylic acid {5- (benzoyl-methyl-amino)-1-[3-(2-oxo- pyrrolidin-1-yl)-propyl]-1H-benzoimidazol-2- yl}-amide |
| 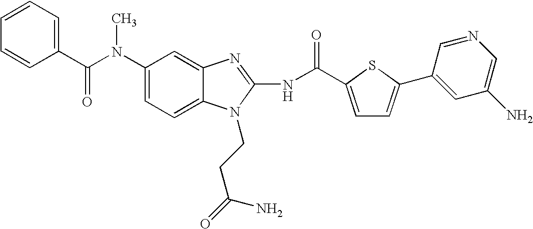 | 5-(5-Amino-pyridin-3-yl)-thiophene-2- carboxylic acid [5-(benzoyl-methyl-amino)-1- (2-carbamoyl-ethyl)-1H-benzoimidazol-2-yI]- amide |
| 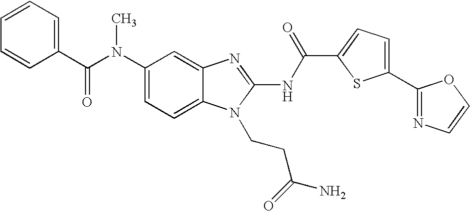 | 5-Oxazol-2-yl-thiophene-2-carboxylic acid [5- (benzoyl-methyl-amino)-1-(2-carbamoyl-ethyl)- 1H-benzoimidazol-2-yl]-amide |
| 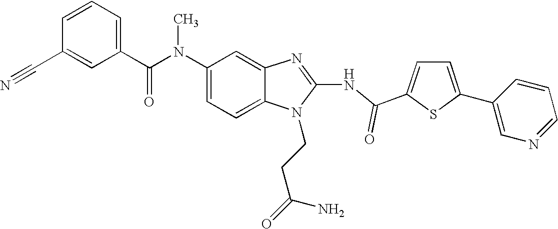 | 5-Pyridin-3-yl-thiophene-2-carboxylic acid {1- (2-carbamoyl-ethyl)-5-[(3-cyano-benzoyl)- methyl-amino]-1H-benzoimidazol-2-yl }-amide |
| 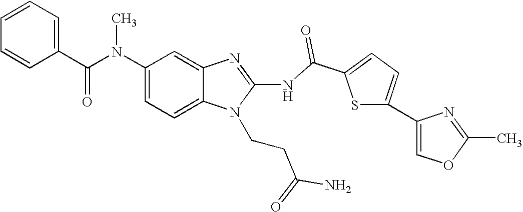 | 5-(2-Methyl-oxazol-4-yl)-thiophene-2- carboxylic acid [5-(benzoyl-methyl-amino)-1- (2-carbamoyl-ethyl)-1H-benzoimidazol-2-yl]- amide |
| 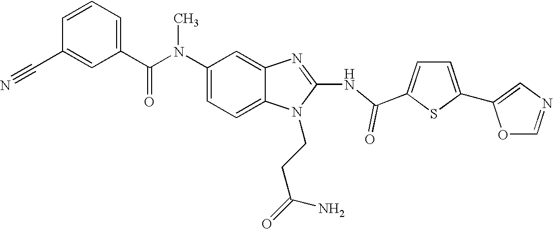 | 5-Oxazol-5-yl-thiophene-2-carboxylic acid {1- (2-carbamoyl-ethyl)-5- [(3-cyano-benzoyl)- methyl-amino]-1H-benzoimidazol-2-yl}-amide |
| 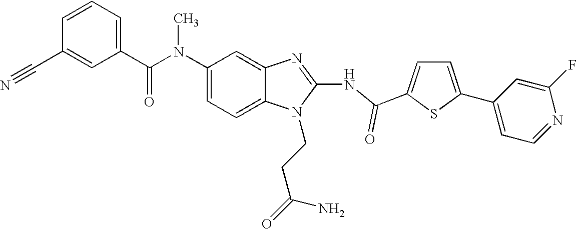 | 5-(2-Fluoro-pyridin-4-yl)-thiophene-2- carboxylic acid {1-(2-carbamoyl-ethyl)-5-[(3- cyano-benzoyl)-methyl-amino]-1H- benzoimidazol-2-yl}-amide |
| 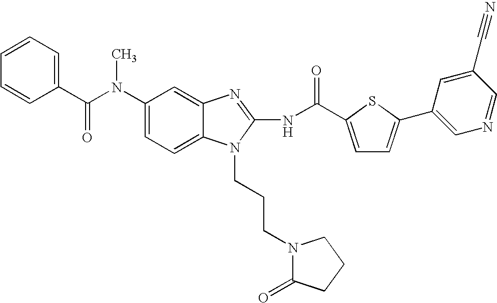 | 5-(5-Cyano-pyridin-3-yl)-thiophene-2- carboxylic acid {5-(benzoyl-methyl-amino)-1- [3-(2-oxo-pyrrolidin-1-yl)-propyl]-1H- benzoimidazol-2-yl}-amide |
| 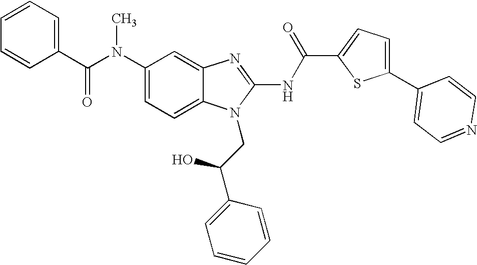 | 5-Pyridin-4-yl-thiophene-2-carboxylic acid [5- (benzoyl-methyl-amino)-1-(2R-hydroxyl- 2phenyl-ethyl)-1H-benzoimidazol-2-yl]-amide |
| 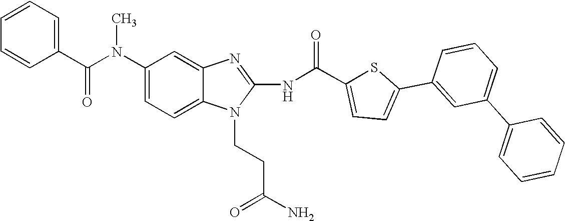 | 5-Biphenyl-3-yl-thiophene-2-carboxylic acid [5-(benzoyl-methyl-amino)-1-(2-carbamoyl- ethyl)-1H-benzoimidazol-2-yl]-amide |
| 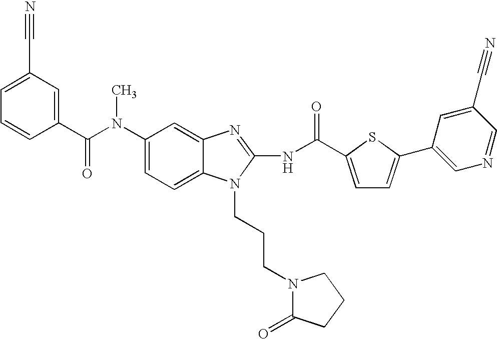 | 5-(5-Cyano-pyridin-3-yl)-thiophene-2- carboxylic acid {5-[(3-cyano-benzoyl)-methyl- amino]-1-[3-(2-oxo-pyrrolidin-1-yl)-propyl]- 1H-benzoimidazol-2-yl}-amide |
| 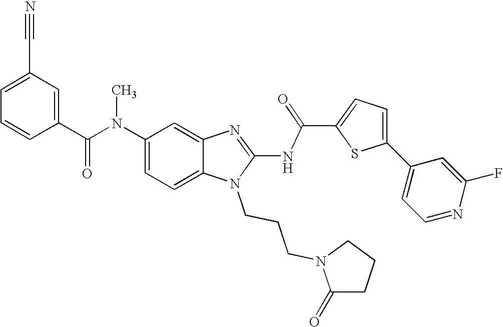 | 5-(2-Fluoro-pyridin-4-yl)-thiophene-2- carboxylic acid {5-[(3-cyano-benzoyl)-methyl- amino]-1-[3-(2-oxo-pyrrolidin-1-yl)-propyl]- 1H-benzoimidazol-2-yl}-amide |
| 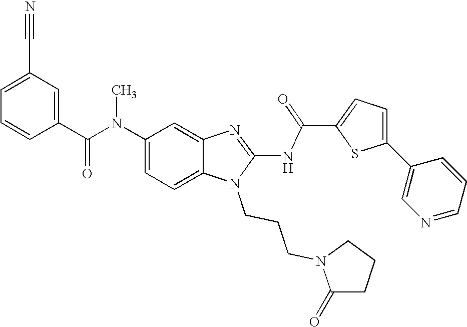 | 5-Pyridin-3-yl-thiophene-2-carboxylic acid {5- [(3-cyano-benzoyl)-methyl-amino]-1-[3-(2-oxo- pyrrolidin-1-yl)-propyl]-1H-benzoimidazol-2- yl}-amide |
| 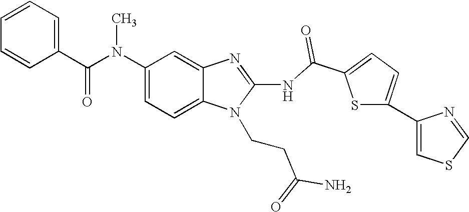 | 5-Thiazol-4-yl-thiophene-2-carboxylic acid [5- (benzoyl-methyl-amino)-1-(2-carbamoyl-ethyl)- 1H-benzoimidazol-2-yl]-amide |
| 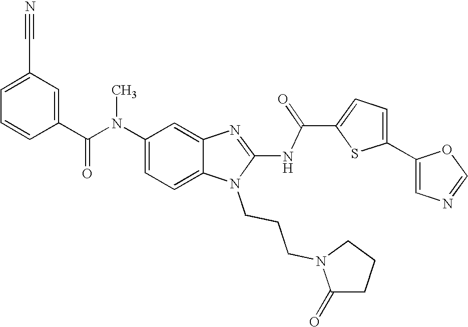 | 5-Oxazol-5-yl-thiophene-2-carboxylic acid {5- [(3-cyano-benzoyl)-methyl-aminoll-1-[3-(2-oxo- pyrrolidin-1-yl)-propyl]-1H-benzoimidazol-2- yl}-amide |
| 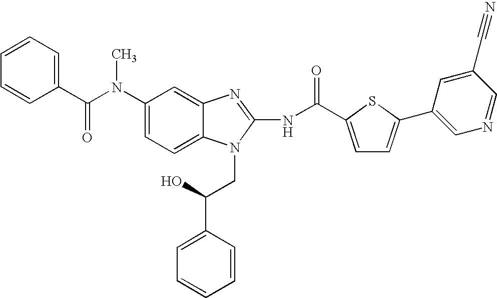 | 5-(5-Cyano-pyridin-3-yl)-thiophene-2- carboxylic acid [5-(benzoyl-methyl-amino)-1- ((R)-2-hydroxy-2-phenyl-ethyl)-1H- benzoimidazol-2-yl]-amide |
| 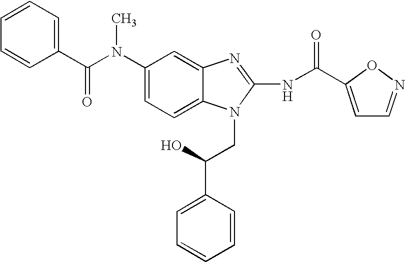 | Isoxazole-5-carboxylic acid [5-(benzoyl- methyl-amino)-1-((R)-2-hydroxy-2-phenyl- ethyl)-1H-benzoimidazol-2-yl]-amide |
| 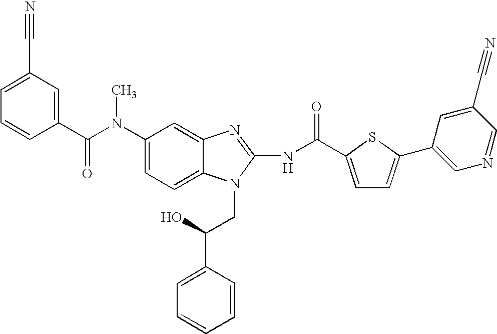 | 5-(5-Cyano-pyridin-3-yl)-thiophene-2- carboxylic acid [5-[(3-cyano-benzoyl)-methyl- amino]-1-((R)-2-hydroxy-2-phenyl-ethyl)-1H- benzoimidazol-2-yl]-amide |
| 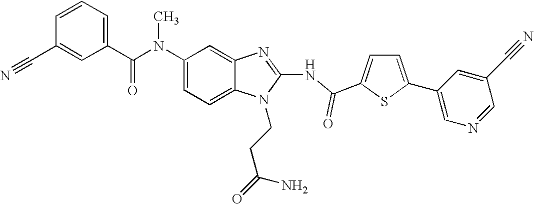 | 5-(5-Cyano-pyridin-3-yl)-thiophene-2- carboxylic acid {1-(2-carbamoyl-ethyl)-5-[(3- cyano-benzoyl)-methyl-amino]-1H- benzoimidazol-2-yl }-amide |
| 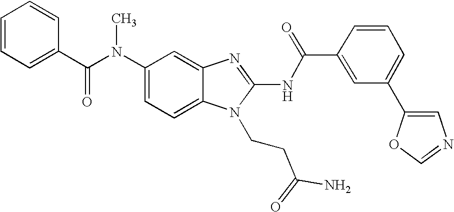 | N-[5-(Benzoyl-methyl-amino)-1-(2-carbamoyl- ethyl)-1H-benzoimidazol-2-yl]-3-oxazol-5- yl-benzamide |
| 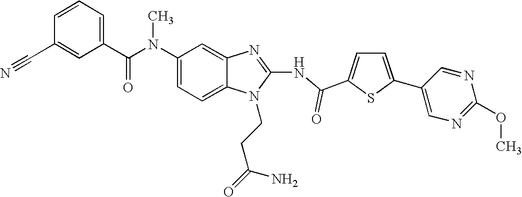 | 5-(2-Methoxy-pyrimidin-5-yl)-thiophene-2- carboxylic acid {1-(2-carbamoyl-ethyl)-5-[(3- cyano-benzoyl)-methyl-amino]-1H- benzoimidazol-2-yl}-amide |
| 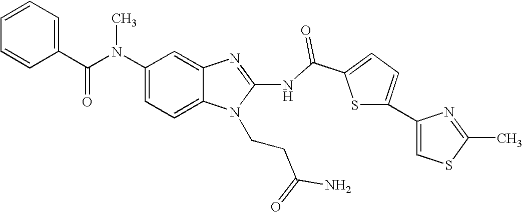 | 5-(2-Methyl-thiazol-4-yl)-thiophene-2- carboxylic acid [5-(benzoyl-methyl-amino)-1- (2-carbamoyl-ethyl)-1H-benzoimidazol-2-yl]- amide |
| 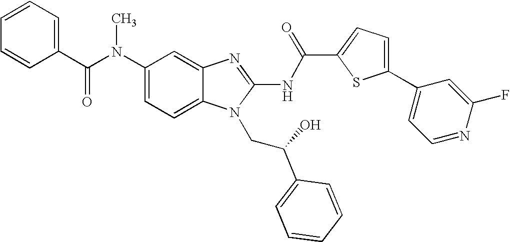 | 5-(2-Fluoro-pyridin-4-yl)-thiophene-2- carboxylic acid [5-(benzoyl-methyl-amrno)-1- ((R)-2-hydroxy-2-phenyl-ethyl)-1H- benzoimidazol-2-yl]-amide |
| 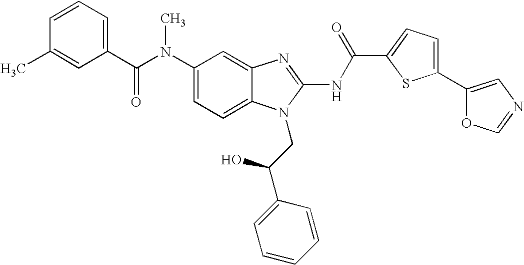 | 5-Oxazol-5-yl-thiophene-2-carboxylic acid {1- ((R)-2-hydroxy-2-phenyl-ethyl)-5- [methyl-(3- methyl-benzoyl)-amino]-1H-benzoimidazol-2- yl}-amide |
| 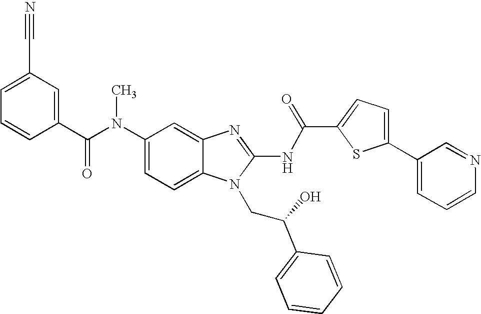 | 5-Pyridin-3-yl-thiophene-2-carboxylic acid [5- [(3-cyano-benzoyl)-methyl-amino]-1-((R)-2- hydroxy-2-phenyl-ethyl)-1H-benzoimidazol-2- yl]-amide |
| 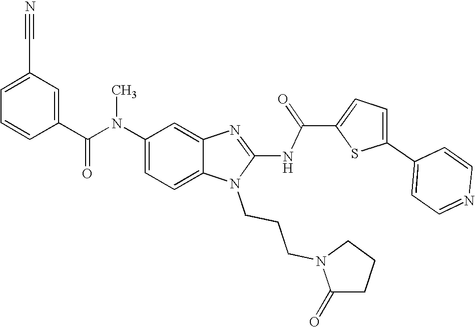 | 5-Pyridin-4-yl-thiophene-2-carboxylic acid {5- [(3-cyano-benzoyl)-methyl-amino]-1-[3-(2-oxo- pyrrolidin-1-yl)-propyl]-1H-benzoimidazol-2- yl}-amide |
| 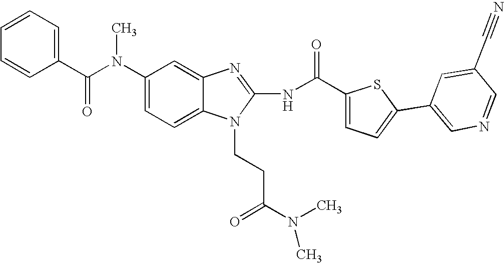 | 5-(5-Cyano-pyridin-3-yl)-thiophene-2- carboxylic acid [5-(benzoyl-methyl-amino)-1- (2-dimethylcarbamoyl-ethyl)-1H- benzoimidazol-2-yl]-amide |
| 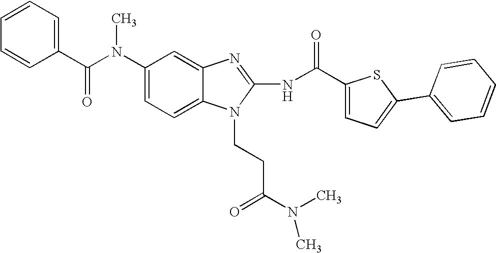 | 5-Phenyl-thiophene-2-carboxylic acid [5- (benzoyl-methyl-amino)-1-(2- dimethylcarbamoyl-ethyl)-1H-benzoimidazol- 2-yl]-amide |
| 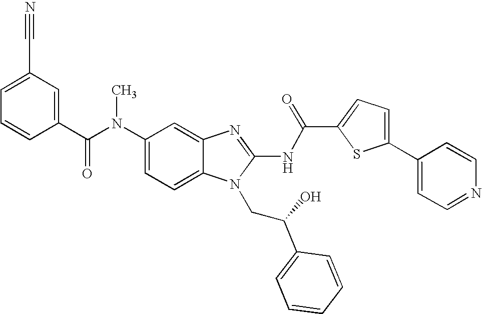 | 5-Pyridin-4-yl-thiophene-2-carboxylic acid [5- [(3-cyano-benzoyl)-methyl-amino]-1-((R)-2- hydroxy-2-phenyl-ethyl)-1H-benzoimidazol-2- yl]-amide |
| 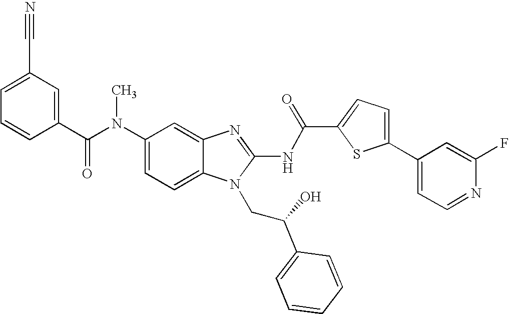 | 5-(2-Fluoro-pyridin-4-yl)-thiophene-2- carboxylic acid [5-[(3-cyano-benzoyl)-methyl- amino]-1-((R)-2-hydroxy-2-phenyl-ethyl)-1H- benzoimidazol-2-yl]-amide |
| 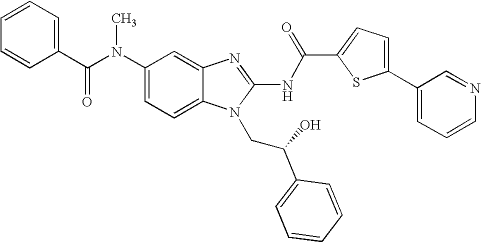 | 5-Pyridin-3-yl-thiophene-2-carboxylic acid [5- (benzoyl-methyl-amino)-1-((R)-2-hydroxy-2- phenyl-ethyl)-1H-benzoimidazol-2-yl]-amide |
| 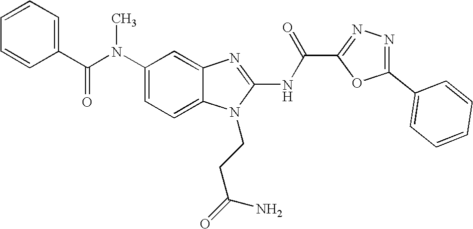 | 5-Phenyl-[1,3,4]oxadiazole-2-carboxylic acid [5-(benzoyl-methyl-amino)-1-(2-carbamoyl- ethyl)-1H-benzoimidazol-2-yl]-amide |
| 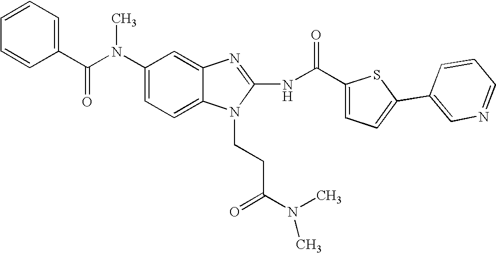 | 5-Pyridin-3-yl-thiophene-2-carboxylic acid [5- (benzoyl-methyl-amino)-1-(2- dimethylcarbamoyl-ethyl)-1H- benzoimidazol-2-yl]-amide |
| 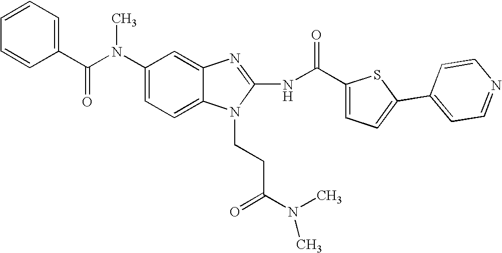 | 5-Pyridin-4-yl-thiophene-2-carboxylic acid [5- (benzoyl-methyl-amino)-1-(2- dimethylcarbamoyl-ethyl)-1H- benzoimidazol-2-yl]-amide |
| 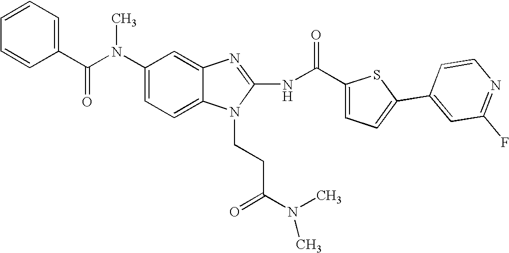 | 5-(2-Fluoro-pyridin-4-yl)-thiophene-2- carboxylic acid [5-(benzoyl-methyl-amino)-1- (2-dimethylcarbamoyl-ethyl)-1H- benzoimidazol-2-yl]-amide |
| 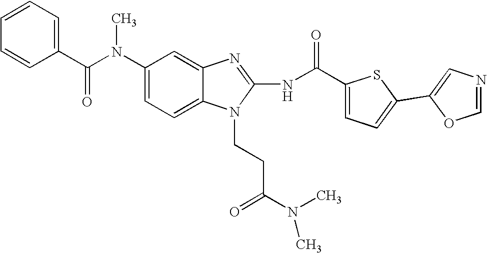 | 5-Oxazol-5-yl-thiophene-2-carboxylic acid [5- (benzoyl-methyl-amino)-1-(2- dimethylcarbamoyl-ethyl)-1H- benzoimidazol-2-yl]-amide |
| 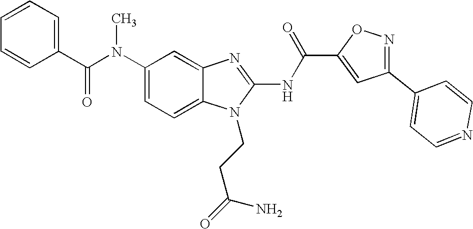 | 3-Phenyl-isoxazole-5-carboxylic acid [5- (benzoyl-methyl-amino)-1-(2-carbamoyl-ethyl)- 1H-benzoimidazol-2-yl]-amide |
| 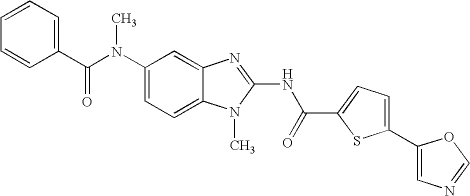 | 5-Oxazol-5-yl-thiophene-2-carboxylic [5- (benzoyl-methyl-amino)-1-methyl-1H- benzoimidazol-2-yl]-amide |
| 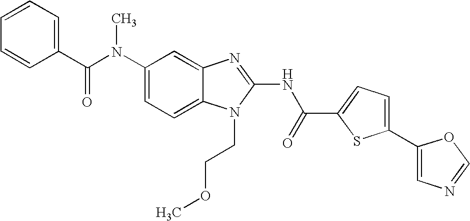 | 5-Oxazol-5-yl-thiophene-2-carboxylic acid [5- (benzoyl-methyl-amino)-1-(2-methoxy-ethyl)- 1H-benzoimidazol-2-yl]-amide |
| 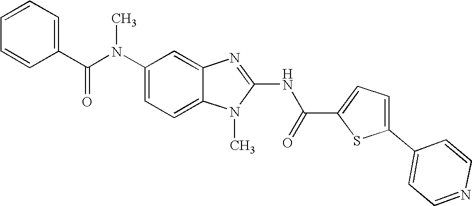 | 5-Pyridin-4-yl-thiophene-2-carboxylic acid [5- (benzoyl-methyl-amino)-1-methyl-1H- benzoimidazol-2-yl]-amide |
| 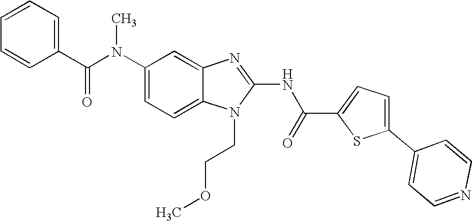 | 5-Pyridin-4-yl-thiophene-2-carboxylic acid [5- (benzoyl-methyl-amino)-1-(2-methoxy-ethyl)- 1H-benzoimidazol-2-yl]-amide |
| 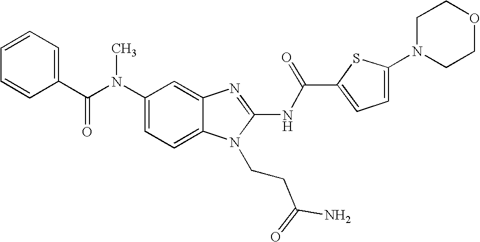 | N-{1-(2-Carbamoyl-ethyl)-2-[2-(5-morpholin- 4-yl-thiophen-2-yl)-2-oxo-ethyl]-1H- benzoimidazol-5-yl}-N-methyl-benzamide |
| 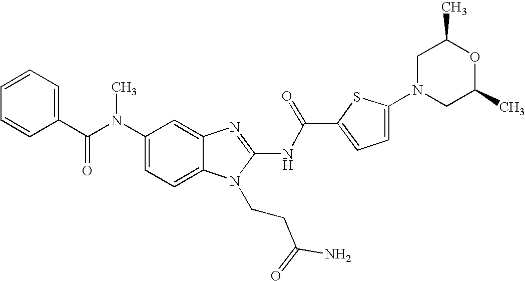 | N-(1-(2-Carbamoyl-ethyl)-2-{2-[5-((2R,6S)- 2,6-dimethyl-morpholin-4-yl)-thiophen-2-yl]-2- oxo-ethyl}-1 FI-benzoimidazol-5-yl)-N-methyl- benzamide |
| 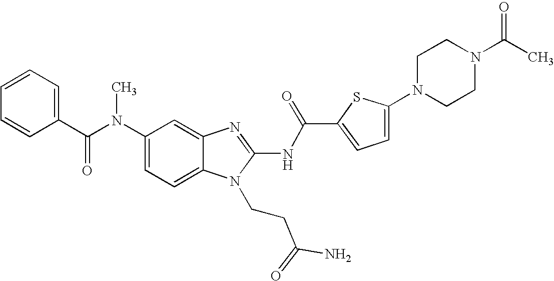 | N-[2-{2-[5-(4-Acetyl-piperazin-1-yl)-thiophen- 2-yl]-2-oxo-ethyl}-1-(2-carbamoyl-ethyl)-1H- benzoimidazol-5-yl]-N-methyl-benzamide |
| 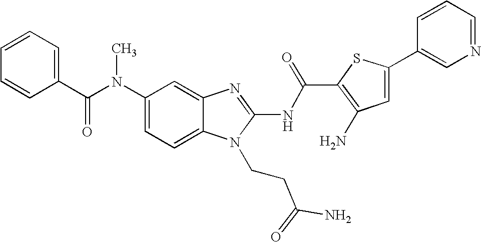 | N-[2-[2-(3-Amino-5-pyridin-3-yl-thiophen-2- yl)-2-oxo-ethyl]-1-(2 -carbamoyl-ethyl)-1H- benzoimidazol-5-yl]-N-methyl-benzamide |
| 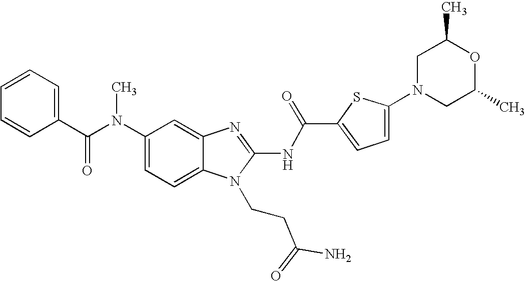 | N-(1-(2-Carbamoyl-ethyl)-2-{2-[5-((2R,6R)- 2,6-dimethyl-morpholin-4-yl)-thiophen-2-yl]-2- oxo-ethyl}-1H-benzoimidazol-5-yl)-N-methyl- benzamide |
| 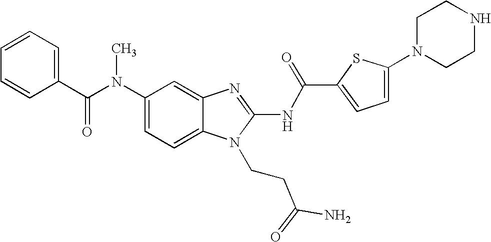 | N-{1-(2-Carbamoyl-ethyl)-2-[2-oxo-2-(5- piperazrn-1-yl-thiophen-2-yl)-ethyl]-1H- benzoimidazol-5-yl}-N-methyl-benzamide |
| 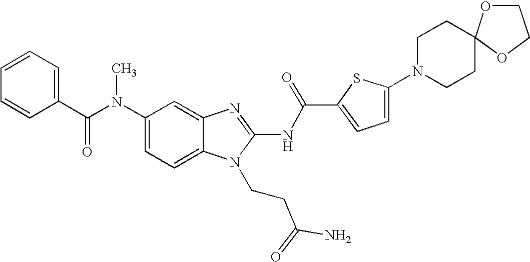 | N-(1-(2-Carbamoyl-ethyl)-2-{2-[5-(1,4-dioxa- 8-aza-spiro[4.5]dec-8-yl)-thiophen-2-yl]-2-oxo- ethyl}-1H-benzoimidazol-5-yl)-N-methyl- benzamide |
| 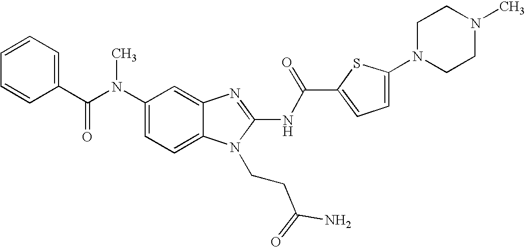 | N-(1-(2-Carbamoyl-ethyl)-2-{2-[5-(4-methyl- piperazin-1-yl)-thiophen-2-yl]-2-oxo-ethyl}- 1H-benzoimidazol-5-yl)-N-methyl-benzamide |
| 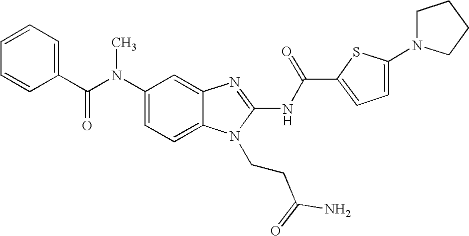 | N-{1-(2-Carbamoyl-ethyl)-2-[2-oxo-2-(5- pyrrolidin-1-yl-thiophen-2-yl)-ethyl]-1H- benzoimidazol-5-yl}-N-methyl-benzamide |
| 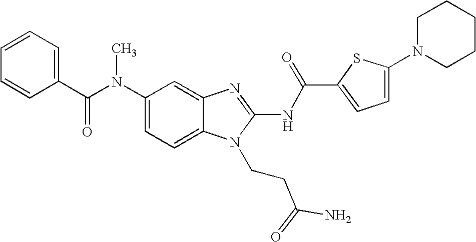 | N-{1-(2-Carbamoyl-ethyl)-2-[2-oxo-2-(5- piperidin-1-yl-thiophen-2-yl)-ethyl]-1H- benzoimidazol-5-yl }-N-methyl-benzamide |
| 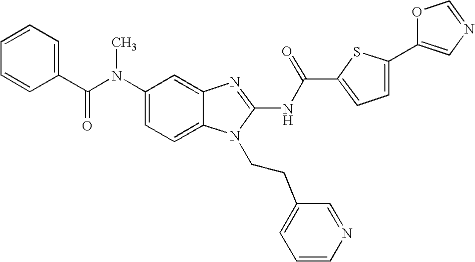 | N-Methyl-N-[2-[2-(5-oxazol-5-yl-thiophen-2- yl)-2-oxo-ethyl]-1-(2-pyridin-3-yl-ethyl)-1H- benzoimidazol-5-yl]-benzamide |
| 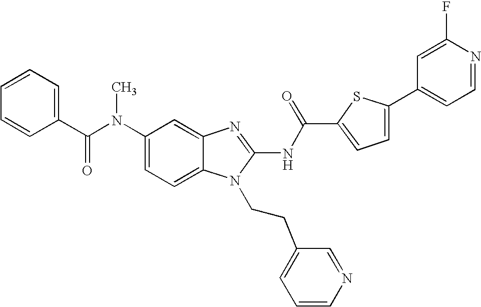 | N-[2-{2-[5-(2-Fluoro-pyridin-4-yl)-thiophen-2- yl]-2-oxo-ethyl}-1-(2-pyridin-3-yl-ethyl)-1H- benzoimidazol-5-yl]-N-methyl-benzamide |
| 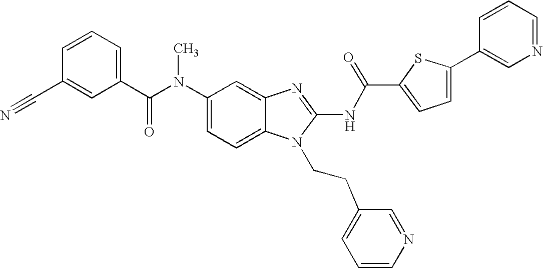 | 3-Cyano-N-methyl-N-[2-[2-oxo-2-(5-pyridin-3- yl-thiophen-2-yl)-ethyl]-1-(2-pyridin-3-yl- ethyl)-1H-benzoimidazol-5-yl]-benzamide |
| 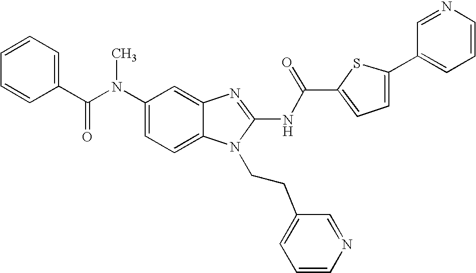 | N-Methyl-N-[2-[2-oxo-2-(5-pyridin-3-yl- thiophen-2-yl)-ethyl]-1-(2-pyridin-3-yl-ethyl)- 1H-benzoimidazol-5-yl]-benzamide |
| 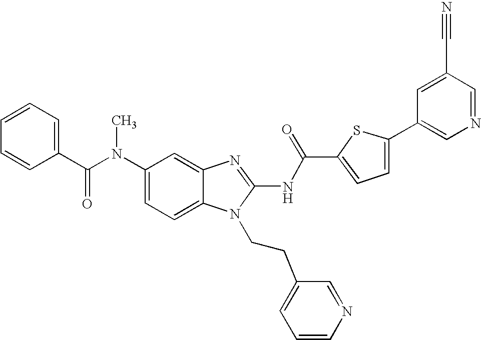 | N-[2-{2-[5-(5-Cyano-pyridin-3-yl)-thiophen-2- yl]-2-oxo-ethyl }-1-(2-pyridin-3-yl-ethyl)-1H- benzoimidazol-5-yl]-N-methyl-benzamide |
| 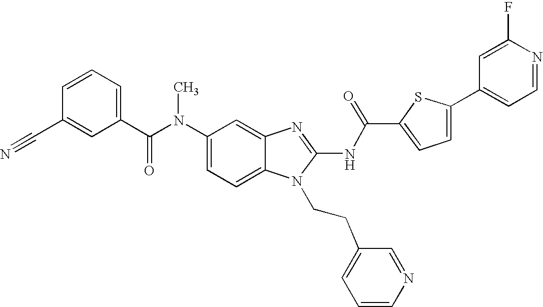 | 5-(2-Fluoro-pyridin-4-yl)-thiophene-2- carboxylic acid [5-[(3-cyano-benzoyl)-methyl- amino]-1-(2-pyridin-3-yl-ethyl)-1H- benzoimidazol-2-yl]-amide |
| 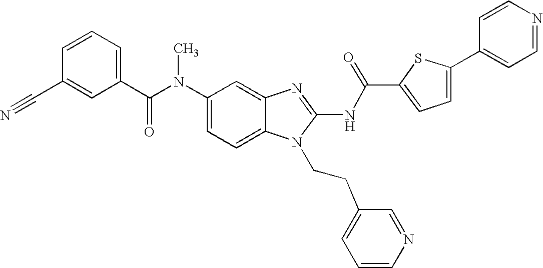 | 5-Pyridin-4-yl-thiophene-2-carboxylic acid [5- [(3-cyano-benzoyl)-methyl-amino]-1-(2- pyridin-3-yl-ethyl)-1H-benzoimidazol-2-yl]- amide |
| 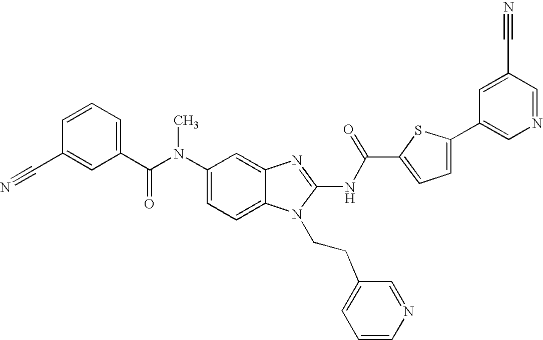 | 5-(5-Cyano-pyridin-3-yl)-thiophene-2- carboxylic acid [5-[(3-cyano-benzoyl)-methyl- amino]-1-(2-pyridin-3-yl-ethyl)-1H- benzoimidazol-2-yl]-amide |
| 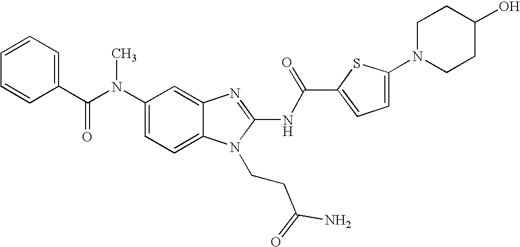 | 5-(4-Hydroxy-piperidin-1-yl)-thiophene-2- carboxylic acid [5-(benzoyl-methyl-amino)-1- (2-carbamoyl-ethyl)-1H-benzoimidazol-2-yl]- amide |
| 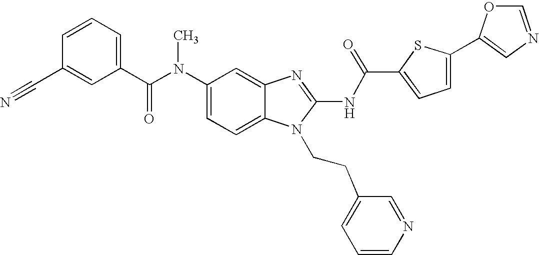 | 5-Oxazol-5-yl-thiophene-2-carboxylic acid [5- [(3-cyano-benzoyl)-methyl-amino]-1-(2- pyridin-3-yl-ethyl)-1H-benzoimidazol- 2-yl]-amide |
| 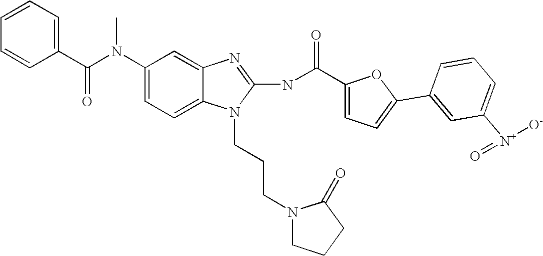 | 5-(3-Nitro-phenyl)-furan-2-carboxylic acid [5- (benzoyl-methyl-amino)-1-(2-carbamoyl-ethyl)- 1H-benzoimidazol-2-yl]-amide |
| 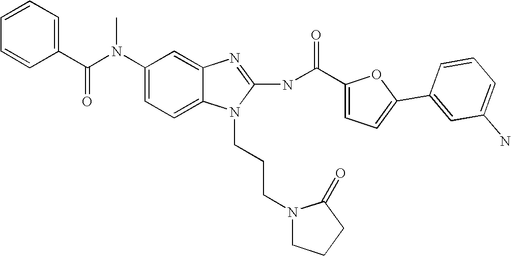 | 5-(3-Amino-phenyl)-furan-2-carboxylic acid [5- (benzoyl-methyl-amino)-1-(2-carbamoyl-ethyl)- 1H-benzoimidazol-2-yl]-amide |
| 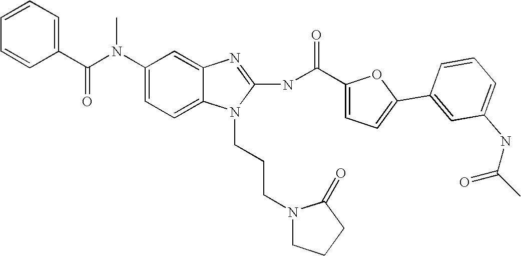 | 5-(3-Acetylamino-phenyl)-furan-2-carboxylic acid [5-(benzoyl-methyl-amino)-1-(2- carbamoyl-ethyl)-1H-benzoimidazol-2-yl]- carbamoyl-ethyl)-1H-benzoimidazol- 2-yl]-amide |

or the pharmaceutically acceptable salts, esters, acids, isomers or tautomers thereof.

In another embodiment there is provided representative compounds of the invention which can be made in accordance with the general schemes and working examples presented below:

TABLE I

| | |
|---|---|
| 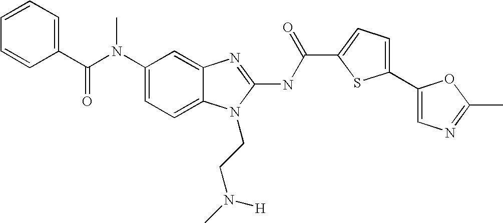 | 5-(2-Methyl-oxazol-5-yl)-thiophene-2- carboxylic acid [5-(benzoyl-methyl-amino)- 1-(2-methylamino-ethyl)-1H-benzoimidazol- 2-yl]-amide |
| 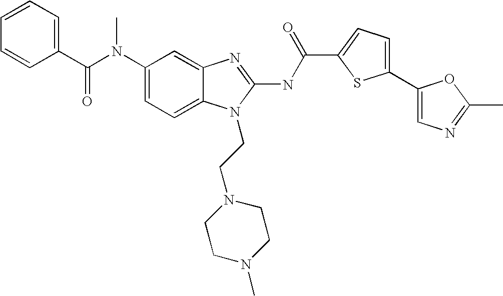 | 5-(2-Methyl-oxazol-5-yl)-thiophene-2- carboxylic acid {5-(benzoyl-methyl-amino)- 1-[2-(4-methyl-piperazin-1-yl)-ethyl]-1H- benzoimidazol-2-yl}-amide |
| 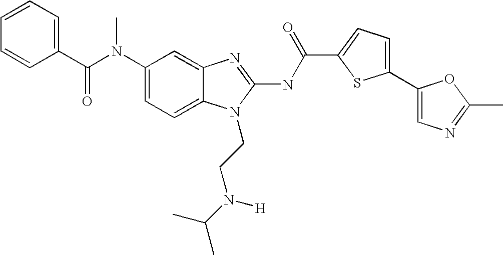 | 5-(2-Methyl-oxazol-5-yl)-thiophene-2- carboxylic acid [5-(benzoyl-methyl-amino)- benzoimidazol-2-yl]-amide |
| 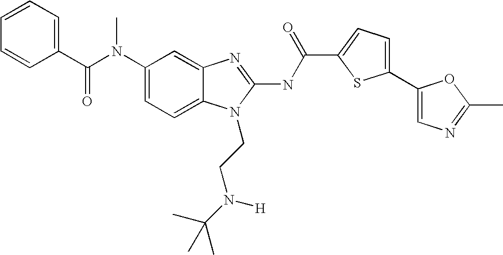 | 5-(2-Methyl-oxazol-5-yl)-thiophene-2- carboxylic acid [5-(benzoyl-methyl-amino)- 1-(2-tert-butylamino-ethyl)-1H- benzoimidazol-2-yl]-amide |
| 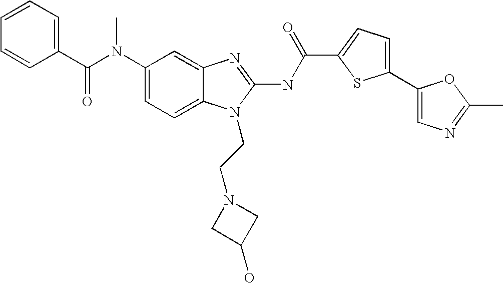 | 5-(2-Methyl-oxazol-5-yl)-thiophene-2- carboxylic acid {5-(benzoyl-methyl-amino)- 1-[2-(3-hydroxy-azetidin-1-yl)-ethyl]-1H- benzoimidazol-2-yl}-amide |
| 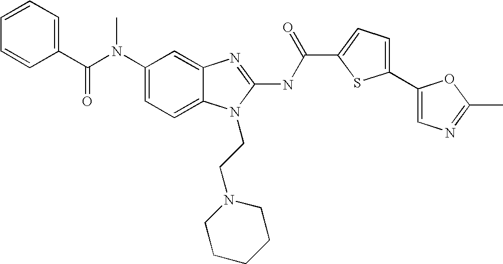 | 5-(2-Methyl-oxazol-5-yl)-thiophene-2- carboxylic acid [5-(benzoyl-methyl-amino)- 1-(2-piperidin-1-yl-ethyl)-1 H-benzoimidazol- 2-yl]-amide |
| 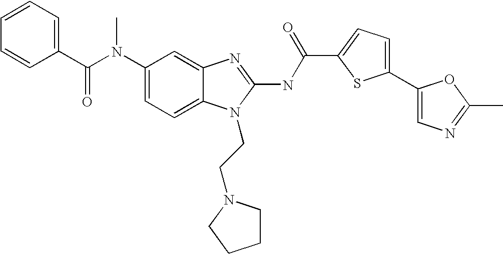 | 5-(2-Methyl-oxazol-5-yl)-thiophene-2- carboxylic acid [5-(benzoyl-methyl-amino)- 1-(2-pyrrolidin-1-yl-ethyl)-1H- benzoimidazol-2-yl]-amide |
| 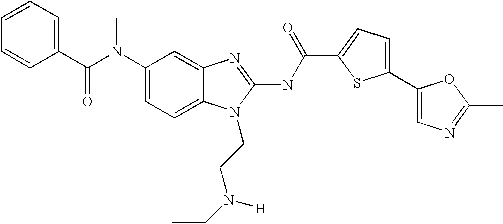 | 5-(2-Methyl-oxazol-5-yl)-thiophene-2- carboxylic acid [5-(benzoyL-methyl-amino)- 1-(2-ethylamino-ethyl)-1H-benzoimidazol- 2-yl]-amide |
| 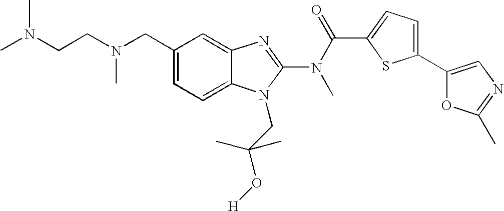 | 5-(2-Methyl-oxazol-5-yl)-thiophene-2- carboxylic acid [5-{[(2-dimethylamino- ethyl)-methyl-amino]-methyl}-1-(2-hydroxy- 2-methyl-propyl)-1H-benzoimidazol-2- yl]-amide |
| 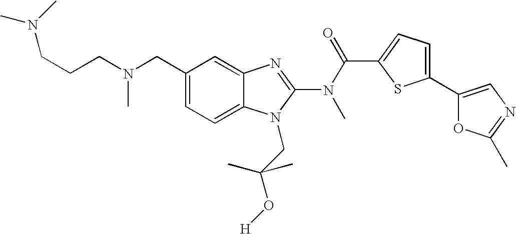 | 5-(2-Methyl-oxazol-5-yl)-thiophene-2- carboxylic acid [5-{[(3-dimethylamino- propyl)-methyl-amino]-methyl}-1-(2- hydroxy-2-methyl-propyl)-1H- benzoimidazol-2-yl]-amide |
| 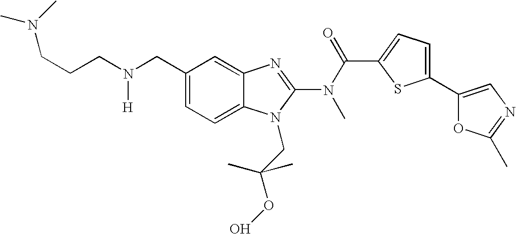 | 5-(2-Methyl-oxazol-5-yl)-thiophene-2- carboxylic acid [5-[(3-dimethylamino- propylamino)-methyl]-1-(2-hydroxy-2- methyl-propyl)-1H-benzoimidazol- 2-yl]-amide |
| 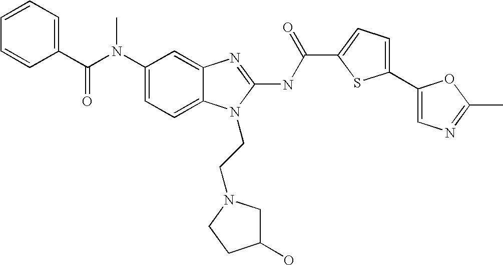 | 5-(2-Methyl-oxazol-5-yl)-thiophene-2- carboxylic acid {5-(benzoyl-methyl-amino)- 1-[2-(3-hydroxy-pyrrolidin-1-yl)-ethyl]-1H- benzoimidazol-2-yl }-amide |
| 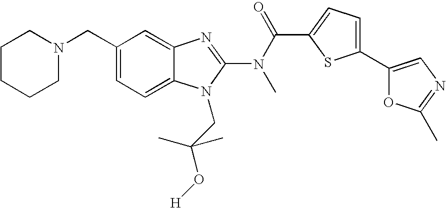 | 5-(2-Methyl-oxazol-5-yl)-thiophene-2- carboxylic acid [1-(2-hydroxy-2-methyl- propyl)-5-piperidin-1-ylmethyl-1H- benzoimidazol-2-yl]-amide |
| 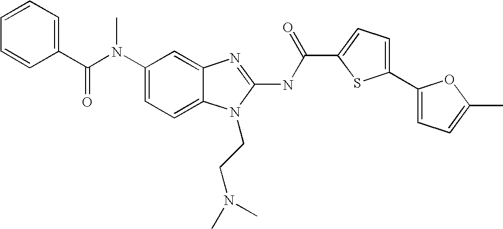 | 5-(2-Methyl-oxazol-5-yl)-thiophene-2- carboxylic acid [5-(benzoyl-methyl-amino)- 1-(2-dimethylamino-ethyl)-1H- benzoimidazol-2-yl]-amide |
| 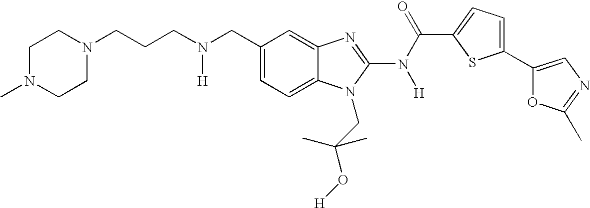 | 5-(2-Methyl-oxazol-5-yl)-thiophene-2- carboxylic acid (1-(2-hydroxy-2-methyl- propyl)-5-{[3-(4-methyl-piperazin-1-yl)- propylamino]-methyl}-1H-benzoimidazol-2- yl)-amide |
| 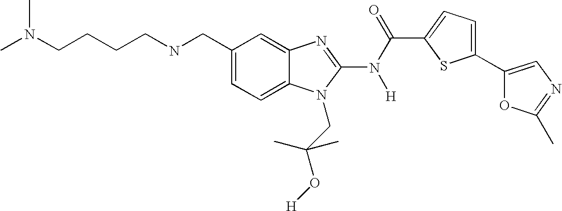 | 5-(2-Methyl-oxazol-5-yl)-thiophene-2- carboxylic acid [5-[(4-dimethylamino- butylamino)-methyl]-1-(2-hydroxy-2-methyl- propyl)-1H-benzoimidazol-2-yl]-amide |
| 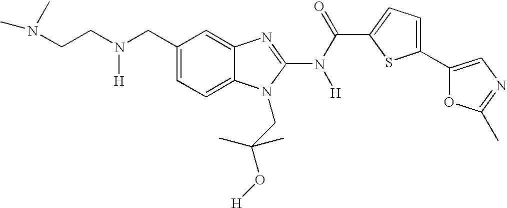 | 5-(2-Methyl-oxazol-5-yl)-thiophene-2- carboxylic acid [5-[(2-dimethylamino- ethylamino)-methyl]-1-(2-hydroxy-2-methyl- propyl)-1H-benzoimidazol-2-yl]-amide |
| 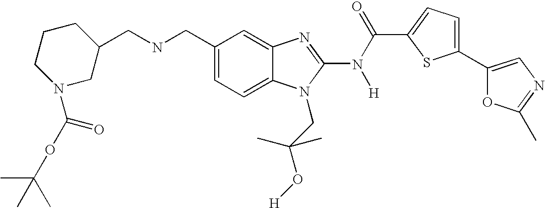 | 3-{[(1-(2-Hydroxy-2-methyl-propyl)-2-{[5- (2-methyl-oxazol-5-yl)-thiophene-2- carbonyl]-amino}-1H-benzoimidazol-5- ylmethyl)-amino]-methyl}-piperidine-1- carboxylic acid tert-butyl ester |
| 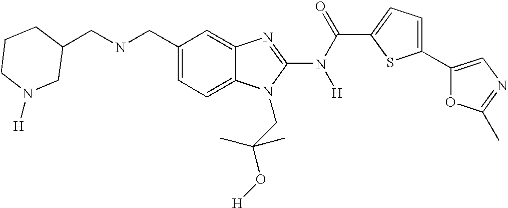 | 5-(2-Methyl-oxazol-5-yl)-thiophene-2- carboxylic acid (1-(2-hydroxy-2-methyl- propyl)-5-{[(piperidin-3-ylmethyl)-amino]- methyl}-1H-benzoimidazol-2-yl)-amide |
| 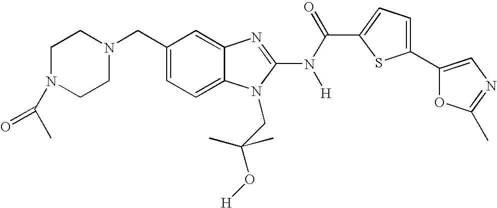 | 5-(2-Methyl-oxazol-5-yl)-thiophene-2- carboxylic acid [5-(4-acetyl-piperazin-1- ylmethyl)-1-(2-hydroxy-2-methyl-propyl)- 1H-benzoimidazol-2-yl]-amide |
| 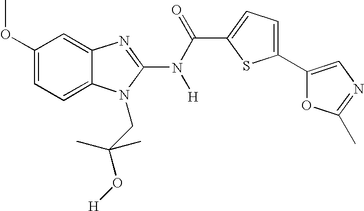 | 5-(2-Methyl-oxazol-5-yl)-thiophene-2- carboxylic acid [1-(2-hydroxy-2-methyl- propyl)-5-methoxy-1H-benzoimidazol-2-yl]- amide |
| 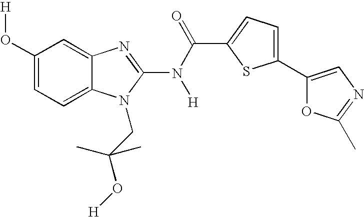 | 5-(2-Methyl-oxazol-5-yl)-thiophene-2- carboxylic acid [5-hydroxy-1-(2-hydroxy-2- methyl-propyl)-1H-benzoimidazol-2-yl]- amide |
| 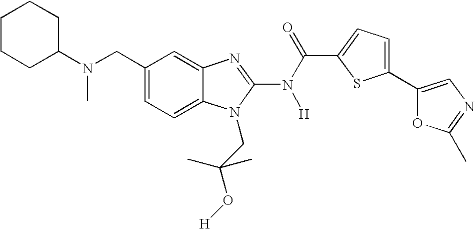 | 5-(2-Methyl-oxazol-5-yl)-thiophene-2- carboxylic acid [5-[(cyclohexyl-methyl- amino)-methyl]-1-(2-hydroxy-2-methyl- propyl)-1H-benzoimidazol-2-yl]-amide |
| 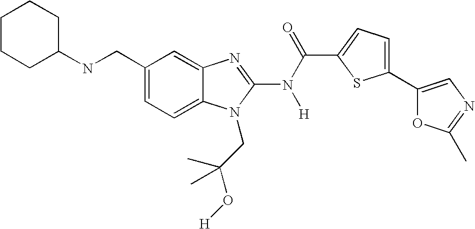 | 5-(2-Methyl-oxazol-5-yl)-thiophene-2- carboxylic acid [5-cyclohexylaminomethyl- 1-(2-hydroxy-2-methyl-propyl)-1H- benzoimidazol-2-yl]-amide |
| 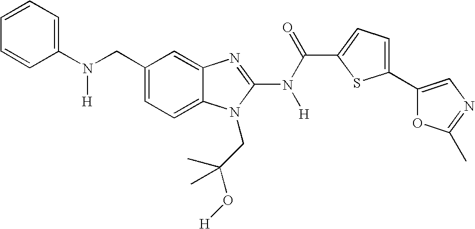 | 5-(2-Methyl-oxazol-5-yl)-thiophene-2- carboxylic acid [1-(2-hydroxy-2-methyl- propyl)-5-phenylaminomethyl-1H- benzoimidazol-2-yl]-amide |
| 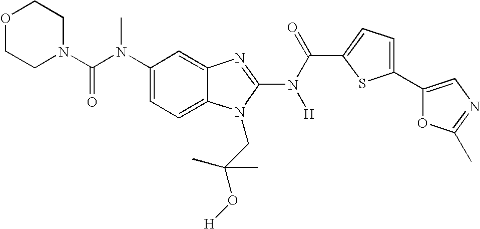 | Morpholine-4-carboxylic acid (1-(2-hydroxy- 2-methyl-propyl)-2-{[5-(2-methyl-oxazol-5- yl)-thiophene-2-carbonyl]-amino}-1H- benzoimidazol-5-yl)-methyl-amide |
| 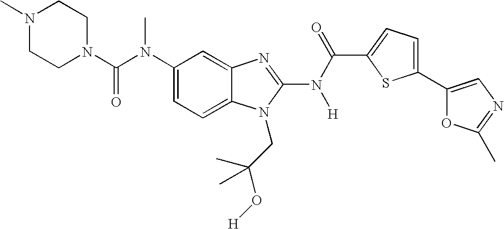 | 4-Methyl-piperazine-1-carboxylic acid (1-(2- hydroxy-2-methyl-propyl)-2-{[5-(2-methyl- oxazol-5-yl)-thiophene-2-carbonyl]-amino}- 1H-benzoimidazol-5-yl)-methyl-amide |
| 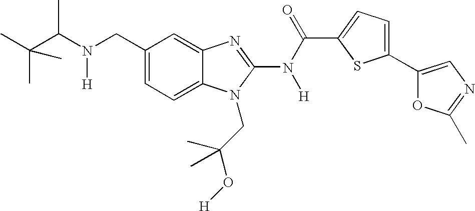 | 5-(2-Methyl-oxazol-5-yl)-thiophene-2- carboxylic acid {1-(2-hydroxy-2-methyl- propyl)-5-[(1,2,2-trimethyl-propylamino)- methyl]-1H-benzoimidazol-2-yl}-amide |
| 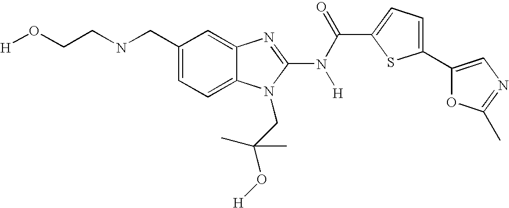 | 5-(2-Methyl-oxazol-5-yl)-thiophene-2- carboxylic acid {5-[(2-hydroxy-ethylamino)- methyl]-1-(2-hydroxy-2-methyl-propyl)-1H- benzoimidazol-2-yl]-amide |
| 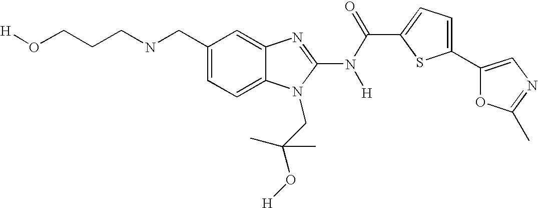 | 5-(2-Methyl-oxazol-5-yl)-thiophene-2- carboxylic acid {1-(2-hydroxy-2-methyl- propyl)-5-[(3-hydroxy-propylamino)- methyl]-1H-benzoimidazol-2-yl}-amide |
| 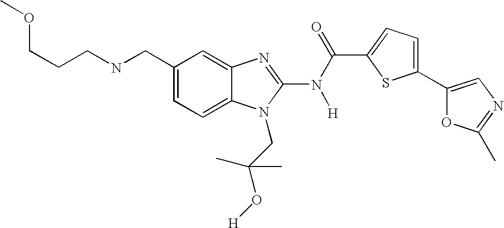 | 5-(2-Methyl-oxazol-5-yl)-thiophene-2- carboxylic acid {1-(2-hydroxy-2-methyl- propyl)-5-[(3-methoxy-propylamino)- methyl]-1H-benzoimidazol-2-yl}-amide |
| 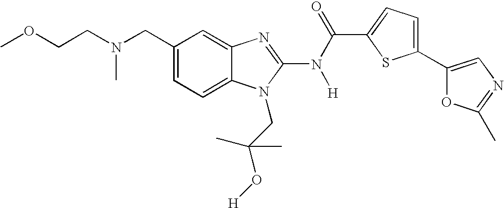 | 5-(2-Methyl-oxazol-5-yl)-thiophene-2- carboxylic acid (1-(2-hydroxy-2-methyl- propyl)-5-{[(2-methoxy-ethyl)-methyl- amino]-methyl}-1H-benzoimidazol-2-yl)- amide |
| 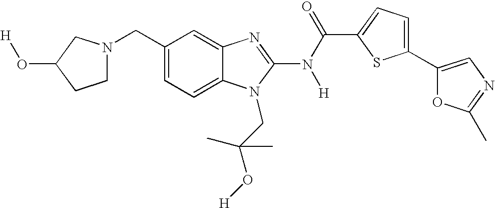 | 5-(2-Methyl-oxazol-5-yl)-thiophene-2- carboxylic acid [1-(2-hydroxy-2-methyl- propyl)-5-(3-hydroxy-pyrrolidin-1- ylmethyl)-1H-benzoimidazol-2-yl]-amide |
| 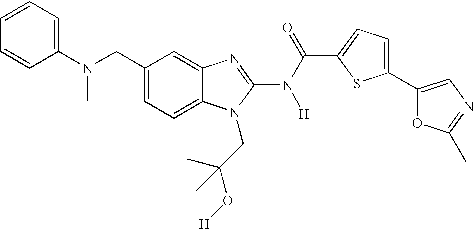 | 5-(2-Methyl-oxazol-5-yl)-thiophene-2- carboxylic acid {1-(2-hydroxy-2-methyl- propyl)-5-[(methyl-phenyl-amino)-methyl]- 1H-benzoimidazol-2-yl}-amide |
| 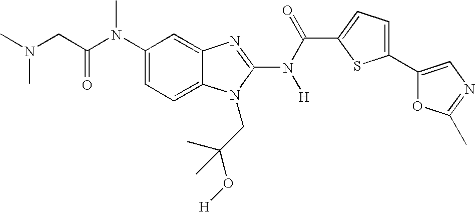 | 5-(2-Methyl-oxazol-5-yl)-thiophene-2- carboxylic acid [5-[(2-dimethylamino- acetyl)-methyl-amino]-1-(2-hydroxy-2- methyl-propyl)-1H-benzoimidazol-2-yl]- amide |
| 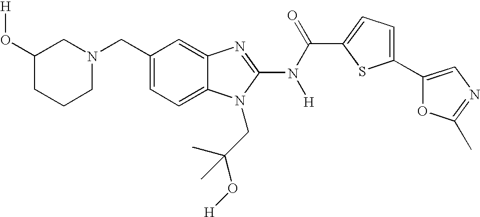 | 5-(2-Methyl-oxazol-5-yl)-thiophene-2- carboxylic acid [1-(2-hydroxy-2-methyl- propyl)-5-(3-hydroxy-piperidin-1- ylmethyl)-1H-benzoimidazol-2-yl]-amide |
| 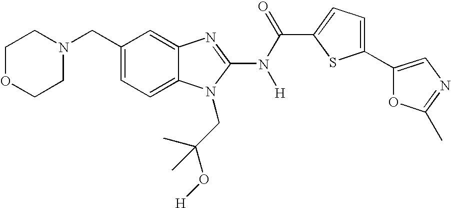 | 5-(2-Methyl-oxazol-5-yl)-thiophene-2- carboxylic acid [1-(2-hydroxy-2-methyl- propyl)-5-morpholin-4-ylmethyl-1H- benzoimidazol-2-yl]-amide |
| 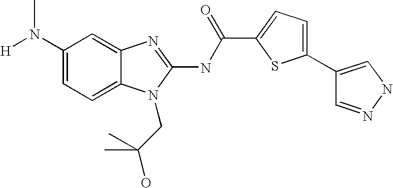 | 5-(1H-Pyrazol-4-yl)-thiophene-2-carboxylic acid [1-(2-hydroxy-2-methyl-propyl)-5- methylamino-1H-benzoimidazol-2-yl]- amide |
| 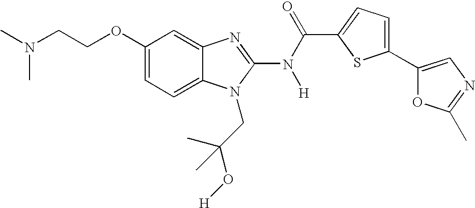 | 5-(2-Methyl-oxazol-5-yl)-thiophene-2- carboxylic acid [5-(2-dimethylamino- ethoxy)-1-(2-hydroxy-2-methyl-propyl)-1H- benzoimidazol-2-yl]-amide |
| 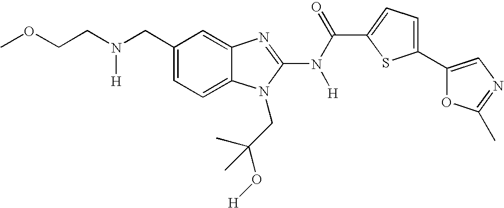 | 5-(2-Methyl-oxazol-5-yl)-thiophene-2- carboxylic acid {1-(2-hydroxy-2-methyl- propyl)-5-[(2-methoxy-ethylamino)-methyl]- 1H-benzoimidazol-2-yl}-amide |
| 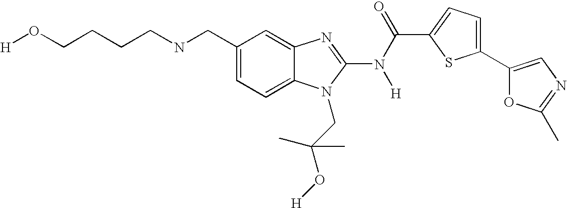 | 5-(2-Methyl-oxazol-5-yl)-thiophene-2- carboxylic acid [5-[(4-hydroxy-butylamino)- methyl]-1-(2-hydroxy-2-methyl-propyl)-1H- benzoimidazol-2-yl]-amide |
| 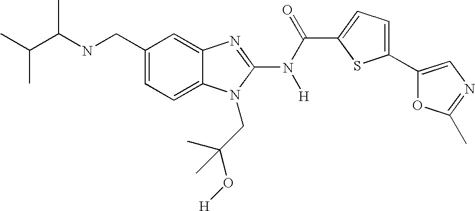 | 5-(2-Methyl-oxazol-5-yl)-thiophene-2- carboxylic acid [5-[(1,2-dimethyl- propylamino)-methyl]-1-(2-hydroxy-2- methyl-propyl)-1H-benzoimidazol-2- yl]-amide |
| 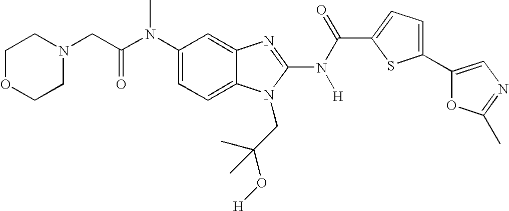 | 5-(2-Methyl-oxazol-5-yl)-thiophene-2- carboxylic acid {1-(2-hydroxy-2-methyl- propyl)-5-[methyl-(2-morpholin-4-yl- acetyl)-amino]-1H-benzoimidazol-2- yl}-amide |
| 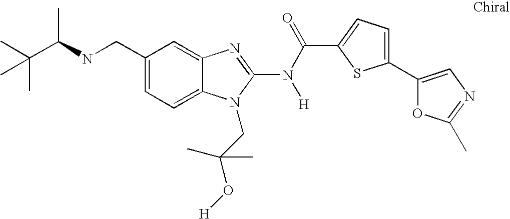 | 5-(2-Methyl-oxazol-5-yl)-thiophene-2- carboxylic acid {1-(2-hydroxy-2-methyl- propyl)-5-[((R)-1,2,2-trimethyl- propylamino)-methyl]-1H-benzoimidazol- 2-yl}-amide |
| 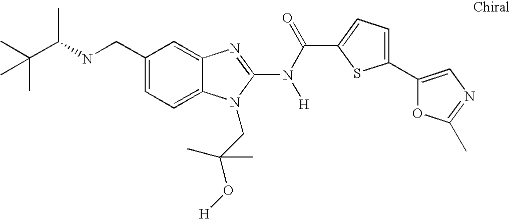 | 5-(2-Methyl-oxazol-5-yl)-thiophene-2- carboxylic acid {1-(2-hydroxy-2-methyl- propyl)-5-[((S)-1,2,2-trimethyl- propylamino)-methyl]-1H-benzoimidazol- 2-yl}-amide |
| 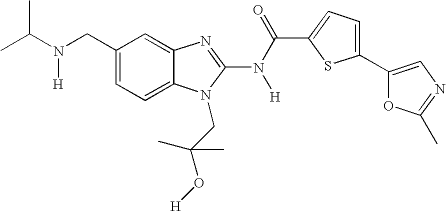 | 5-(2-Methyl-oxazol-5-yl)-thiophene-2- carboxylic acid [1-(2-hydroxy-2-methyl- propyl)-5-(isopropylamino-methyl)-1H- benzoimidazol-2-yl]-amide |
| 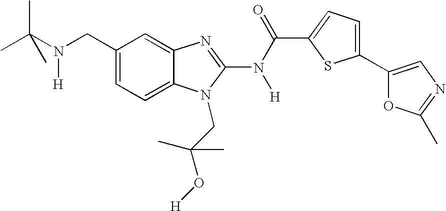 | 5-(2-Methyl-oxazol-5-yl)-thiophene-2- carboxylic acid [5-(tert-butylamino-methyl)- 1-(2-hydroxy-2-methyl-propyl)-1H- benzoimidazol-2-yl]-amide |
| 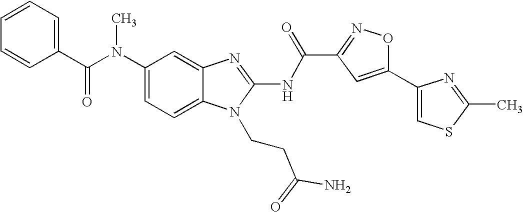 | 5-(2-Methyl-thiazol-4-yl)-isoxazole-3- carboxylic acid [5-(benzoyl-methyl-amino)- 1-(2-carbamoyl-ethyl)-1H-benzoimidazol-2- yl]-amide |
| 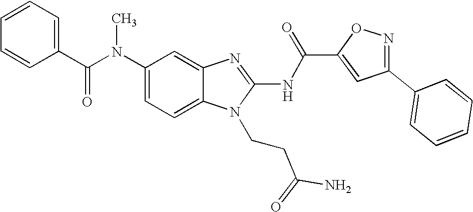 | 3-Phenyl-isoxazole-5-carboxylic acid [5- (benzoyl-methyl-amino)-1-(2-carbamoyl- ethyl)-1H-benzoimidazol-2-yl]-amide |
| 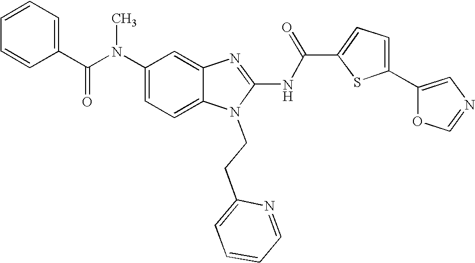 | 5-Oxazol-5-yl-thiophene-2-carboxylic acid [5-(benzoyl-methyl-amino)-1-(2-pyridin-2- yl-ethyl)-1H-benzoimidazol-2-yl]-amide |
| 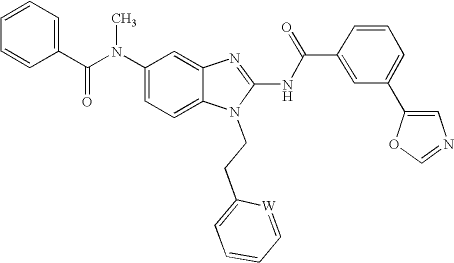 | N-[5-(Benzoyl-methyl-amino)-1-(2-pyridin- 2-yl-ethyl)-1H-benzoimidazol-2-yl]-3- oxazol-5-yl-benzamide |
| 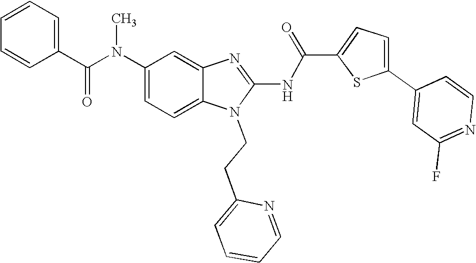 | 5-(2-Fluoro-pyridin-4-yl)-thiophene-2- carboxylic acid [5-(benzoyl-methyl-amino)- 1-(2-pyridin-2-yl-ethyl)-1#H!- benzoimidazol-2-yl]-amide |
| 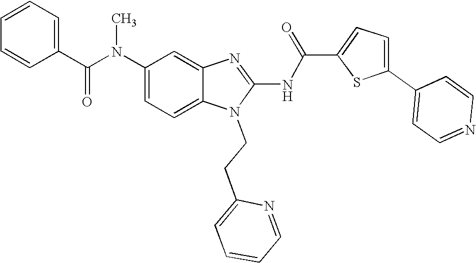 | 5-Pyridin-4-yl-thiophene-2-carboxylic acid [5-(benzoyl-methyl-amino)-1-(2-pyridin-2- yl-ethyl)-1H-benzoimidazol-2-yl]-amide |
| 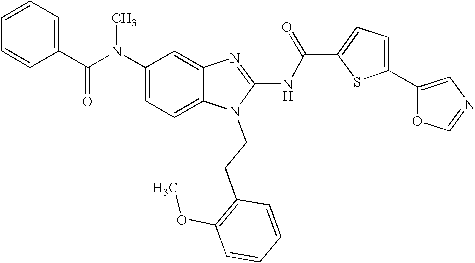 | 5-Oxazol-5-yl-thiophene-2-carboxylic acid {5-(benzoyl-methyl-amino)-1-[2-(2- methoxy-phenyl)-ethyl]-1H-benzoimidazol- 2-yl}-amide |
| 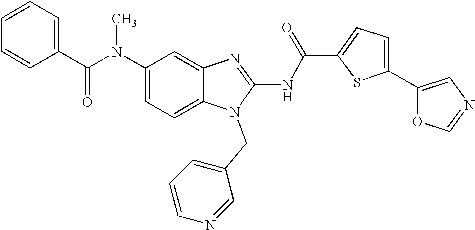 | 5-Oxazol-5-yl-thiophene-2-carboxylic acid [5-(benzoyl-methyl-amino)-1-pyridin-3- ylmethyl-1H-benzoimidazol-2-yl]-amide |
| 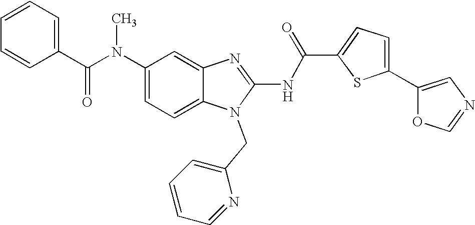 | 5-Oxazol-5-yl-thiophene-2-carboxylic acid [5-(benzoyl-methyl-amino)-1-pyridin-2- ylmethyl-1H-benzoimidazol-2-yl]-amide |
| 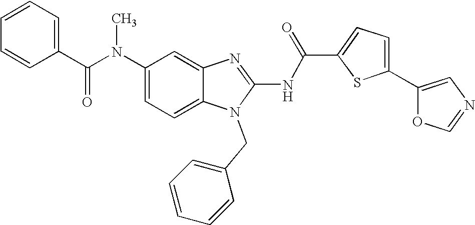 | 5-Oxazol-5-yl-thiophene-2-carboxylic acid [5-(benzoyl-methyl-amino)-1-benzyl-1H- benzoimidazol-2-yl]-amide |
| 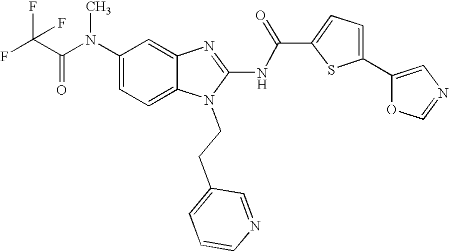 | 5-Oxazol-5-yl-thiophene-2-carboxylic acid [5-[methyl-(2,2,2-trifluoro-acetyl)-amino]- 1-(2-pyridin-3-yl-ethyl)-1H-benzoimidazol- 2-yl]-amide |
| 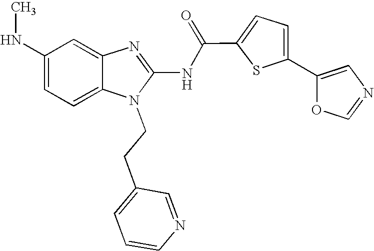 | 5-Oxazol-5-yl-thiophene-2-carboxylic acid [5-methylamino-1-(2-pyridin-3-yl-ethyl)- 1H-benzoimidazol-2-yl]-amide |
| 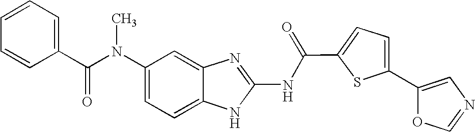 | 5-Oxazol-5-yl-thiophene-2-Carboxylic acid [5-(benzoyl-methyl-amino)-1H- benzoimidazol-2-yl]-amide |
| 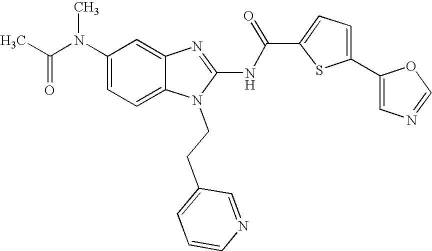 | 5-Oxazol-5-yl-thiophene-2-Carboxylic acid [5-(acetyl-methyl-aminO)-1-(2-pyridin-3-yl- ethyl)-1H-benzoimidazol-2-yl]-amide |
| 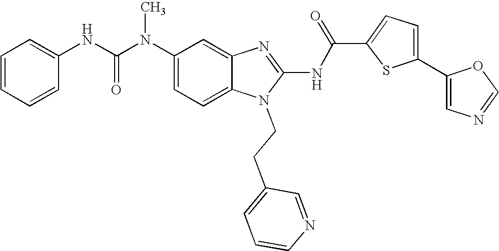 | 5-Oxazol-5-yl-thiophene-2-carboxylic acid [5-(1-methyl-3-phenyl-ureido)-1-(2-pyridin- 3-yl-ethyl)-1H-benzoimidazol-2-yl]-amide |
| 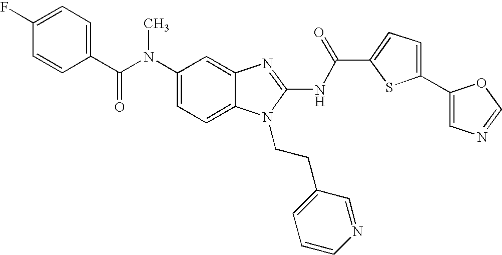 | 5-Oxazol-5-yl-thiophene-2-carboxylic acid [5-[(4-fluoro-benzoyl)-methyl-amino]-1-(2- pyridin-3-yl-ethyl)-1H-benzoimidazol-2- yl]-amide |
| 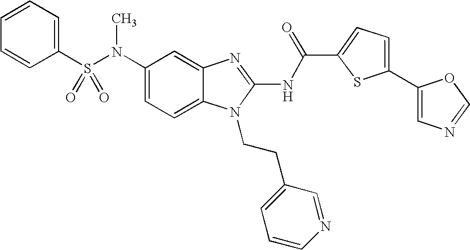 | 5-Oxazol-5-yl-thiophene-2-carboxylic acid [5-(benzenesulfonyl-methyl-amino)-1-(2- pyridin-3-yl-ethyl)-1H-benzoimidazol-2- yl]-amide |
| 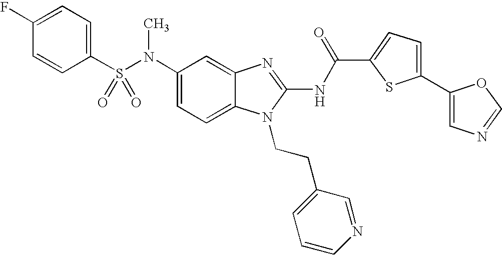 | 5-Oxazol-5-yl-thiophene-2-Carboxylic acid [5-[(4-fluoro-benzenesulfonyl)-methyl- amino]-1-(2-pyridin-3-yl-ethyl)-1H- benzoimidazol-2-yl]-amide |
| 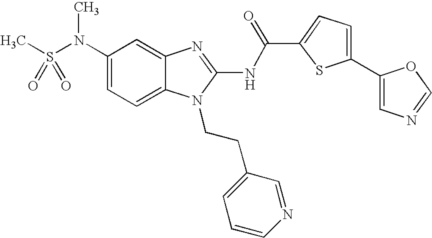 | 5-Oxazol-5-yl-thiophene-2-Carboxylic acid [5-(methanesulfonyl-methyl-amino)-1-(2- pyridin-3-yl-ethyl)-1H-benzoimidazol-2- yl]-amide |
| 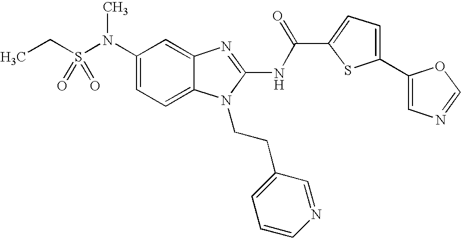 | 5-Oxazol-5-yl-thiophene-2-carboxylic acid [5-(methyl-propionyl-amino)-1-(2-pyridin- 3-yl-ethyl)-1H-benzoimidazol-2-yl]-amide |
| 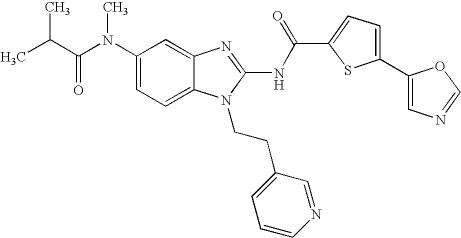 | 5-Oxazol-5-yl-thiophene-2-Carboxylic acid [5-(isobutyryl-methyl-amino)-1-(2-pyridin- 3-yl-ethyl)-1H-benzoimidazol-2-yl]-amide |
| 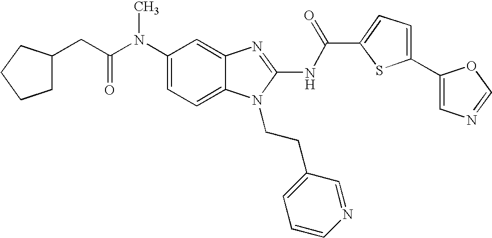 | 5-Oxazol-5-yl-thiophene-2-carboxylic acid [5-[(2-cyclopentyl-acetyl)-methyl-amino]-1- (2-pyridin-3-yl-ethyl)-1H-benzoimidazol-2- yl]-amide |
| 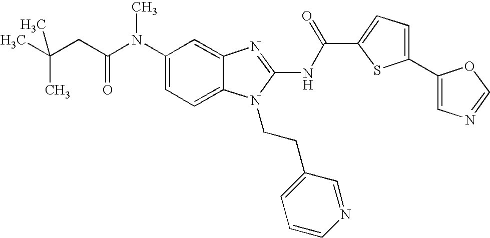 | 5-Oxazol-5-yl-thiophene-2-carboxylic acid [5-[(3,3-dimethyl-butyryl)-methyl-amino]- 1-(2-pyridin-3-yl-ethyl)-1H-benzoimidazol- 2-yl]-amide |
| 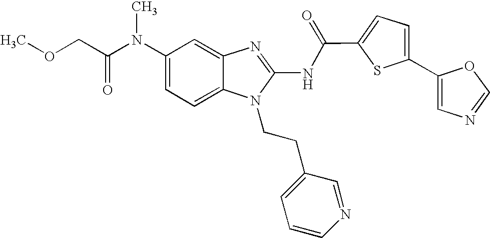 | 5-Oxazol-5-yl-thiophene-2-Carboxylic acid [5-[(2-methoxy-acetyl)-methyl-amino]-1-(2- pyridin-3-yl-ethyl)-1H-benzoimidazol-2- yl]-amide |
| 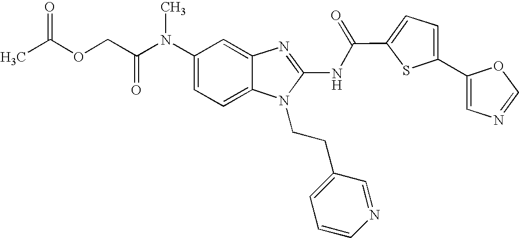 | Acetic acid {methyl-[2-[(5-oxazol-5-yl- thiophene-2-carbonyl)-amino]-1-(2-pyridin- 3-yl-ethyl)-1H-benzoimidazol-5-yl]- carbamoyl}-methyl ester |
| 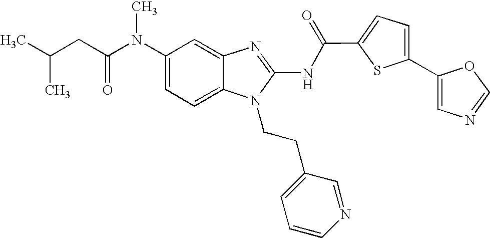 | 5-Oxazol-5-yl-thiophene-2-carboxylic acid [5-[methyl-(3-methyl-butyryl)-amino]-1-(2- pyridin-3-yl-ethyl)-1H-benzoimidazol-2- yl]-amide |
| 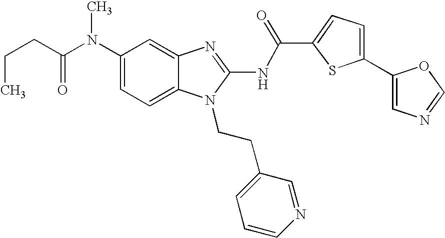 | 5-Oxazol-5-yl-thiophene-2-carboxylic acid [5-(butyryl-methyl-amino)-1-(2-pyridin-3- yl-ethyl)-1H-benzoimidazol-2-yl]-amide |
| 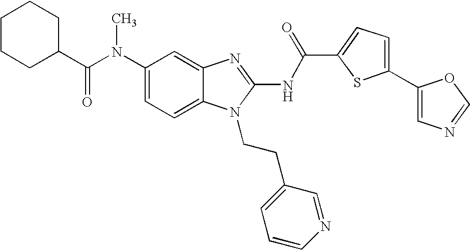 | 5-Oxazol-5-yl-thiophene-2-carboxylic acid [5-(cyclohexanecarbonyl-methyl-amino)-1- (2-pyridin-3-yl-ethyl)-1H-benzoimidazol-2- yl]-amide |
| 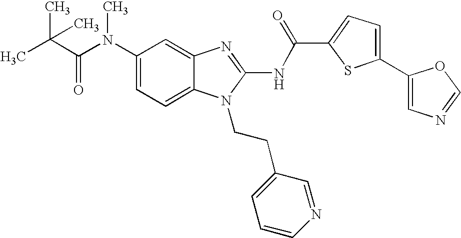 | 5-Oxazol-5-yl-thiophene-2-carboxylic acid [5-[(2,2-dimethyl-propionyl)-methyl- amino]-1-(2-pyridin-3-yl-ethyl)-1H- benzoimidazol-2-yl]-amide |
| 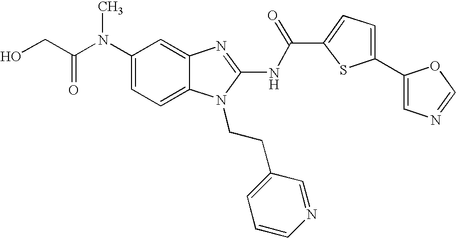 | 5-Oxazol-5-yl-thiophene-2-carboxylic acid [5-[(2-hydroxy-acetyl)-methyl-amino]-1-(2- pyridin-3-yl-ethyl)-1H-benzoimidazol-2- yl]-amide |
| 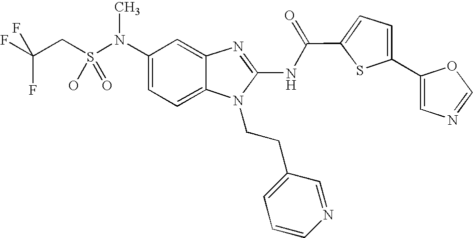 | 5-Oxazol-5-yl-thiophene-2-carboxylic acid [5-[methyl-(2,2,2-trifluoro-ethanesulfonyl)- amino]-1-(2-pyridin-3-yl-ethyl)-1H- benzoimidazol-2-yl]-amide |
| 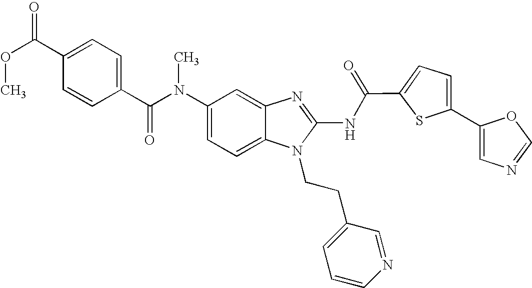 | N-Methyl-N-[2-[(5-oxazol-5-yl-thiophene- 2-carbonyl)-amino]-1-(2-pyridin-3-yl- ethyl)-1H-benzoimidazol-5-yl]- terephthalamic acid methyl ester |
| 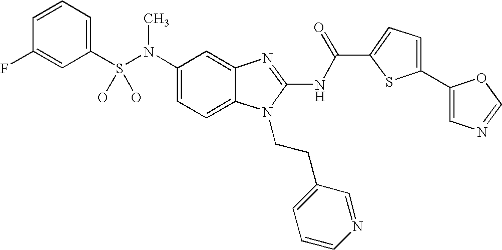 | 5-Oxazol-5-yl-thiophene-2-carboxylic acid [5-[(3-fluoro-benzenesulfonyl)-methyl- amino]-1-(2-pyridin-3-yl-ethyl)-1H- benzoimidazol-2-yl]-amide |
| 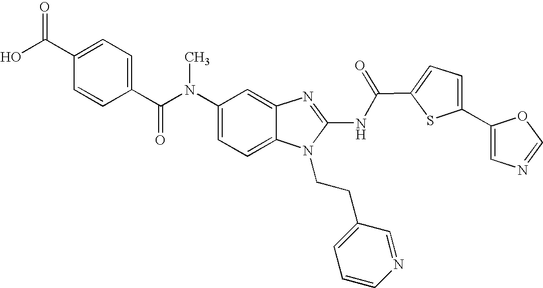 | N-Methyl-N-[2-[(5-oxazol-5-yl-thiophene- 2-carbonyl)-amino]-1-(2-pyridin-3-yl- ethyl)-1H-benzoimidazol-5-yl]- terephthalamic acid |
| 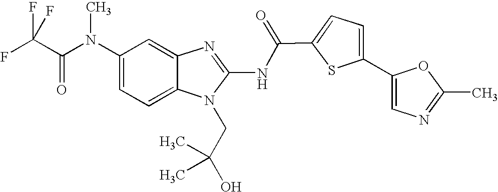 | 5-(2-Methyl-oxazol-5-yl)-thiophene-2- carboxylic acid {1-(2-hydroxy-2-methyl- propyl)-5-[methyl-(2,2,2-trifluoro-acetyl)- amino]-1H-benzoimidazol-2-yl}-amide |
| 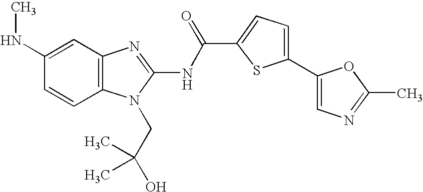 | 5-(2-Methyl-oxazol-5-yl)-thiophene-2- carboxylic acid [1-(2-hydroxy-2-methyl- propyl)-5-methylamino-1H-benzoimidazol- 2-yl]-amide |
| 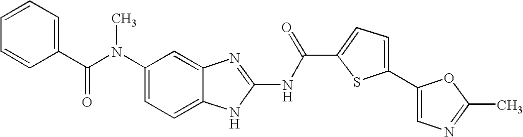 | 5-(2-Methyl-oxazol-5-yl)-thiophene-2- carboxylic acid [5-(benzoyl-methyl-amino)- 1H-benzoimidazol-2-yl]-amide |
| 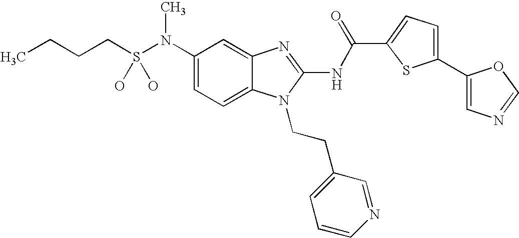 | 5-Oxazol-5-yl-thiophene-2-carboxylic acid [5-[(butane-1-sulfonyl)-methyl-amino]-1- (2-pyridin-3-yl-ethyl)-1H-benzoimidazol- 2-yl]-amide |
| 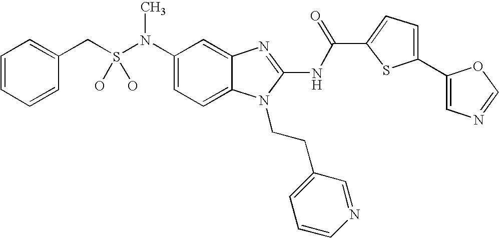 | 5-Oxazol-5-yl-thiophene-2-carboxylic acid [5-(methyl-phenylmethanesulfonyl-amino)- 1-(2-pyridin-3-yl-ethyl)-1H-benzoimidazol- 2-yl]-amide |
| 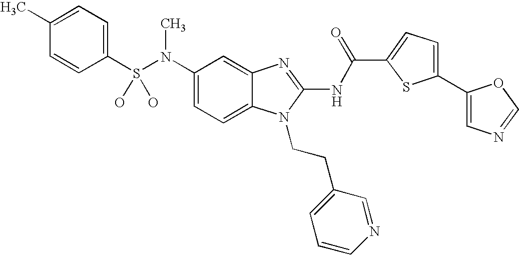 | 5-Oxazol-5-yl-thiophene-2-carboxylic acid [5-[methyl-(toluene-4-sulfonyl)-amino]-1- (2-pyridin-3-yl-ethyl)-1H-benzoimidazol-2- yl]-amide |
| 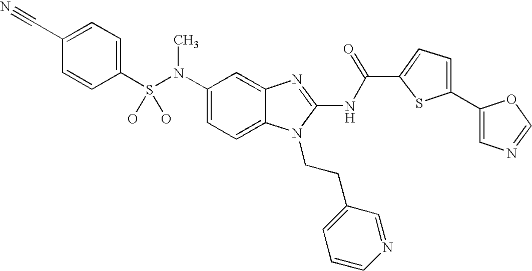 | 5-Oxazol-5-yl-thiophene-2-carboxylic acid [5-[(4-cyano-benzenesulfonyl)-methyl- amino]-1-(2-pyridin-3-yl-ethyl)-1H- benzoimidazol-2-yl]-amide |
| 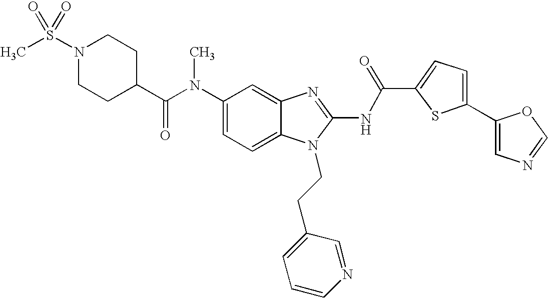 | 1-Methanesulfonyl-piperidine-4-carboxylic acid methyl-[2-[(5-oxazol-5-yl-thiophene-2- carbonyl)-amino]-1-(2-pyridin-3-yl-ethyl)- 1H-benzoimidazol-5-yl]-amide |
| 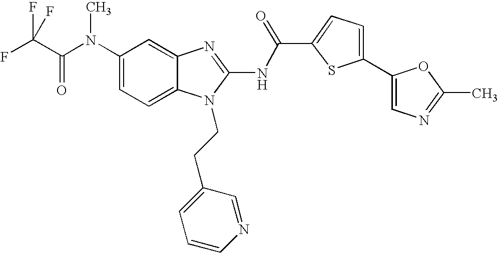 | 5-(2-Methyl-oxazol-5-yl)-thiophene-2- carboxylic acid [5-[methyl-(2,2,2-trifluoro- acetyl)-amino]-1-(2-pyridin-2-yl-ethyl)-1H- benzoimidazol-2-yl]-amide |
| 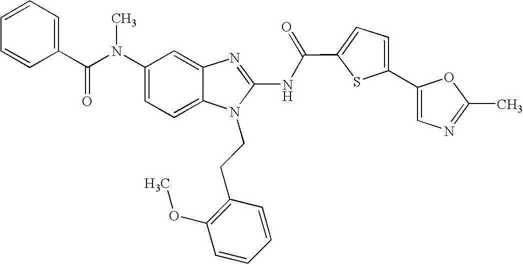 | 5-(2-Methyl-oxazol-5-yl)-thiophene-2- carboxylic acid {5-(benzoyl-methyl-amino)- 1-[2-(2-methoxy-phenyl)-ethyl]-1H- benzoimidazol-2-yl}-amide |
| 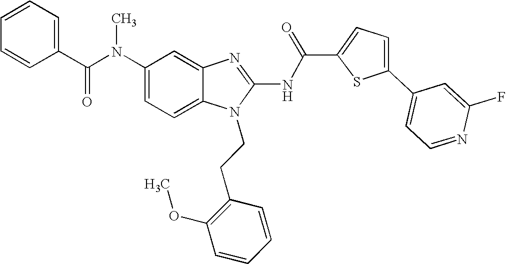 | 5-(2-Fluoro-pyridin-4-yl)-thiophene-2- carboxylic acid {5-(benzoyl-methyl-amino)- 1-[2-(2-methoxy-phenyl)-ethyl]-1H- benzoimidazol-2-yl}-amide |
| 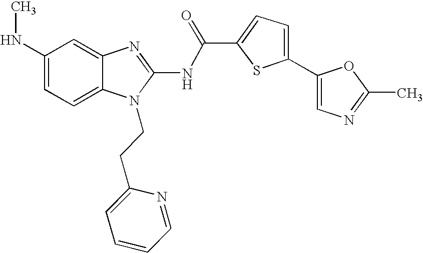 | 5-(2-Methyl-oxazol-5-yl)-thiophene-2- carboxylic acid [5-methylamino-1-(2- pyridin-2-yl-ethyl)-1H-benzoimidazol-2- yl]-amide |
| 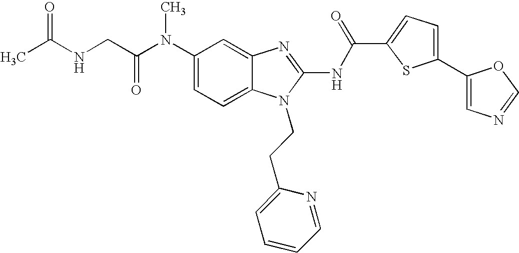 | 5-Oxazol-5-yl-thiophene-2-carboxylic acid [5-[(2-acetylamino-acetyl)-methyl-amino]- 1-(2-pyridin-3-yl-ethyl)-1H-benzoimidazol- 2-yl]-amide |
| 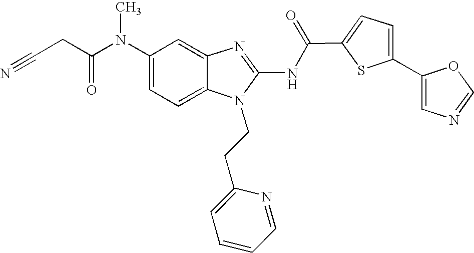 | 5-Oxazol-5-yl-thiophene-2-carboxylic acid [5-[(2-cyano-acetyl)-methyl-amino]-1-(2- pyridin-3-yl-ethyl)-1H-benzoimidazol-2- yl]-amide |
| 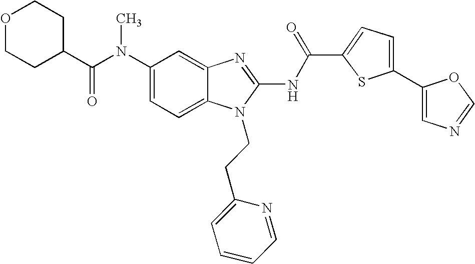 | Tetrahydro-pyran-4-carboxylic acid methyl- [2-[(5-oxazol-5-yl-thiophene-2-carbonyl)- amino]-1-(2-pyridin-3-yl-ethyl)-1H- benzoimidazol-5-yl]-amide |
| 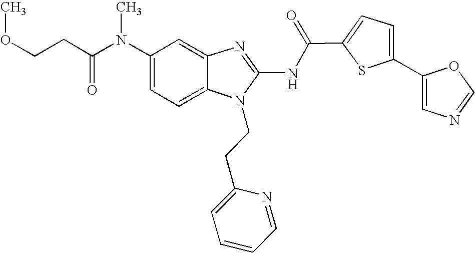 | 5-Oxazol-5-yl-thiophene-2-carboxylic acid [(3-methoxy-propionyl)-methyl-amino]- 1-(2-pyridin-3-yl-ethyl)-1H-benzoimidazol- 2-yl]-amide |
| 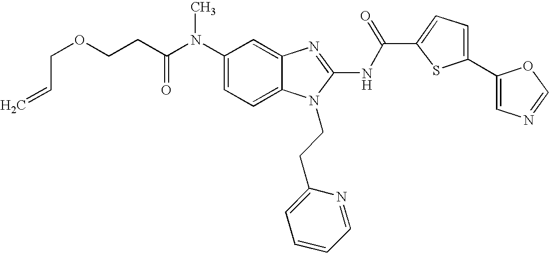 | 5-Oxazol-5-yl-thiophene-2-carboxylic acid [5-[(3-allyloxy-propionyl)-methyl-amino]- 1-(2-pyridin-3-yl-ethyl)-1H-benzoimidazol- 2-yl]-amide |
| 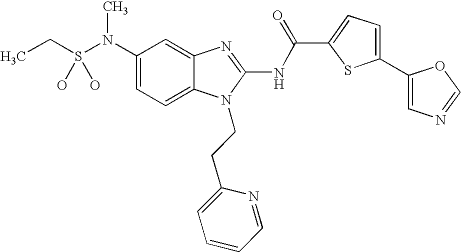 | 5-Oxazol-5-yl-thiophene-2-carboxylic acid [5-(ethanesulfonyl-methyl-amino)-1-(2- pyridin-3-yl-ethyl)-1H-benzoimidazol-2- yl]-amide |
| 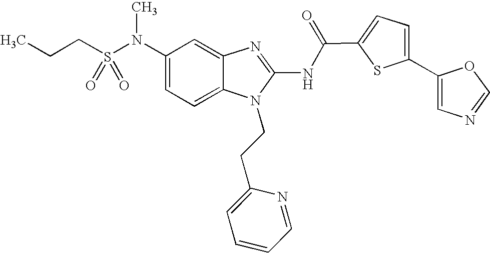 | 5-Oxazol-5-yl-thiophene-2-carboxylic acid [5-[methyl-(propane-1-sulfonyl)-amino]-1- (2-pyridin-3-yl-ethyl)-1H-benzoimidazol-2- yl]-amide |
| 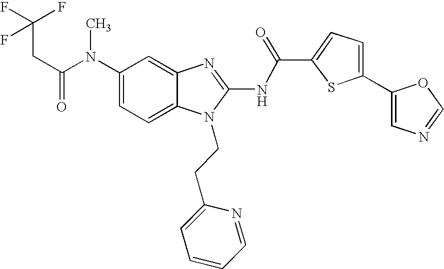 | 5-Oxazol-5-yl-thiophene-2-carboxylic acid [5-[methyl-(3,3,3-trifluoro-propionyl)- amino]-1-(2-pyridin-3-yl-ethyl)-1H- benzoimidazol-2-yl]-amide |
| 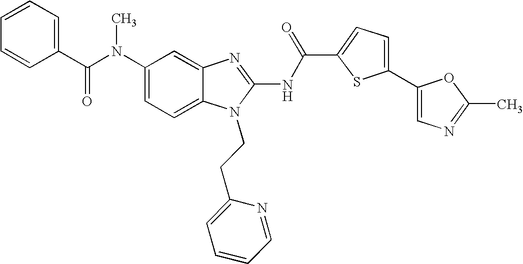 | 5-(2-Methyl-oxazol-5-yl)-thiophene-2- carboxylic acid [5-(benzoyl-methyl-amino)- 1-(2-pyridin-2-yl-ethyl)-1H-benzoimidazol- 2-yl]-amide |
| 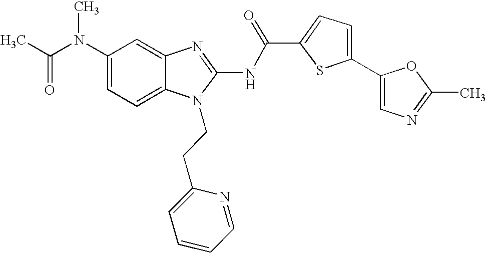 | 5-(2-Methyl-oxazol-5-yl)-thiophene-2- carboxylic acid [5-(acetyl-methyl-amino)-1- (2-pyridin-2-yl-ethyl)-1H-benzoimidazol-2- yl]-amide |
| 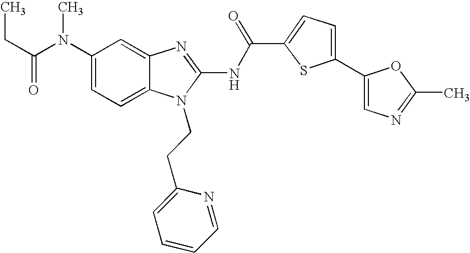 | 5-(2-Methyl-oxazol-5-yl)-thiophene-2- carboxylic acid [5-(methyl-propionyl- amino)-1-(2-pyridin-2-yl-ethyl)-1H- benzoimidazol-2-yl]-amide |
| 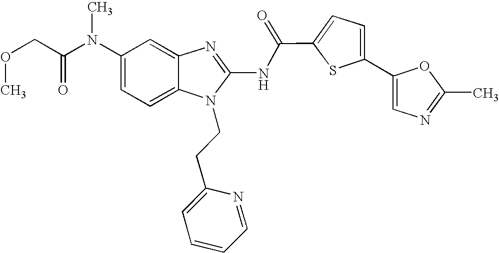 | 5-(2-Methyl-oxazol-5-yl)-thiophene-2- carboxylic acid [5-[(2-methoxy-acetyl)- methyl-amino]-1-(2-pyridin-2-yl-ethyl)-1H- benzoimidazol-2-yl]-amide |
| 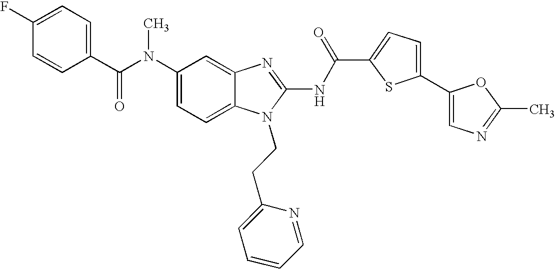 | 5-(2-Methyl-oxazol-5-yl)-thiophene-2- carboxylic acid [5-[(4-fluoro-benzoyl)- methyl-amino]-1-(2-pyridin-2-yl-ethyl)-1H- benzoimidazol-2-yl]-amide |
| 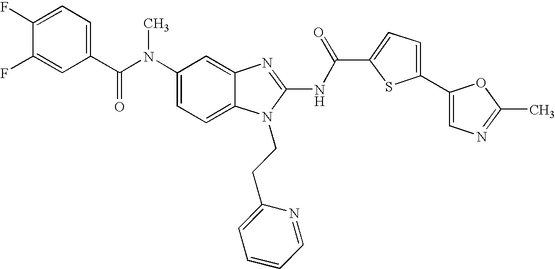 | 5-(2-Methyl-oxazol-5-yl)-thiophene-2- carboxylic acid [5-[(3 ,4-difluoro-benzoyl)- methyl-amino]-1-(2-pyridin-2-yl-ethyl)-1H- benzoimidazol-2-yl]-amide |
| 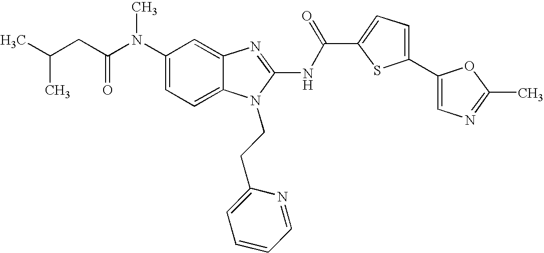 | 5-(2-Methyl-oxazol-5-yl)-thiophene-2- carboxylic acid [5-[methyl-(3-methyl- butyryl)-amino]-1-(2-pyridin-2-yl-ethyl)- 1H-benzoimidazol-2-yl]-amide |
| 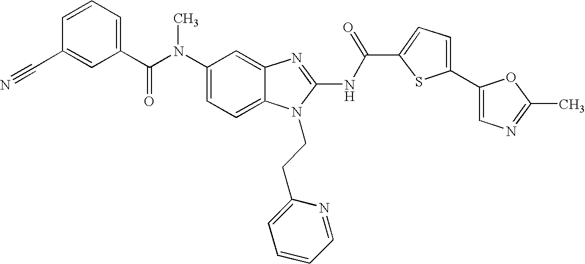 | 5-(2-Methyl-oxazol-5-yl)-thiophene-2- carboxylic acid [5-[(3-cyano-benzoyl)- methyl-amino]-1-(2-pyridin-2-yl-ethyl)-1H- benzoimidazol-2-yl]-amide |
| 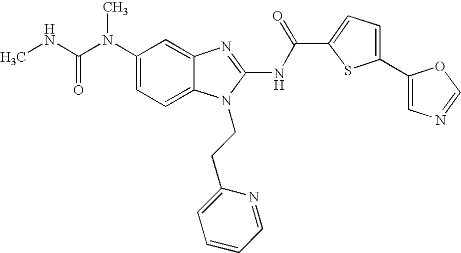 | 5-Oxazol-5-yl-thiophene-2-carboxylic acid [5-(1,3-dimethyl-ureido)-1-(2-pyridin-3-yl- ethyl)-1H-benzoimidazol-2-yl]-amide |
| 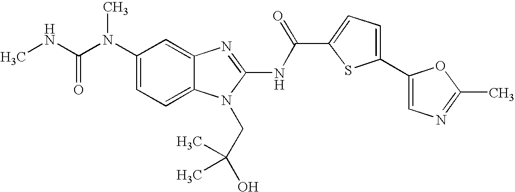 | 5-(2-Methyl-oxazol-5-yl)-thiophene-2- carboxylic acid [5-(1,3-dimethyl-ureido)-1- (2-hydroxy-2-methyl-propyl)-1H- benzoimidazol-2-yl]-amide |
| 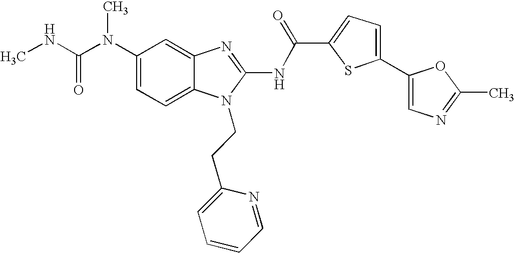 | 5-(2-Methyl-oxazol-5-yl)-thiophene-2- carboxylic acid [5-(1,3-dimethyl-ureido)-1- (2-pyridin-2-yl-ethyl)-1H-benzoimidazol-2- yl]-amide |
| 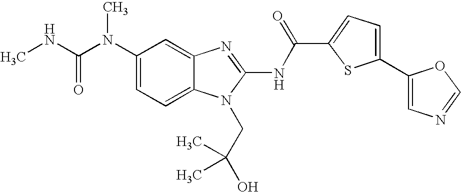 | 5-Oxazol-5-yl-thiophene-2-carboxylic acid [5-(1,3-dimethyl-ureido)-1-(2-hydroxy-2- methyl-propyl)-1H-benzoimidazol-2-yl]- amide |
| 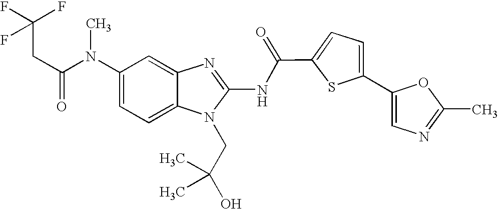 | 5-(2-Methyl-oxazol-5-yl)-thiophene-2- carboxylic acid {1-(2-hydroxy-2-methyl- propyl)-5-[methyl-(3,3,3-trifluoro- propionyl)-amino]-1H-benzoimidazol-2- yl}-amide |
| 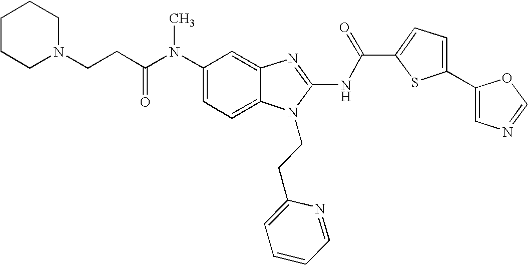 | 5-Oxazol-5-yl-thiophene-2-carboxylic acid [5-[methyl-(3-piperidin-1-yl-propionyl)- amino]-1-(2-pyridin-3-yl-ethyl)-1H- benzoimidazol-2-yl]-amide |
| 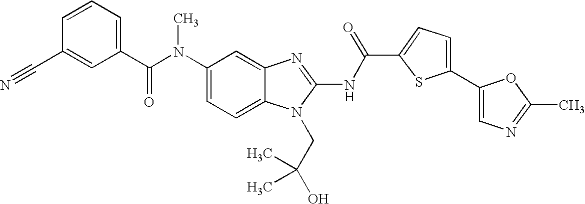 | 5-(2-Methyl-oxazol-5-yl)-thiophene-2- carboxylic acid [5-[(3-cyano-benzoyl)- methyl-amino]-1-(2-hydroxy-2-methyl- propyl)-1H-benzoimidazol-2-yl]-amide |
| 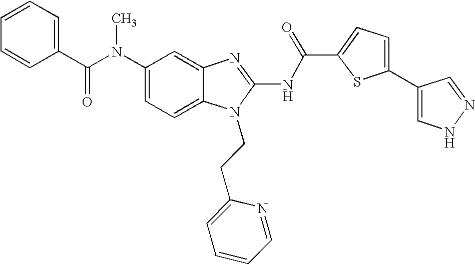 | 5-(1H-Pyrazol-4-yl)-thiophene-2-carboxylic acid [5-(benzoyl-methyl-amino)-1-(2- pyridin-2-yl-ethyl)-1H-benzoimidazol-2- yl]-amide |
| 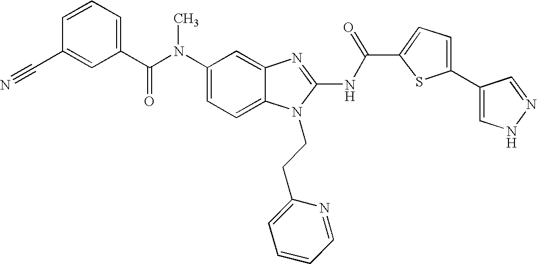 | 5-(1H-Pyrazol-4-yl)-thiophene-2-carboxylic acid [5-[(3-cyano-benzoyl)-methyl-amino]- 1-(2-pyridin-2-yl-ethyl)-1H-benzoimidazol- 2-yl]-amide |
| 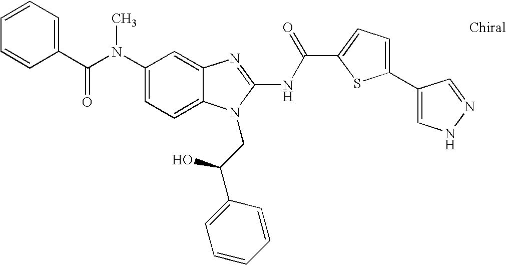 | 5-(1H-Pyrazol-4-yl)-thiophene-2-carboxylic acid [5-(benzoyl-methyl-amino)-1-((R)-2- hydroxy-2-phenyl-ethyl)-1H- benzoimidazol-2-yl]-amide |
| 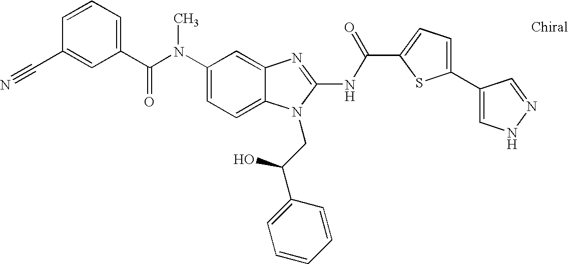 | 5-(1H-Pyrazol-4-yl)-thiophene-2-carboxylic acid [5-[(3-cyano-benzoyl)-methyl-amino]- 1-((R)-2-hydroxy-2-phenyl-ethyl)-1H- benzoimidazol-2-yl]-amide |
| 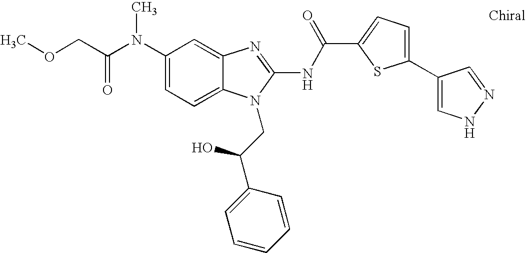 | 5-(1H-Pyrazol-4-yl)-thiophene-2-carboxylic acid {1-((R)-2-hydroxy-2-phenyl-ethyl)-5- [(2-methoxy-acetyl)-methyl-amino]-1H- benzoimidazol-2-yl}-amide |
| 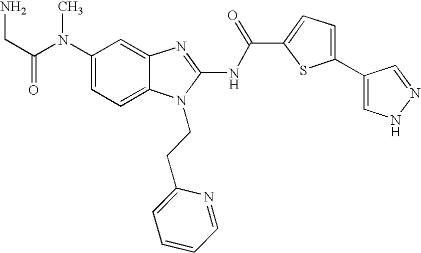 | 5-Oxazol-5-yl-thiophene-2-carboxylic acid [5-[(2-amino-acetyl)-methyl-amino]-1-(2- pyridin-3-yl-ethyl)-1H-benzoimidazol-2- yl]-amide |
| 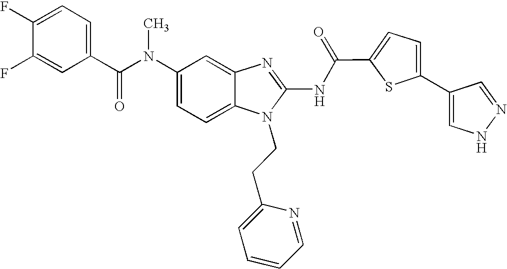 | 5-(1H-Pyrazol-4-yl)-thiophene-2-carboxylic acid [5-[(3,4-difluoro-benzoyl)-methyl- amino]-1-(2-pyridin-2-yl-ethyl)-1H- benzoimidazol-2-yl]-amide |
| 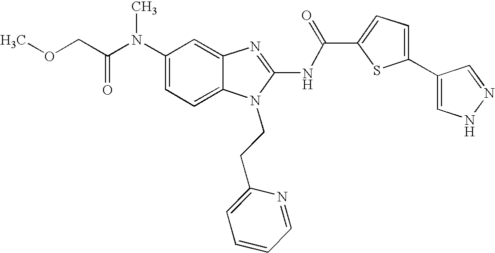 | 5-(1H-Pyrazol-4-yl)-thiophene-2-carboxylic acid [5-[(2-methoxy-acetyl)-methyl-amino]- 1-(2-pyridin-2-yl-ethyl)-1H-benzoimidazol- 2-yl]-amide |
| 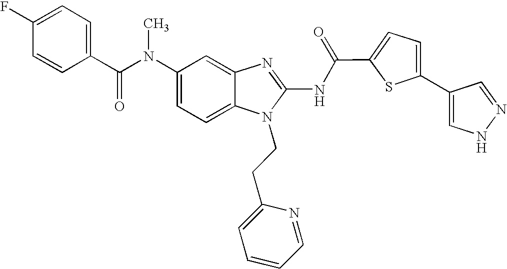 | 5-(1H-Pyrazol-4-yl)-thiophene-2-carboxylic acid [5-[(4-fluoro-benzoyl)-methyl-amino]- 1-(2-pyridin-2-yl-ethyl)-1H-benzoimidazol- 2-yl]-amide |
| 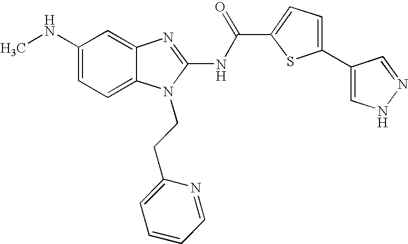 | 5-(1H-Pyrazol-4-yl)-thiophene-2-carboxylic acid [5-methylamino-1-(2-pyridin-2-yl- ethyl)-1H-benzoimidazol-2-yl]-amide |
| 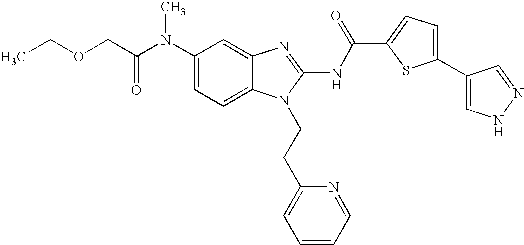 | 5-(1H-Pyrazol-4-yl)-thiophene-2-carboxylic acid [5-[(2-ethoxy-acetyl)-methyl-amino]-1- (2-pyridin-2-yl-ethyl)-1H-benzoimidazol-2- yl]-amide |
| 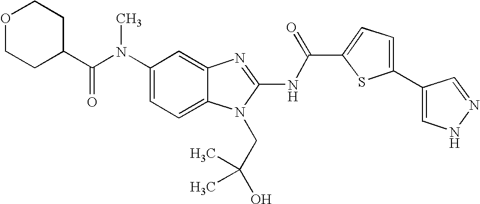 | Tetrahydro-pyran-4-carboxylic acid (1-(2- hydroxy-2-methyl-propyl)-2-{[5-(1H- pyrazol-4-yl)-thiophene-2-carbonyl]- amino}-1H-benzoimidazol-5-yl)-methyl- amide |
| 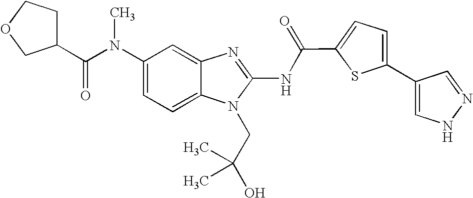 | Tetrahydro-furan-3-carboxylic acid (1-(2- hydroxy-2-methyl-propyl)-2-{[5-(1H- pyrazol-4-yl)-thiophene-2-carbonyl]- amino}-1H-benzoimidazol-5-yl)-methyl- amide |
| 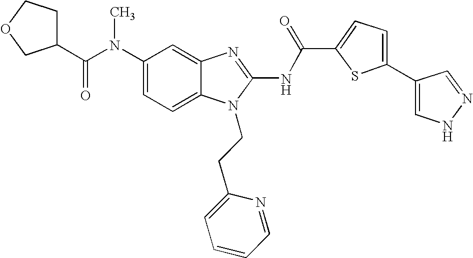 | Tetrahydro-furan-3-carboxylic acid methyl- [2-{[5-(1H-pyrazol-4-yl)-thiophene-2- carbonyl]-amino}-1-(2-pyridin-2-yl-ethyl)- 1H-benzoimidazol-5-yl]-amide |
| 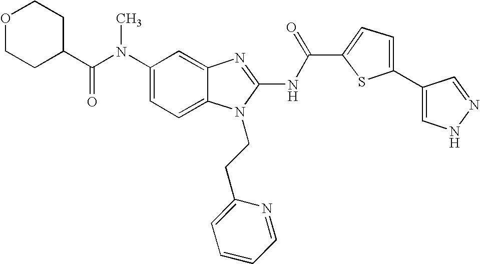 | Tetrahydro-pyran-4-carboxylic acid methyl- [2-{[5-(1H-pyrazol-4-yl)-thiophene-2- carbonyl]-amino}-1-(2-pyridin-2-yl-ethyl)- 1H-benzoimidazol-5-yl]-amide |
| 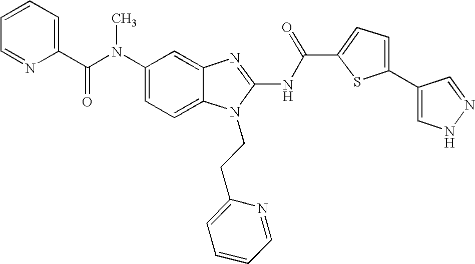 | Pyridine-2-carboxylic acid methyl-[2-{[5- (1H-pyrazol-4-yl)-thiophene-2-carbonyl]- amino}-1-(2-pyridin-2-yl-ethyl)-1H- benzoimidazol-5-yl]-amide |
| 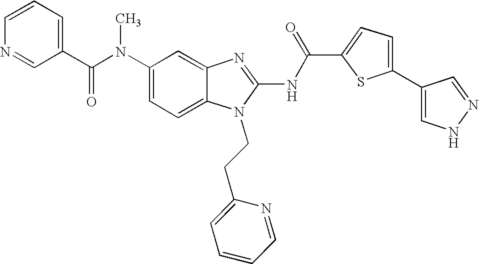 | N-Methyl-N-[2-{[5-(1H-pyrazol-4-yl)- thiophene-2-carbonyl]-amino}-1-(2-pyridin- 2-yl-ethyl)-1H-benzoimidazol-5-yl]- nicotinamide |
| 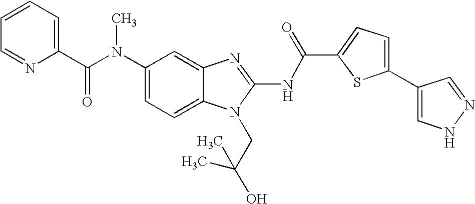 | Pyridine-2-carboxylic acid (1-(2-hydroxy-2- methyl-propyl)-2-{[5-(1FI-pyrazol-4-yl)- thiophene-2-carbonyl]-amino}-1H- benzoimidazol-5-yl)-methyl-amide |
| 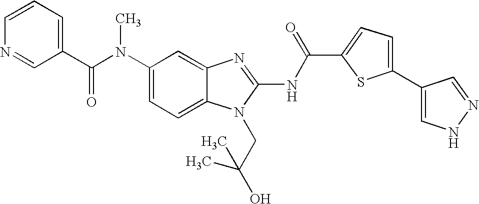 | N-(1-(2-Hydroxy-2-methyl-propyl)-2-{[5- (1H-pyrazol-4-yl)-thiophene-2-carbonyl]- amino}-1H-benzoimidazol-5-yl)-N-methyl- nicotinamide |
| 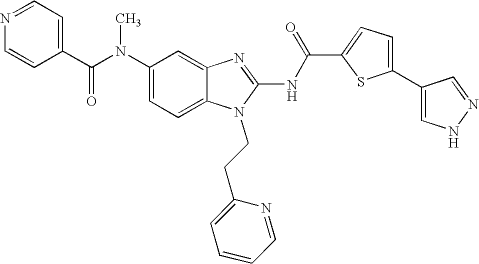 | N-Methyl-N-[2-{[5-(1H-pyrazol-4-yl)- thiophene-2-carbonyl]-amino}-1-(2-pyridin- 2-yl-ethyl)-1H-benzoimidazol-5-yl]- isonicotinamide |
| 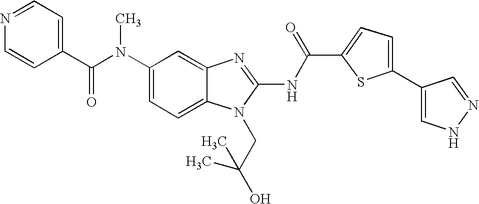 | N-(1-(2-Hydroxy-2-methyl-propyl)-2-{[5- (1H-pyrazol-4-yl)-thiophene-2-carbonyl]- amino}-1H-benzoimidazol-5-yl)-N-methyl- isonicotinamide |
| 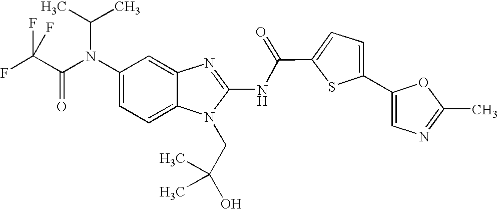 | 5-(2-Methyl-oxazol-5-yl)-thiophene-2- carboxylic acid {1-(2-hydroxy-2-methyl- propyl)-5-[isopropyl-(2,2,2-trifluoro- acetyl)-amino]-1H-benzoimidazol-2-yl}- amide |
| 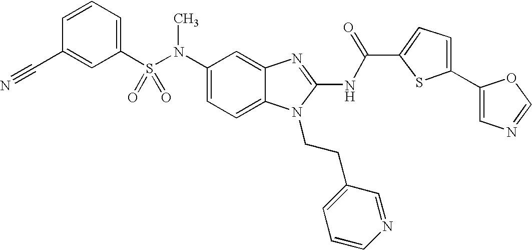 | 5-Oxazol-5-yl-thiophene-2-carboxylic acid [5-[(3-cyano-benzenesulfonyl)-methyl- amino]-1-(2-pyridin-3-yl-ethyl)-1H- benzoimidazol-2-yl]-amide |
| 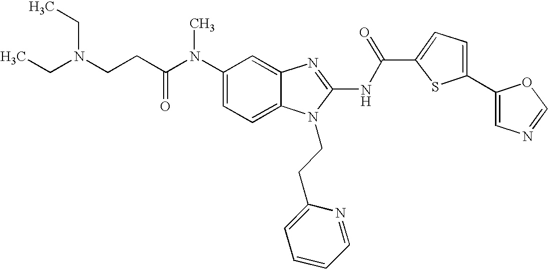 | 5-Oxazol-5-yl-thiophene-2-Carboxylic acid [5-[(3-diethylamino-propionyl)-methyl- amino]-1-(2-pyridin-3-yl-ethyl)-1H- benzoimidazol-2-yl]-amide |
| 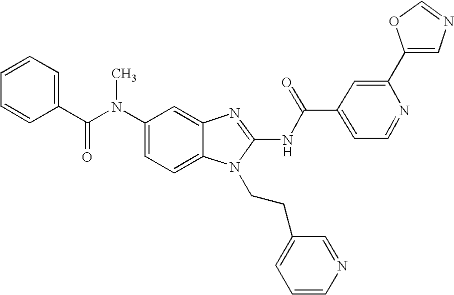 | N-{5-(Benzoyl-methyl-amino)-1-(2-pyridin- 3-yl-ethyl)-1H-benzoimidazol-2-yl]-2- oxazol-5-yl-isonicotinamide |
| 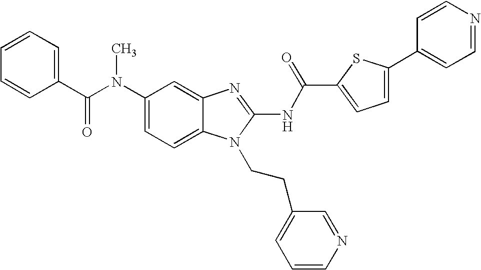 | 5-Pyridin-4-yl-thiophene-2-carboxylic acid [5-(benzoyl-methyl-amino)-1-(2-pyridin-3- yl-ethyl)-1H-benzoimidazol-2-yl]-amide |
| 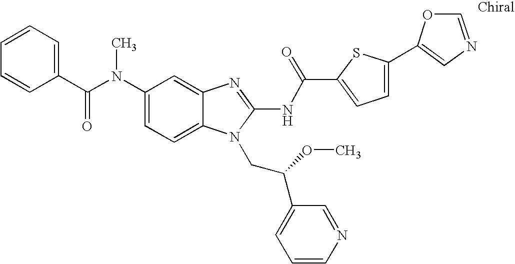 | 5-Oxazol-5-yl-thiophene-2-carboxylic acid [5-(benzoyl-methyl-amino)-1-((R)-2- methoxy-2-phenyl-ethyl)-1H- benzoimidazol-2-yl]-amide |
| 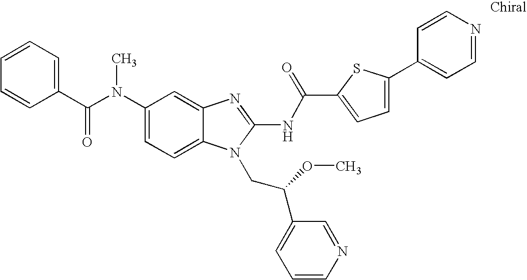 | 5-Pyridin-4-yl-thiophene-2-carboxylic acid [5-(benzoyl-methyl-amino)-1-((R)-2- methoxy-2-phenyl-ethyl)-1H- benzoimidazol-2-yl]-amide |
| 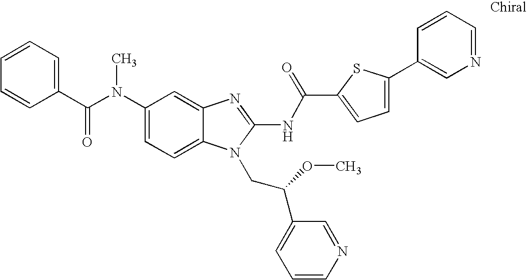 | 5-Pyridin-3-yl-thiophene-2-carboxylic acid [5-(benzoyl-methyl-amino)-1-((R)-2- methoxy-2-phenyl-ethyl)-1H- benzoimidazol-2-yl]-amide |
| 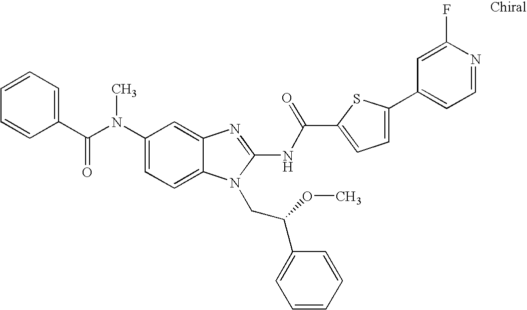 | 5-(2-Fluoro-pyridin-4-yl)-thiophene-2- carboxylic acid [5-(benzoyl-methyl-amino)- 1-((R)-2-methoxy-2-phenyl-ethyl)-1H- benzoimidazol-2-yl]-amide |
| 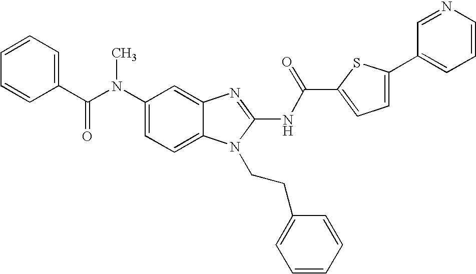 | 5-Pyridin-3-yl-thiophene-2-carboxylic acid [5-(benzoyl-methyl-amino)-1-phenethyl- 1H-benzoimidazol-2-yl]-amide |
| 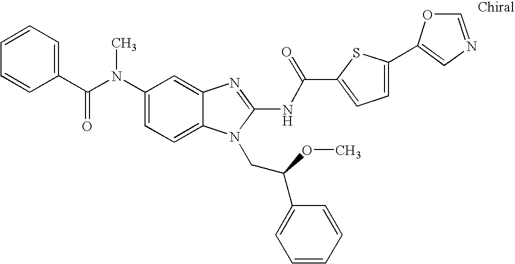 | 5-Oxazol-5-yl-thiophene-2-carboxylic acid [5-(benzoyl-methyl-amino)-1-((S)-2- methoxy-2-phenyl-ethyl)-1H- benzoimidazol-2-yl]-amide |
| 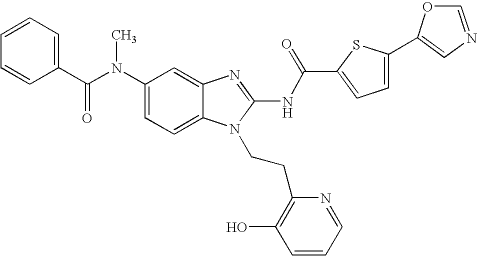 | 5-Oxazol-5-yl-thiophene-2-carboxylic acid {5-(benzoyl-methyl-amino)-1-[2-(3- hydroxy-pyridin-2-yl)-ethyl]-1H- benzoimidazol-2-yl}-amide |
| 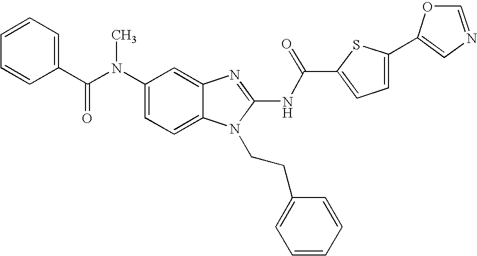 | 5-Oxazol-5-yl-thiophene-2-carboxylic acid [5-(benzoyl-methyl-amino)-1-phenethyl- 1H-benzoimidazol-2-yl]-amide |
| 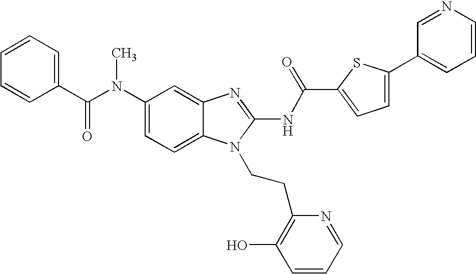 | 5-Pyridin-3-yl-thiophene-2-carboxylic acid {5-(benzoyl-methyl-amino)-1-[2-(3- hydroxy-pyridin-2-yl)-ethyl]-1H- benzoimidazol-2-yl}-amide |
| 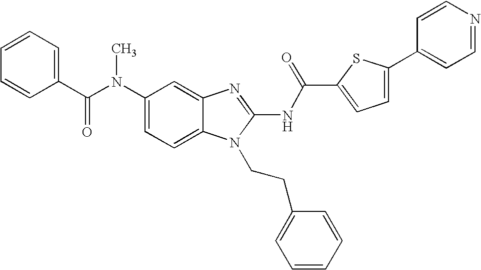 | 5-Pyridin-4-yl-thiophene-2-carboxylic acid [5-(benzoyl-methyl-amino)-1-phenethyl- 1H-benzoimidazol-2-yl]-amide |
| 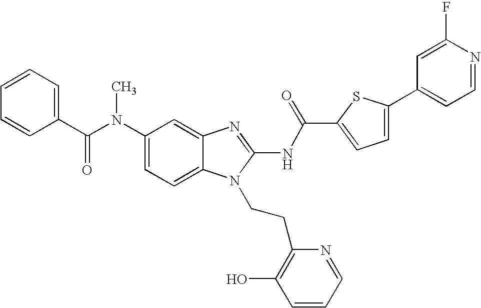 | 5-(2-Fluoro-pyridin-4-yl)-thiophene-2- carboxylic acid {5-(benzoyl-methyl-amino)- 1-[2-(3-hydroxy-pyridin-2-yl)-ethyl]-1H- benzoimidazol-2-yl}-amide |
| 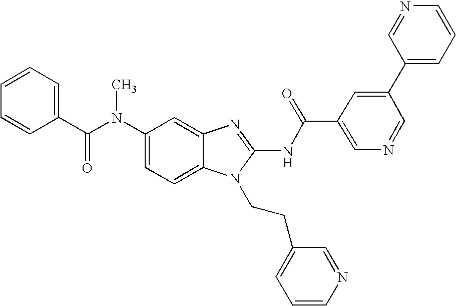 | [3,3′]Bipyridinyl-5-carboxylic acid [5- (benzoyl-methyl-amino)-1-(2-pyridin-3-yl- ethyl)-1H-benzoimidazol-2-yl]-amide |
| 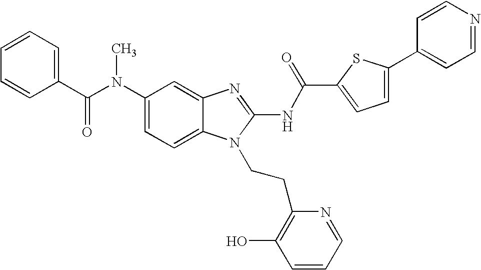 | 5-Pyridin-4-yl-thiophene-2-carboxylic acid {5-(benzoyl-methyl-amino)-1-[2-(3- hydroxy-pyridin-2-yl)-ethyl]-1H- benzoimidazol-2-yl}-amide |
| 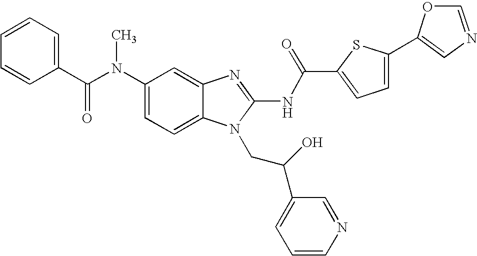 | 5-Oxazol-5-yl-thiophene-2-carboxylic acid [5-(benzoyl-methyl-amino)-1-(2-hydroxy-2- pyridin-3-yl-ethyl)-1H-benzoimidazol-2- yl]-amide |
| 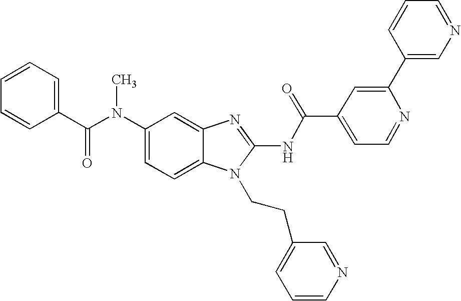 | [2,3′]Bipyridinyl-4-carboxylic acid [5- (benzoyl-methyl-amino)-1-(2-pyridin-3-yl- ethyl)-1H-benzoimidazol-2-yl]-amide |
| 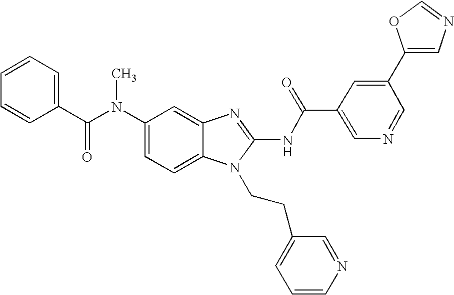 | N-{5-(Benzoyl-methyl-amino)-1-(2-pyridin- 3-yl-ethyl)-1H-benzoimidazol-2-yl]-5- oxazol-5-yl-nicotinamide |
| 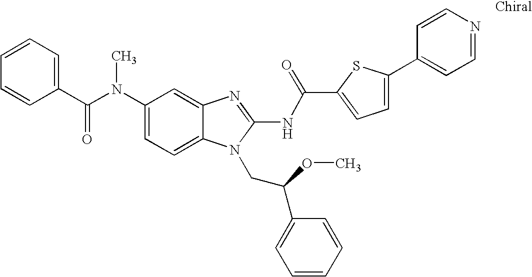 | 5-Pyridin-4-yl-thiophene-2-carboxylic acid [5-(benzoyl-methyl-amino)-1-((S)-2- methoxy-2-phenyl-ethyl)-1H- benzoimidazol-2-yl]-amide |
| 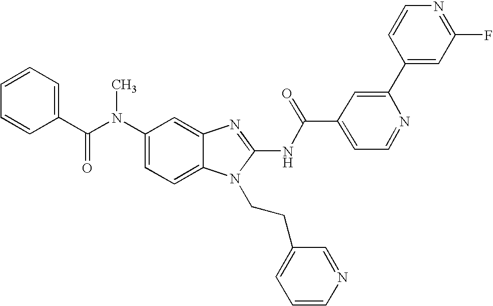 | 2′-Fluoro-[2,4′]bipyridinyl-4-carboxylic acid [5-(benzoyl-methyl-amino)-1-(2- pyridin-3-yl-ethyl)-1H-benzoimidazol- 2-yl]-amide |
| 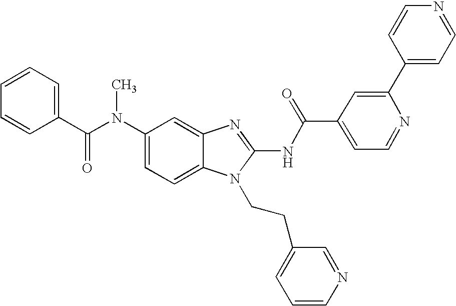 | [2,4′]Bipyridinyl-4-carboxylic acid [5- (benzoyl-methyl-amino)-1-(2-pyridin-3-yl- ethyl)-1H-benzoimidazol-2-yl]-amide |
| 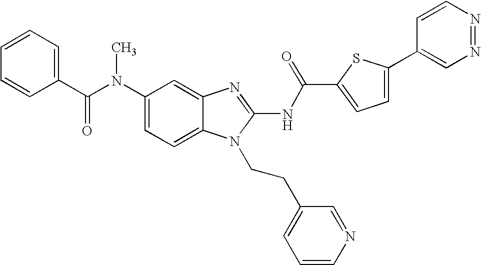 | 5-Pyridazin-4-yl-thiophene-2-carboxylic acid [5-(benzoyl-methyl-amino)-1-(2- pyridin-3-yl-ethyl)-1H-benzoimidazol-2- yl]-amide |
| 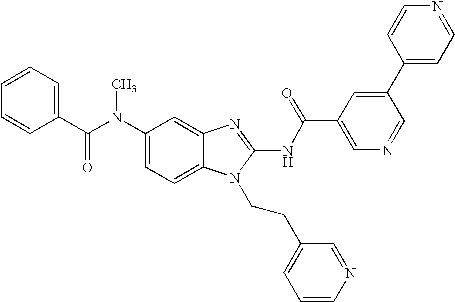 | [3,4′]Bipyridinyl-5-carboxylic acid [5- (benzoyl-methyl-amino)-1-(2-pyridin-3-yl- ethyl)-1H-benzoimidazol-2-yl]-amide |
| 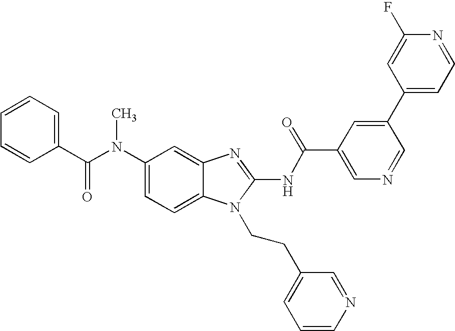 | 2′-Fluoro-[3,4′]bipyridinyl-5-carboxylic acid [5-(benzoyl-methyl-amino)-1-(2- pyridin-3-yl-ethyl)-1H-benzoimidazol- 2-yl]-amide |
| 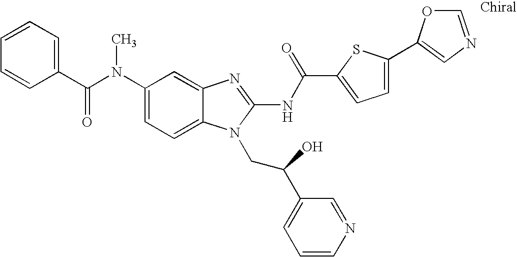 | 5-Oxazol-5-yl-thiophene-2-carboxylic acid [5-(benzoyl-methyl-amino)-1-((S)-2- hydroxy-2-pyridin-3-yl-ethyl)-1H- benzoimidazol-2-yl]-amide |
| 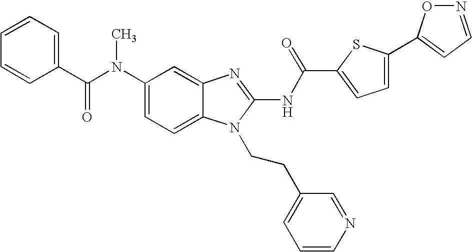 | 5-Isoxazol-5-yl-thiophene-2-carboxylic acid [5-(benzoyl-methyl-amino)-1-(2-pyridin-3- yl-ethyl)-1H-benzoimidazol-2-yl]-amide |
| 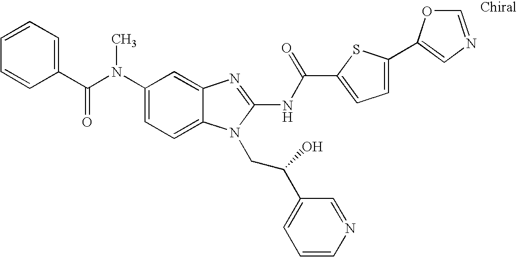 | 5-Oxazol-5-yl-thiophene-2-carboxylic acid [5-(benzoyl-methyl-amino)-1-((R)-2- hydroxy-2-pyridin-3-yl-ethyl)-1H- benzoimidazol-2-yl]-amide |
| 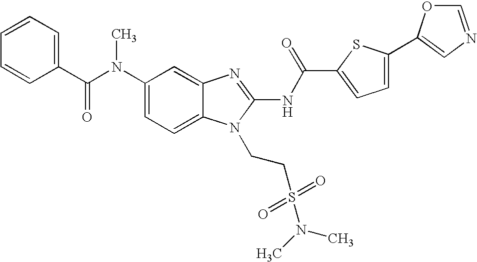 | 5-Oxazol-5-yl-thiophene-2-carboxylic acid [5-(benzoyl-methyl-amino)-1-(2- dimethylsulfamoyl-ethyl)-1H- benzoimidazol-2-yl]-amide |
| 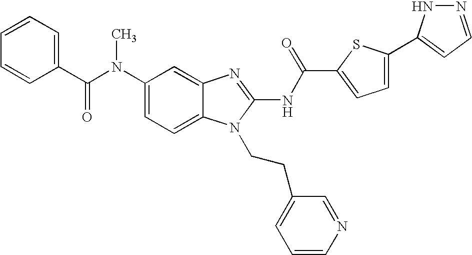 | 5-(2H-Pyrazol-3-yl)-thiophene-2-carboxylic acid [5-(benzoyl-methyl-amino)-1-(2- pyridin-3-yl-ethyl)-1H-benzoimidazol-2- yl]-amide |
| 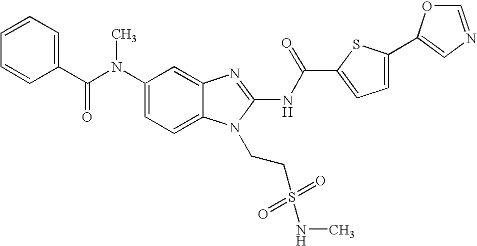 | 5-Oxazol-5-yl-thiophene-2-carboxylic acid [5-(benzoyl-methyl-amino)-1-(2- methylsulfamoyl-ethyl)-1H-beiizoimidazol- 2-yl]-amide |
| 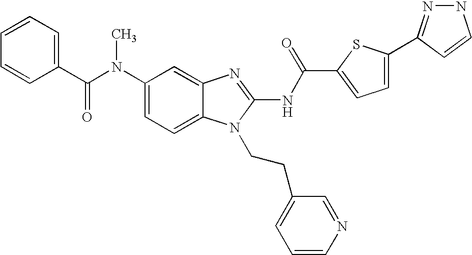 | 5-Isoxazol-3-yl-thiophene-2-carboxylic acid [5-(benzoyl-methyl-amino)-1-(2-pyridin-3- yl-ethyl)-1H-benzoimidazol-2-yl]-amide |
| 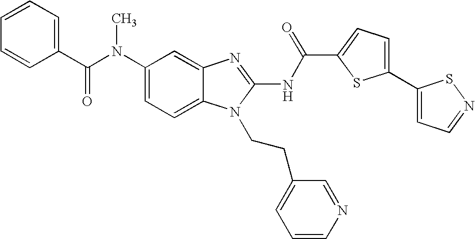 | 5-Isothiazol-5-yl-thiophene-2-carboxylic acid [5-(benzoyl-methyl-amino)-1-(2- pyridin-3-yl-ethyl)-1H-benzoimidazol-2- yl]-amide |
| 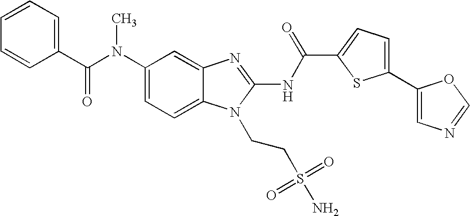 | 5-Oxazol-5-yl-thiophene-2-carboxylic acid [5-(benzoyl-methyl-amino)-1-(2-sulfamoyl- ethyl)-1H-benzoimidazol-2-yl]-amide |
| 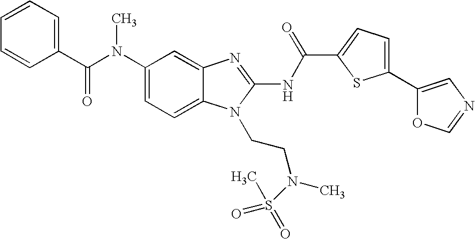 | 5-Oxazol-5-yl-thiophene-2-carboxylic acid {5-(benzoyl-methyl-amino)-1-[2- (methanesulfonyl-methyl-amino)-ethyl]- 1H-benzoimidazol-2-yl}-amide |
| 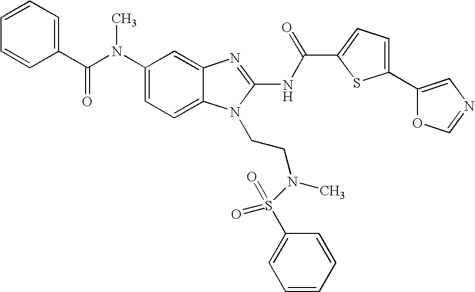 | 5-Oxazol-5-yl-thiophene-2-carboxylic acid [1-[2-(benzenesulfonyl-methyl-amino)- ethyl]-5-(benzoyl-methyl-amino)-1H- benzoimidazol-2-yl]-amide |
| 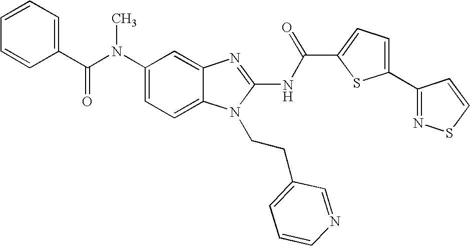 | 5-Isothiazol-3-yl-thiophene-2-carboxylic acid [5-(benzoyl-methyl-amino)-1-(2- pyridin-3-yl-ethyl)-1H-benzoimidazol-2- yl]-amide |
| 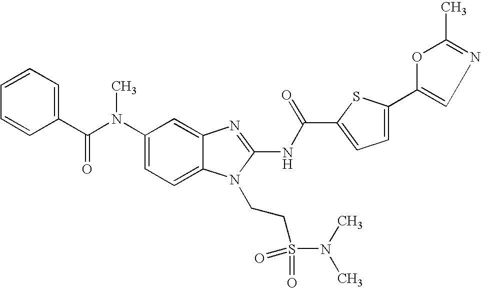 | 5-(2-Methyl-oxazol-5-yl)-thiophene-2- carboxylic acid [5-(benzoyl-methyl-amino)- 1-(2-dimethylsulfamoyl-ethyl)-1H- benzoimidazol-2-yl]-amide |
| 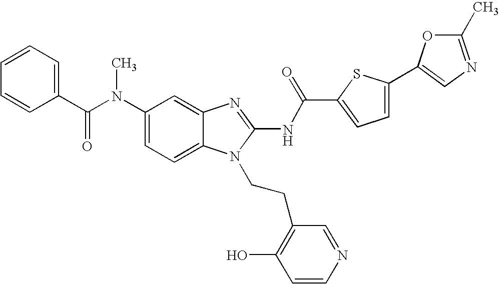 | 5-(2-Methyl-oxazol-5-yl)-thiophene-2- carboxylic acid {5-(benzoyl-methyl-amino)- 1-[2-(3-hydroxy-pyridin-2-yl)-ethyl]-1H- benzoimidazol-2-yl}-amide |
| 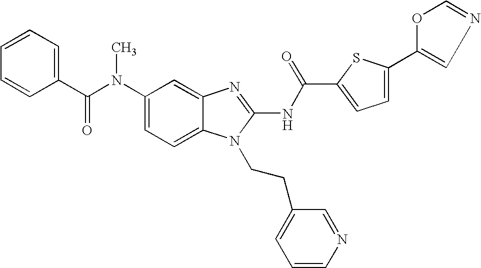 | 2-Oxazol-5-yl-thiazole-5-carboxylic acid [5-(benzoyl-methyl-amino)-1-(2-pyridin-3- yl-ethyl)-1H-benzoimidazol-2-yl]-amide |
| 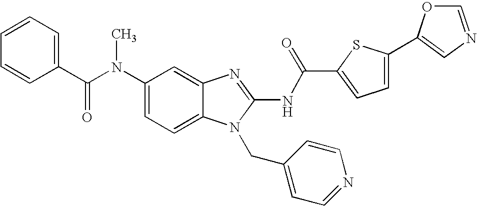 | 5-Oxazol-5-yl-thiophene-2-carboxylic acid [5-(benzoyl-methyl-amino)-1-pyridin-4- ylmethyl-1H-benzoimidazol-2-yl]-amide |
| 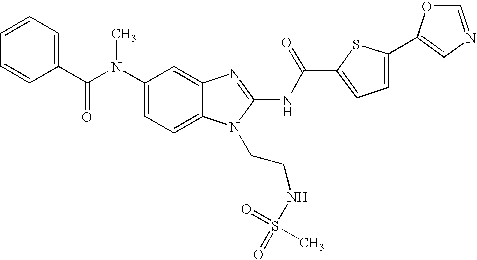 | 5-Oxazol-5-yl-thiophene-2-carboxylic acid [5-(benzoyl-methyl-amino)-1-(2- methanesulfonylamino-ethyl)-1H- benzoimidazol-2-yl]-amide |
| 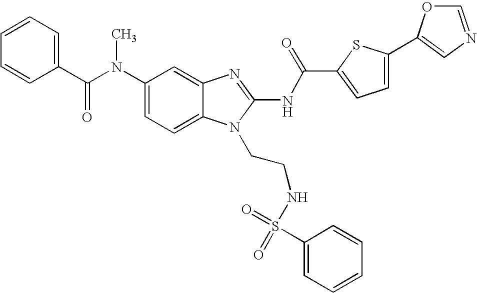 | 5-Oxazol-5-yl-thiophene-2-carboxylic acid [1-(2-benzenesulfonylamino-ethyl)-5- (benzoyl-methyl-amino)-1H- benzoimidazol-2-yl]-amide |
| 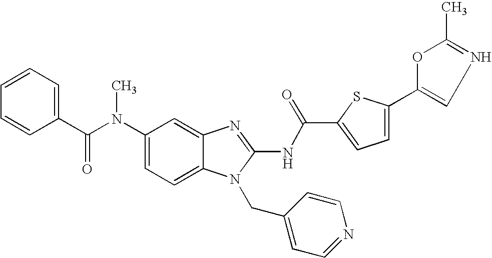 | 5-(2-Methyl-oxazol-5-yl)-thiophene-2- carboxylic acid [5-(benzoyl-methyl-amino)- 1-pyridin-4-ylmethyl-1H-benzoimidazol-2- yl]-amide |
| 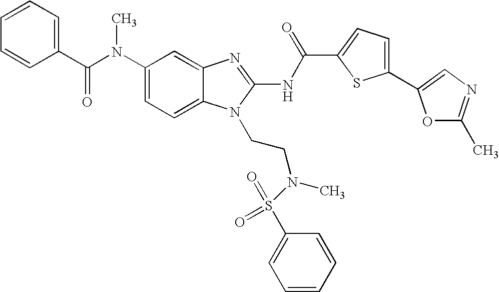 | 5-(2-Methyl-oxazol-5-yl)-thiophene-2- carboxylic acid [1-[2-(benzenesulfonyl- methyl-amino)-ethyl]-5-(benzoyl-methyl- amino)-1H-benzoimidazol-2-yl]-amide |
| 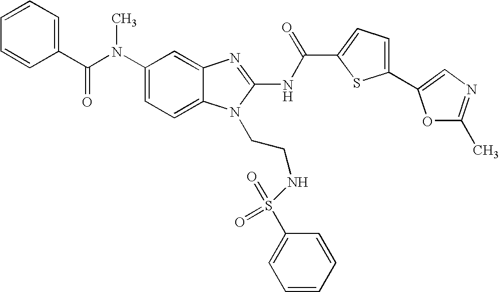 | 5-(2-Methyl-oxazol-5-yl)-thiophene-2- carboxylic acid [1-(2- benzenesulfonylamino-ethyl)-5-(benzoyl- methyl-amino)-1H-benzoimidazol-2-yl]- amide |
| 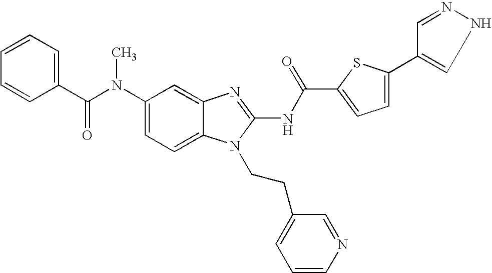 | 5-(1H-Pyrazol-4-yl)-thiophene-2-carboxylic acid [5-(benzoyl-methyl-amino)-1-(2- pyridin-3-yl-ethyl)-1H-benzoimidazol-2- yl]-amide |
| 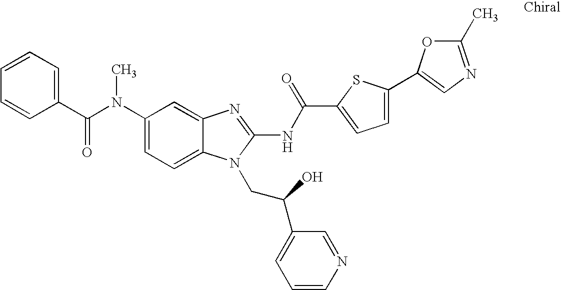 | 5-(2-Methyl-oxazol-5-yl)-thiophene-2- carboxylic acid [5-(benzoyl-methyl-amino)- 1-((S)-2-hydroxy-2-pyridin-3-yl-ethyl)-1H- benzoimidazol-2-yl]-amide |
| 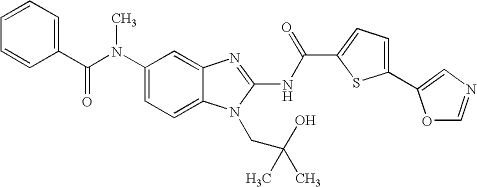 | 2-Oxazol-5-yl-thiazole-5-carboxylic acid [5-(benzoyl-methyl-amino)-1-(2-hydroxy-2- methyl-propyl)-1H-benzoimidazol-2-yl]- amide |
| 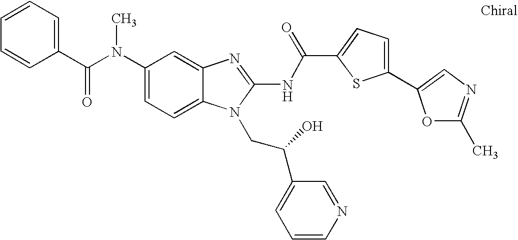 | 5-(2-Methyl-oxazol-5-yl)-thiophene-2- carboxylic acid [5-(benzoyl-methyl-amino)- 1-((R)-2-hydroxy-2-pyridin-3-yl-ethyl)-1H- benzoimidazol-2-yl]-amide |
| 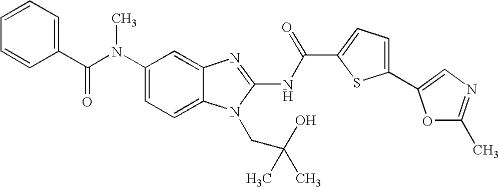 | 5-(2-Methyl-oxazol-5-yl)-thiophene-2- carboxylic acid [5-(benzoyl-methyl-amino)- 1-(2-hydroxy-2-methyl-propyl)-1H- benzoimidazol-2-yl]-amide |
| 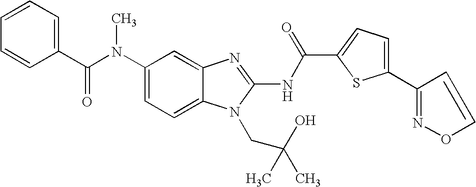 | 5-Isoxazol-3-yl-thiophene-2-carboxylic acid [5-(benzoyl-methyl-amino)-1-(2-hydroxy- 2-methyl-propyl)-1H-benzoimidazol- 2-yl]-amide |
| 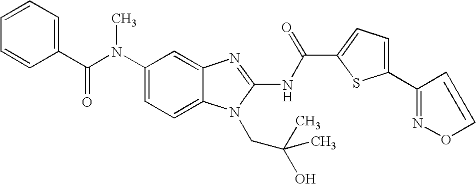 | 5-Isothiazol-3-yl-thiophene-2-carboxylic acid [5-(benzoyl-methyl-amino)-1-(2- hydroxy-2-methyl-propyl)-1H- benzoimidazol-2-yl]-amide |
| 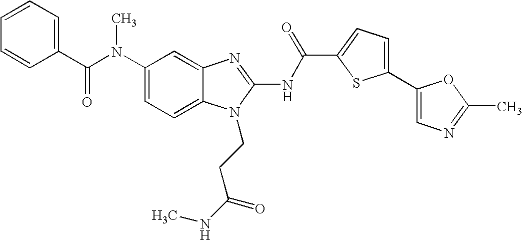 | 5-(2-Methyl-oxazol-5-yl)-thiophene-2- carboxylic acid [5-(benzoyl-methyl-amino)- 1-(2-methylcarbamoyl-ethyl)-1H- benzoimidazol-2-yl]-amide |
| 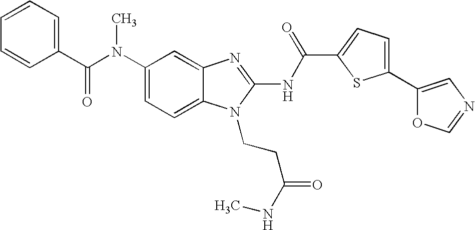 | 5-Oxazol-5-yl-thiophene-2-carboxylic acid [5-(benzoyl-methyl-amino)-1-(2- methylcarbamoyl-ethyl)-1H-benzoimidazol- 2-yl]-amide |
| 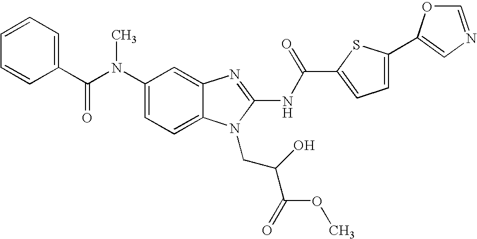 | 3-{5-(Benzoyl-methyl-amino)-2-[(5-oxazol- 5-yl-thiophene-2-carbonyl)-amino]- benzoimidazol-1-yl}-2-hydroxy-propionic acid methyl ester |
| 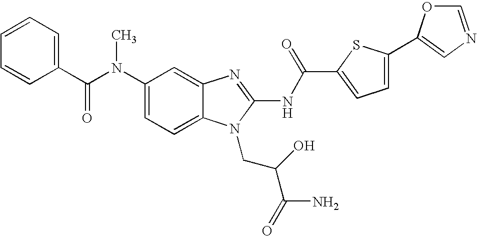 | 5-Oxazol-5-yl-thiophene-2-carboxylic acid [5-(benzoyl-methyl-amino)-1-(2-carbamoyl- 2-hydroxy-ethyl)-1H-benzoimidazol-2-yl]- amide |
| 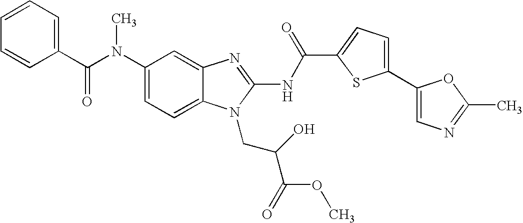 | 3-(5-(Benzoyl-methyl-amino)-2-{[5-(2- methyl-oxazol-5-yl)-thiophene-2-carbonyl]- amino}-benzoimidazol-1-yl)-2-hydroxy- propionic acid methyl ester |
| 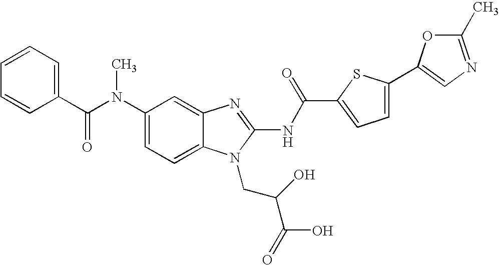 | 3-(5-(Benzoyl-methyl-amino)-2-{[5-(2- methyl-oxazol-5-yl)-thiophene-2-carbonyl]- amino}-benzoimidazol-1-yl)-2-hydroxy- propionic acid |
| 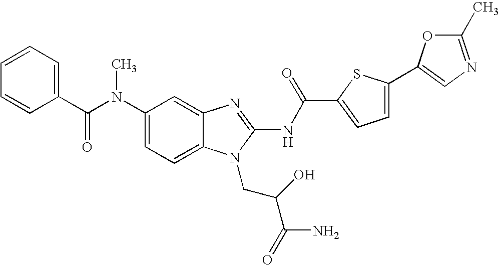 | 5-(2-Methyl-oxazol-5-yl)-thiophene-2- carboxylic acid [5-(benzoyl-methyl-amino)- 1-(2-carbamoyl-2-hydroxy-ethyl)-1H- benzoimidazol-2-yl]-amide |
| 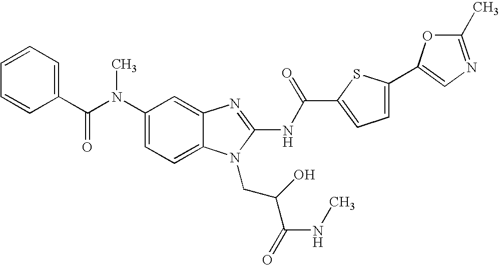 | 5-(2-Methyl-oxazol-5-yl)-thiophene-2- carboxylic acid [5-(benzoyl-methyl-amino)- 1-(2-hydroxy-2-methylcarbamoyl-ethyl)- 1H-benzoimidazol-2-yl]-amide |
| 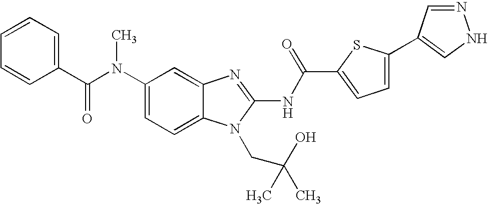 | 5-(1H-Pyrazol-4-yl)-thiophene-2-carboxylic acid [5-(benzoyl-methyl-amino)-1-(2- hydroxy-2-methyl-propyl)-1H- benzoimidazol-2-yl]-amide |
| 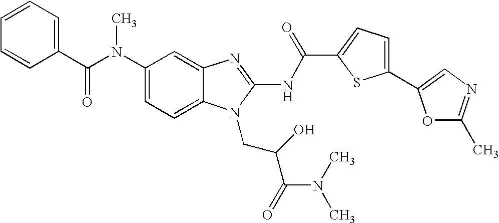 | 5-(2-Methyl-oxazol-5-yl)-thiophene-2- carboxylic acid [5-(benzoyl-methyl-amino)- 1-(2-dimethylcarbamoyl-2-hydroxy-ethyl)- 1H-benzoimidazol-2-yl]-amide |
| 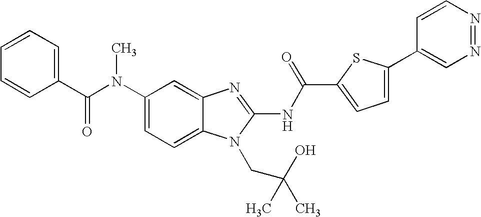 | 5-Pyridazin-4-yl-thiophene-2-carboxylic acid [5-(benzoyl-methyl-amino)-1-(2- hydroxy-2-methyl-propyl)-1H- benzoimidazol-2-yl]-amide |
| 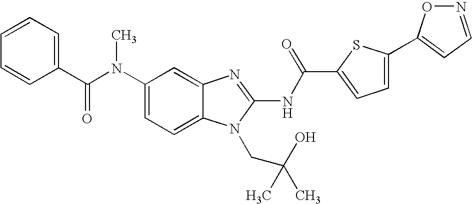 | 5-Isoxazol-5-yl-thiophene-2-carboxylic acid [5-(benzoyl-methyl-amino)-1-(2-hydroxy-2- methyl-propyl)-1H-benzoimidazol-2-yl]- amide |
| 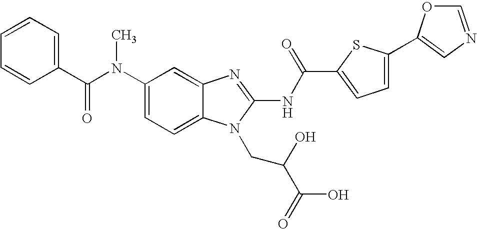 | 3-{5-(Benzoyl-methyl-amino)-2-[(5-oxazol- 5-yl-thiophene-2-carbonyl)-amino]- benzoimidazol-1-yl}-2-hydroxy- propionic acid |
| 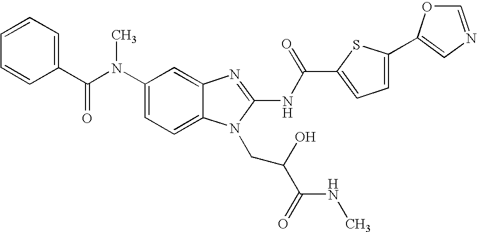 | 5-Oxazol-5-yl-thiophene-2-carboxylic acid [5-(benzoyl-methyl-amino)-1-(2-hydroxy-2- methylcarbamoyl-ethyl)-1H-benzoimidazol- 2-yl]-amide |
| 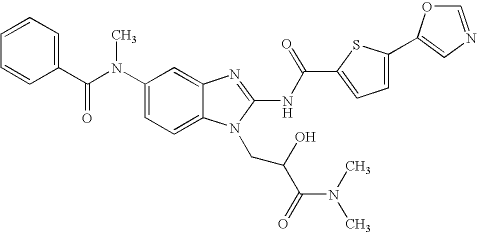 | 5-Oxazol-5-yl-thiophene-2-carboxylic acid [5-(benzoyl-methyl-amino)-1-(2- dimethylcarbamoyl-2-hydroxy-ethyl)-1H- benzoimidazol-2-yl]-amide |
| 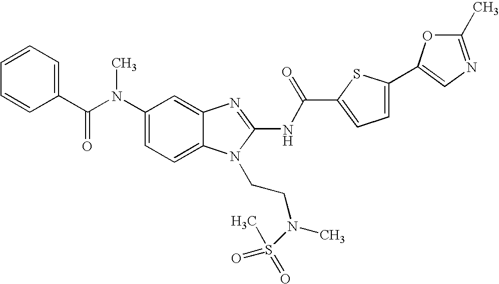 | 5-(2-Methyl-oxazol-5-yl)-thiophene-2- carboxylic acid {5-(benzoyl-methyl-amino)- 1-[2-(methanesulfonyl-methyl-amino)- ethyl]-1H-benzoimidazol-2-yl}-amide |
| 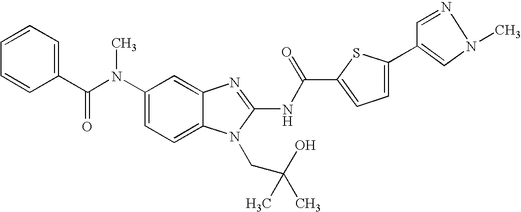 | 5-(Methyl-1H-pyrazol-4-yl)-thiophene-2- carboxylic acid [5-(benzoyl-methyl-amino)- 1-(2-hydroxy-2-methyl-propyl)-1H- benzoimidazol-2-yl]-amide |
| 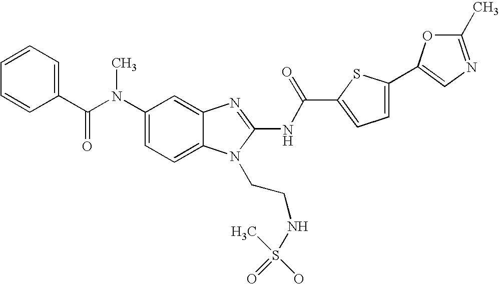 | 5-(2-Methyl-oxazol-5-yl)-thiophene-2- carboxylic acid [5-(benzoyl-methyl-amino)- 1-(2-methanesulfonylamino-ethyl)-1H- benzoimidazol-2-yl]-amide |
| 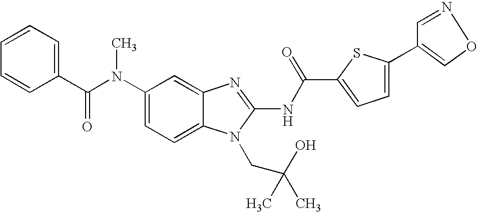 | 5-Isoxazol-4-yl-thiophene-2-carboxylic acid [5-(benzoyl-methyl-amino)-1-(2-hydroxy-2- methyl-propyl)-1H-benzoimidazol-2-yl]- amide |
| 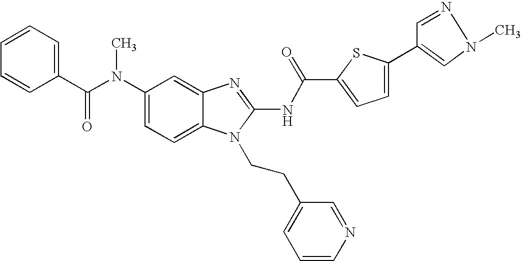 | 5-(1-Methyl-1H-pyrazol-4-yl)-thiophene-2- carboxylic acid [5-(benzoyl-methyl-amino)- 1-(2-pyridin-3-yl-ethyl)-1H-benzoimidazol- 2-yl]-amide |
| 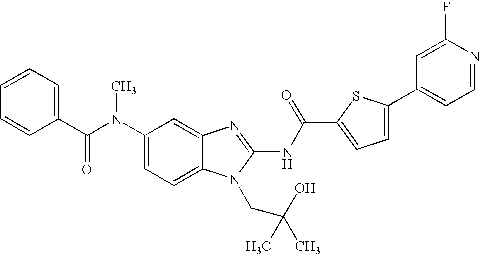 | 2-(2-Fluoro-pyridin-4-yl)-thiazole-5- carboxylic acid [5-(benzoyl-methyl-amino)- 1-(2-hydroxy-2-methyl-propyl)-1H- benzoimidazol-2-yl]-amide |
| 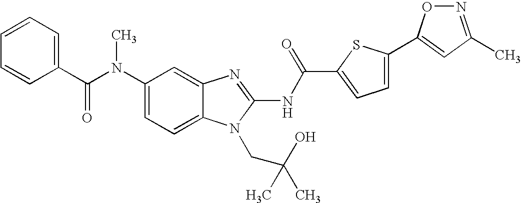 | 5-(3-Methyl-isoxazol-5-yl)-thiophene-2- carboxylic acid [5-(benzoyl-methyl-amino)- 1-(2-hydroxy-2-methyl-propyl)-1H- benzoimidazol-2-yl]-amide |
| 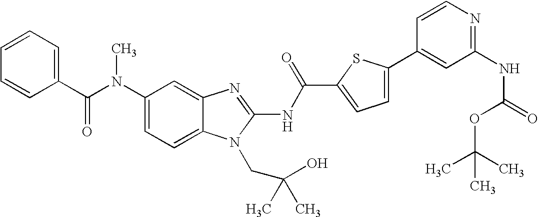 | (4-{5-[5-(Benzoyl-methyl-amino)-1-(2- hydroxy-2-methyl-propyl)-1H- benzoimidazol-2-ylcarbamoyl]-thiophen-2- yl}-pyridin-2-yl)-carbamic acid tert- butyl ester |
| 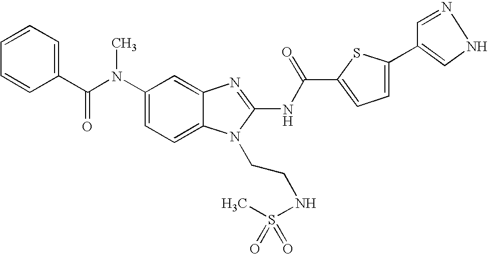 | 5-(1H-Pyrazol-4-yl)-thiophene-2-carboxylic acid [5-(benzoyl-methyl-amino)-1-(2- methanesulfonylamino-ethyl)-1H- benzoimidazol-2-yl]-amide |
| 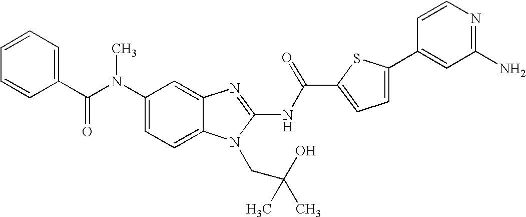 | 5-(2-Amino-pyridin-4-yl)-thiophene-2- carboxylic acid [5-(benzoyl-methyl-amino)- 1-(2-hydroxy-2-methyl-propyl)-1H- benzoimidazol-2-yl]-amide |
| 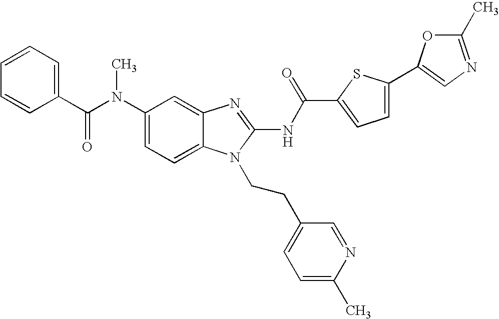 | 5-(2-Methyl-oxazol-5-yl)-thiophene-2- carboxylic acid {5-(benzoyl-methyl-amino)- 1-[2-(6-methyl-pyridin-3-yl)-ethyl]-1H- benzoimidazol-2-yl}-amide |
| 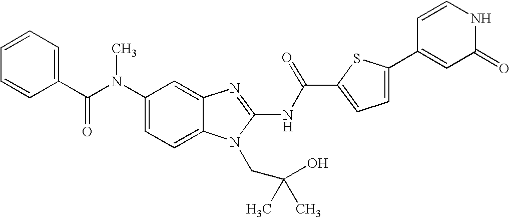 | 5-(2-Oxo-1,2-dihydro-pyridin-4-yl)- thiophene-2-carboxylic acid [5-(benzoyl- methyl-amino)-1-(2-hydroxy-2-methyl- propyl)-1H-benzoimidazol-2-yl]-amide |
| 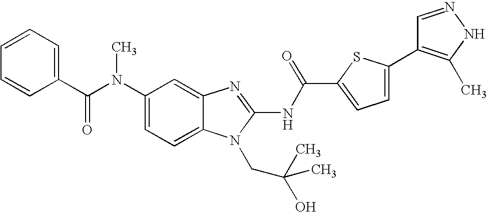 | 5-(5-Methyl-1H-pyrazol-4-yl)-thiophene-2- carboxylic acid [5-(benzoyl-methyl-amino)- 1-(2-hydroxy-2-methyl-propyl)-1H- benzoimidazol-2-yl]-amide |
| 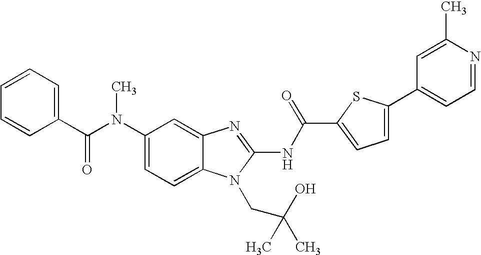 | 5-(2-Methyl-pyridin-4-yl)-thiophene-2- carboxylic acid [5-(benzoyl-methyl-amino)- 1-(2-hydroxy-2-methyl-propyl)-1H- benzoimidazol-2-yl]-amide |
| 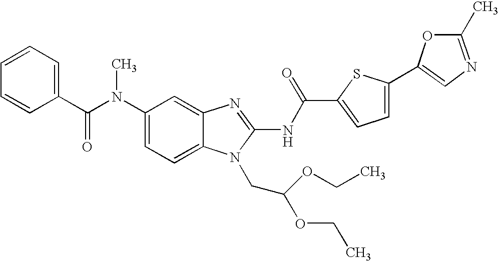 | 5-(2-Methyl-oxazol-5-yl)-thiophene-2- carboxylic acid [5-(benzoyl-methyl-amino)- 1-(2,2-diethoxy-ethyl)-1H-benzoimidazol- 2-yl]-amide |
| 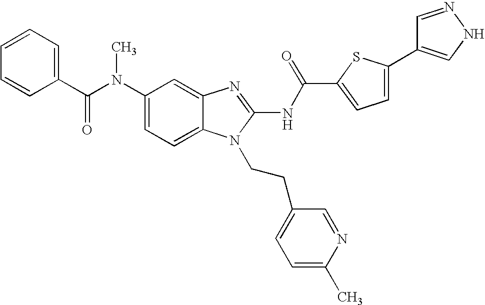 | 5-(1H-Pyrazol-4-yl)-thiophene-2-carboxylic acid {5-(benzoyl-methyl-amino)-1-[2-(6- methyl-pyridin-3-yl)-ethyl]-1H- benzoimidazol-2-yl}-amide |
| 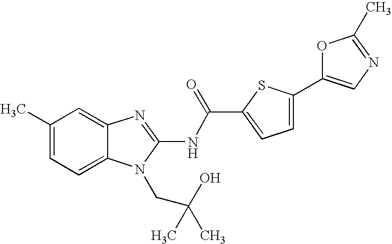 | 5-(2-Methyl-oxazol-5-yl)-thiophene-2- carboxylic acid [1-(2-hydroxy-2-methyl- propyl)-5-methyl-1H-benzoimidazol-2-yl]- amide |
| 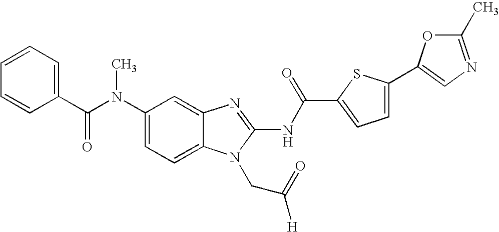 | 5-(2-Methyl-oxazol-5-yl)-thiophene-2- carboxylic acid [5-(benzoyl-methyl-amino)- 1-(2-oxo-ethyl)-1H-benzoimidazo1-2-yl]- amide |
| 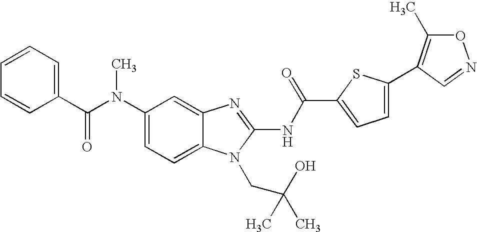 | 5-(5-Methyl-isoxazol-4-yl)-thiophene-2- carboxylic acid [5-(benzoyl-methyl-amino)- 1-(2-hydroxy-2-methyl-propyl)-1H- benzoimidazol-2-yl]-amide |
| 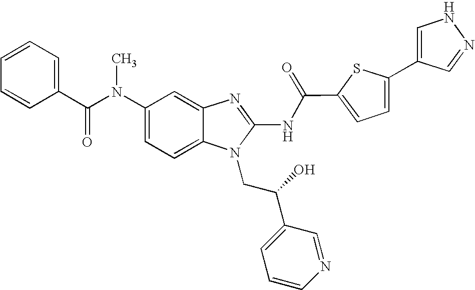 | 5-(1H-Pyrazol-4-yl)-thiophene-2-carboxylic acid [5-benzoyl-methyl-amino)-1-((R)-2- hydroxy-2-pyridin-3-yl-ethyl)-1H- benzoimidazol-2-yl]-amide |
| 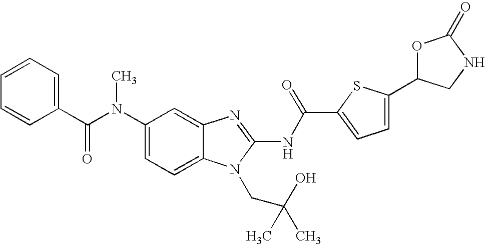 | 5-(2-Oxo-oxazolidin-5-yl)-thiophene-2- carboxylic acid [5-(benzoyl-methyl-amino)- 1-(2-hydroxy-2-methyl-propyl)-1H- benzoimidazol-2-yl]-amide |
| 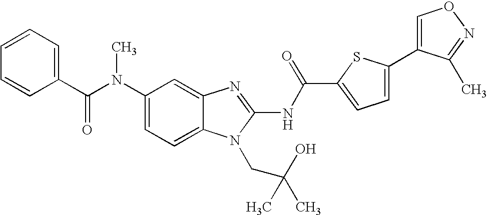 | 5-(3-Methyl-isoxazol-4-yl)-thiophene-2- carboxylic acid [5-(benzoyl-methyl-amino)- 1-(2-hydroxy-2-methyl-propyl)-1H- benzoimidazol-2-yl]-amide |
| 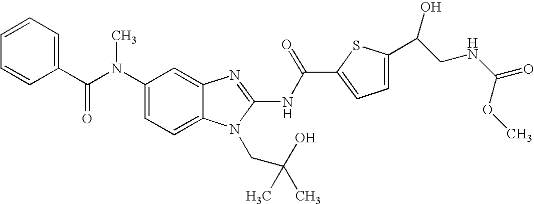 | (2-{5-[5-(Benzoyl-methyl-amino)-1-(2- hydroxy-2-methyl-propyl)-1H- benzoimidazol-2-ylcarbamoyl]-thiophen-2- yl}-2-hydroxy-ethyl)-carbamic acid methyl ester |
| 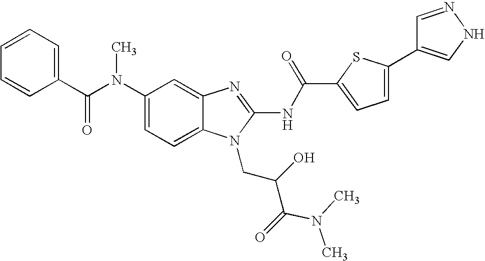 | 5-(1H-Pyrazol-4-yl)-thiophene-2-carboxylic acid [5-(benzoyl-methyl-amino)-1-(2- dimethylcarbamoyl-2-hydroxy-ethyl)-1H- benzoimidazol-2-yl]-amide |
| 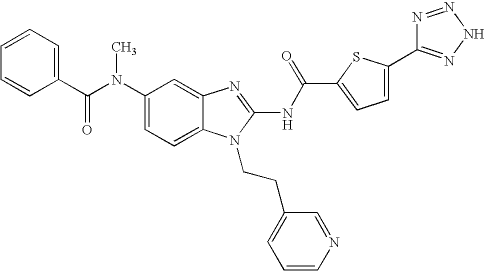 | 5-(2H-Tetrazol-5-yl)-thiophene-2- carboxylic acid [5-(benzoyl-methyl-amino)- 1-(2-pyridin-3-yl-ethyl)-1H-benzoimidazol- 2-yl]-amide |
| 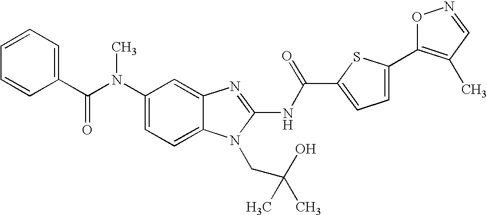 | 5-(4-Methyl-isoxazol-5-yl)-thiophene-2- carboxylic acid [5-(benzoyl-methyl-amino)- 1-(2-hydroxy-2--methyl-propyl)-1H- benzoimidazol-2-yl]-amide |
| 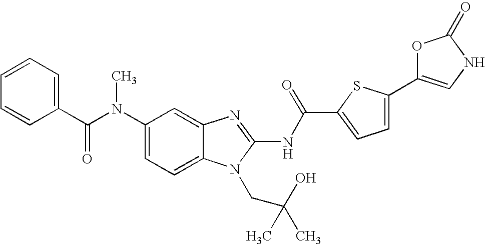 | 5-(2-Oxo-2,3-dihydro-oxazol-5-yl)- thiophene-2-carboxylic acid [5-(benzoyl- methyl-amino)-1-(2-hydroxy-2-methyl- propyl)-1H-benzoimidazol-2-yl]-amide |
| 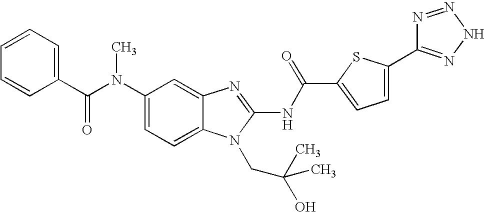 | 5-(2H-Tetrazol-5-yl)-thiophene-2- carboxylic acid [5-(benzoyl-methyl-amino)- 1-(2-hydroxy-2-methyl-propyl)-1H- benzoimidazol-2-yl]-amide |
| 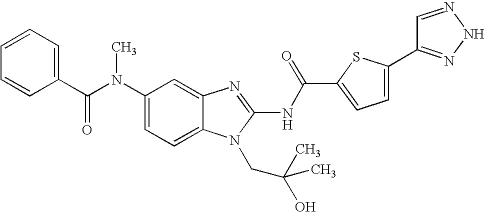 | 5-(2H-[1,2,3]Triazol-4-yl)-thiophene-2- carboxylic acid [5-(benzoyl-methyl-amino)- 1-(2-hydroxy-2-methyl-propyl)-1H- benzoimidazol-2-yl]-amide |
| 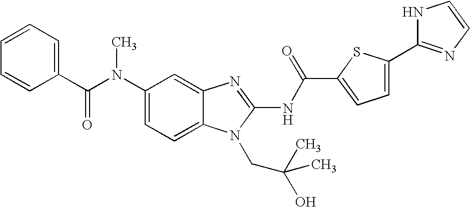 | 5-(1H-Imidazol-2-yl)-thiophene-2- carboxylic acid [5-(benzoyl-methyl-amino)- 1-(2-hydroxy-2-methyl-propyl)-1H- benzoimidazol-2-yl]-amide |
| 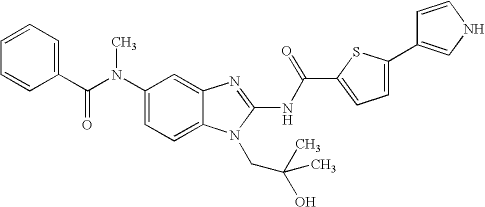 | 5-(1H-Imidazol-4-yl)-thiophene-2- carboxylic acid [5-(benzoyl-methyl-amino)- 1-(2-hydroxy-2-methyl-propyl)-1H- benzoimidazol-2-yl]-amide |
| 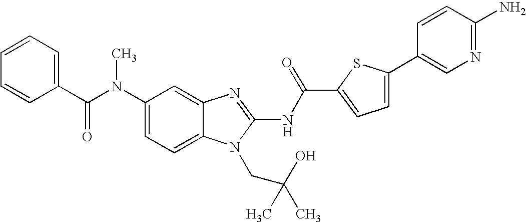 | 5-(6-Amino-pyridin-3-yl)-thiophene-2- carboxylic acid [5-(benzoyl-methyl-amino)- 1-(2-hydroxy-2-methyl-propyl)-1H- benzoimidazol-2-yl]-amide |
| 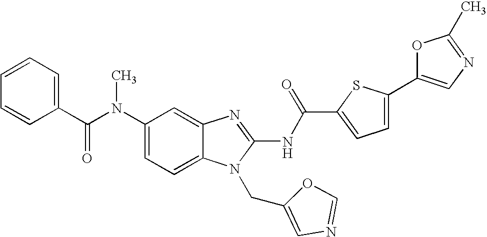 | 5-(2-Methyl-oxazol-5-yl)-thiophene-2- carboxylic acid [5-(benzoyl-methyl-amino)- 1-oxazol-5-ylmethyl-1H-benzoimidazol-2- yl]-amide |
| 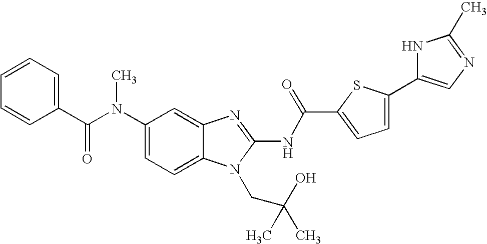 | 5-(2-Methyl-3H-imidazol-4-yl)-thiophene- 2-carboxylic acid [5-(benzoyl-methyl- amino)-1-(2-hydroxy-2-methyl-propyl)-1H- benzoimidazol-2-yl]-amide |
| 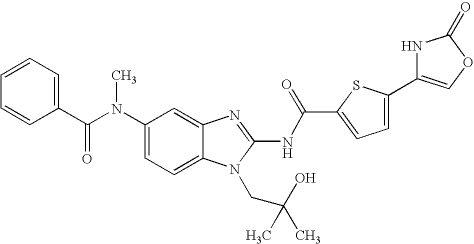 | 5-(2-Oxo-2,3-dihydro-oxazol-4-yl)- thiophene-2-carboxylic acid [5-(benzoyl- methyl-amino)-1-(2-hydroxy-2-methyl- propyl)-1H-benzoimidazol-2-yl]-amide |
| 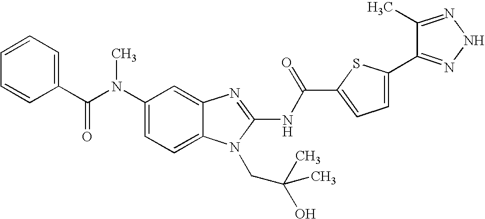 | 5-(5-Methyl-2H-[1,2,3]triazol-4-yl)- thiophene-2-carboxylic acid [5-(benzoyl- methyl-amino)-1-(2-hydroxy-2-methyl- propyl)-1H-benzoimidazol-2-yl]-amide |
| 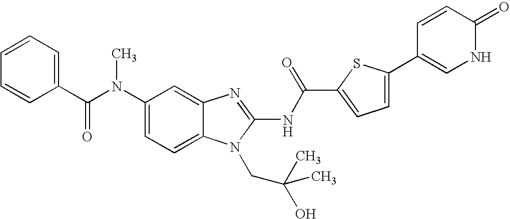 | 5-(6-Oxo-1,6-dihydro-pyridin-3-yl)- thiophene-2-carboxylic acid [5-(benzoyl- methyl-amino)-1-(2-hydroxy-2-methyl- propyl)-1H-benzoimidazol-2-yl]-amide |
| 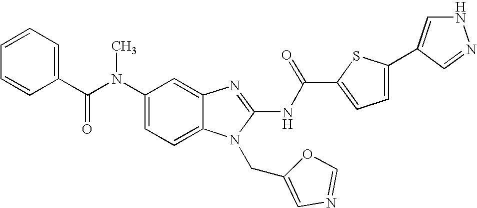 | 5-(1H-Pyrazol-4-yl)-thiophene-2-carboxylic acid [5-(benzoyl-methyl-amino)-1-oxazol- 5-ylmethyl-1H-benzoimidazol-2-yl]-amide |
| 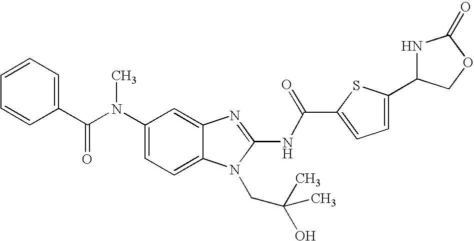 | 5-(2-Oxo-oxazolidin-4-yl)-thiophene-2- carboxylic acid [5-(benzoyl-methyl-amino)- 1-(2-hydroxy-2-methyl-propyl)-1H- benzoimidazol-2-yl]-amide |
| 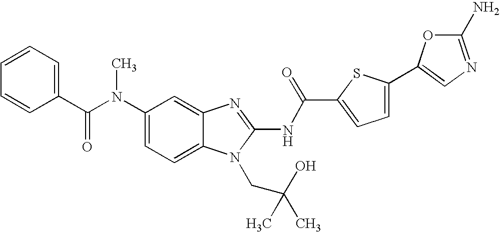 | 5-(2-Amino-oxazol-5-yl)-thiophene-2- carboxylic acid [5-(benzoyl-methyl-amino)- 1-(2-hydroxy-2-methyl-propyl)-1H- benzoimidazol-2-yl]-amide |
| 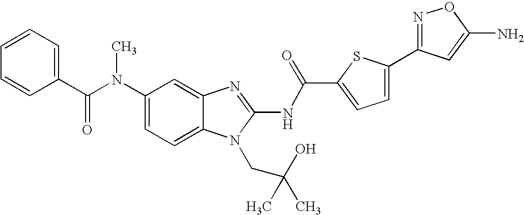 | 5-(5-Amino-isoxazol-3-yl)-thiophene-2- carboxylic acid [5-(benzoyl-methyl-amino)- 1-(2-hydroxy-2-methyl-propyl)-1H- benzoimidazol-2-yl]-amide |
| 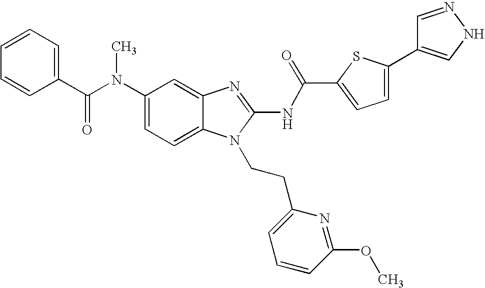 | 5-(1H-Pyrazol-4-yl)-thiophene-2-carboxylic acid {5-(benzoyl-methyl-amino)-1-[2-(6- methoxy-pyridin-2-yl)-ethyl]-1H- benzoimidazol-2-yl}-amide |
| 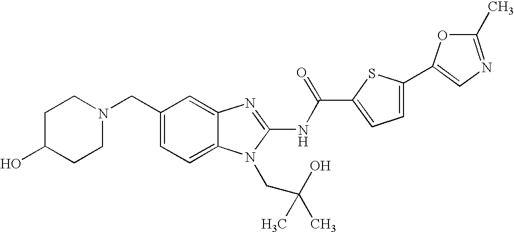 | 5-(2-Methyl-oxazol-5-yl)-thiophene-2- carboxylic acid [1-(2-hydroxy-2-methyl- propyl)-5-(4-hydroxy-piperidin-1- ylmethyl)-1H-benzoimidazol-2-yl]-amide |
| 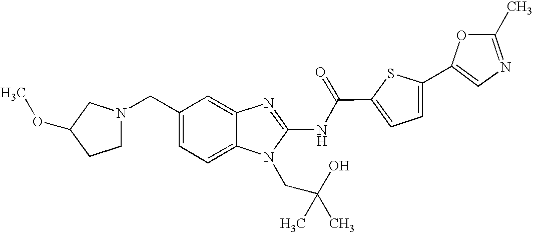 | 5-(2-Methyl-oxazol-5-yl)-thiophene-2- carboxylic acid [1-(2-hydroxy-2-methyl- propyl)-5-(3-methoxy-pyrrolidin-1- ylmethyl)-1H-benzoimidazol-2-yl]-amide |
| 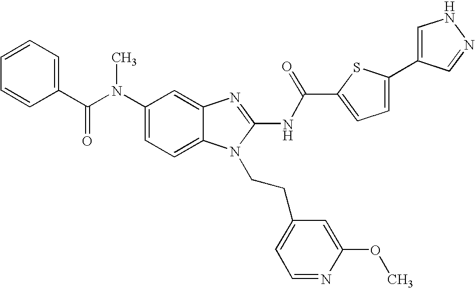 | 5-(1H-Pyrazol-4-yl)-thiophene-2-carboxylic acid {5-(benzoyl-methyl-amino)-1-[2-(2- methoxy-pyridin-4-yl)-ethyl]-1H- benzoimidazol-2-yl}-amide |
| 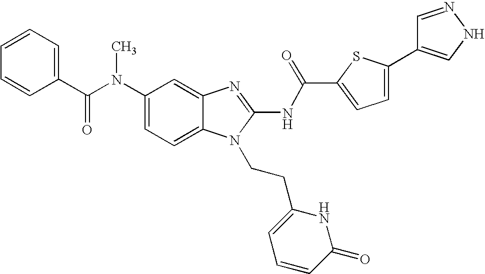 | 5-(1H-Pyrazol-4-yl)-thiophene-2-carboxylic acid {5-(benzoyl-methyl-amino)-1-[2-(6- oxo-1,6-dihydro-pyridin-2-yl)-ethyl]1H- benzoimidazol-2-yl}-amide |
| 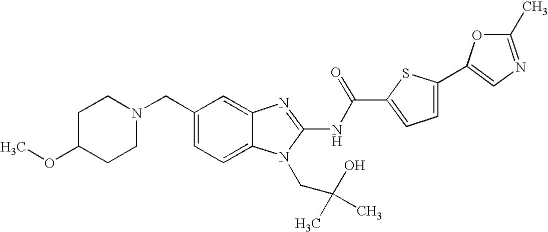 | 5-(2-Methyl-oxazol-5-yl)-thiophene-2- carboxylic acid [1-(2-hydroxy-2-methyl- propyl)-5-(4-methoxy-piperidin-1- ylmethyl)-1H-benzoimidazol-2-yl]-amide |
| 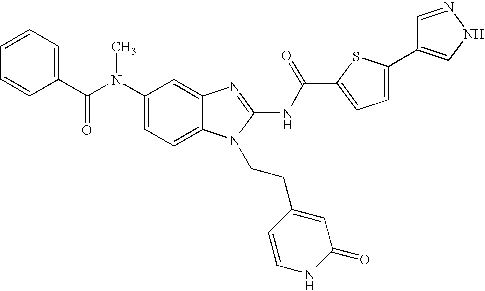 | 5-(1H-Pyrazol-4-yl)-thiophene-2-carboxylic acid {5-(benzoyl-methyl-amino)-1-[2-(2- oxo-1,2-dihydro-pyridin-4-yl)-ethyl]-1H- benzoimidazol-2-yl}-amide |
| 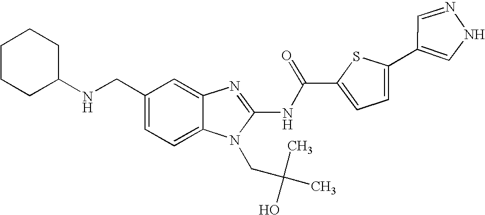 | 5-(1H-Pyrazol-4-yl)-thiophene-2-carboxylic acid [5-cyclohexylaminomethyl-1-(2- hydroxy-2-methyl-propyl)-1H- benzoimidazol-2-yl]-amide |
| 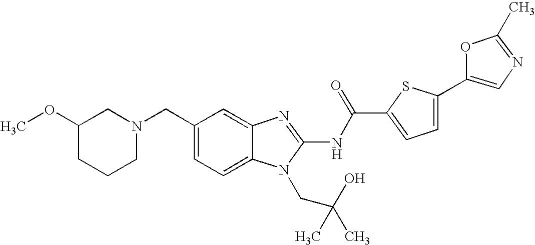 | 5-(2-Methyl-oxazol-5-yl)-thiophene-2- carboxylic acid [1-(2-hydroxy-2-methyl- propyl)-5-(3-methoxy-piperidin-1- ylmethyl)-1H-benzoimidazol-2-yl]-amide |
| 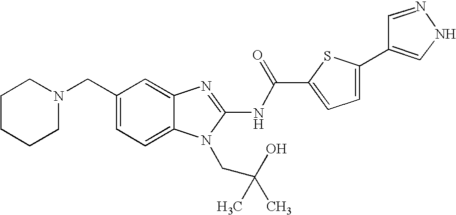 | 5-(1H-Pyrazol-4-yl)-thiophene-2-carboxylic acid [1-(2-hydroxy-2-methyl-propyl)-5- piperidin-1-ylmethyl-1H-benzoimidazol-2- yl]-amide |
| 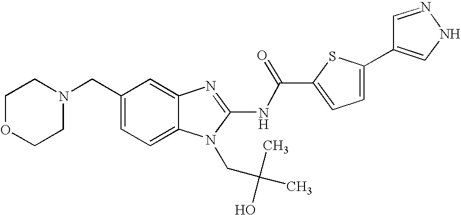 | 5-(1H-Pyrazol-4-yl)-thiophene-2-carboxylic acid [1-(2-hydroxy-2-methyl-propyl)-5- morpholin-4-ylmethyl-1H-benzoimidazol-2- yl]-amide |
| 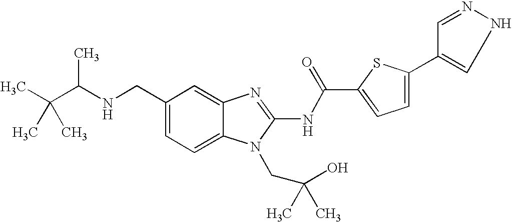 | 5-(1H-Pyrazol-4-yl)-thiophene-2-carboxylic acid (1-(2-hydroxy-2-methyl-propyl)-5- [(1,2,2-trimethyl-propylamino)-methyl]-1H- benzoimidazol-2-yl}-amide |
| 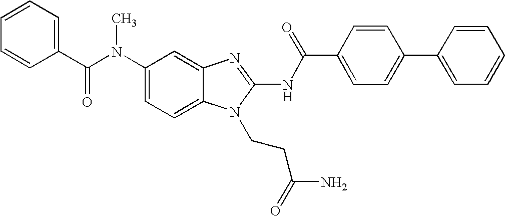 | Biphenyl-4-carboxylic acid [5-(benzoyl- methyl-amino)-1-(2-carbamoyl-ethyl)-1H- benzoimidazol-2-yl]-amide |
| 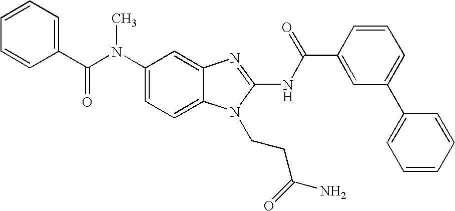 | Biphenyl-3-carboxylic acid [5-(benzoyl- methyl-amino)-1-(2-carbamoyl-ethyl)-1H- benzoimidazol-2-yl]-amide |
| 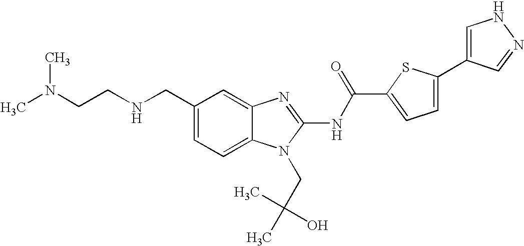 | 5-(1H-Pyrazol-4-yl)-thiophene-2-carboxylic acid [5-[(2-dimethylamino-ethylamino)- methyl]-1-(2-hydroxy-2-methyl-propyl)- 1H-benzoimidazol-2-yl]-amide |
| 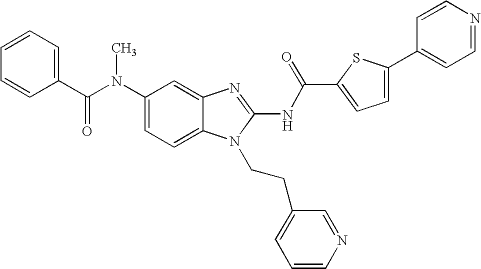 | 5-Pyridin-4-yl-thiophene-2-carboxylic acid [5-(benzoyl-methyl-amino)-1-(2-pyridin-3- yl-ethyl)-1H-benzoimidazol-2-yl]-amide |
| 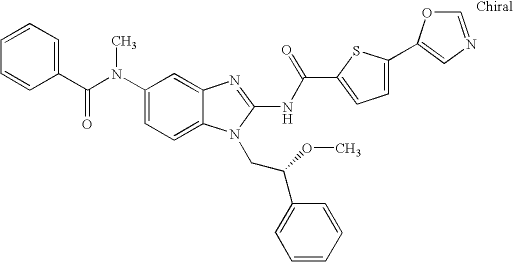 | 5-Oxazol-5-yl-thiophene-2-carboxylic acid [5-(benzoyl-methyl-amino)-1-((R)-2- methoxy-2-phenyl-ethyl)-1H- benzoimidazol-2-yl]-amide |
| 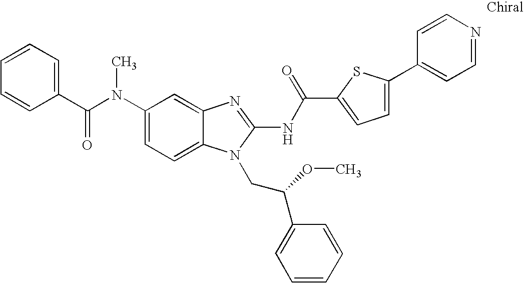 | 5-Pyridin-4-yl-thiophene-2-carboxylic acid [5-(benzoyl-methyl-amino)-1-((R)-2- methoxy-2-phenyl-ethyl)-1H- benzoimidazol-2-yl]-amide |
| 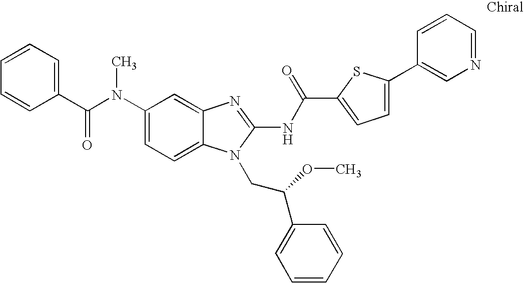 | 5-Pyridin-3-yl-thiophene-2-carboxylic acid [5-(benzoyl-methyl-amino)-1-((R)-2- methoxy-2-phenyl-ethyl)-1H- benzoimidazol-2-yl]-amide |
| 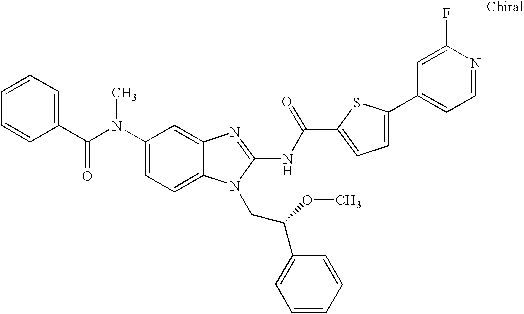 | 5-(2-Fluoro-pyridin-4-yl)-thiophene-2- carboxylic acid [5-(benzoyl-methyl-amino)- 1-((R)-2-methoxy-2-phenyl-ethyl)-1H- benzoimidazol-2-yl]-amide |
| 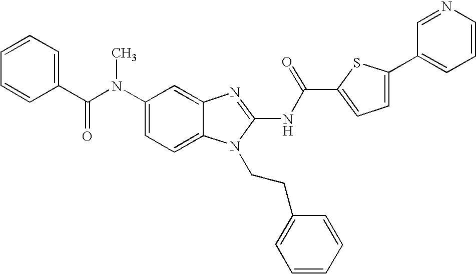 | 5-Pyridin-3-yl-thiophene-2-carboxylic acid [5-(benzoyl-methyl-amino)-1-phenethyl- 1H-benzoimidazol-2-yl]-amide |
| 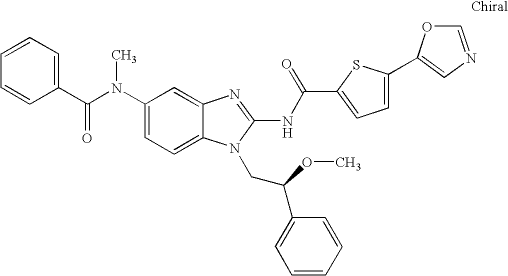 | 5-Oxazol-5-yl-thiophene-2-carboxylic acid [5-(benzoyl-methyl-amino)-1-((S)-2- methoxy-2-phenyl-ethyl)-1H- benzoimidazol-2-yl]-amide |
| 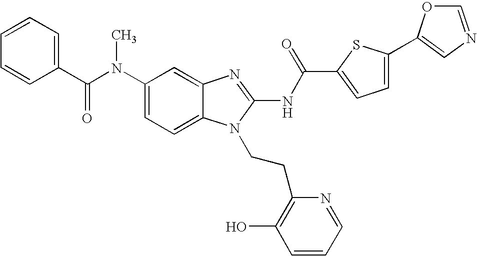 | 5-Oxazol-5-yl-thiophene-2-carboxylic acid {5-(benzoyl-methyl-amino)-1-[2-(3- hydroxy-pyridin-2-yl)-ethyl]-1H- benzoimidazol-2-yl}-amide |
| 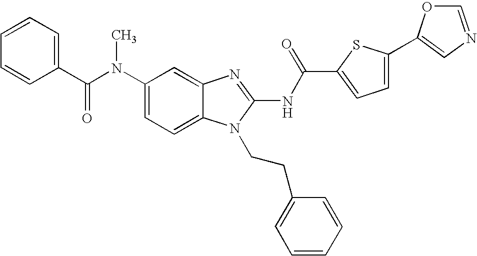 | 5-Oxazol-5-yl-thiophene-2-carboxylic acid [5-(benzoyl-methyl-amino)-1-phenethyl- 1H-benzoimidazol-2-yl]-amide |
| 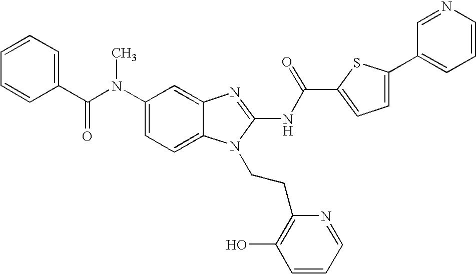 | 5-Pyridin-3-yl-thiophene-2-carboxylic acid {5-(benzoyl-methyl-amino)-1-[2-(3- hydroxy-pyridin-2-yl)-ethyl]-1H- benzoimidazol-2-yl}-amide |
| 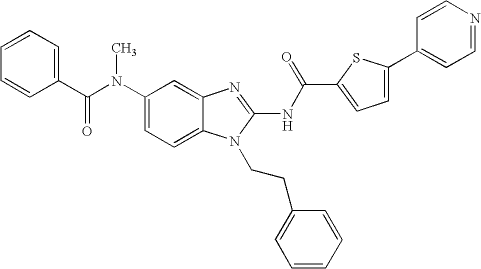 | 5-Pyridin-4-yl-thiophene-2-carboxylio acid [5-(benzoyl-methyl-amino)-1-phenethyl- 1H-benzoimidazol-2-yl]-amide |
| 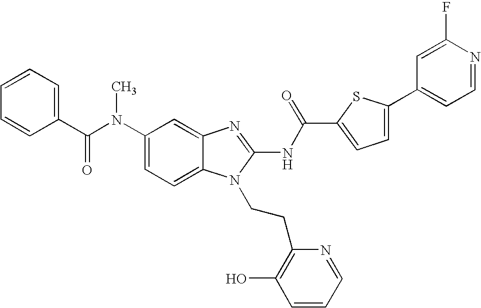 | 5-(2-Fluoro-pyridin-4-yl)-thiophene-2- carboxylic acid {5-(benzoyl-methyl-amino)- 1-[2-(3-hydroxy-pyridin-2-yl)-ethyl]-1H- benzoimidazol-2-yl}-amide |
| 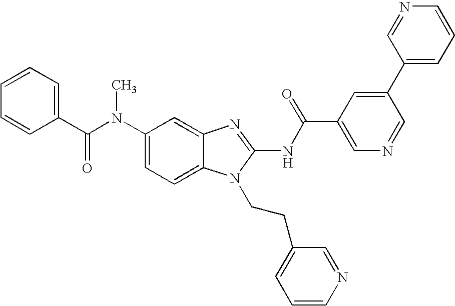 | [3,3′]Bipyridinyl-5-carboxylic acid [5- (benzoyl-methyl-amino)-1-(2-pyridin-3-yl- ethyl)-1H-benzoimidazol-2-yl]-amide |
| 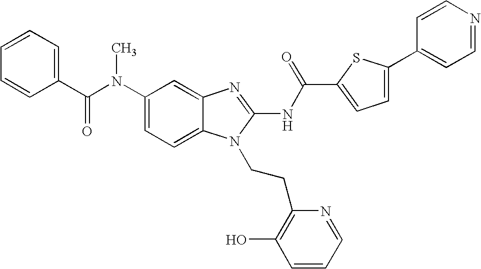 | 5-Pyridin-4-yl-thiophene-2-carboxylic acid {5-(benzoyl-methyl-amino)-1-[2-(3- hydroxy-pyridin-2-yl)-ethyl]-1H- benzoimidazol-2-yl}-amide |
| 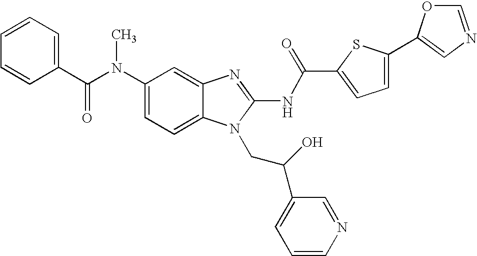 | 5-Oxazol-5-yl-thiophene-2-carboxylic acid [5-(benzoyl-methyl-amino)-1-(2-hydroxy-2- pyridin-3-yl-ethyl)-1H-benzoimidazol-2- yl]-amide |
| 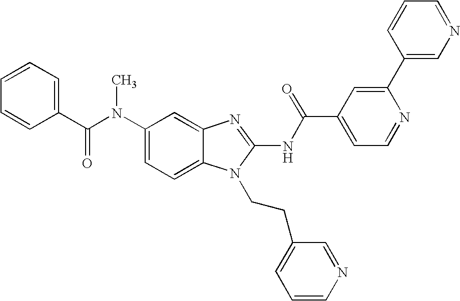 | [2,3′]Bipyridinyl-4-carboxylic acid [5- (benzoyl-methyl-amino)-1-(2-pyridin-3-yl- ethyl)-1H-benzoimidazol-2-yl]-amide |
| 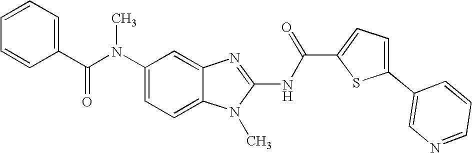 | 5-Pyridin-3-yl-thiophene-2-carboxylic acid [5-(benzoyl-methyl-amino)-1-methyl-1H- benzoimidazol-2-yl]-amide |
| 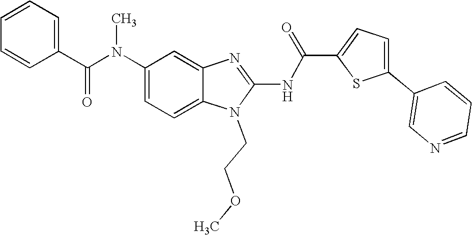 | 5-Pyridin-3-yl-thiophene-2-carboxylic acid [5-(benzoyl-methyl-amino)-1-(2-methoxy- ethyl)-1H-benzoimidazol-2-yl]-amide |
| 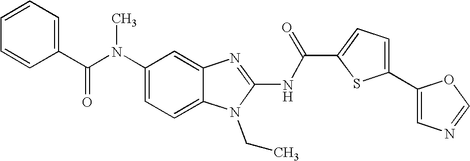 | 5-Oxazol-5-yl-thiophene-2-carboxylic acid [5-(benzoyl-methyl-amino)-1-ethyl-1H- benzoimidazol-2-yl]-amide |
| 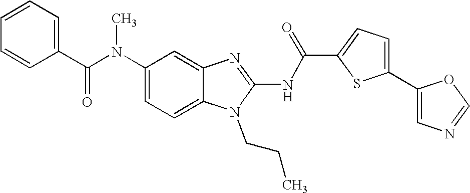 | 5-Oxazol-5-yl-thiophene-2-carboxylic acid [5-(benzoyl-methyl-amino)-1-propyl-1H- benzoimidazol-2-yl]-amide |
| 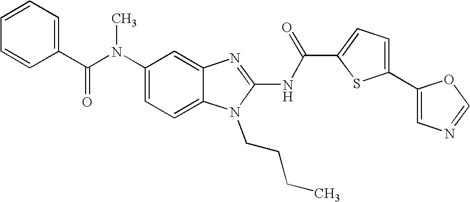 | 5-Oxazol-5-yl-thiophene-2-carboxylic acid [5-(benzoyl-methyl-amino)-1-butyl-1H- benzoimidazol-2-yl]-amide |
| 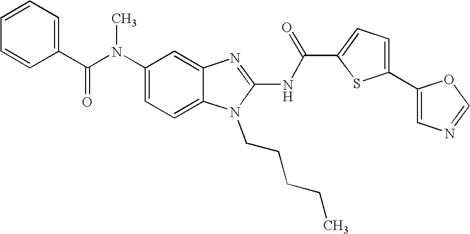 | 5-Oxazol-5-yl-thiophene-2-carboxylic acid [5-(benzoyl-methyl-amino)-1-pentyl-1H- benzoimidazol-2-yl]-amide |
| 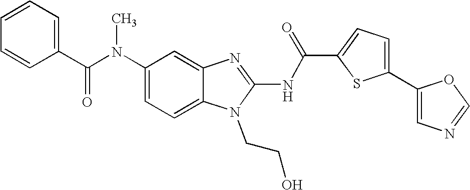 | 5-Oxazol-5-yl-thiophene-2-carboxylic acid [5-(benzoyl-methyl-amino)-1-(2-hydroxy- ethyl)-1H-benzoimidazol-2-yl]-amide |
| 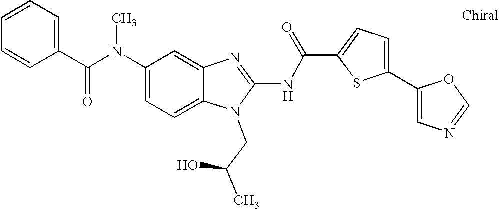 | 5-Oxazol-5-yl-thiophene-2-carboxylic acid [5-(benzoyl-methyl-amino)-1-((R)-2- hydroxy-propyl)-1H-benzoimidazol-2- yl]-amide |
| 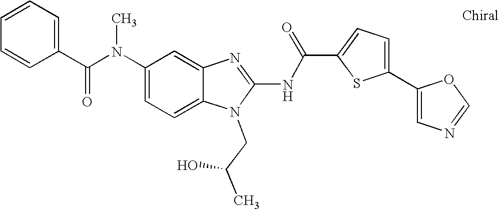 | 5-Oxazol-5-yl-thiophene-2-carboxylic acid [5-(benzoyl-methyl-amino)-1-((S)-2- hydroxy-propyl)-1H-benzoimidazol-2- yl]-amide |
| 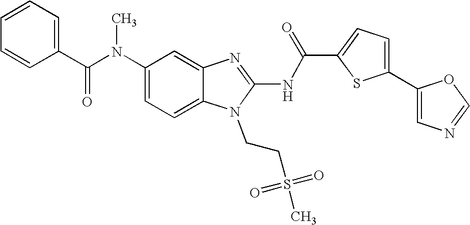 | 5-Oxazol-5-yl-thiophene-2-carboxylic acid [5-(benzoyl-methyl-amino)-1-(2- methanesulfonyl-ethyl)-1H- benzoimidazol-2-yl]-amide |
| 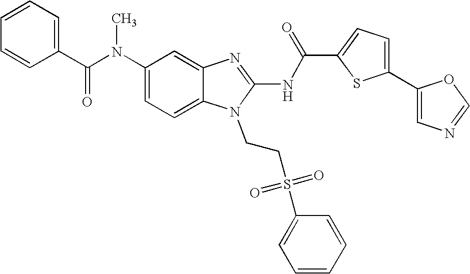 | 5-Oxazol-5-yl-thiophene-2-carboxylic acid [1-(2-benzenesulfonyl-ethyl)-5-(benzoyl- methyl-amino)-1H-benzoimidazol-2-yl]- amide |
| 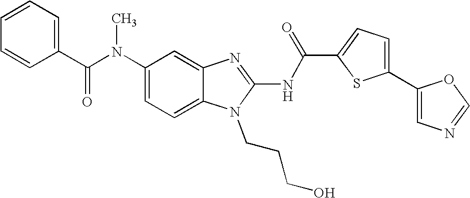 | 5-Oxazol-5-yl-thiophene-2-carboxylic acid [5-(benzoyl-methyl-amino)-1-(3-hydroxy- propyl)-1H-benzoimidazol-2-yl]-amide |
| 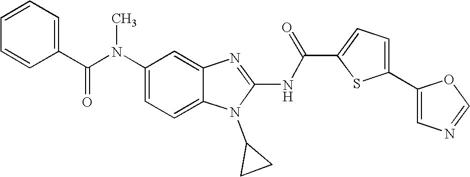 | 5-Oxazol-5-yl-thiophene-2-carboxylic acid [5-(benzoyl-methyl-amino)-1-cyclopropyl- 1H-benzoimidazol-2-yl]-amide |
| 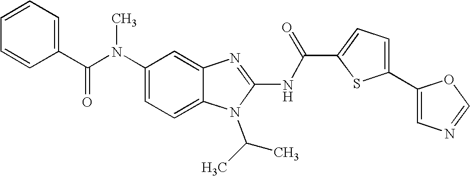 | 5-Oxazol-5-yl-thiophene-2-carboxylic acid [5-(benzoyl-methyl-amino)-1-isopropyl-1H- benzoimidazol-2-yl]-amide |
| 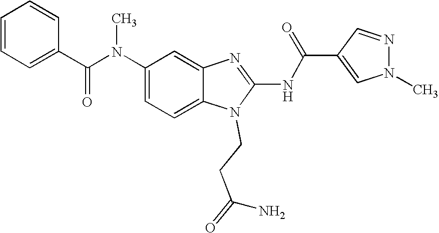 | 1-Methyl-1H-pyrazole-4-carboxylic acid [5- (benzoyl-methyl-amino)-1-(2-carbamoyl- ethyl)-1H-benzoimidazol-2-yl]-amide |
| 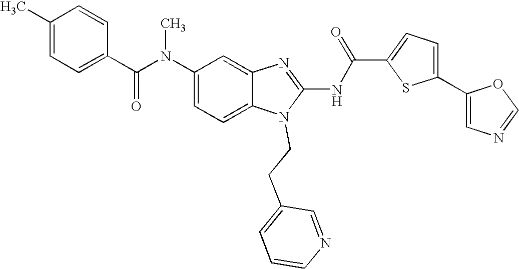 | 5-Oxazol-5-yl-thiophene-2-carboxylic acid [5-[methyl-(4-methyl-benzoyl)-amino]-1- (2-pyridin-3-yl-ethyl)-1H-benzoimidazol- 2-yl]-amide |
| 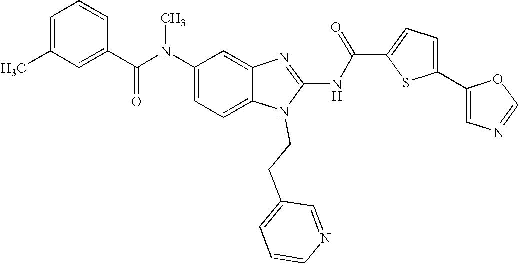 | 5-Oxazol-5-yl-thiophene-2-carboxylic acid [5-[methyl-(3-methyl-benzoyl)-amino]-1- (2-pyridin-3-yl-ethyl)-1H-benzoimidazol- 2-yl]-amide |
| 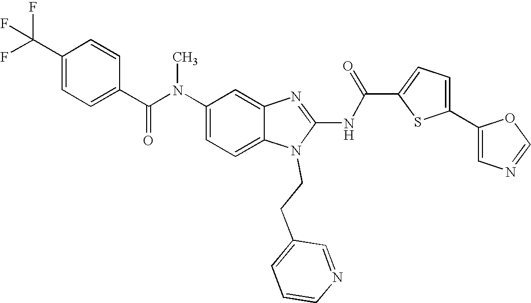 | 5-Oxazol-5-yl-thiophene-2-carboxylic acid [5-[methyl-(4-trifluoromethyl-benzoyl)- amino]-1-(2-pyridin-3-yl-ethyl)-1H- benzoimidazol-2-yl]-amide |
| 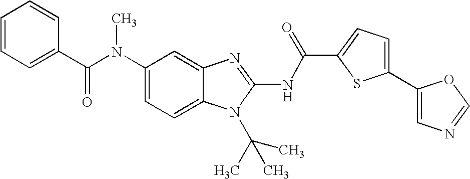 | 5-Oxazol-5-yl-thiophene-2-carboxylic acid [5-(benzoyl-methyl-amino)-1-tert-butyl-1H- benzoimidazol-2-yl]-amide |
| 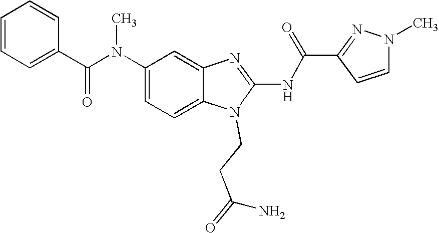 | 1-Methyl-1H-pyrazole-3-carboxylic acid [5- (benzoyl-methyl-amino)-1-(2-carbamoyl- ethyl)-1H-benzoimidazol-2-yl]-amide |
| 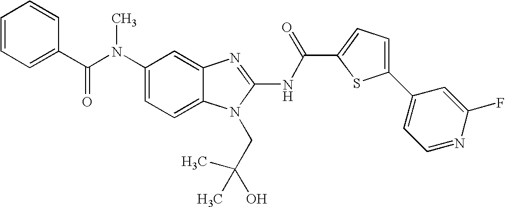 | 5-(2-Fluoro-pyridin-4-yl)-thiophene-2- carboxylic acid [5-(benzoyl-methyl-amino)- 1-(2-hydroxy-2-methyl-propyl)-1H- benzoimidazol-2-yl]-amide |
| 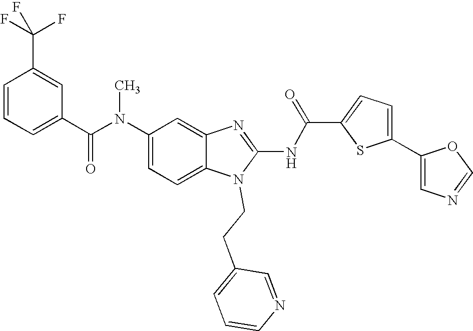 | 5-Oxazol-5-yl-thiophene-2-carboxylic acid [5-[methyl-(3-trifluoromethyl-benzoyl)- amino]-1-(2-pyridin-3-yl-ethyl)-1H- benzoimidazol-2-yl]-amide |
| 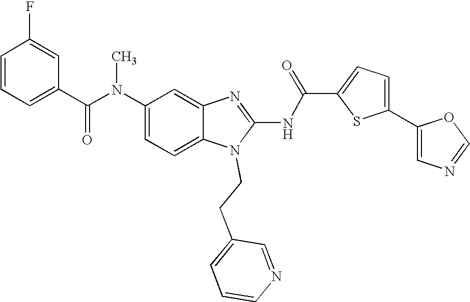 | 5-Oxazol-5-yl-thiophene-2-carboxylic acid [5-[(3-fluoro-benzoyl)-methyl-amino]-1-(2- pyridin-3-yl-ethyl)-1H-benzoimidazol-2- yl]-amide |
| 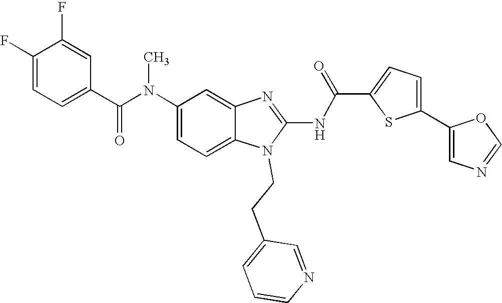 | 5-Oxazol-5-yl-thiophene-2-carboxylic acid [5-[(3,4-difluoro-benzoyl)-methyl-amino]- 1-(2-pyridin-3-yl-ethyl)-1H- benzoimidazol-2-yl]-amide |
| 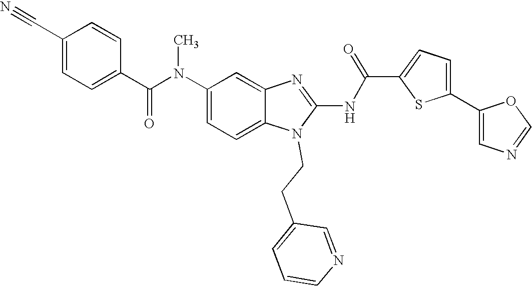 | 5-Oxazol-5-yl-thiophene-2-carboxylic acid [5-[(4-cyano-benzoyl)-methyl-amino]-1-(2- pyridin-3-yl-ethyl)-1H-benzoimidazol-2- yl]-amide |
| 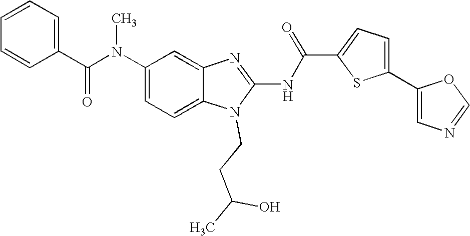 | 5-Oxazol-5-yl-thiophene-2-carboxylic acid [5-(benzoyl-methyl-amino)-1-(3-hydroxy- butyl)-1H-benzoimidazol-2-yl]-amide |
| 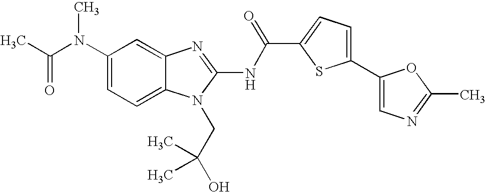 | 5-(2-Methyl-oxazol-5-yl)-thiophene-2- carboxylic acid [5-(acetyl-methyl-amino)-1- (2-hydroxy-2-methyl-propyl)-1H- benzoimidazol-2-yl]-amide |
| 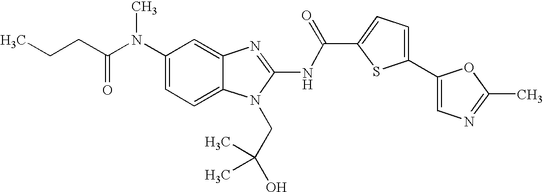 | 5-(2-Methyl-oxazol-5-yl)-thiophene-2- carboxylic acid [5-(butyryl-methyl-amino)- 1-(2-hydroxy-2-methyl-propyl)-1H- benzoimidazol-2-yl]-amide |
| 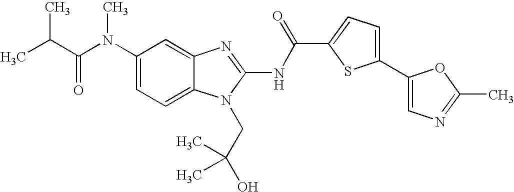 | 5-(2-Methyl-oxazol-5-yl)-thiophene-2- carboxylic acid [1-(2-hydroxy-2-methyl- propyl)-5-(isobutyryl-methyl-amino)-1H- benzoimidazol-2-yl]-amide |
| 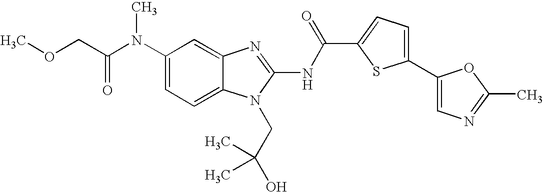 | 5-(2-Methyl-oxazol-5-yl)-thiophene-2- carboxylic acid {1-(2-hydroxy-2-methyl- propyl)-5-[(2-methoxy-acetyl)-methyl- amino]-1H-benzoimidazol-2-yl}-amide |
| 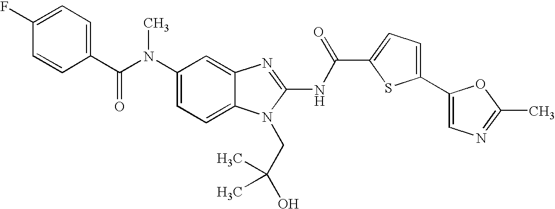 | 5-(2-Methyl-oxazol-5-yl)-thiophene-2- carboxylic acid [5-[(4-fluoro-benzoyl)- methyl-amino]-1-(2-hydroxy-2-methyl- propyl)-1H-benzoimidazol-2-yl]-amide |
| 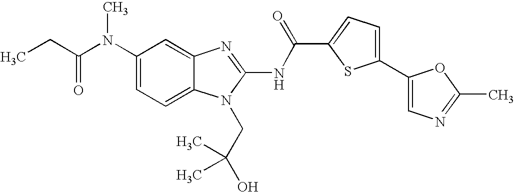 | 5-(2-Methyl-oxazol-5-yl)-thiophene-2- carboxylic acid [1-(2-hydroxy-2-methyl- propyl)-5-(methyl-propionyl-amino)-1H- benzoimidazol-2-yl]-amide |
| 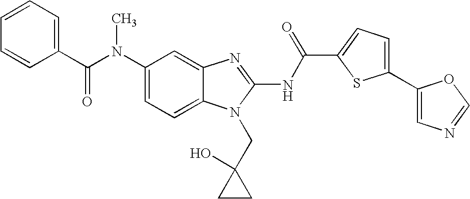 | 5-Oxazol-5-yl-thiophene-2-carboxylic acid [5-(benzoyl-methyl-amino)-1-(1-hydroxy- cyclopropylmethyl)-1H-benzoimidazol-2- yl]-amide |
| 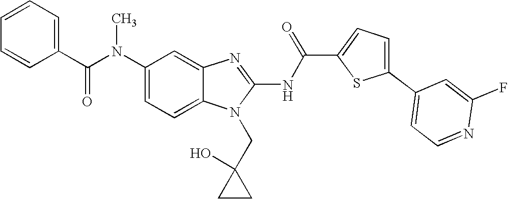 | 5-(2-Fluoro-pyridin-4-yl)-thiophene-2- carboxylic acid [5-(benzoyl-methyl-amino)- 1-(1-hydroxy-cyclopropylmethyl)-1H- benzoimidazol-2-yl]-amide |
| 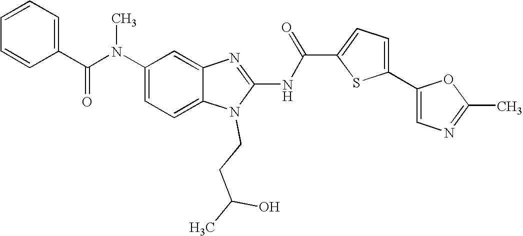 | 5-(2-Methyl-oxazol-5-yl)-thiophene-2- carboxylic acid [5-(benzoyl-methyl-amino)- 1-(3-hydroxy-butyl)-1H-benzoimidazol-2- yl]-amide |
| 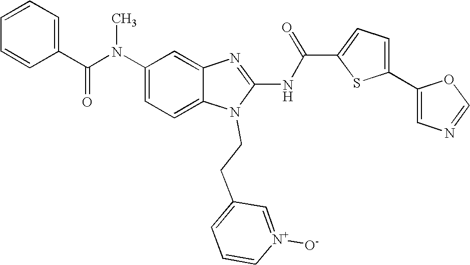 | 5-Oxazol-5-yl-thiophene-2-carboxylic acid {5-(benzoyl-methyl-amino)-1-[2-(1-oxy- pyridin-3-yl)-ethyl]-1H-benzoimidazol-2- yl}-amide |
| 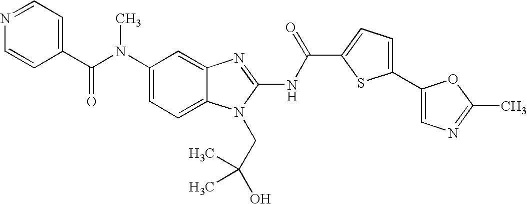 | N-(1-(2-Hydroxy-2-methyl-propyl)-2-{[5- (2-methyl-oxazol-5-yl)-thiophene-2- carbonyl]-amino}-1H-benzoimidazol-5-yl)- N-methyl-isonicotinamide |
| 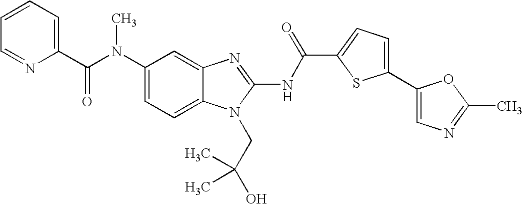 | Pyridine-2-carboxylic acid (1-(2-hydroxy-2- methyl-propyl)-2-{[5-(2-methyl-oxazol-5- yl)-thiophene-2-carbonyl]-amino}-1H- benzoimidazol-5-yl)-methyl-amide |
| 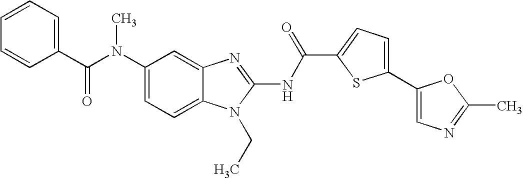 | 5-(2-Methyl-oxazol-5-yl)-thiophene-2- carboxylic acid [5-(benzoyl-methyl-amino)- 1-ethyl-1H-benzoimidazol-2-yl]-amide |
| 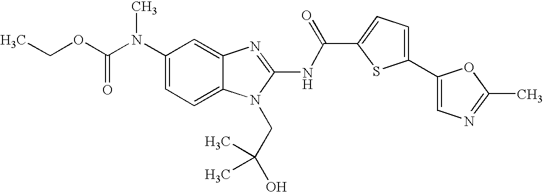 | (1-(2-Hydroxy-2-methyl-propyl)-2-{[5-(2- methyl-oxazol-5-yl)-thiophene-2-carbonyl]- amino}-1H-benzoimidazol-5-yl)-methyl- carbamic acid ethyl ester |
| 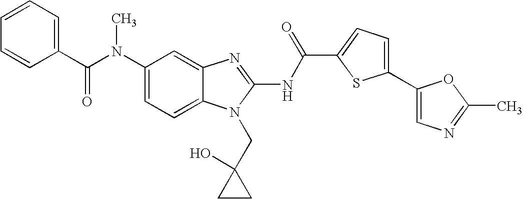 | 5-(2-Methyl-oxazol-5-yl)-thiophene-2- carboxylic acid [5-(benzoyl-methyl-amino)- 1-(1-hydroxy-cyclopropylmethyl)-1H- benzoimidazol-2-yl]-amide |
| 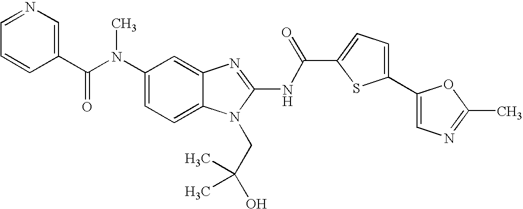 | N-(1-(2-Hydroxy-2-methyl-propyl)-2-{[5- (2-methyl-oxazol-5-yl)-thiophene-2- carbonyl]-amino}-1H-benzoimidazol- 5-yl)-N-methyl-nicotinamide |
| 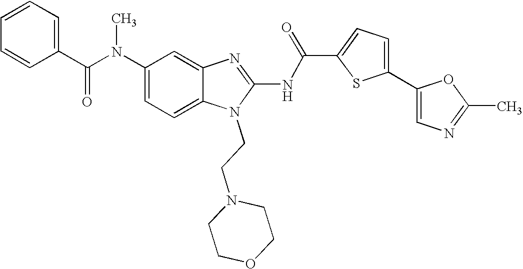 | 5-(2-Methyl-oxazol-5-yl)-thiophene-2- carboxylic acid [5-(benzoyl-methyl-amino)- 1-(2-morpholin-4-yl-ethyl)-1H- benzoimidazol-2-yl]-amide |
| 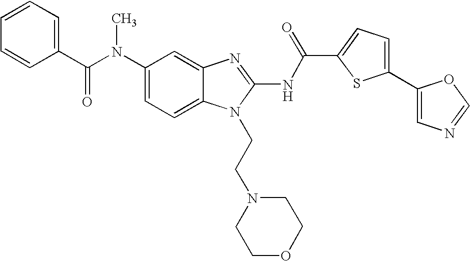 | 5-Oxazol-5-yl-thiophene-2-carboxylic acid [5-(benzoyl-methyl-amino)-1-(2-morpholin- 4-yl-ethyl)-1H-benzoimidazol-2-yl]-amide |
| 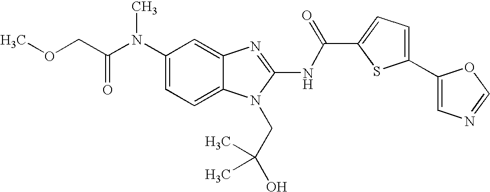 | 5-Oxazol-5-yl-thiophene-2-carboxylic acid {1-(2-hydroxy-2-methyl-propyl)-5-[(2- methoxy-acetyl)-methyl-amino]-1H- benzoimidazol-2-yl}-amide |
| 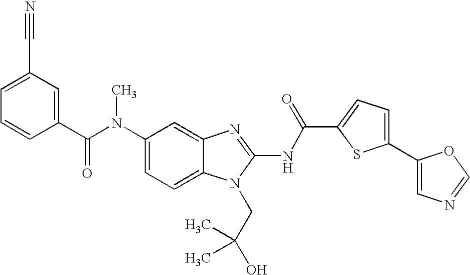 | 5-Oxazol-5-yl-thiophene-2-carboxylic acid [5-[(3-cyano-benzoyl)-methyl-amino]-1-(2- hydroxy-2-methyl-propyl)-1H- benzoimidazol-2-yl]-amide |
| 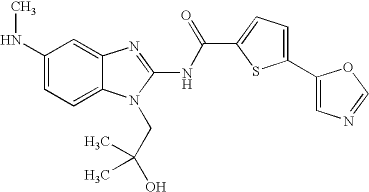 | 5-Oxazol-5-yl-thiophene-2-carboxylic acid [1-(2-hydroxy-2-methyl-propyl)-5- methylamino-1H-benzoimidazol-2- yl]-amide |
| 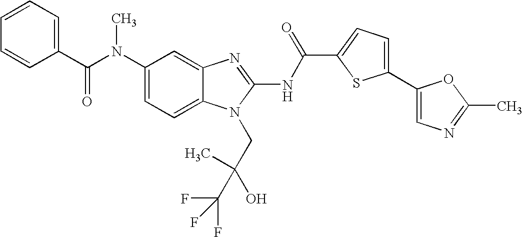 | 5-(2-Methyl-oxazol-5-yl)-thiophene-2- carboxylic acid [5-(benzoyl-methyl-amino)- 1-(3,3,3-trifluoro-2-hydroxy-2-methyl- propyl)-1H-b enzoimidazol-2-yl]-amide |
| 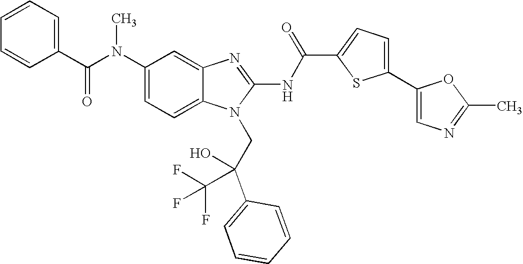 | 5-(2-Methyl-oxazol-5-yl)-thiophene-2- carboxylic acid [5-(benzoyl-methyl-amino)- 1-(3,3,3-trifluoro-2-hydroxy-2-phenyl- propyl)-1H-benzoimidazol-2-yl]-amide |
| 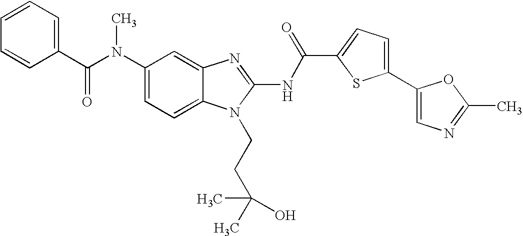 | 5-(2-Methyl-oxazol-5-yl)-thiophene-2- carboxylic acid [5-(benzoyl-methyl-amino)- 1-(3-hydroxy-3-methyl-butyl)-1H- benzoimidazol-2-yl]-amide |
| 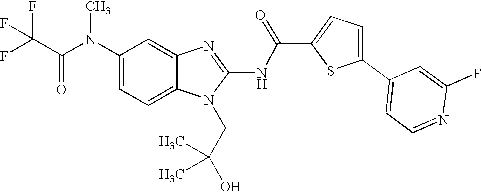 | 5-(2-Fluoro-pyridin-4-yl)-thiophene-2- carboxylic acid {1-(2-hydroxy-2-methyl- propyl)-5-[methyl-(2,2,2-trifluoro-acetyl)- amino]-1H-benzoimidazol-2-yl}-amide |
| 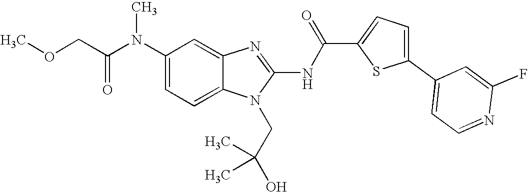 | 5-(2-Fluoro-pyridin-4-yl)-thiophene-2- carboxylic acid {1-(2-hydroxy-2-methyl- propyl)-5-[(2-methoxy-acetyl)-methyl- amino]-1H-benzoimidazol-2-yl}-amide |
| 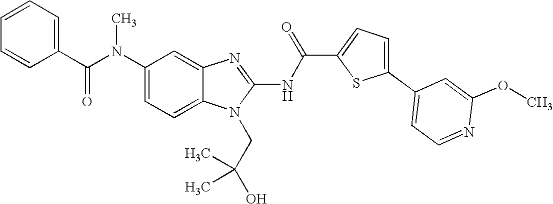 | 5-(2-Methoxy-pyridin-4-yl)-thiophene-2- carboxylic acid [5-(benzoyl-methyl-amino)- 1-(2-hydroxy-2-methyl-propyl)-1H- benzoimidazol-2-yl]-amide |
| 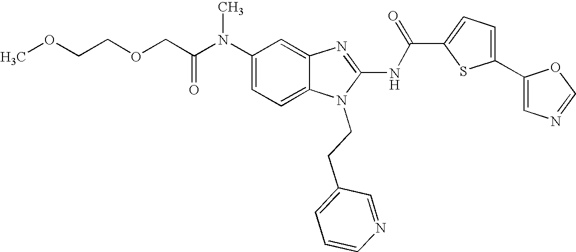 | 5-Oxazol-5-yl-thiophene-2-carboxylic acid [5-{[2-(2-methoxy-ethoxy)-acetyl]-methyl- amino}-1-(2-pyridin-3-yl-ethyl)-1H- benzoimidazol-2-yl]-amide |
| 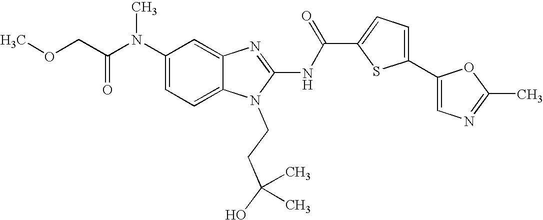 | 5-(2-Methyl-oxazol-5-yl)-thiophene-2- carboxylic acid {1-(3-hydroxy-3-methyl- butyl)-5-[(2-methoxy-acetyl)-methyl- amino]-1H-benzoimidazol-2-yl}-amide |
| 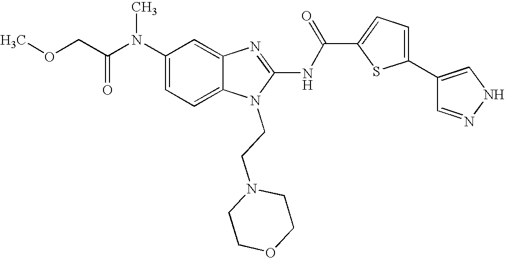 | 5-(1H-Pyrazol-4-yl)-thiophene-2-carboxylic acid [5-[(2-methoxy-acetyl)-methyl-amino]- 1-(2-morpholin-4-yl-ethyl)-1H- benzoimidazol-2-yl]-amide |
| 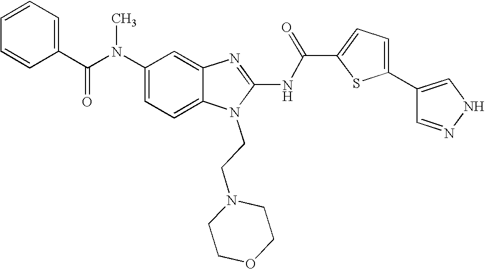 | 5-(1H-Pyrazol-4-yl)-thiophene-2-carboxylic acid [5-(benzoyl-methyl-amino)-1-(2- morpholin-4-yl-ethyl)-1H-benzoimidazol- 2-yl]-amide |
| 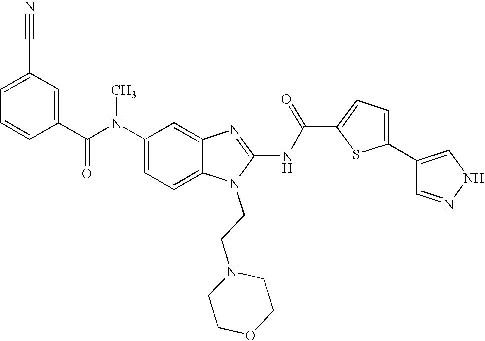 | 5-(1H-Pyrazol-4-yl)-thiophene-2-carboxylic acid [5-[(3-cyano-benzoyl)-methyl-amino]- 1-(2-morpholin-4-yl-ethyl)-1H- benzoimidazol-2-yl]-amide |
| 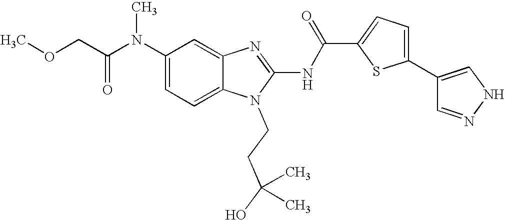 | 5-(1H-Pyrazol-4-yl)-thiophene-2-carboxylic acid {1-(3-hydroxy-3-methyl-butyl)-5-[(2- methoxy-acetyl)-methyl-amino]-1H- benzoimidazol-2-yl}-amide |
| 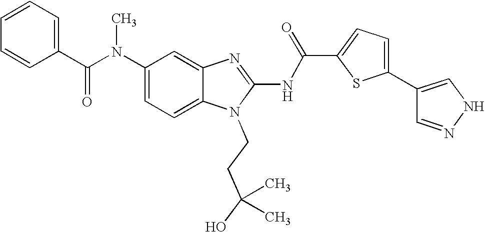 | 5-(1H-Pyrazol-4-yl)-thiophene-2-carboxylic acid [5-(benzoyl-methyl-amino)-1-(3- hydroxy-3-methyl-butyl)-1H- benzoimidazol-2-yl]-amide |
| 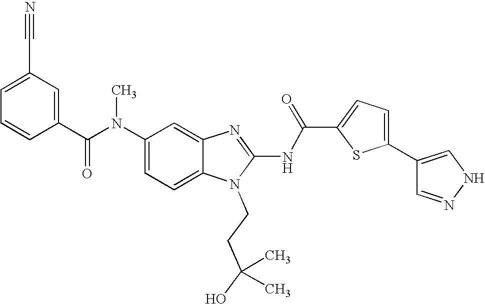 | 5-(1H-Pyrazol-4-yl)-thiophene-2-carboxylic acid [5-[(3-cyano-benzoyl)-methyl-amino]- 1-(3-hydroxy-3-methyl-butyl)-1H- benzoimidazol-2-yl]-amide |
| 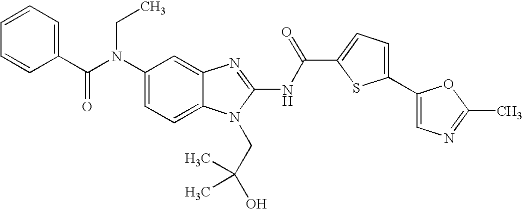 | 5-(2-Methyl-oxazol-5-yl)-thiophene-2- carboxylic acid [5-(benzoyl-ethyl-amino)-1- (2-hydroxy-2-methyl-propyl)-1H- benzoimidazol-2-yl]-amide |
| 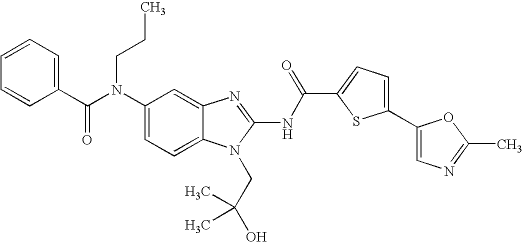 | 5-(2-Methyl-oxazol-5-yl)-thiophene-2- carboxylic acid [5-(benzoyl-propyl-amino)- 1-(2-hydroxy-2-methyl-propyl)-1H- benzoimidazol-2-yl]-amide |
| 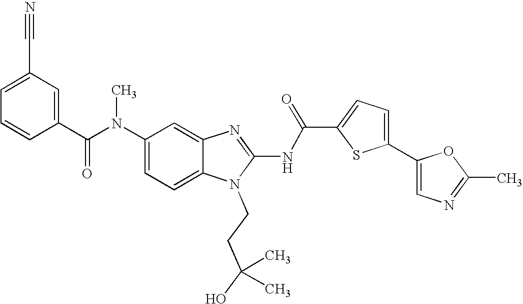 | 5-(2-Methyl-oxazol-5-yl)-thiophene-2- carboxylic acid [5-[(3-cyano-benzoyl)- methyl-amino]-(3-hydroxy-3-methyl- butyl)-1H-benzoimidazol-2-yl]-amide |
| 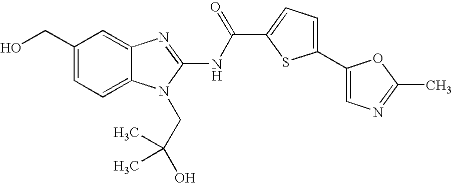 | 5-(2-Methyl-oxazol-5-yl)-thiophene-2- carboxylic acid [5-hydroxymethyl-1-(2- hydroxy-2-methyl-propyl)-1H- benzoimidazol-2-yl]-amide |
| 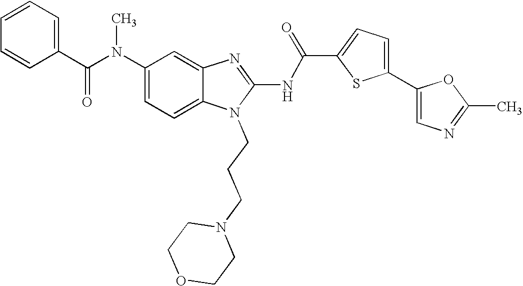 | 5-(2-Methyl-oxazol-5-yl)-thiophene-2- carboxylic acid [5-(benzoyl-methyl-amino)- 1-(3-morpholin-4-yl-propyl)-1H- benzoimidazol-2-yl]-amide |
| 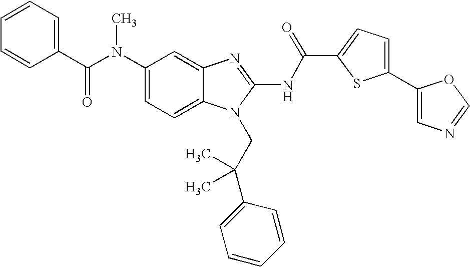 | 5-Oxazol-5-yl-thiophene-2-carboxylic acid [5-(benzoyl-methyl-amino)-1-(2-hydroxy-2- phenyl-propyl)-1H-benzoimidazol-2- yl]-amide |
| 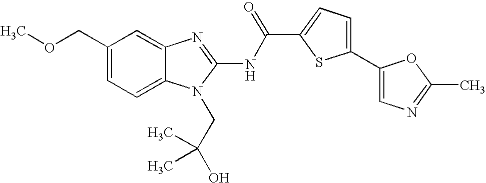 | 5-(2-Methyl-oxazol-5-yl)-thiophene-2- carboxylic acid [1-(2-hydroxy-2-methyl- propyl)-5-methoxymethyl-1H- benzoimidazol-2-yl]-amide |
| 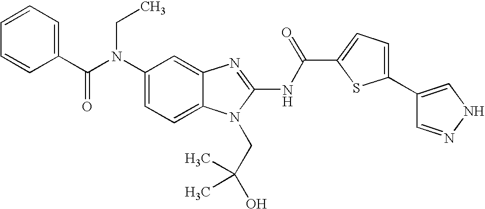 | 5-(1H-Pyrazol-4-yl)-thiophene-2-carboxylic acid [5-(benzoyl-ethyl-amino)-1-(2- hydroxy-2-methyl-propyl)-1H- benzoimidazol-2-yl]-amide |
| 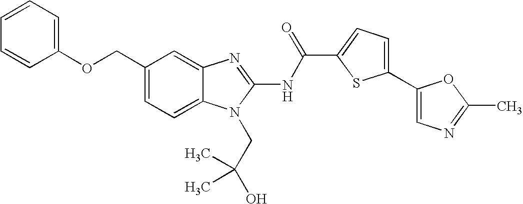 | 5-(2-Methyl-oxazol-5-yl)-thiophene-2- carboxylic acid [1-(2-hydroxy-2-methyl- propyl)-5-phenoxymethyl′-1H- benzoimidazol-2-yl]-amide |
| 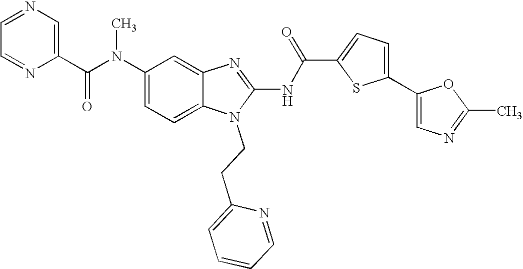 | Pyrazine-2-carboxylic acid methyl-[2-{[5- (2-methyl-oxazol-5-yl)-thiophene-2- carbonyl]-amino}-1-(2-pyridin-2-yl- ethyl)-1H- benzoimidazol-5-yl]-amide |
| 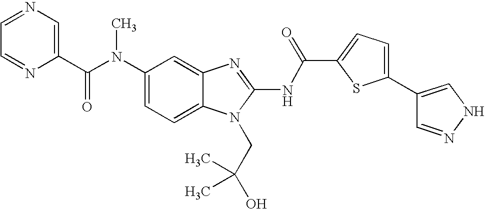 | Pyrazine-2-carboxylic acid (1-(2-hydroxy- 2-methyl-propyl)-2-{[5-(1H-pyrazol-4-yl)- thiophene-2-carbonyl]-amino}-1H- benzoimidazol-5-yl)-methyl-amide |
| 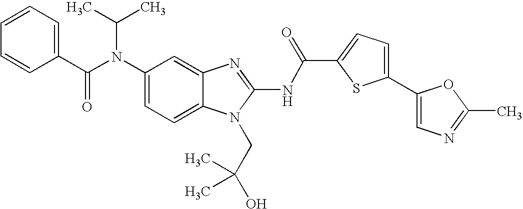 | 5-(2-Methyl-oxazol-5-yl)-thiophene-2- carboxylic acid [5-(benzoyl-isopropyl- amino)-1-(2-hydroxy-2-methyl-propyl)- 1H-benzoimidazol-2-yl]-amide |
| 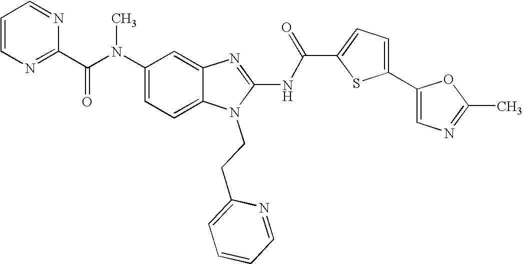 | Pyrimidine-2-carboxylic acid methyl-[2- {[5-(2-methyl-oxazol-5-yl)-thiophene-2- carbonyl]-amino}-1-(2-pyridin-2-yl-ethyl)- 1H-benzoimidazol-5-yl]-amide |
| 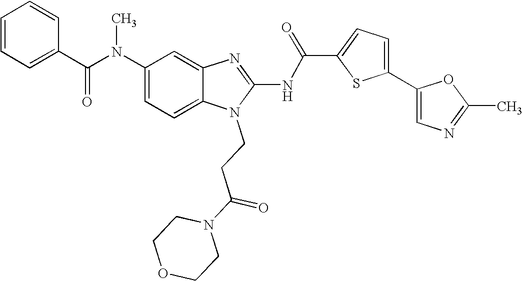 | 5-(2-Methyl-oxazol-5-yl)-thiophene-2- carboxylic acid [5-(benzoyl-methyl-amino)- 1-(3-morpholin-4-yl-3-oxo-propyl)-1H- benzoimidazol-2-yl]-amide |
| 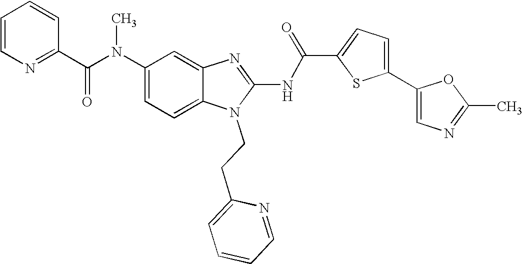 | Pyridine-2-carboxylic acid methyl-[2-{[5- (2-methyl-oxazol-5-yl)-thiophene-2- carbonyl]-amino}-1-(2-pyridin-2-yl-ethyl)- 1H-benzoimidazol-5-yl]-amide |
| 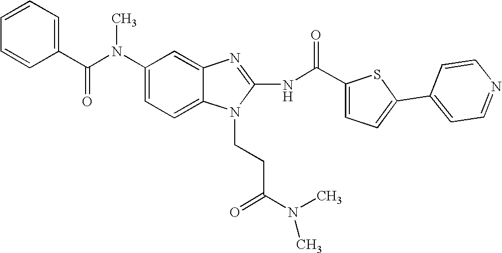 | 5-Pyridin-4-yl-thiophene-2-carboxylic acid [5-(benzoyl-methyl-amino)-1-(2- dimethylcarbamoyl-ethyl)-1H- benzoimidazol-2-yl]-amide |
| 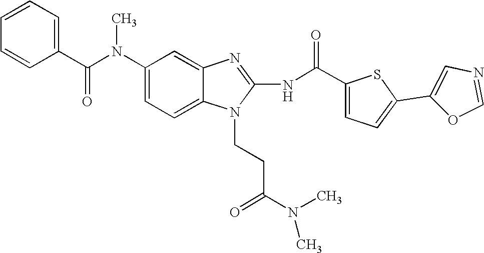 | 5-Oxazol-5-yl-thiophene-2-carboxylic acid [5-(benzoyl-methyl-amino)-1-(2- dimethylcarbamoyl-ethyl)-1H- benzoimidazol-2-yl]-amide |
| 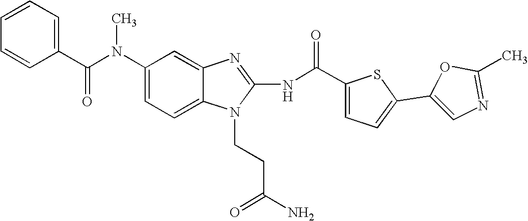 | 5-(2-Methyl-oxazol-5-yl)-thiophene-2- carboxylic acid [5-(benzoyl-methyl-amino)- 1-(2-carbamoyl-ethyl)-1H-benzoimidazol-2- yl]-amide |
| 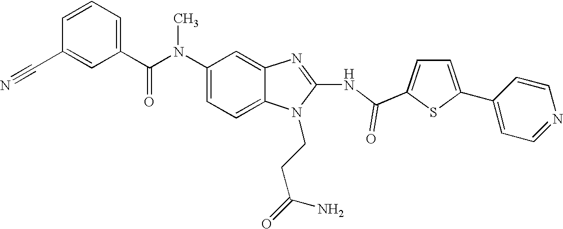 | 5-Pyridin-4-yl-thiophene-2-carboxylic acid {1-(2-carbamoyl-ethyl)-5-[(3-cyano- benzoyl)-methyl-amino]-1H- benzoimidazol-2-yl}-amide |
| 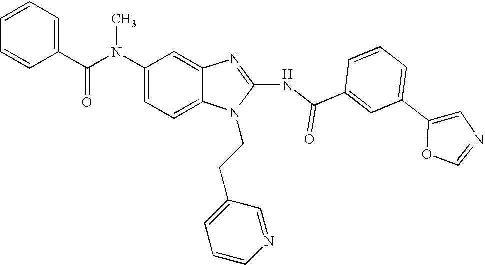 | N-[5-(Benzoyl-methyl-amino)-1-(2-pyridin- 3-yl-ethyl)-1H-benzoimidazol-2-yl]-3- oxazol-5-yl-benzamide |
| 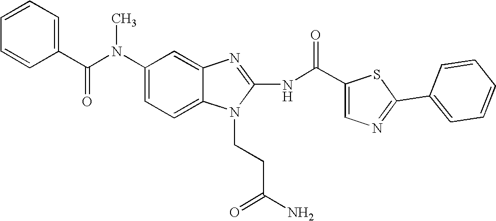 | 2-Phenyl-thiazole-5-carboxylic acid [5- (benzoyl-methyl-amino)-1-(2-carbamoyl- ethyl)-1H-benzoimidazol-2-yl]-amide |
| 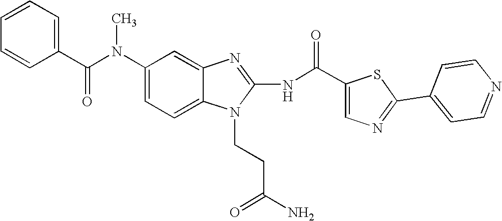 | 2-Pyridin-4-yl-thiazole-5-carboxylic acid [5-(benzoyl-methyl-amino)-1-(2-carbamoyl- ethyl)-1H-benzoimidazol-2-yl]-amide |
| 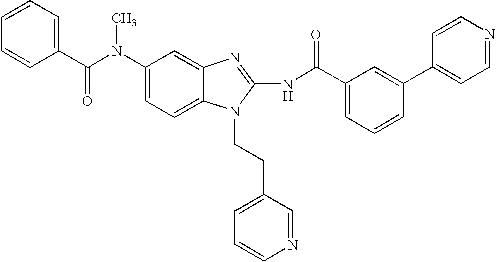 | N-[5-(Benzoyl-methyl-amino)-1-(2-pyridin- 3-yl-ethyl)-1H-benzoimidazol-2-yl]-3- pyridin-4-yl-benzamide |
| 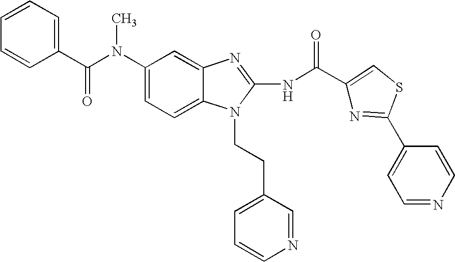 | 2-Pyridin-4-yl-thiazole-4-carboxylic acid [5-(benzoyl-methyl-amino)-1-(2-pyridin-3- yl-ethyl)-1H-benzoimidazol-2-yl]-amide |
| 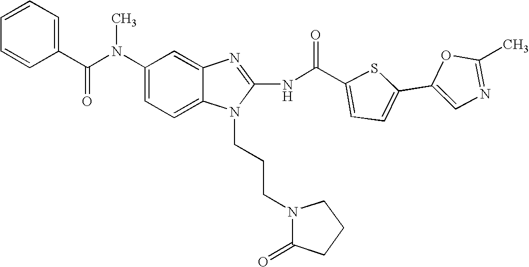 | 5-(2-Methyl-oxazol-5-yl)-thiophene-2- carboxylic acid {5-(benzoyl-methyl-amino)- 1-[3-(2-oxo-pyrrolidin-1-yl)-propyl]-1H- benzoimidazol-2-yl}-amide |
| 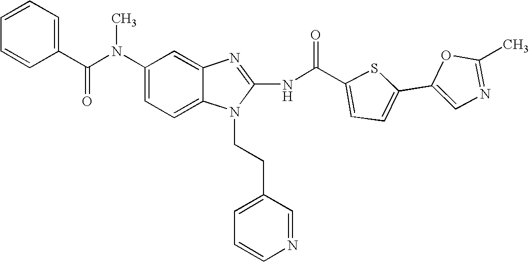 | 5-(2-Methyl-oxazol-5-yl)-thiophene-2- carboxylic acid [5-(benzoyl-methyl-amino)- 1-(2-pyridin-3-yl-ethyl)-1H-benzoimidazol- 2-yl]-amide |
| 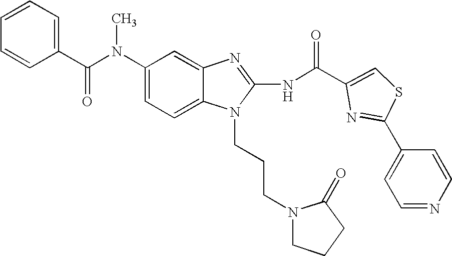 | 2-Pyridin-4-yl-thiazole-4-carboxylic acid {5-(benzoyl-methyl-amino)-1-[3-(2-oxo- pyrrolidin-1-yl)-propyl]-1H-benzoimidazol- 2-yl}-amide |
| 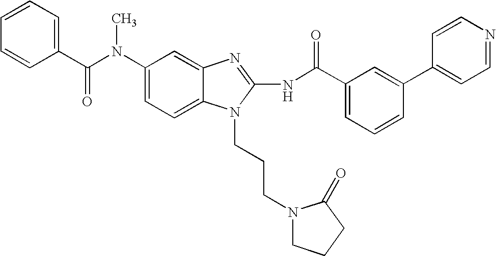 | N-{5-(Benzoyl-methyl-amino)-1-[3-(2-oxo- pyrrolidin-1-yl)-propyl]-1H-benzoimidazol- 2-yl}-3-pyridin-4-yl-benzamide |
| 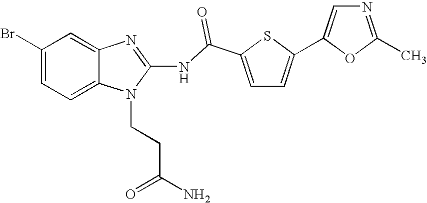 | 5-(2-Methyl-oxazol-5-yl)-thiophene-2- carboxylic acid [5-bromo-1-(2-carbamoyl- ethyl)-1H-benzoimidazol-2-yl]-amide |
| 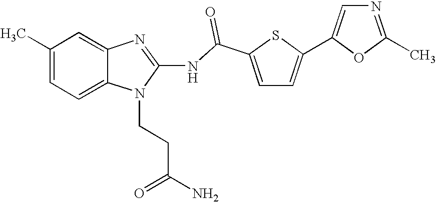 | 5-(2-Methyl-oxazol-5-yl)-thiophene-2- carboxylic acid [1-(2-carbamoyl-ethyl)-5- methyl-1H-benzoimidazol-2-yl]-amide |
| 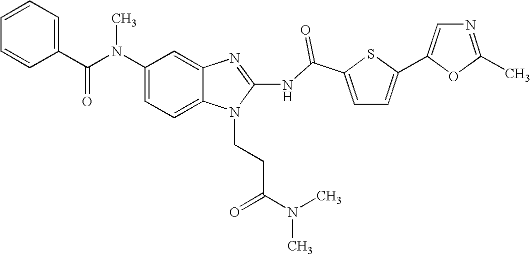 | 5-(2-Methyl-oxazol-5-yl)-thiophene-2- carboxylic acid [5-(benzoyl-methyl-amino)- 1-(2-dimethylcarbamoyl-ethyl)-1H- benzoimidazol-2-yl]-amide |
| 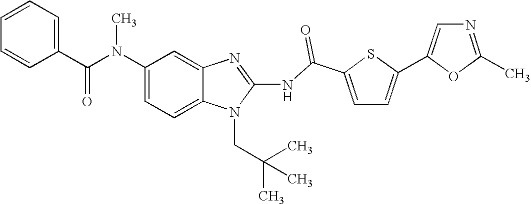 | 5-(2-Methyl-oxazol-5-yl)-thiophene-2- carboxylic acid [5-(benzoyl-methyl-amino)- 1-(2-hydroxy-2-methyl-propyl)-1H- benzoimidazol-2-yl]-amide |
| 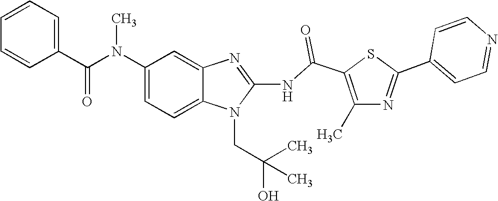 | 4-Methyl-2-pyridin-4-yl-thiazole-5- carboxylic acid [5-(benzoyl-methyl-amino)- 1-(2-hydroxy-2-methyl-propyl)-1H- benzoimidazol-2-yl]-amide |
| 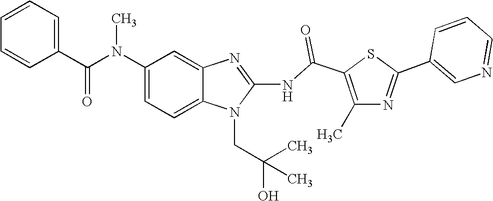 | 4-Methyl-2-pyridin-3-yl-thiazole-5- carboxylic acid [5-(benzoyl-methyl-amino)- 1-(2-hydroxy-2-methyl-propyl)-1H- benzoimidazol-2-yl]-amide |
| 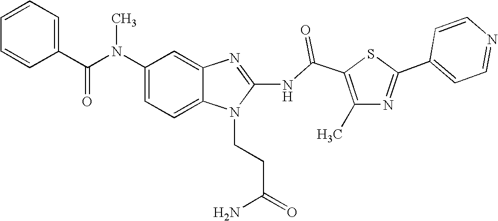 | 4-Methyl-2-pyridin-4-yl-thiazole-5- carboxylic acid [5-(benzoyl-methyl-amino)- 1-(2-carbamoyl-ethyl)-1H-benzoimidazol- 2-yl]-amide |
| 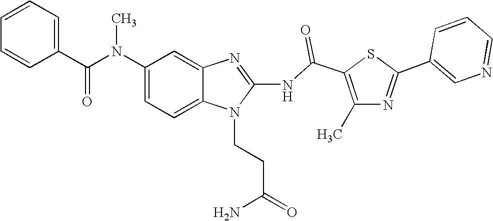 | 4-Methyl-2-pyridin-3-yl-thiazole-5- carboxylic acid [5-(benzoyl-methyl-amino)- 1-(2-carbamoyl-ethyl)-1H-benzoimidazol-2- yl]-amide |
| 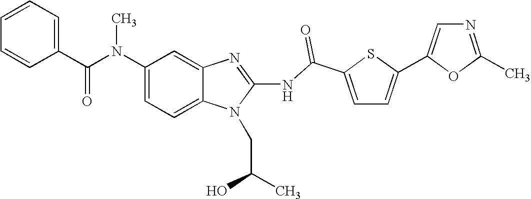 | 5-(2-Methyl-oxazol-5-yl)-thiophene-2- carboxylic acid [5-(benzoyl-methyl-amino)- 1-((R)-2-hydroxy-propyl)-1H- benzoimidazol-2-yl]-amide |
| 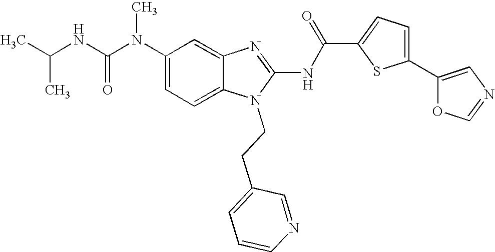 | 5-Oxazol-2-yl-thiophene-2-carboxylic acid [5-(3-isopropyl-1-methyl-ureido)-1-(2- pyridin-3-yl-ethyl)-1H-benzoimidazol- 2-yl]-amide |
| 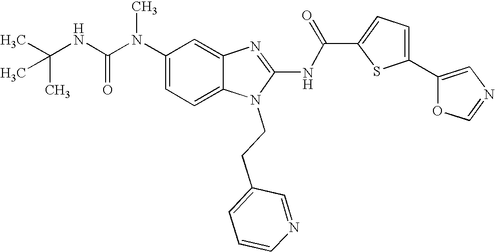 | 5-Oxazol-5-yl-thiophene-2-carboxylic acid [5-(3-tert-butyl-1-methyl-ureido)-1-(2- pyridin-3-yl-ethyl)-1H-benzoimidazol- 2-yl]-amide |
| 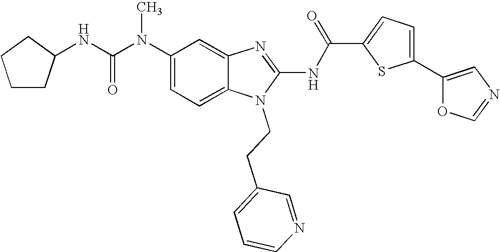 | 5-Oxazol-5-yl-thiophene-2-carboxylic acid [5-(3-cyclopentyl-1-methyl-ureido)-1-(2- pyridin-3-yl-ethyl)-1H-benzoimidazol-2- yl]-amide |
| 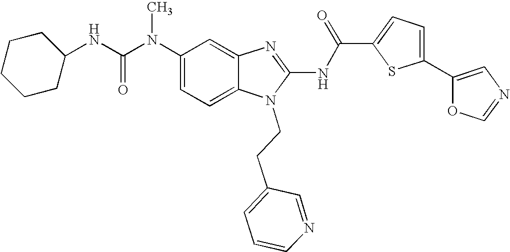 | 5-Oxazol-5-yl-thiophene-2-carboxylic acid [5-(3-cyclohexyl-1-methyl-ureido)-1-(2- pyridin-3-yl-ethyl)-1H-benzoimidazol-2- yl]-amide |
| 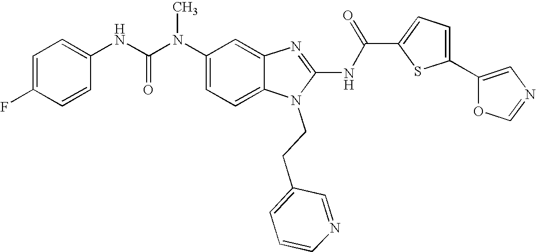 | 5-Oxazol-5-yl-thiophene-2-carboxylic acid [5-[3-(4-fluoro-phenyl)-1-methyl-ureido]-1- (2-pyridin-3-yl-ethyl)-1H-benzoimidazol-2- yl]-amide |
| 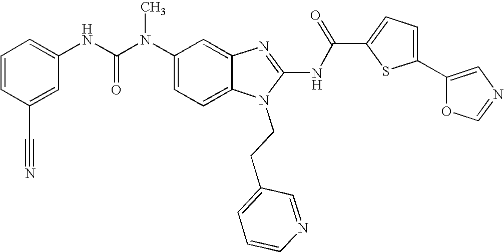 | 5-Oxazol-5-yl-thiophene-2-carboxylic acid [5-[3-(3-cyano-phenyl)-1-methyl-ureido]-1- (2-pyridin-3-yl-ethyl)-1H-benzoimidazol-2- yl]-amide |
| 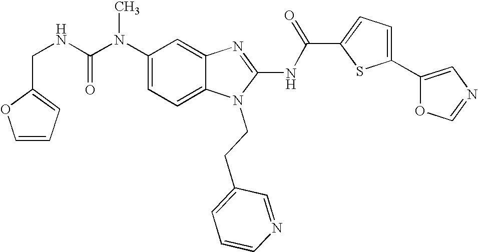 | 5-Oxazol-5-yl-thiophene-2-carboxylic acid [5-(3-furan-2-ylmethyl-1-methyl-ureido)-1- (2-pyridin-3-yl-ethyl)-1H-benzoimidazol-2- yl]-amide |
| 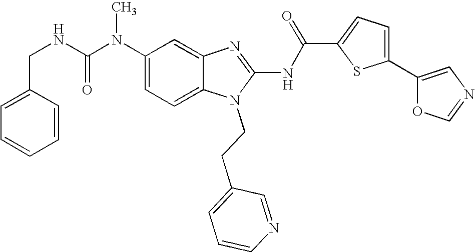 | 5-Oxazol-5-yl-thiophene-2-carboxylic acid [5-(3-benzyl-1-methyl-ureido)-1-(2-pyridin- 3-yl-ethyl)-1H-benzoimidazol-2-yl]-amide |
| 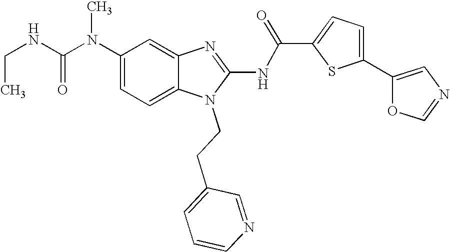 | 5-Oxazol-5-yl-thiophene-2-carboxylic acid [5-(3-ethyl-1-methyl-ureido)-1-(2-pyridin- 3-yl-ethyl)-1H-benzoimidazol-2-yl]-amide |
| 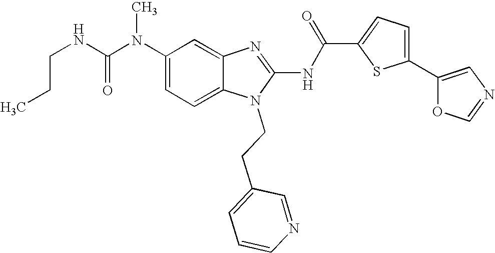 | 5-Oxazol-5-yl-thiophene-2-carboxylic acid [5-(1-methyl-3-propyl-ureido)-1-(2-pyridin- 3-yl-ethyl)-1H-benzoimidazol-2-yl]-amide |
| 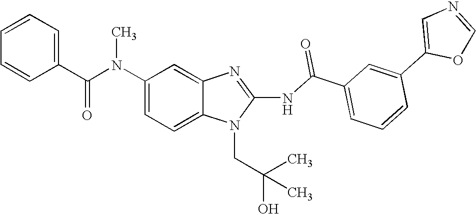 | N-[5-(Benzoyl-methyl-amino)-1-(2- hydroxy-2-methyl-propyl)-1H- benzoimidazol-2-yl]-3-oxazol-5-yl- benzamide |
| 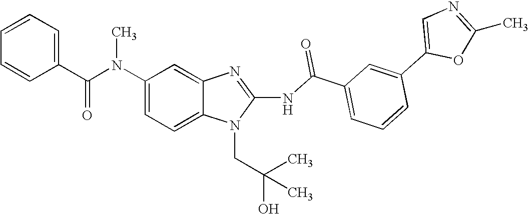 | N-[5-(Benzoyl-methyl-amino)-1-(2- hydroxy-2-methyl-propyl)-1H- benzoimidazol-2-yl]-3-(2-methyl-oxazol-5- yl)-benzamide |
| 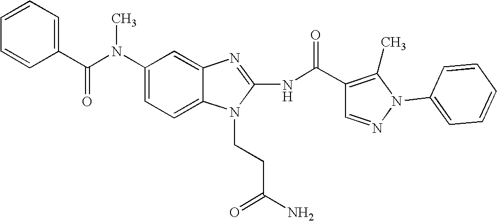 | 5-Methyl-1-phenyl-1H-pyrazole-4- carboxylic acid [5-(benzoyl-methyl-amino)- 1-(2-carbamoyl-ethyl)-1H-benzoimidazol-2- yl]-amide |
| 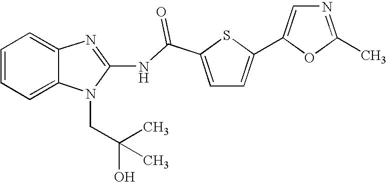 | 5-(2-Methyl-oxazol-5-yl)-thiophene-2- carboxylic acid [1-(2-hydroxy-2-methyl- propyl)-1H-benzoimidazol-2-yl]-amide |
| 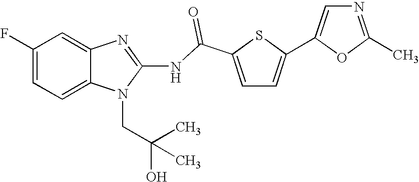 | 5-(2-Methyl-oxazol-5-yl)-thiophene-2- carboxylic acid [5-fluoro-1-(2-hydroxy-2- methyl-propyl)-1H-benzoimidazol-2-yl]- amide |
| 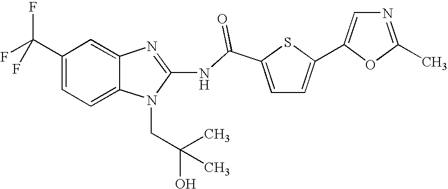 | 5-(2-Methyl-oxazol-5-yl)-thiophene-2- carboxylic acid [1-(2-hydroxy-2-methyl- propyl)-5-trifluoromethyl-1H- benzoimidazol-2-yl]-amide |
| 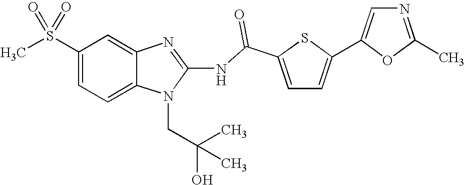 | 5-(2-Methyl-oxazol-5-yl)-thiophene-2- carboxylic acid [1-(2-hydroxy-2-methyl- propyl)-5-methanesulfonyl-1H- benzoimidazol-2-yl]-amide |
| 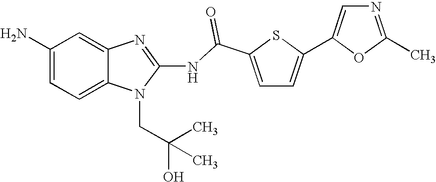 | 5-(2-Methyl-oxazol-5-yl)-thiophene-2- carboxylic acid [5-amino-1-(2-hydroxy-2- methyl-propyl)-1H-benzoimidazol-2-yl]- amide |
| 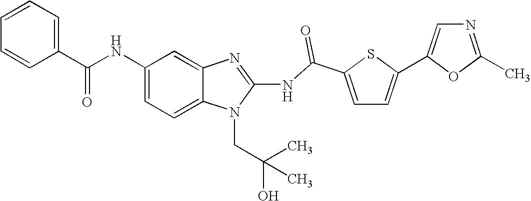 | 5-(2-Methyl-oxazol-5-yl)-thiophene-2- carboxylic acid [5-benzoylamino-1-(2- hydroxy-2-methyl-propyl)-1H- benzoimidazol-2-yl]-amide |
| 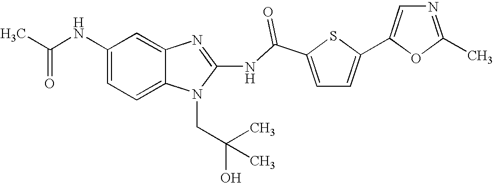 | 5-(2-Methyl-oxazol-5-yl)-thiophene-2- carboxylic acid [5-acetylamino-1-(2- hydroxy-2-methyl-propyl)-1H- benzoimidazol-2-yl]-amide |
| 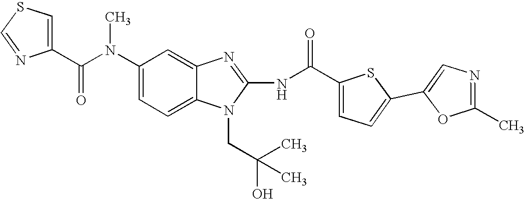 | Thiazole-4-carboxylic acid (1-(2-hydroxy- 2-methyl-propyl)-2-{[5-(2-methyl-oxazol-5- yl)-thiophene-2-carbonyl]-amino}-1H- benzoimidazol-5-yl)-methyl-amide |
| 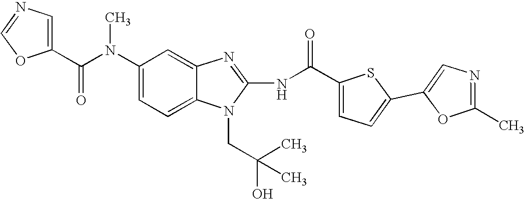 | Oxazole-5-carboxylic acid (1-(2-hydroxy-2- methyl-propyl)-2-{[-5-(2-methyl-oxazol-5- yl)-thiophene-2-carbonyl]-amino}-1H- benzoimidazol-5-yl)-methyl-amide |
| 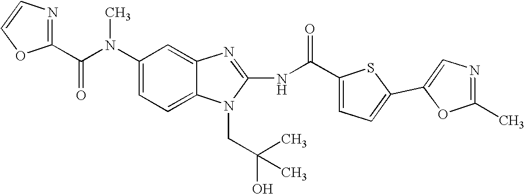 | Oxazole-2-carboxylic acid (1-(2-hydroxy-2- methyl-propyl)-2-{[5-(2-methyl-oxazol-5- yl)-thiophene-2-carbonyl]-amino}-1H- benzoimidazol-5-yl)-methyl-amide |
| 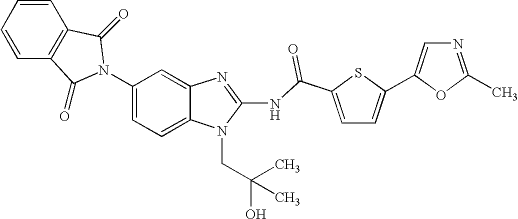 | 5-(2-Methyl-oxazol-5-yl)-thiophene-2- carboxylic acid [5-(1,3-dioxo-1,3-dihydro- isoindol-2-yl)-1-(2-hydroxy-2-methyl- propyl)-1H-benzoimidazol-2-yl]-amide |
| 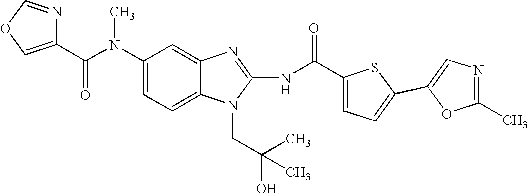 | Oxazole-4-carboxylic acid (1-(2-hydroxy-2- methyl-propyl)-2-{[5-(2-methyl-oxazol-5- yl)-thiophene-2-carbonyl]-amino}-1H- benzoimidazol-5-yl)-methyl-amide |
| 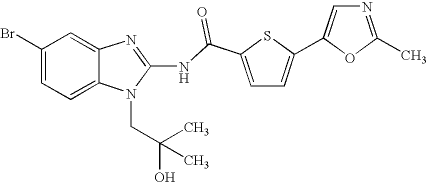 | 5-(2-Methyl-oxazol-5-yl)-thiophene-2- carboxylic acid [5-bromo-1-(2-hydroxy-2- methyl-propyl)-1H-benzoimidazol-2-yl]- amide |
| 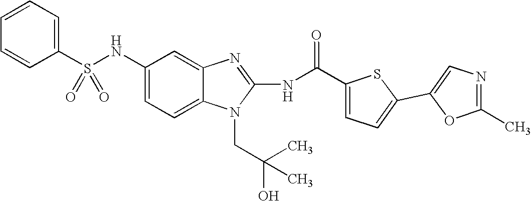 | 5-(2-Methyl-oxazol-5-yl)-thiophene-2- carboxylic acid [5-benzenesulfonylamino-1- (2-hydroxy-2-methyl-propyl)-1H- benzoimidazol-2-yl]-amide |
| 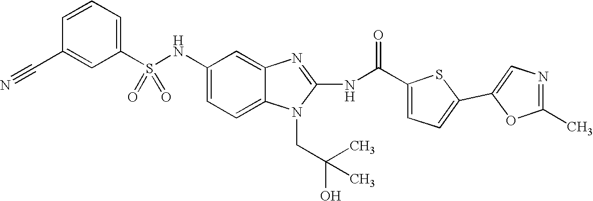 | 5-(2-Methyl-oxazol-5-yl)-thiophene-2- carboxylic acid [5-(3-cyano- benzenesulfonylamino)-1-(2-hydroxy-2- methyl-propyl)-1H-benzoimidazol-2-yl]- amide |
| 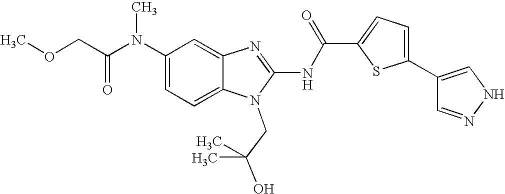 | 5-(1H-Pyrazol-4-yl)-thiophene-2-carboxylic acid {1-(2-hydroxy-2-methyl-propyl)-5-[(2- methoxy-acetyl)-methyl-amino]-1H- benzoimidazol-2-yl}-amide |
| 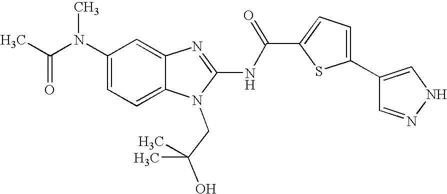 | 5-(1H-Pyrazol-4-yl)-thiophene-2-carboxylic acid [5-(acetyl-methyl-amino)-1-(2- hydroxy-2-methyl-propyl)-1H- benzoimidazol-2-yl]-amide |
| 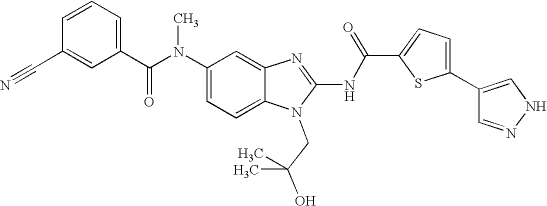 | 5-(1H-Pyrazol-4-yl)-thiophene-2-carboxylic acid [5-[(3-cyano-benzoyl)-methyl-amino]- 1-(2-hydroxy-2-methyl-propyl)-1H- benzoimidazol-2-yl]-amide |
| 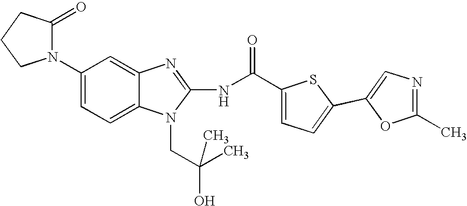 | 5-(2-Methyl-oxazol-5-yl)-thiophene-2- carboxylic acid [1-(2-hydroxy-2-methyl- propyl)-5-(2-oxo-pyrrolidin-1-yl)-1H- benzoimidazol-2-yl]-amide |
| 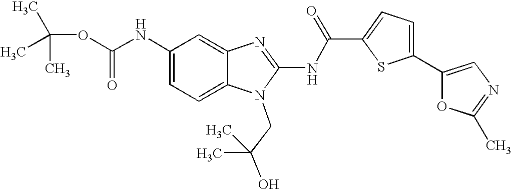 | (1-(2-Hydroxy-2-methyl-propyl)-2-{[5-(2- methyl-oxazol-5-yl)-thiophene-2-carbonyl]- amino}-1H-benzoimidazol-5-yl)-carbamic acid tert-butyl ester |
| 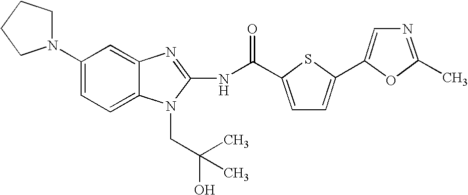 | 5-(2-Methyl-oxazol-5-yl)-thiophene-2- carboxylic acid [1-(2-hydroxy-2-methyl- propyl)-5-pyrrolidin-1-yl-1H- benzoimidazol-2-yl]-amide |
| 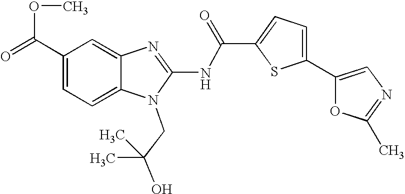 | 1-(2-Hydroxy-2-methyl-propyl)-2-{[5-(2- methyl-oxazol-5-yl)-thiophene-2-carbonyl]- amino}-1H-benzoimidazole-5-carboxylic acid methyl ester |
| 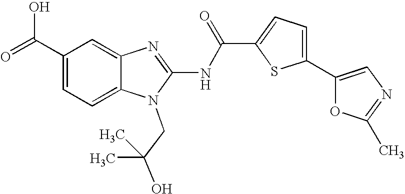 | 1-(2-Hydroxy-2-methyl-propyl)-2-{[5-(2- methyl-oxazol-5-yl)-thiophene-2-carbonyl]- amino}-1H-benzoimidazole-5-carboxylic acid |
| 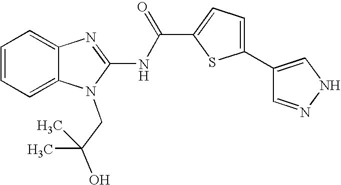 | 5-(1H-Pyrazol-4-yl)-thiophene-2-carboxylic acid [1-(2-hydroxy-2-methyl-propyl)-1H- benzoimidazol-2-yl]-amide |
| 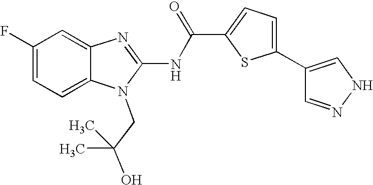 | 5-(1H-Pyrazol-4-yl)-thiophene-2-carboxylic acid [5-fluoro-1-(2-hydroxy-2-methyl- propyl)-1H-benzoimidazol-2-yl]-amide |
| 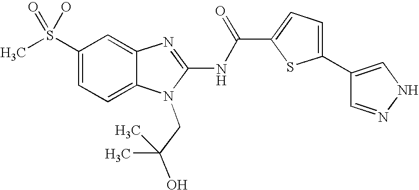 | 5-(1H-Pyrazol-4-yl)-thiophene-2-carboxylic acid [1-(2-hydroxy-2-methyl-propyl)-5- methanesulfonyl-1H-benzoimidazol-2-yl]- amide |
| 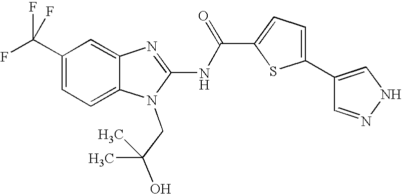 | 5-(1H-Pyrazol-4-yl)-thiophene-2-carboxylic acid [1-(2-hydroxy-2-methyl-propyl)-5- trifluoromethyl-1H-benzoimidazol-2-yl]- amide |
| 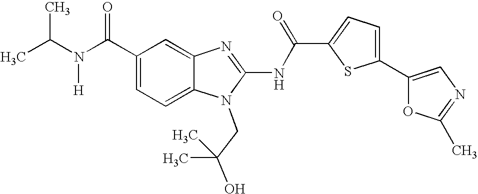 | 1-(2-Hydroxy-2-methyl-propyl)-2-{[5-(2- methyl-oxazol-5-yl)-thiophene-2-carbonyl]- amino}-1H-benzoimidazole-5-carboxylic acid isopropylamide |
| 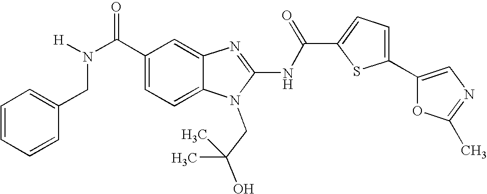 | 1-(2-Hydroxy-2-methyl-propyl)-2-{[5-(2- methyl-oxazol-5-yl)-thiophene-2-carbonyl]- amino}-1H-benzoimidazole-5-carboxylic acid benzylamide |
| 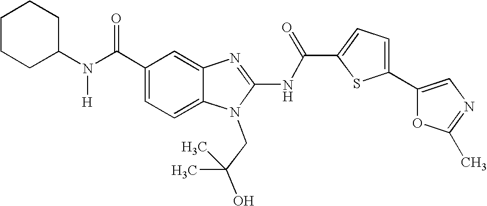 | 1-(2-Hydroxy-2-methyl-propyl)-2-([5-(2- methyl-oxazol-5-yl)-thiophene-2-carbonyl]- amino}-1H-benzoimidazole-5-carboxylic acid cyclohexylamide |
| 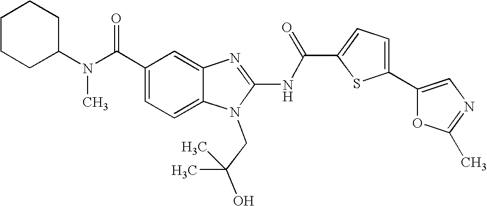 | 1-(2-Hydroxy-2-methyl-propyl)-2-{[5-(2- amino}-1H-benzoimidazole-5-carboxylic acid cyclohexyl-methyl-amide |
| 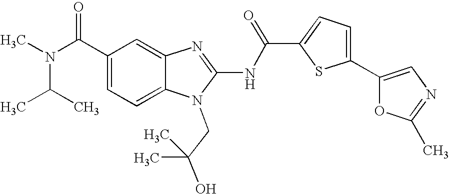 | 1-(2-Hydroxy-2-methyl-propyl)-2-{[5-(2- methyl-oxazol-5-yl)-thiophene-2-carbonyl]- amino}-1H-benzoimidazole-5-carboxylic acid isopropyl-methyl-amide |
| 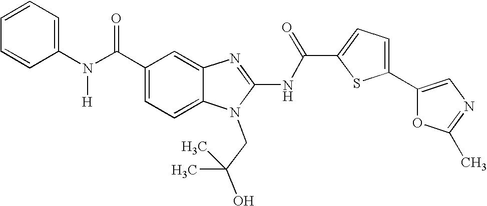 | 1-(2-Hydroxy-2-methyl-propyl)-2-{[5-(2- methyl-oxazol-5-yl)-thiophene-2-carbonyl]- amino}-1H-benzoimidazole-5-carboxylic acid phenylamide |
| 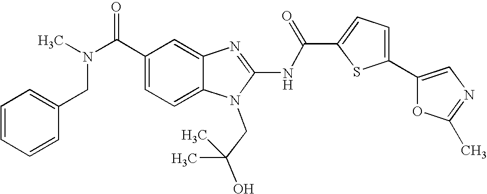 | 1-(2-Hydroxy-2-methyl-propyl)-2-{[5-(2- methyl-oxazol-5-yl)-thiophene-2-carbonyl]- amino}-1H-benzoimidazole-5-carboxylic acid benzyl-methyl-amide |
| 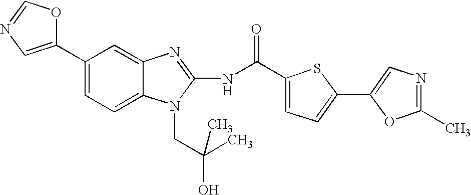 | 5-(2-Methyl-oxazol-5-yl)-thiophene-2- carboxylic acid [1-(2-hydroxy-2-methyl- propyl)-5-oxazol-5-yl-1H-benzoimidazol-2- yl]-amide |
| 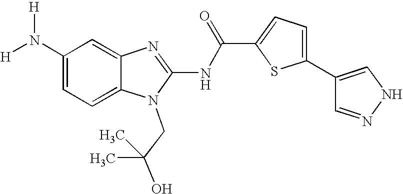 | 5-(1H-Pyrazol-4-yl)-thiophene-2-carboxylic acid [5-amino-i-(2-hydroxy-2-methyl- propyl)-1H-benzoimidazol-2-yl]-amide |
| 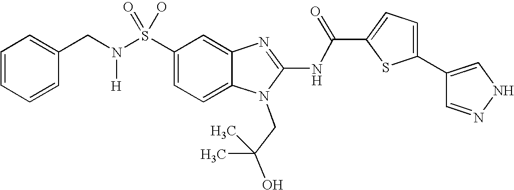 | 5-(1H-Pyrazol-4-yl)-thiophene-2-carboxylic acid [5-benzylsulfamoyl-1-(2-hydroxy-2- methyl-propyl)-1H-benzoimidazol-2-yl]- amide |
| 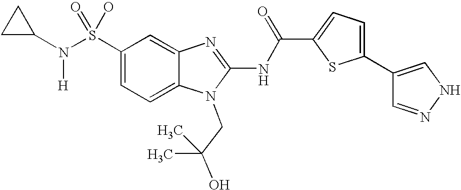 | 5-(1H-Pyrazol-4-yl)-thiophene-2-carboxylic acid [5-cyclopropylsulfamoyl-1-(2- hydroxy-2-methyl-propyl)-1H- benzoimidazol-2-yl]-amide and |
| 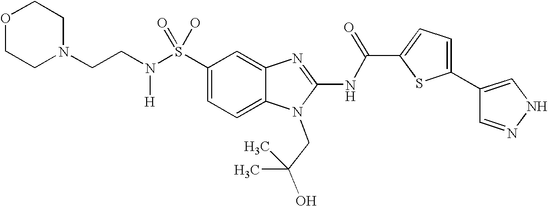 | 5-(1H-Pyrazol-4-yl)-thiophene-2-carboxylic acid [1-(2-hydroxy-2-methyl-propyl)-5-(2- morpholin-4-yl-ethylsulfamoyl)-1H- benzoimidazol-2-yl]-amide |

or the pharmaceutically acceptable salts, esters, acids, isomers or tautomers thereof.

Any of the aforementioned embodiments disclosed above may have Rₐ, R_{b} or R_{c} also being defined as azido. Such compounds are useful as photolabeling probes and include, for example, 4-azido-phenyl moieties.

In all the compounds disclosed herein above in this application, in the event the nomenclature is in conflict with the structure, it shall be understood that the compound is defined by the structure.

The invention includes the use of any compounds described above containing one or more asymmetric carbon atoms which may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Of particular importance according to the invention are compounds of formula (I), wherein Ar₁, Ar₂, R₁, R₂, R₃, R₄ and Xₐ have the meaning indicated, for use as pharmaceutical compositions with an anti-Tec kinase activity.

The invention also relates to the use of a compound of formula (I), wherein Ar₁, Ar₂, R₁, R₂, R₃, R₄ and Xₐ have the meaning indicated, for preparing a pharmaceutical composition for the treatment and/or prevention of a Tec kinase mediated disease or condition.

The invention also relates to pharmaceutical preparations, containing as active substance one or more compounds of formula (I), wherein Ar₁, Ar₂, R₁, R₂, R₃, R₄ and Xₐ have the meanings indicated, or the pharmaceutically acceptable derivatives thereof, optionally combined with conventional excipients and/or carriers.

Compounds of the invention also include their isotopically-labelled forms. An isotopically-labelled form of an active agent of a combination of the present invention is identical to said active agent but for the fact that one or more atoms of said active agent have been replaced by an atom or atoms having an atomic mass or mass number different from the atomic mass or mass number of said atom which is usually found in nature. Examples of isotopes which are readily available commercially and which can be incorporated into an active agent of a combination of the present invention in accordance with well established procedures, include isotopes of hydrogen, carbon, nitrogen, oxygen, phosphorous, fluorine and chlorine, e.g. ²H, ³H, ¹³C, ¹⁴C, ¹⁵N, ¹⁸O, ¹⁷O, ³¹P, ³²P, ³⁵S, ¹⁸F, and ³⁶Cl, respectively. An active agent of a combination of the present invention, a prodrug thereof, or a pharmaceutically acceptable salt of either which contains one or more of the above-mentioned isotopes and/or other isotopes of other atoms is contemplated to be within the scope of the present invention.

Some of the compounds of formula (I) can exist in more than one tautomeric form. The invention includes methods using all such tautomers.

All terms as used herein in this specification, unless otherwise stated, shall be understood in their ordinary meaning as known in the art.

Alkyl, alkenyl, alkynyl, alkoxy, alkylthio, acyl, alkoxycarbonyl, acyloxy, acylamino, alkylsulfonyl and all other alkyl containing groups shall be understood unless otherwise specified as being C1-10, branched or unbranched where structurally possible, and optionally partially or fully halogenated. For ‘C₀₋ₙ alkyl’, where n is an integer 1,2,3 etc, shall be understood to be a bond when the definition is ‘C₀’, and alkyl when n is greater than or equal to 1. Other more specific definitions are as follows:
BOC or t-BOC is tertiary-butoxycarbonyl.
t-Bu is tertiary-butyl.
DMF is dimethylformamide.
EtOAc is ethyl acetate.
EtOH and MeOH are ethanol and methanol, respectively.
TFA is trifluoroacetic acid.
THF is tetrahydrofuran.
DMSO is dimethylsulfoxide.
TBTU is O-(1H-benzotriazol-1-yl)-N,N,N′,N′-tetramethyluronium tetrafluoroborate.
FMOC is 9-fluorenylmethoxycarbonyl.

The term “aroyl” as used in the present specification shall be understood to mean “benzoyl” or “naphthoyl”.

The term “carbocycle” shall be understood to mean an aliphatic hydrocarbon radical containing from three to twelve carbon atoms. Carbocycles include hydrocarbon rings containing from three to ten carbon atoms. These carbocycles may be either aromatic and non-aromatic ring systems, and optionally or fully halogenated. The non-aromatic ring systems may be mono- or polyunsaturated. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms selected from nitrogen, oxygen and sulfur. The heterocycle may be attached by any atom of the cycle, which results in the creation of a stable structure. Unless otherwise stated, heterocycles include but are not limited to, pyrrolidinyl, morpholinyl, thiomorpholinyl, dioxalanyl, piperidinyl, piperazinyl, aziridinyl and tetrahydrofuranyl.

The term “heteroaryl” shall be understood to mean an aromatic 5-8 membered monocyclic or 8-11 membered bicyclic ring containing 1-4 heteroatoms such as N,O and S. Unless otherwise stated, such heteroaryls include but are not limited to thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl and indazolyl.

The term “heteroatom” as used herein shall be understood to mean atoms other than carbon such as O, N, S and P.

In all alkyl groups or carbon chains within cycloalkyl groups, where one or more carbon atoms are optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain.

Substitution on a carbon such as a methylene carbon by groups such as oxo result in definitions such as: alkoxycarbonyl, acyl, and amido , or if substituted on a ring can, for example, replace a methylene group —CH₂— with a carbonyl >C═O.

The term “aryl” as used herein shall be understood to mean aromatic carbocycle or heteroaryl as defined herein. Each aryl or heteroaryl unless otherwise specified includes its partially or fully hydrogenated derivative. For example, quinolinyl may include decahydroquinolinyl and tetrahydroquinolinyl, naphthyl may include its hydrogenated derivatives such as tetrahydranaphthyl. Each may be partially or fully halogenated. Other partially or fully hydrogenated derivatives of the aryl and heteroaryl compounds described herein will be apparent to one of ordinary skill in the art.

Terms which are analogs of the above cyclic moieties such as aryloxy or heteroaryl amine shall be understood to mean an aryl, heteroaryl, heterocycle as defined above attached to it's respective functional group.

As used herein, “nitrogen” and “sulfur” include any oxidized form of nitrogen and sulfur and the quaternized form of any basic nitrogen. For example, for an alkylthio radical such as —S—C₁₋₆ alkyl, unless otherwise specified, this shall be understood to include —S(O)—C₁₋₆ alkyl and —S(O)₂—C₁₋₆ alkyl.

The term “halogen” as used in the present specification shall be understood to mean bromine, chlorine, fluorine or iodine. The definitions “partially or fully halogenated” “substituted by one or more halogen atoms” includes for example, mono, di or tri halo derivatives on one or more carbon atoms. A non-limiting example would be a halogenated alkyl such as —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The term “patient” refers to a warm-blooded mammal and preferably, a human.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed herein above, thereby imparting the desired pharmacological effect.

### Methods of Therapeutic Use

The compounds of the invention are effective inhibitors of Tec kinase family activity, especially of Itk. Therefore, in one embodiment of the invention, there is provided methods of treating immunological disorders using compounds of the invention. In another embodiment, there is provided methods of treating inflammatory disorders using compounds of the invention. In yet another embodiment, there is provided methods of treating allergic disorders using compounds of the invention. In yet still another embodiment, there is provided methods of enhancing memory cell generation for vaccines using compounds of the invention. In a further embodiment, there is provided methods of treating cell proliferative disorders using compounds of the invention.

Without wishing to be bound by theory, the compounds of this invention modulate T cell and mast cell activation via effective inhibition of Itk. The inhibition of T cell activation is therapeutically useful for selectively suppressing immune function. Thus, the inhibition of Itk is an attractive means for preventing and treating a variety of immune disorders, including inflammatory diseases, autoimmune diseases, organ and bone marrow transplant rejection and other disorders associated with T cell mediated immune response. In particular, the compounds of the invention may be used to prevent or treat acute or chronic inflammation, allergies, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease (and other forms of organ or bone marrow transplant rejection) and lupus erythematosus.

The compounds of the invention are also effective inhibitors of Tec family kinases other than Itk including Txk, Tec, Btk, and Bmx and would thus be useful in treating diseases associated with the activity of one or more of these Tec family kinases.

Inhibitors of mast cell activation and degranulation block the release of allergic and pro-inflammatory mediators and cytokines. Thus inhibitors of Itk have potential utility in treating inflammatory and allergic disorders, including asthma, chronic obstructive pulmonary disease (COPD), adult respiratory distress syndrome (ARDS), bronchitis, conjunctivitis, dermatitis and allergic rhinitis. Other disorders associated with T cell or mast cell mediated immune response will be evident to those of ordinary skill in the art and can also be treated with the compounds and compositions of this invention.

Inhibitors of Itk and other Tec family kinases have potential utility in combination with other therapies for the treatment of immune, inflammatory, proliferative, and allergic disorders. Examples, though not all encompassing, include co-administration with steroids, leukotriene antagonists, anti-histamines, cyclosporin, or rapamycin.

One strategy to improve vaccination methods is to increase the number of memory T cells generated. As described in the Background, in the absence of Itk in mice, increased numbers of memory cells are generated. Thus, within the scope of the invention is the use of the present compounds in the formulation of improved vaccines that generate increased numbers of memory T cells.

For therapeutic use, the compounds of the invention may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds of this invention may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the compounds of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

### Biological Activity

### Tec Family Kinase Assay

Itk, Txk, Tec, Btk, and Bmx are purified as a GST-fusion protein. The kinase activity is measured using DELFIA (Dissociation Enhanced Lanthanide Fluoroimmunoassay) which utilizes europium chelate-labeled anti-phosphotyrosine antibodies to detect phosphate transfer to a random polymer, poly Glu₄: Tyr₁ (PGTYR). The screen is run on the Zymark Allegro robot system to dispense reagents, buffers and samples for assay, and also to wash and read plates. The kinase assay is performed in kinase assay buffer (50 mM HEPES, pH 7.0, 25 mM MgCl₂, 5 mM MnCl₂, 50 mM KCl, 100 μM Na₃VO₄, 0.2% BSA, 0.01% CHAPS, 200 μM TCEP). Test samples initially dissolved in DMSO at 1 mg/mL, are pre-diluted for dose response (10 doses with starting final concentration of 3 μg/mL, 1 to 3 serial dilutions) with the assay buffer in 96-well polypropylene microtiter plates. A 50 μL volume/well of a mixture of substrates containing ATP (final ATP concentration in each kinase assay is equal to its apparent ATP Kₘ) and 3.6 ng/μL PGTYR-biotin (CIS Bio International) in kinase buffer is added to neutravidin coated 96-well white plate (PIERCE), followed by 25 μL/well test sample solution and 25 μL/well of diluted enzyme (1-7 nM final conc.). Background wells are incubated with buffer, rather than 25 μL enzyme. The assay plates are incubated for 30 min at room temperature. Following incubation, the assay plates are washed three times with 250 μL DELFIA wash buffer. A 100 μL aliquot of 1 nM europium-labeled anti-phosphotyrosine (Eu³⁺-PT66, Wallac CR₀₄-100) diluted in DELFIA assay buffer is added to each well and incubated for 30 min at room temperature. Upon completion of the incubation, the plate is washed four times with 250 μL of wash buffer and 100 μL of DELFIA Enhancement Solution (Wallac) is added to each well. After 15 min of longer, time-resolved fluorescence is measured (excitation at 360 nm, emission at 620 nm) after a delay time of 250 μs.

Preferred compounds of the invention have an activity of 1 microMolar or less.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the schemes below are either commercially available or easily prepared from commercially available materials by those skilled in the art.

### General Synthetic Methods

The invention also provides processes for making compounds of formula I. In all schemes, unless specified otherwise, R or Ar substituents in the formulas below shall have the meaning of R or Ar substituents in the formula I of the invention described herein above. Intermediates used in the preparation of compounds of the invention are either commercially available or readily prepared by methods known to those skilled in the art. Further reference in this regard may be made to WO 03/041708 corresponding to U.S. publication U.S. 2003-0144281, and PCT application PCT/US03/24024 corresponding to U.S. application Ser. No. 10/632,888, and U.S. provisional No. 60/536,362.

Compounds of formula I in which R₄ is in the 5-position and is —N(R₇)C(O)R₅, Xₐ is O and R₁ is H may be prepared by the method outlined in Scheme I.

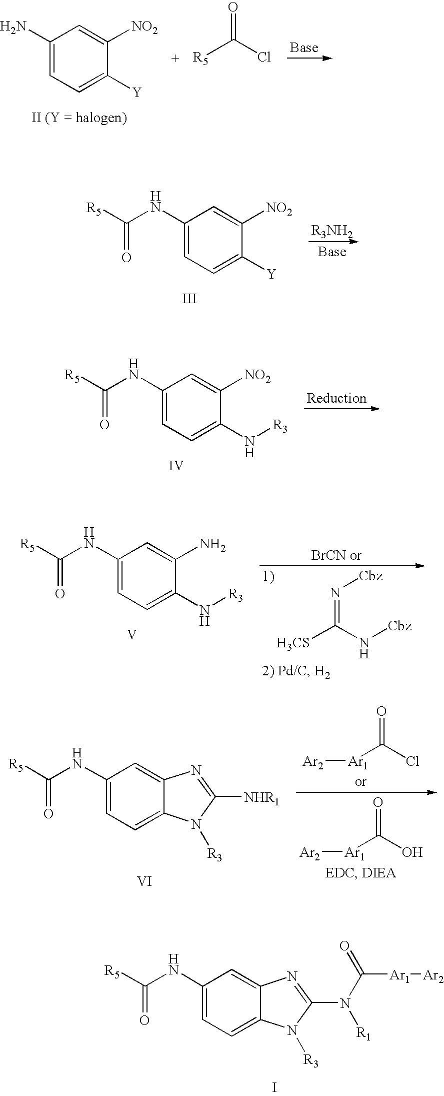

As illustrated in Scheme I, a 4-halo-3-nitroaniline II, preferably 4-fluoro-3-nitroaniline, is reacted with R₅C(O)Cl in the presence of a suitable base such as pyridine to form amide III. This intermediate is then reacted with R₃NH₂ in the presence of a base such as triethylamine to form IV. Reduction of the nitro group by methods known in the art, for example by treatment with hydrogen or a hydrogen source such as ammonium carbonate in the presence of a catalyst such as palladium on carbon provides V. Reaction of V with cyanogen bromide in a suitable solvent such as ethanol provides benzimidazole VI. Alternatively, reaction of V with thiopseudourea in the presence of catalytic amount of acid such as p-toluene sulfonic acid (pTSA), followed by a deprotection of the carbamate group provides benzimidazole VI. Reaction of VI with Ar₂—Ar₁C(O)Cl in the presence of a base such as pyridine provides the desired compound of formula (I). Alternatively, reaction of VI with Ar₂—Ar₁COOH in the presence of a suitable coupling reagent such as 1-(3-dimethylaminopropyl)-3-ethylcarbodiimide (EDC) and a base such as diisopropylethylamine provides the desired compound formula (I).

If one desires a compound of formula (I) in which R₇ is alkyl, one may react intermediate III with an alkyl halide in the presence of a suitable base such as sodium bistrimethylsilylamide to produce VII as illustrated in Scheme II. One may then proceed with the displacement of the ring halogen with R₃NH₂ and subsequent steps as described in Scheme I to produce the desired compound of formula (I).

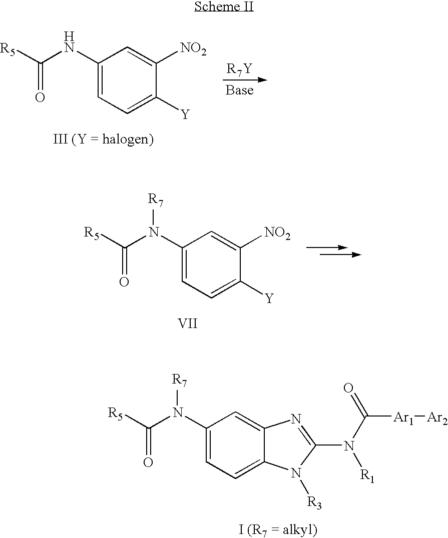

Compounds of formula (I) in which R₄ is in the 6-position and is —N(R₇)C(O)R₅, Xₐ is O and R₁ is H may be prepared as described in Scheme III. A 4-nitro-3-halotoluene (VIII), preferably 4-nitro-3-fluorotoluene is treated with a suitable oxidizing agent such as sodium dichromate to provide benzoic acid derivative IX. This is converted to an aniline derivative by methods known in the art, for example by refluxing with diphenylphosphorylazide in a mixture of tert-butyl alcohol and dioxane to provide the tert-butoxycarbonyl-protected aniline X. Deprotection of the aniline, in this case by treatment with acid such as trifluoroacetic acid provides XI. This may then be reacted further as described in Schemes I and II to provide the desired compounds of formula (I) having R₄ in the 6-position.

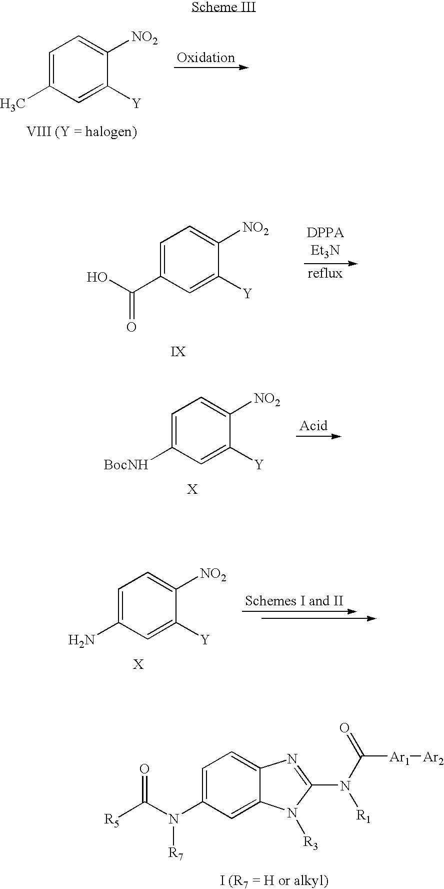

Compounds of formula (I) in which R₄ is —CH₂N(R₇)C(O)R₅ and is in the 5-position, Xₐ is O and R₁ is H may be prepared as described in Scheme IV. As illustrated below, a 4-halo-3-nitrobenzoic acid (XII), preferably a 4-fluoro-3-nitrobenzoic acid is coupled with R₇NH₂ using a suitable coupling reagent such as 1-(3-dimethylaminopropyl)-3-ethylcarbodiimide (EDC) to form XIII. Reaction of XIII with R₃NH₂ in the presence of a suitable base such as triethylamine provides XIV. Reduction of the amide functionality with a suitable reducing agent such as borane-tetrahydrofuran complex gives the benzylamine XV. Reaction of XV with R₅C(O)Cl, in the presence of a base such as diisopropylethylamine provides XVI. This may then be reduced as described for intermediate IV in Scheme I, and further reacted as described in Scheme I to provide the desired compounds of formula (I) with R₄ being —CH₂N(R₇)C(O)R₅ and in the 5-position.

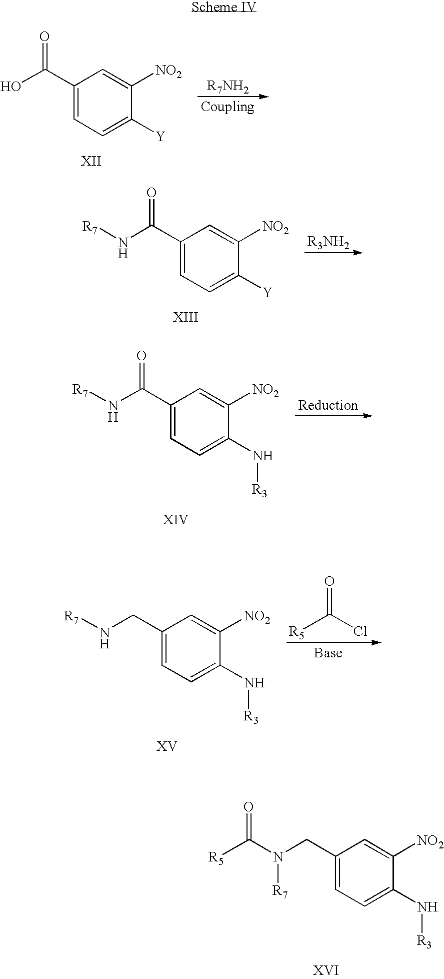

Compounds of formula (I) in which R₄ is —CH₂N(R₇)C(O)R₅ and is in the 6-position, Xₐ is O and R₁ is H may be prepared starting with intermediate IX in Scheme III. Treatment of IX in the manner described in Scheme IV for intermediate XII will provide the desired compounds.

Compounds of formula (I) in which R₄ is in the 5-position and is —CH₂N(R₅)(R₇) may be prepared by the method outlined in Scheme V.

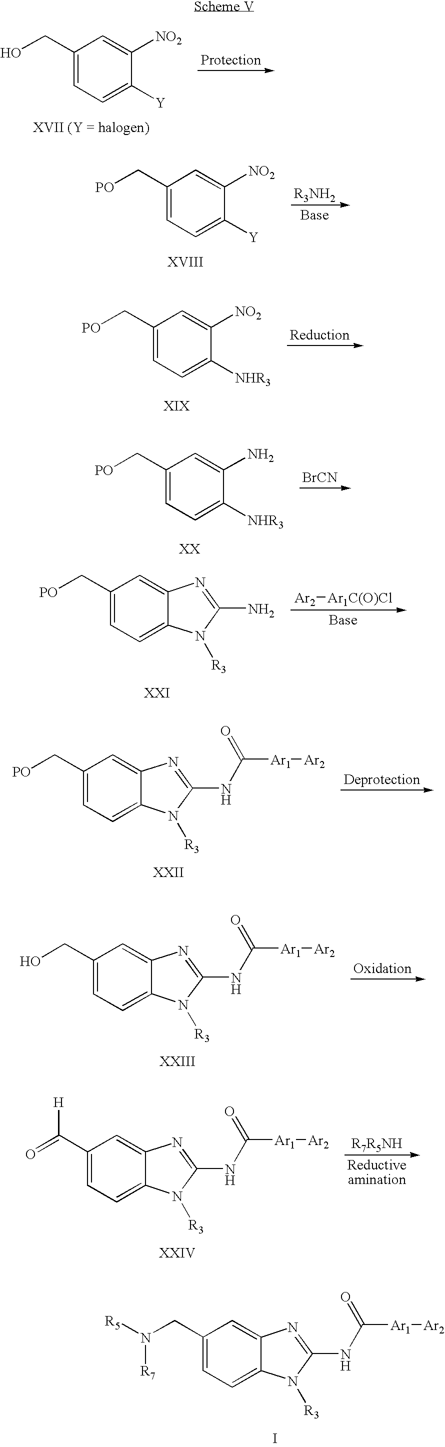

As illustrated in Scheme V, a 4-halo-3-nitro benzyl alcohol (XVII), preferably a 4-fluoro-3-nitro benzyl alcohol is protected with a suitable protecting group such as a triisopropylsilyl group, to provide XVIII, where P is a protecting group. Reaction of XVIII with R₃NH₂ in the presence of a suitable base such as triethylamine provides XIX. Reduction of the nitro group, for example by treatment with a hydrogen source such as ammonium formate in the presence of a catalyst such as palladium on carbon provides XX. Reaction of XX with cyanogen bromide in a suitable solvent such as ethanol provides benzimidazole intermediate XXI. Reaction of XXI with Ar₂—Ar₁C(O)Cl in the presence of a base such as diisopropylethylamine produces amide XXII. Deprotection of the benzyl alcohol, for example by treatment with dilute acid if P is a triisopropylsilyl group, gives XXIII. The benzyl alcohol is then treated with a suitable oxidizing reagent such as MnO₂ to provide the aldehyde XXIV. Reaction of XXIV with R₇R₅NH under reductive amination conditions provides the desired compound of formula (I) in which R₄ is —CH₂N(R₅)(R₇) and is in the 5-position.

### SYNTHETIC EXAMPLES

Examples 1-7 Illustrate Syntheses of 5-aryl-2 Carboxylic Acid Intermediates Useful in the Preparation of Compounds of Formula (I):

### Example 1

### Preparation of 5-quinolin-8-yl-thiophene-2-carboxylic acid

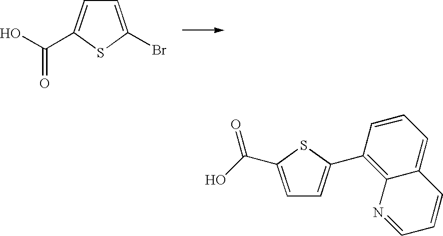

A mixture of 200 mg (0.97 mmol) of 5-bromo-2-thiophenecarboxylic acid, 200 mg (1.16 mmol, 1.2 eq.) of 8-quinoline boronic acid and 34 mg (0.05 mmol, 5 mol %) of bistriphenylphosphinepalladium(II) chloride in 1.5 mL of 2M Na₂CO₃ solution and 3 mL of DMF was heated at 100° C. for 10 min under microwave conditions. The resulting mixture was diluted with 1N HCl, and the precipitate was collected. This solid was dissolved in MeOH and acetone, and this solution was treated with (MgSO₄) and activated charcoal. The solution was filtered through diatomaceous earth and concentrated. The residual solid was triturated with EtOAc and hexanes to give 220 mg (89%) of desired compound as a light orange solid.

### Example 2

### Preparation of 5-oxazol-2-yl-thiophene-2-carboxylic acid

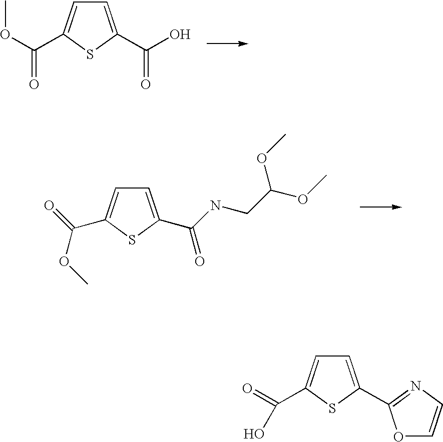

To a stirred suspension of thiophene-2,5-dicarboxylic acid monomethyl ester (0.7 g) in dichloromethane (40 mL) was added oxalyl chloride (3.8 mL, 2 eq.) at room temperature followed by DMF (one drop) under nitrogen. After 2.5 h the reaction was concentrated and the residue was diluted with dichloromethane. Amino acetaldehyde dimethylacetal (1 g, 2.7 eq.) was added and the reaction was stirred over night. The reaction was poured into 1M HCl (150 mL) and the layers were separated. The organic phase was dried (MgSO₄), filtered and concentrated to give 5-(2,2-dimethoxy-ethylcarbamoyl)-thiophene-2-carboxylic acid methyl ester as a solid (0.8 g) which was used without further purification.

The above ester (0.7 g) was dissolved in dichloromethane (40 mL) and treated with trifluoroacetic acid (TFA) (1.5 mL). The reaction was monitored by TLC. Upon complete consumption of the starting material the reaction was concentrated to dryness. The remaining residue was dissolved into dichloromethane and treated again with dichloromethane. The solution was concentrated to dryness and the residue purified via CombiFlash (10 g SiO₂, 20% EtOAc/dichloromethane, 20 mL/min, UV detection at 254 nm). The isolated product was identified as the amide-aldehyde (cleavage of acetal). The aldehyde (0.2 g) was dissolved in THF (20 mL) and treated with Burgess Reagent (enough to drive reaction to completion, >2 eq.; the Burgess reagent should be either recrystallized/purified/or freshly prepared) and warmed to 70° C. Upon complete consumption of the starting material, the reaction mixture was treated with silica gel and concentrated. The remaining solid was purified via CombiFlash (10 g SiO₂, dichloromethane, 20 mL/min, UV detection at 254 nm). The product-containing fractions were combined and concentrated to give 20 mg of the desired product (by LC-MS, not recorded) which was used without further purification.

The above ester was hydrolyzed using lithium hydroxide monohydrate in THF and water at 5° C. The reaction was allowed to warm to ambient temperature. After 3 h, the reaction appeared complete by LC/MS (not recorded). The reaction was diluted with EtOAc and the layers were separated. The aqueous phase was washed with EtOAc (2×) and acidified using 1M HCl. The product was extracted into EtOAc (3×). The combined organics were concentrated to give the title compound which was used without further purification.

### Example 3

### 5-(2-fluoro-pyridin-4-yl)-thiophene-2-carboxylic acid

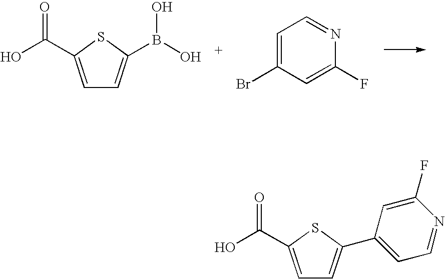

A degassed solution of sodium carbonate (3.0 mL; 2.0 M in H₂O) was added to a stirred solution of 5-di(hydroxyboryl)-2-thiophene carboxylic acid (300 mg), 4-bromo-2-fluoropyridine (307 mg), dichlorobis(triphenylphosphine) palladium (II) (60 mg) in degassed DMF (7.5 mL). The reaction mixture was heated at 100° C. for 6 h. The reaction mixture was allowed to cool to room temperature, acidified with 1M HCl, and filtered. The solid was dissolved in a 1:1 mixture of MeOH:acetone, decolorizing charcoal was added, and the solution was filtered through a diatomaceous earth pad. The solution was dried with sodium sulfate and the sample concentrated under educed pressure to give 310 mg (80%) of title compound.

### Example 4

### Preparation of 5-oxazol-5-yl-thiophene-2-carboxylic acid

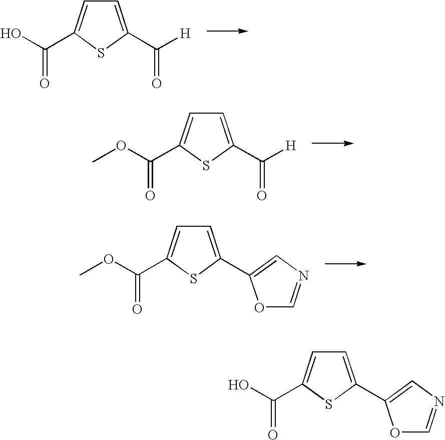

A mixture of 4.5 g (29 mmol) of 5-formyl-2-thiophene carboxylic acid and 12.6 g (102 mmol, 3.5 eqiv.) of Na₂CO₃ in 50 mL of DMF was treated with 2.2 mL of iodomethane (35 mmol, 1.2 eqiv.) at room temperature for 20 h. The mixture was quenched with H₂O with some addition of sat.NH₄Cl sol. 5-Formyl-thiophene-2-carboxylic acid methyl ester formed as a precipitate and was collected by filtration (quantitative yield), used directly for next step.

A mixture of 170 mg (1 mmol) of 5-formyl-thiophene-2-carboxylic acid methyl ester, 197 mg (1 mmol) of tosylmethyl isocyanide and 138 mg (1 mmol) of K₂CO₃ in MeOH was refluxed for 0.5 hr. The solvent was evaporated under reduced pressure. The resulting residue was poured into ice-water, and extracted with EtOAc. The extract was washed with saturated aqueous NH₄Cl, water, brine and dried over MgSO₄. The organic solvent was evaporated under reduced pressure and the residue was purified by combiflash to yield 170 mg of 5-oxazol-5-yl-thiophene-2-carboxylic acid methyl ester. Yield 81%.

The above ester (170 mg) was dissolved in 9 mL of THF, 3 mL of MeOH and 3 mL of 1N LiOH solution and stirred overnight. The organic solvent was removed. Acetic acid was added to pH˜4-5 and a light yellow precipitate formed. The suspension was diluted with water and the precipitate collected by filtration to yield 105 mg of the title compound. Yield 66%.

### Example 5

### Preparation of 5-oxazol-4-yl-thiophene-2-carboxylic acid

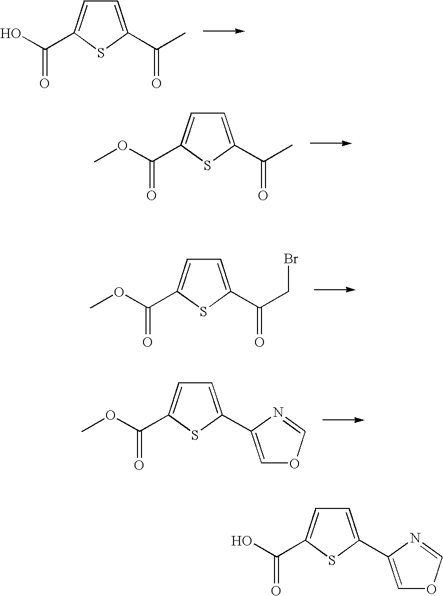

A mixture of 1.7 g (10 mmol) of 5-acetyl-thiophene-2-carboxylic acid, 0.75 mL MeI (12 mmol), 4.3 g of Na₂CO₃ (35 mmol) in DMF 10 mL was allowed to stir at room temperature overnight. Quench with water with some addition of sat. NH₄Cl sol. Precipitate formed and was collected to yield 1.48 g of 5-acetyl-thiophene-2-carboxylic acid methyl ester as a white solid. Yield 80%.

A mixture of 5.1 g of 5-acetyl-thiophene-2-carboxylic acid methyl ester, 11 g of CuBr₂ in 50 mL of EtOAc was refluxed for 6 hrs. The reaction mixture was cooled to rt and filtered through diatomaceous earth and purified by combiflash to yield 5 g of 5-(2-bromo-acetyl)-thiophene-2-carboxylic acid methyl ester. Yield 69%.

A mixture of 263 mg (1 mmol) of the above ester and 0.4 mL of formamide was heated to 120° C. Then a mixture of 0.2 mL and 0.1 g of concentrated H₂SO₄ was added. The reaction mixture was heated at 120° C. for 1 h. The reaction mixture was cooled down to room temperature and poured into water, and extracted with ether. The ethereal extract was dried over MgSO₄, and concentrated in vacuo. The residue was purified by combiflash to yield 60 mg of 5-oxazol-4-yl-thiophene-2-carboxylic acid methyl ester as light yellow solid. Yield 30%.

The above ester was dissolved in 3 mL THF, 1 mL MeOH and 1 mL 1N LiOH solution and stirred overnight. The solvent was removed. Acetic acid was added and a light yellow precipitate formed which was collected by filtration to yield 20 mg of the title compound. Yield 33%.

### Example 6

### Preparation of 5-pyridin-4-yl-thiophene-2-carboxylic acid

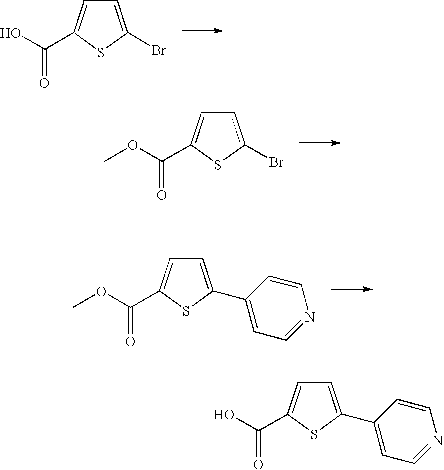

5-Bromo-thiophene-2-carboxylic acid (10 g, 48 mmol) was taken up in CH₃OH and HCl was bubbled through the solution for 10 min at room temperature. The reaction was then stirred for 16 h at room temperature. The CH₃OH/HCl was removed under reduced pressure and the residue was taken up in EtOAc and the organic layer was washed with NaHCO₃. The organic layer was washed with water, with brine then dried over anhydrous MgSO₄. The solution was removed under reduced pressure providing 8.7 g of 5-bromo-thiophene-2-carboxylic acid methyl ester as a white solid, pure enough to use in next step. Yield 87%.

Pd(PPh₃)₄ (132 mg, 10%) was added to a degassed solution of 5-bromo-thiophene-2-carboxylic acid methyl ester (220 mg, 1 mmol) and pyridine-4-ylboronic acid (246 mg, 2 mmol) in 3 mL of DMF. Sodium carbonate (530 mg, 5 mmol) and a few drops of water were added. The reaction vessel was microwave heated at 140° C. for 20 min. The Pd catalyst was separated from the reaction mixture by filtration. The mixture was diluted with EtOAc and water. The combined organic extracts were washed with a NaHCO₃ saturated solution, brine, dried with MgSO₄, filtered and concentrated. The residue was purified to yield 130 mg of 5-pyridin-4-yl-thiophene-2-carboxylic acid methyl ester. Yield 30%.

5-Pyridin-4-yl-thiophene-2-carboxylic acid methyl ester was dissolved in 6 mL THF, 2 mL MeOH and added 1N LiOH and stirred overnight. The solvent was removed. 1N HCl was added and a yellow precipitate formed. LC/MS showed only product. The suspension was diluted with water and the precipitate collected by filtration to yield 130 mg of the title compound as a light yellow solid. Yield 68%.

### Example 7

### Preparation of 5-(2-tert-butoxycarbonylamino-thiazol-4-yl)-thiophene-2-carboxylic acid

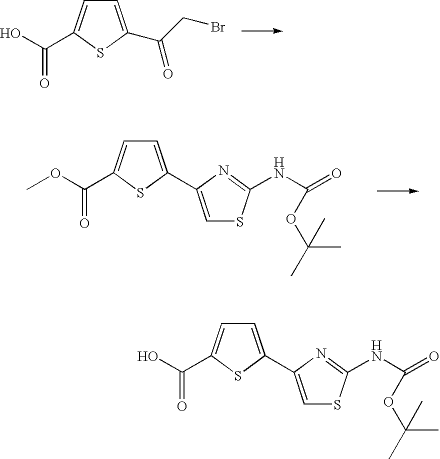

A mixture of 332 mg (1.3 mmol) of 5-(2-bromo-acetyl)-thiophene-2-carboxylic acid methyl ester, 110 mg (1.3 mmol) of N-Boc thiourea, 103 mg (1.3 mmol) of NaOAc in 10 mL EtOH was refluxed for 4 hr. The crude mixture was purified by combiflash to yield 200 mg of 5-(2-tert-butoxycarbonylamino-thiazol-4-yl)-thiophene-2-carboxylic acid methyl ester. Yield 26%.

The above ester was dissolved in 9 mL of THF, 3 mL of MeOH and 3 mL of 1N LiOH solution and stirred overnight. The organic solvent was removed. Acetic acid was added and white precipitate formed. The precipitate was collected by filtration to yield 150 mg of the title compound. Yield 79%.

### Example 8

### Preparation of 1,3-dibenzyloxycarbonylamino-2-methyl-isothiourea

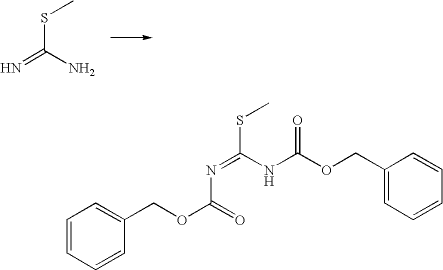

To a suspension of 2-methyl-isothiourea (1.0 g, 3.6 mmol) in methylene chloride (50 mL) was added 1N NaOH (18.0 mL, 18.0 mmol) and benzyl chloroformate (1.5 mL, 10.8 mmol). The mixture was stirred at room temperature for 24 h. The mixture was diluted with methylene chloride (20 mL) and the organic layer was washed with water three times. The combined organic layers were dried over sodium sulfate, filtered, and evaporated under reduced pressure. The crude oil was absorbed onto silica gel and chromatographed (silica gel, 5:1 hexanes/ethylacetate) provide 1,3-dibenzyloxycarbonylamino-2-methyl-isothiourea (0.78 g, 80%) as an oil.

Example 9 and 10 Illustrate the Synthesis of 2-aminobenzimidazole Derivatives Useful in the Preparation of Compounds of Formula (I).

### Example 9

### Preparation of N-[2-amino-1-(2-carbamoyl-ethyl)-1H-benzoimidazol-5-yl]-N-methyl-benzamide

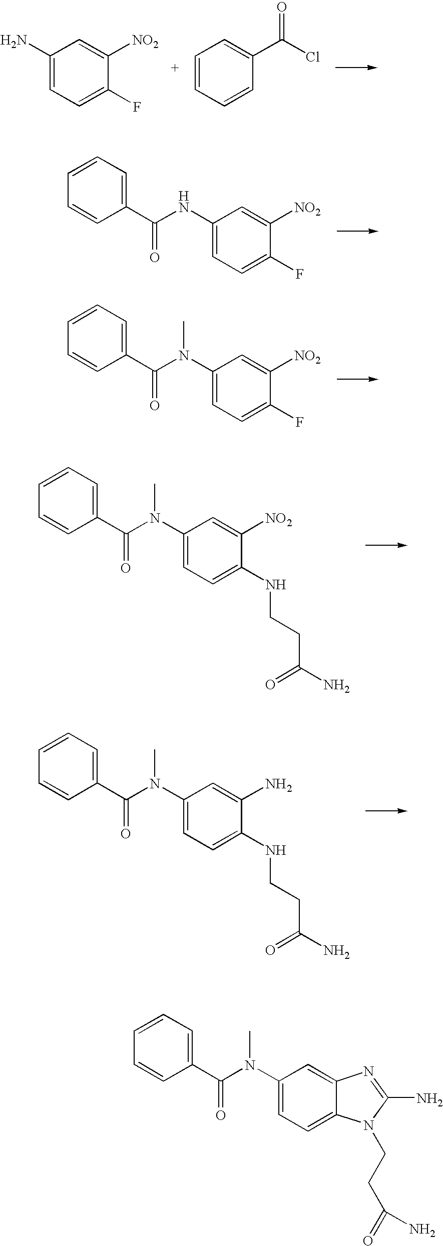

A mixture of 20 g (0.13 mol) of 4-fluoro-3-nitroaniline, 22.3 mL (0.19 mol, 1.5 eq.) of benzoyl chloride and 54 g (0.39 mol, 3.00 eq.) of K₂CO₃ in 300 mL of EtOAc and 300 mL of water was stirred at room temperature for 16 h. The precipitate was collected and washed with hexanes to give a desired compound as a light yellow solid. The organic layer of the filtrate was separated. This organic layer was washed with 10% NaOH solution, water and brine, dried (MgSO₄), filtered and concentrated. The residual solid was collected and washed with hexanes/EtOAc (4:1). This solid was combined with the other solid to give 32 g (96%) of N-(4-fluoro-3-nitro-phenyl)-benzamide as a light yellow solid. The product was pumped for 2 h and used for the next reaction without further purification.

A solution of 32 g (0.12 mol) of N-(4-fluoro-3-nitro-phenyl)-benzamide in 350 mL of THF was treated with 7.4 g (0.185 mol, 1.5 eq.) of NaH (60%) at 0° C. and the resulting mixture was stirred at room temperature for 30 min. To this mixture was added 11.5 mL (0.185 mol, 1.5 eq.) of methyliodide at this temperature. The resulting mixture was stirred at room temperature for 2 h, and quenched with water. The product was extracted into EtOAc. The organic layer was washed with water and brine, dried (MgSO₄) filtered and concentrated. The residue was purified by a short plug of silica gel and the residue was crystalized from EtOAc and hexanes to give 26.6 g (79%) of N-(4-fluoro-3-nitro-phenyl)-N-methyl-benzamide as a yellow solid.

A solution 24.7 g (90.0 mmol) of N-(4-fluoro-3-nitro-phenyl)-N-methyl-benzamide in 300 mL of DMF was added 22.4 g (180 mmol, 2.0 eq.) of β-alanine amide and 47 mL (270 mmol, 3.0 eq.) of Hunig's base. The resulting solution was stirred at 80° C. for 1 h after which time it was cooled to room temperature and poured into water and EtOAc. The layers were separated and the aqueous phase was extracted with EtOAc (2×). The combined organics were washed with water (2×) and dried (MgSO₄). Filtration and concentration gave the desired product which was filtered and washed with EtOAc to give 23.3 g (75%) of N-[4-(2-carbamoyl-ethylamino)-3-nitro-phenyl]-N-methyl-benzamide as an orange solid.

A solution of 23.2 g (67.7 mmol) of N-[4-(2-carbamoyl-ethylamino)-3-nitro-phenyl]-N-methyl-benzamide and 2.5 g of 10% Pd/C in 350 mL of MeOH was stirred at room temperature under hydrogen atmosphere for 3 h. Then the reaction mixture was filtered through diatomaceous earth and the filtrate was concentrated. The yellow foamy residue was pumped for 2 h providing N-[3-amino-4-(2-carbamoyl-ethylamino)-phenyl]-N-methyl-benzamide which was used for the next reaction without further purification (21.1 g, 99%).

To a stirred solution of 9.0 g of N-[3-amino-4-(2-carbamoyl-ethylamino)-phenyl]-N-methyl-benzamide in ethanol was added 3.4 g (32 mmol, 1.1 eq.) of cyanogen bromide. After 2 h the reaction was concentrated and dried on house vac for 24 h. The remaining residue was washed with EtOAc (3×) and treated with NaHCO₃ (sat., 500 mL) and water (200 mL). The precipitated solid was collected via filtration and washed with water. The resulting solid was dried at 60° C. for 12 h to give 9.2 g (95%) of title compound as a crystalline solid.

### Example 10

### Preparation of N-{2-amino-1-[(R)-2-(tert-butyl-dimethyl-silanyloxy)-2-phenyl-ethyl]-1H-benzoimidazol-5-yl}-N-methyl-benzamide

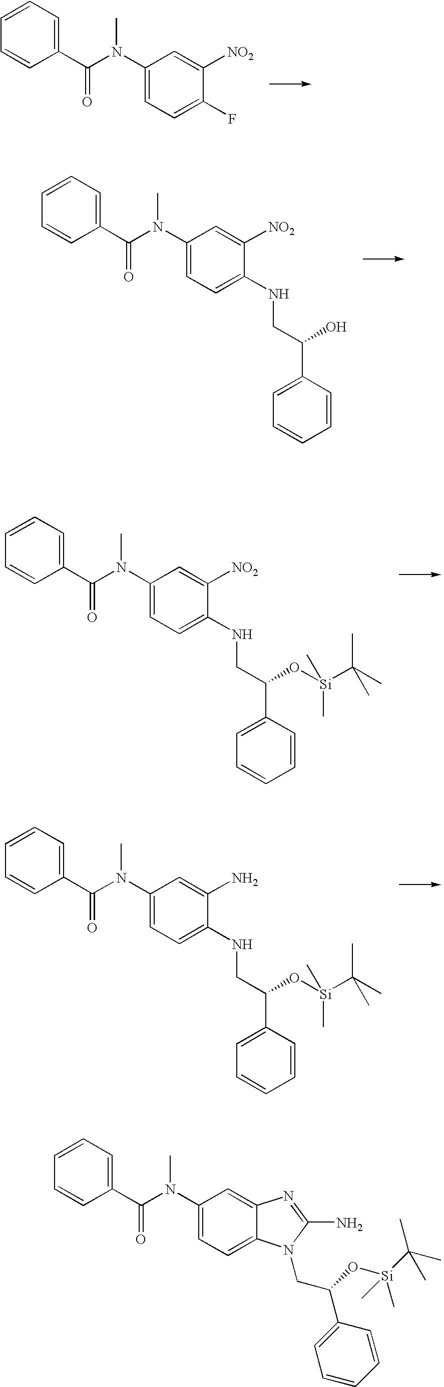

To a solution of [N-(4-fluoro-3-nitro-phenyl)-benzamide] (1.5 g, 5.5 mmol) in DMF (50 mL) were added R-(−)-2-amino-1-phenylethanol (1.3 g, 6.6 mmol) and iPr₂NEt (1.9 mL, 10.9 mmol) at room temperature. The solution was heated to 85° C. for 24 hours. Saturated NH₄Cl (10 mL) was added and the solution was extracted with EtOAc. The combined organic layer was dried with MgSO₄ and was filtered. The filtrate was concentrated and the residue was subjected to LC-MS analysis to confirm the presence of the desired product (2 g, 93.4%). The N-[4-((R)-2-hydroxy-2-phenyl-ethylamino)-3-nitro-phenyl]-N-methyl-benzamide was carried on to the next step without further purification.

To a solution of N-[4-((R)-2-hydroxy-2-phenyl-ethylamino)-3-nitro-phenyl]-N-methyl-benzamide (2.1 g, 5.4 mmol) in CH₂Cl₂ (50 mL) were added TBSCl (0.97 g, 6.4 mmol) and imidazole (0.74 g, 10.7 mmol) at room temperature. The solution was stirred at the same temperature for 24 h. 0.5 M HCl (20 mL) was added and the solution was stirred for 5 min. The solution was extracted with EtOAc and the combined organic layer was dried with MgSO₄ and was filtered. The filtrate was concentrated under reduced pressure and the residue was purified by Combiflash with 10% EtOAc in hexane to afford N-{4-[(R)-2-(tert-butyl-dimethyl-silanyloxy)-2-phenyl-ethylamino]-3-nitro-phenyl}-N-methyl-benzamide (2.7 g, 99.5%) as orange foam.

To a solution of N-{4-[(R)-2-(tert-butyl-dimethyl-silanyloxy)-2-phenyl-ethylamino]-3-nitro-phenyl}-N-methyl-benzamide (1.5 g, 2.9 mmol) in abs EtOH (50 mL) was added Pd/C (500 mg). The solution was degassed and H₂ was introduced. The solution was stirred at room temperature under the H₂ atmosphere for 24 hours. The solution was filtered through diatomaceous earth and was concentrated. The residue was subjected to LC-MS analysis to confirm the presence of the desirable product (1.4 g, 99.2%). The N-{3-amino-4-[(R)-2-(tert-butyl-dimethyl-silanyloxy)-2-phenyl-ethylamino]-phenyl}-N-methyl-benzamide was carried on to the next step of the synthesis without further purification.

To a solution of N-{3-amino-4-[(R)-2-(tert-butyl-dimethyl-silanyloxy)-2-phenyl-ethylamino]-phenyl}-N-methyl-benzamide (1.5 g, 3.1 mmol) in absolute EtOH (50 mL) was added BrCN (0.5 g, 4.7 mmol) at room temperature. The solution was stirred at the same temperature for 24 h. The completion of the reaction was confirmed by LC-MS. The solution was concentrated providing the title compound (1.5 g, 95%) which was used without further purification.

Examples 11-14 Illustrate Syntheses of Compounds of Formula (I) Using Intermediates from the Above Examples or Prepared as Described in the Above Examples.

### Example 11

### Preparation of 5-oxazol-5-yl-thiophene-2-carboxylic acid [5-(benzoyl-methyl-amino)-1-((R)-2-hydroxy-2-phenyl-ethyl)-1H-benzoimidazol-2-yl]-amide

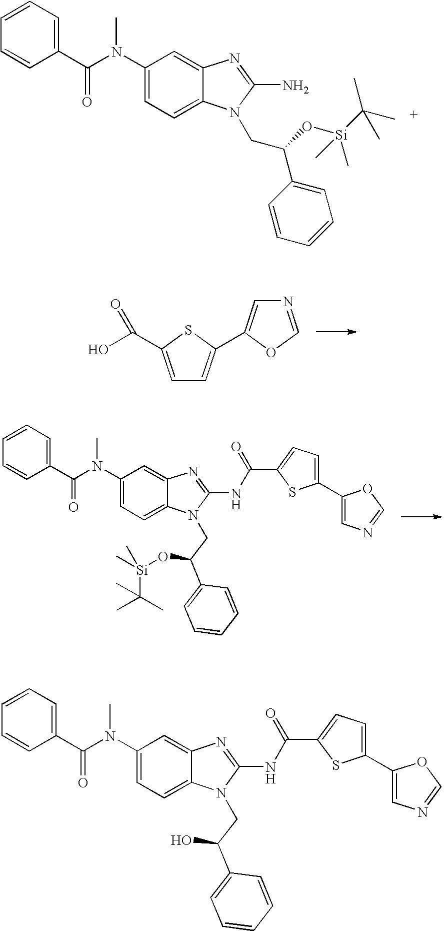

To a solution of N-{2-amino-1-[(R)-2-(tert-butyl-dimethyl-silanyloxy)-2-phenyl-ethyl]-1H-benzoimidazol-5-yl}-N-methyl-benzamide (Example 10) (100 mg, 0.2 mmol) in dry DMF (15 mL) were added 5-oxazol-5-yl-thiophene-2-carboxylic acid (Example 4) (78 mg, 0.4 mmol), EDC (115 mg, 0.6 mmol), HOBt (1-hydroxybenzotriazole hydrate; 54 mg, 0.4 mmol) and i-Pr₂NEt (0.07 mL, 0.4 mmol). The solution was stirred at room temperature for 24 h. Saturated NaHCO₃ solution (10 mL) was added and the solution was extracted with EtOAc (3 portions of 20 mL each). The combined organic layers were dried with MgSO₄ and were filtered. The filtrate was concentrated under reduced pressure. The residue was subjected to silica gel chromatography with 1 to 1 hexane and EtOAc as the eluent to afford 5-oxazol-5-yl-thiophene-2-carboxylic acid {5-(benzoyl-methyl-amino)-1-[(R)-2-(tert-butyl-dimethyl-silanyloxy)-2-phenyl-ethyl]-1H-benzoimidazol-2-yl}-amide (92 mg, 68%) as a colorless oil.

To a solution of the above amide (30 mg, 0.04 mmol) in THF (5 mL) was added TBAF (0.09 mL, 0.08 mmol) at room temperature. The solution was stirred at the same temperature for 24 h. Saturated NH₄Cl (10 mL) was added and the solution was extracted with EtOAc. The combined organic layer was dried with MgSO₄ and was filtered. The filtrate was concentrated under reduced pressure and the residue was purified by CombiFlash with 3% MeOH in CH₂Cl₂ as the eluent to afford the title compound (15.3 mg, 62%) as a yellow foam. MS (ESMS) m/z=564 (M+H)⁺

### Example 12

### Preparation of 5-(2-amino-thiazol-4-yl)-thiophene-2-carboxylic acid [5-(benzoyl-methyl-amino)-1-(2-carbamoyl-ethyl)-1H-benzoimidazol-2-yl]-amide

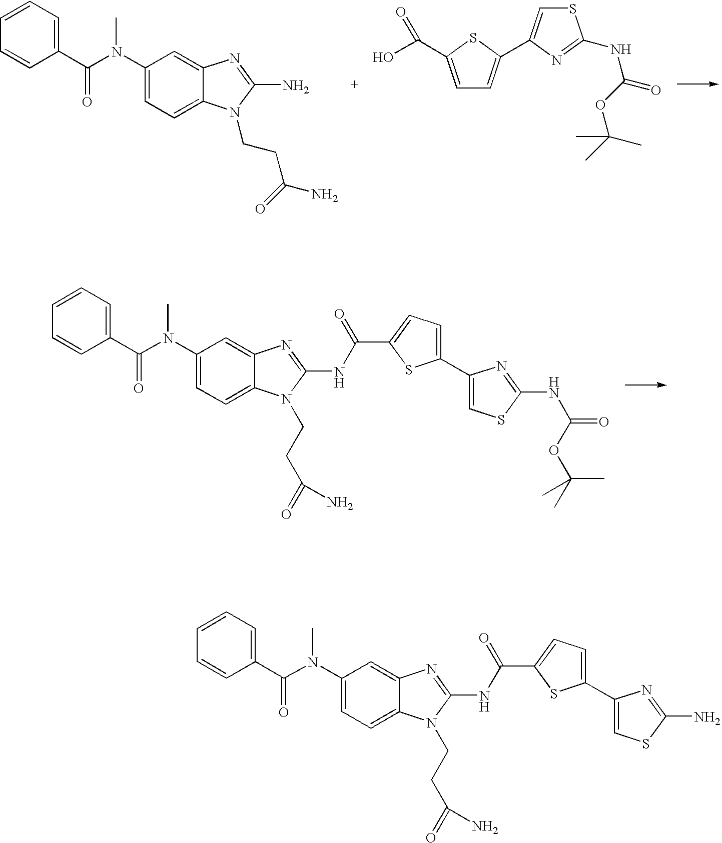

A mixture of 155 mg (0.37 mmol) of N-[2-amino-1-(2-carbamoyl-ethyl)-1H-benzoimidazol-5-yl]-N-methyl-benzamide (Example 9) and 145 mg (0.44 mmol, 1.2 eq.) of 5-(2-tert-butoxycarbonylamino-thiazol-4-yl)-thiophene-2-carboxylic acid (Example 7) in 3 mL DMF was treated with 84 mg (0.44 mmol, 1.2 eq.) of EDC, 60 mg (0.44 mmol, 1.2 eq.) of HOBt and 144 uL (0.66 mol, 2.4 eq.) of i-Pr₂NEt and stir at room temperature overnight. The reaction mixture was diluted with water, precipitate formed which was collected by filtration and purified by combiflash to yield 120 mg (4-{5-[5-(benzoyl-methyl-amino)-1-(2-carbamoyl-ethyl)-1H-benzoimidazol-2-ylcarbamoyl]-thiophen-2-yl}-thiazol-2-yl)-carbamic acid tert-butyl ester. Yield 50%.

A mixture of 80 mg of the above ester in 5 mL of CH₂Cl₂ was treated with 0.1 mL of TFA at room temperature. The reaction mixture was allowed to stir at the same temperature for 2 h. TLC showed no reaction. After stirring overnight, TLC still showed starting material remained. More TFA was added (total 1 mL) until TLC showed starting material was gone. The reaction was quenched with NaHCO₃ solution and extracted into EtOAc. Organics were combined, washed with water, saturated aqueous NaHCO₃, water, saturated aqueous NaCl, dried (MgSO₄), filtered and the solvent evaporated. The residue was purified by combiflash to yield 26 mg of the title compound as light yellow foam. Yield 37%. MS (ESMS) m/z=546 (M+H)⁺

### Example 13

### Preparation of 5-oxazol-5-yl-thiophene-2-carboxylic acid [5-(benzoyl-methyl-amino)-1-(2-pyridin-3-yl-ethyl)-1H-benzoimidazol-2-yl]-amide

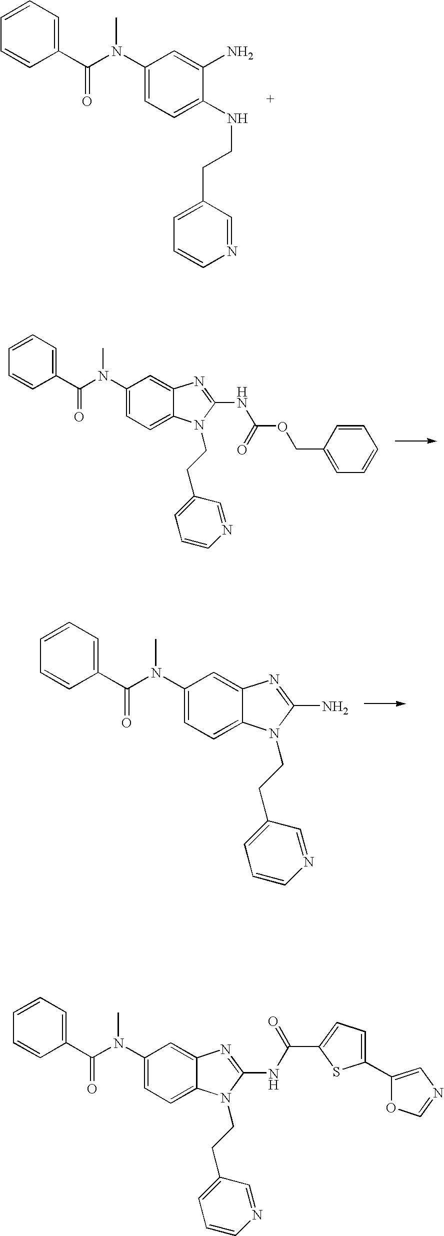

To a solution of N-[3-amino-4-(2-pyridin-3-yl-ethylamino)-phenyl]-N-methyl-benzamide (2.25 g, 6.50 mmol) in methanol (7 mL) was added 1,3-dibenzyloxycarbonylamino-2-methyl-isothiourea (Example 8) (2.10 g, 7.79 mmol) and p-toluenesulfonic acid (0.124 g, 0.65 mmol). The flask was fitted with a water condenser and the reaction was heated for 24 h at 50° C. The reaction mixture was then cooled to room temperature and the solvents removed at reduced pressure. Flash chromatography (silica gel, 75:25 to 33:67 hexanes/EtOAc) afforded [5-(benzoyl-methyl-amino)-1-(2-pyridin-3-yl-ethyl)-1H-benzoimidazol-2-yl]-carbamic acid benzyl ester (1.25 g, 38%) as a solid.

To a solution of the above benzyl ester (1.50 g, 2.97 mmol) in ethanol (100 mL) was added 10% w/w palladium on carbon (wet, 450 mg). The reaction was hydrogenated at 45 psi for 4 h then filtered through diatomaceous earth. The filtrate was concentrated at reduced pressure, and the solids were triturated with dichloromethane to afford N-[2-amino-1-(2-pyridin-3-yl-ethyl)-1H-benzoimidazol-5-yl]-N-methyl-benzamide (1.0 g, 91%) as a white solid.

To a solution of the above benzamide (92.4 mg, 0.249 mmol) and 5-oxazol-5-yl-thiophene-2-carboxylic acid (Example 4) (58.3 mg, 0.299 mmol) in DMF (3 mL) was added HOBt (48.0 mg, 0.348 mmol), EDC (68.0 mg, 0.348 mmol), and N,N-diisopropylethylamine (0.120 mL, 0.682 mmol). After stirring at room temperature overnight, the reaction mixture was diluted with EtOAc and washed with aqueous sodium bicarbonate and brine. The combined aqueous layer was then back extracted with EtOAc. The combined organic layer was washed with brine, dried over sodium sulfate, filtered and concentrated at reduced pressure. The residue was chromatographed (silica flash column, 95:5 dichloromethane/methanol) to afford the title compound (60 mg, 44%). MS (ESMS) m/z=549 (M+H)⁺

### Example 14

### Preparation of 5-Oxazol-5-yl-thiophene-2-carboxylic acid [5-[(3-cyano-benzoyl)-methyl-amino]-1-(2-pyridin-3-yl-ethyl)-1H-benzoimidazol-2-yl]-amide

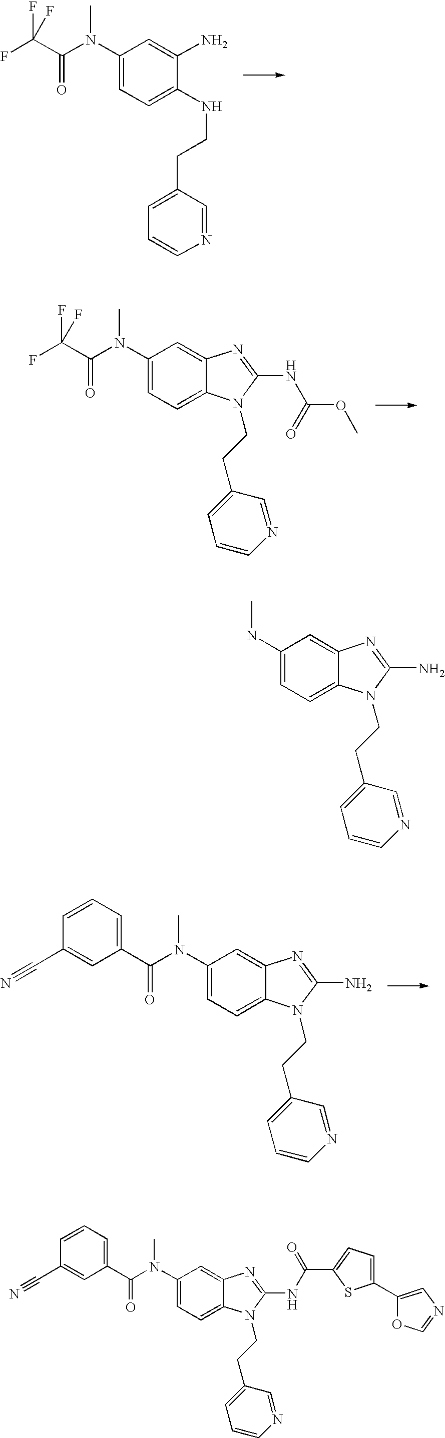

To a solution of N-[3-amino-4-(2-pyridin-3-yl-ethylamino)-phenyl]-2,2,2-trifluoro-N-methyl-acetamide (4.30 g, 12.7 mmol) in methanol (35 mL) was added 1,3-dimethoxycarbonylamino-2-methyl-isothiourea (2.62 g, 12.7 mmol) and p-toluenesulfonic acid (0.24 g, 1.27 mmol). The mixture was heated for 24 h at reflux and then cooled to room temperature and concentrated to dryness at reduced pressure. Flash chromatography (silica gel, 75:25 to 33:67 hexanes/EtOAc) afforded [5-[methyl-(2,2,2-trifluoro-acetyl)-amino]-1-(2-pyridin-3-yl-ethyl)-1H-benzoimidazol-2-yl]-carbamic acid methyl ester (4.54 g, 85%) as a solid.

A sealed tube containing a solution of the above methyl ester (0.75 g, 1.78 mmol), 35% aq. KOH (10 mL) and hydrazine monohydrate (0.86 mL, 17.8 mmol) in methanol (10 mL) was heated at 130° C. for 24 h. The methanol was removed at reduced pressure, and the residue was diluted with dichloromethane and washed with water (2×25 mL) and brine (2×25 mL). The aqueous layer was extracted with 75:25 chloroform/2-propanol, and the organic layers were combined, dried over sodium sulfate and concentrated to a dark oil. Flash chromatography (silica gel, 95:5 to 85:15 dichloromethane/methanol) afforded N⁵-Methyl-1-(2-pyridin-3-yl-ethyl)-1H-benzoimidazole-2,5-diamine (0.4 g, 83%) as a solid.

To a solution of N⁵-Methyl-1-(2-pyridin-3-yl-ethyl)-1H-benzoimidazole-2,5-diamine (1.22 g, 4.56 mmol) and 3-cyanobenzoic acid (740 mg, 5.03 mmol) in DMF (30 mL) was added HOBt (693 mg, 5.03 mmol), EDC (984 mg, 5.03 mmol), and N,N-diisopropylethylamine (1.20 mL, 6.82 mmol). The solution was stirred for 13 h at room temperature. The mixture was diluted with EtOAc and washed with aqueous sodium bicarbonate and brine. The combined aqueous layer was then back extracted with EtOAc. The combined organic layer was washed with brine, dried over sodium sulfate, filtered and concentrated at reduced pressure. The residue was chromatographed (silica flash column, 95:5 to 88:12 dichloromethane/methanol) to afford N-[2-Amino-1-(2-pyridin-3-yl-ethyl)-1H-benzoimidazol-5-yl]-3-cyano-N-methyl-benzamide (1.31 mg, 72%) as a brown foam.

To a solution of N-[2-amino-1-(2-pyridin-3-yl-ethyl)-1H-benzoimidazol-5-yl]-3-cyano-N-methyl-benzamide (205 mg, 0.517 mmol) and 5-oxazol-5-yl-thiophene-2-carboxylic acid (Example 4) (125 mg, 0.64 mmol) in DMF (5 mL) was added EDC (141 mg, 0.735 mmol), HOBt (101 mg, 0.747 mmol) and N,N-diisopropylethylamine (242 μL, 178 mg, 1.39 mmol). The solution was stirred for 14 h at room temperature. Water (3 mL) and saturated sodium bicarbonate (3 mL) were added, and the mixture was stirred for 10 min. The product, which separated as an oil, was extracted with EtOAc (4×20 mL). The organic extracts were combined, washed with brine, dried over sodium sulfate, filtered and concentrated at reduced pressure. The residue was chromatographed (silica flash column, 99:1 to 97:3 dichloromethane/methanol) to afford the title compound (230 mg, 78%) as a yellow foam. MS (ESMS) m/z=574 (M+H)⁺

### Example 15

### Preparation of 5-(2-methyl-oxazol-5-yl)-thiophene-2-carboxylic acid [5-(benzoyl-methyl-amino)-1-(2-methylamino-ethyl)-1H-benzoimidazol-2-yl]-amide

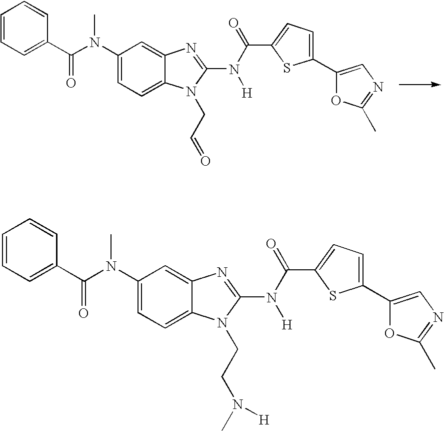

To a solution of 5-(2-methyl-oxazol-5-yl)-thiophene-2-carboxylic acid [5-(benzoyl-methyl-amino)-1-(2-oxo-ethyl)-1H-benzoimidazol-2-yl]-amide (50 mg, 0.1 mmol) in THF (0.2 mL) was added methylamine in THF (0.5 M, 0.25 mL) followed by MP-triacetoxyborohydride (107 mg, 0.215 mmol). The solution was agitated for 16 h. PS-Benzaldehyde (84 mg, 0.1 mmol) and THF (1 mL) was added and the mixture was further agitated 4 h. The solution was filtered and the filtrate concentrated in vacuo. The title compound (10 mg 19%) was obtained as a yellow foam after purification by flash chromatography using a 1-10% MeOH/methylene chloride gradient.

### Example 16

### Preparation of 5-(2-methyl-oxazol-5-yl)-thiophene-2-carboxylic acid [5-[(cyclohexyl-methyl-amino)-methyl]-1-(2-hydroxy-2-methyl-propyl)-1H-benzoimidazol-2-yl]-amide

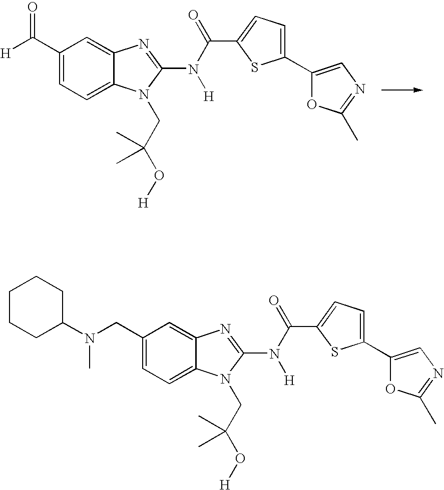

To a solution of 5-(2-methyl-oxazol-5-yl)-thiophene-2-carboxylic acid [5-formyl-1-(2-hydroxy-2-methyl-propyl)-1H-benzoimidazol-2-yl]-amide (0.1 g, 0.236 mmol) in THF (3 mL) was added cyclohexylmethylamine (0.037 mL, 0.283 mmol) and MP-BH(OAc)₃ (0.892 g, 1.61 mmol). The solution was agitated for 16 h at room temperature. PS-Isocyanate resin (0.344 g, 0.236 mmol) was added and the reaction agitated for an additional 4 h. The solution was filtered and the filtrate concentrated in vacuo and purified by flash chromatography to afford the title compound (56 mg, 46%).

### Example 17

### Preparation of 5-(2-methyl-oxazol-5-yl)-thiophene-2-carboxylic acid [1-(2-hydroxy-2-methyl-propyl)-5-phenylaminomethyl-1H-benzoimidazol-2-yl]-amide

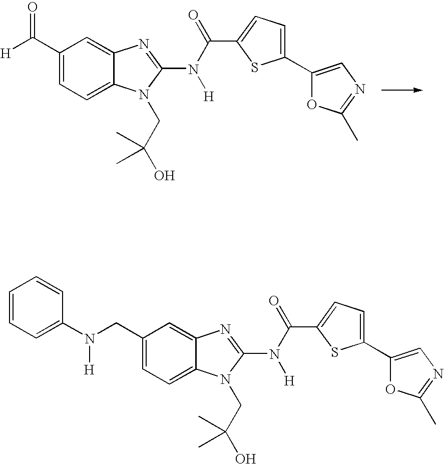

To a solution of 5-(2-methyl-oxazol-5-yl)-thiophene-2-carboxylic acid [5-formyl-1-(2-hydroxy-2-methyl-propyl)-1H-benzoimidazol-2-yl]-amide (0.1 g, 0.236 mmol) in THF (3 mL) was added aniline (0.026 mL, 0.283 mmol) and MP-BH₃CN (1.2 g, 2.89 mmol). The solution was agitated for 16 h at room temperature. PS-Benzaldehyde resin (0.257 g, 0.236 mmol) was added and the reaction was agitated for an additional 4 h. The solution was filtered and the filtrate was concentrated in vacuo and purified by flash chromatography to afford the title compound (131 mg, 100%).

### Example 18

### Preparation of 5-(2-methyl-oxazol-5-yl)-thiophene-2-carboxylic acid [5-(2-dimethylamino-ethoxy)-1-(2-hydroxy-2-methyl-propyl)-1H-benzoimidazol-2-yl]-amide

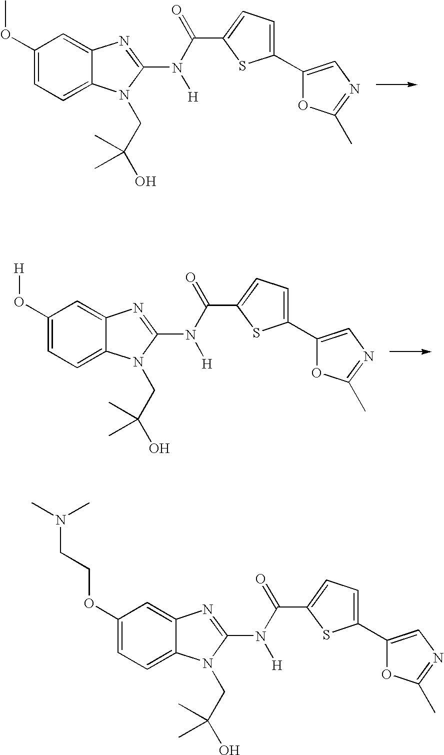

To a solution of 5-(2-methyl-oxazol-5-yl)-thiophene-2-carboxylic acid [1-(2-hydroxy-2-methyl-propyl)-5-methoxy-1H-benzoimidazol-2-yl]-amide (0.760 g, 1.78 mmol) in methylene chloride (160 mL) was added BBr₃ (1 M in methylene chloride, 35.6 mL, 35.64 mmol) at room temperature. The reaction was stirred for 2 h and then quenched with MeOH and concentrated in vacuo. Purification by flash chromatography afforded 5-(2-methyl-oxazol-5-yl)-thiophene-2-carboxylic acid [5-hydroxy-1-(2-hydroxy-2-methyl-propyl)-1H-benzoimidazol-2-yl]-amide (0.6 g, 86%).

To a solution of the above amide (0.05 g, 0.121 mmol) in DMF (2 mL) was added the hydrochloride salt of (2-chloro-ethyl)-dimethyl-amine (0.016 g, 0.101 mmol) and potassium carbonate (0.033 g, 0.242 mmol) and the mixture was allowed to stir for 16 h. Additional potassium carbonate and alkyl chloride were added and the reaction was stirred another 16 h, then diluted with water and extracted with EtOAc. The combined organics were washed with water and brine, dried over sodium sulfate and filtered. The filtrate was concentrated in vacuo and purified via flash chromatography to afford the title compound as a yellow film (0.012 g, 24%).

Examples 19-24 Illustrate the Synthesis of Intermediates Which may be Used in the Preparation of Compounds of the Invention by Methods Described in the Above Synthetic Examples

### Example 19

### Preparation of 2-methyl-1-(2-nitro-phenylamino)-propan-2-ol

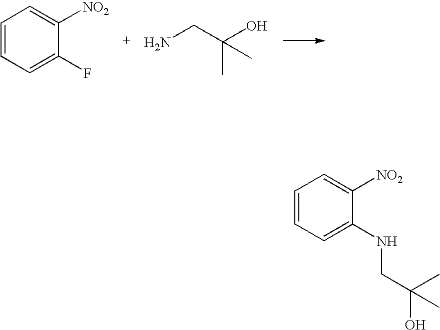

A solution of 1-fluoro-2-nitrobenzene (0.374 mL, 3.51 mmol) and 1,1-dimethylethanolamine (0.375 g, 4.21 mmol) and diisopropylethylamine (1.22 mL, 7.02 mmol) in 10 mL of DMF was heated to 80° C. for 24 h. The reaction mixture was cooled to room temperature and concentrated. The residue was diluted with 20 mL of water and 20 mL of EtOAc, and the organic layer was separated. The aqueous layer was extracted with EtOAc (2×10 mL), and the combined organics were washed with saturated NaHCO₃ (1×20 mL) and brine (1×20 mL), then dried over MgSO₄ to yield 0.772 g of the title compound which was taken on without further purification.

### Example 20

### Preparation of 2-methyl-1-(4-fluoro-2-nitro-phenylamino)-propan-2-ol

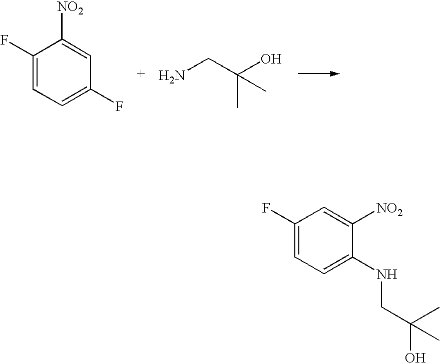

A solution of 1,4-difluoro-2-nitrobenzene (0.392 mL, 3.51 mmol) and 1,1-dimethylethanolamine (0.375 g, 4.21 mmol) and diisopropylethylamine (1.22 mL, 7.02 mmol) in 10 mL of DMF was heated to 80° C. for 24 h. The reaction mixture was cooled to room temperature and concentrated. The residue was diluted with 20 mL of water and 20 mL of EtOAc, and the organic layer was separated. The aqueous layer was extracted with EtOAc (2×10 mL), and the combined organics were washed with saturated NaHCO₃ (1×20 mL) and brine (1×20 mL), then dried over MgSO₄ to yield 0.887 g of the title compound which was taken on without further purification.

### Example 21

### Preparation of 2-methyl-1-(4-bromo-2-nitro-phenylamino)-propan-2-ol

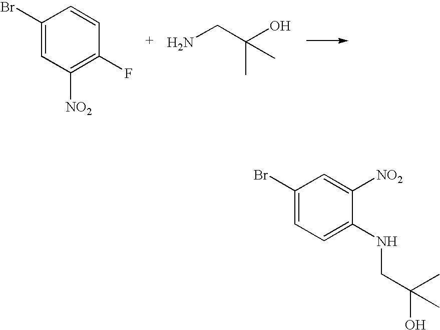

A solution of 4-bromo-1-fluoro-2-nitrobenzene (0.450 mL, 3.51 mmol) and 1,1-dimethylethanolamine (0.375 g, 4.21 mmol) and diisopropylethylamine (1.22 mL, 7.02 mmol) in 10 mL of DMF was heated to 80° C. for 24 h. The reaction mixture was cooled to room temperature and concentrated. The residue was diluted with 20 mL of water and 20 mL of EtOAc, and the organic layer was separated. The aqueous layer was extracted with EtOAc (2×10 mL), and the combined organics were washed with saturated NaHCO₃ (1×20 mL) and brine (1×20 mL), then dried over MgSO₄ to yield 1.21 g of the title compound which was taken on without further purification.

### Example 22

### Preparation of 2-methyl-1-(4-trifluoromethyl-2-nitro-phenylamino)-propan-2-ol

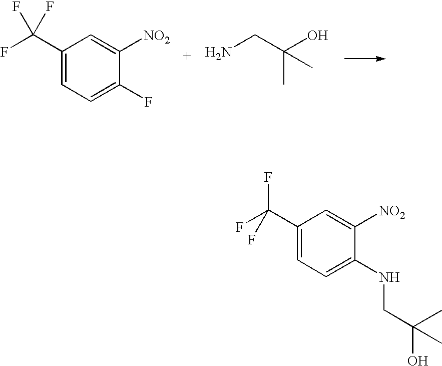

A solution of 1-fluoro-2-nitro-4-trifluoromethylbenzene (0.506 mL, 3.51 mmol) and 1,1-dimethylethanolamine (0.375 g, 4.21 mmol) and diisopropylethylamine (1.22 mL, 7.02 mmol) in 10 mL of DMF was heated to 80° C. for 24 h. The reaction mixture was cooled to room temperature and concentrated. The residue was diluted with 20 mL of water and 20 mL of EtOAc, and the organic layer was separated. The aqueous layer was extracted with EtOAc (2×10 mL), and the combined organics were washed with saturated NaHCO₃ (1×20 mL) and brine (1×20 mL), then dried over MgSO₄ to yield 0.971 g of the title compound which was taken on without further purification.

### Example 23

### Preparation of 2-methyl-1-(4-methanesulfonyl-2-nitro-phenylamino)-propan-2-ol

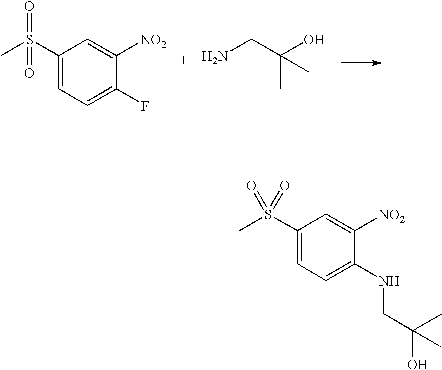

A solution of 1-fluoro-4-methanesulfonyl-2-nitrobenzene (0.810 g, 3.51 mmol) and 1,1-dimethylethanolamine (0.375 g, 4.21 mmol) and diisopropylethylamine (1.22 mL, 7.02 mmol) in 10 mL of DMF was heated to 80° C. for 24 h. The reaction mixture was cooled to room temperature and concentrated. The residue was diluted with 20 mL of water and 20 mL of EtOAc, and the organic layer was separated. The aqueous layer was extracted with EtOAc (2×10 mL), and the combined organics were washed with saturated NaHCO₃ (1×20 mL) and brine (1×20 mL), then dried over MgSO₄ to yield 1.07 g of the title compound which was taken on without further purification.

### Example 24

### Preparation of [4-(2-hydroxy-2-methyl-propylamino)-3-nitro-phenyl]-carbamic acid tert-butyl ester

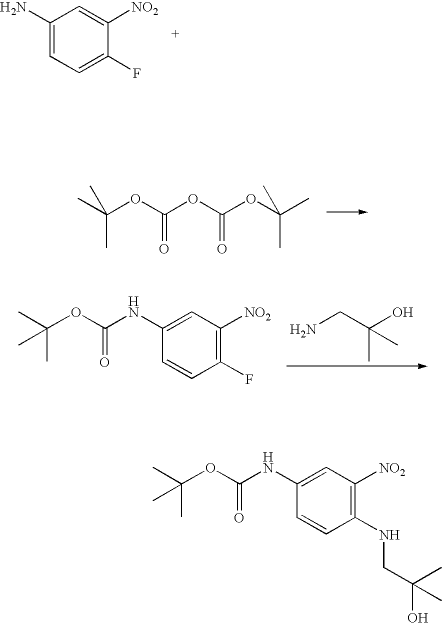

To a solution of 4-fluoro-3-nitroaniline (10 g, 70 mmol) in 100 mL of THF was added Boc₂O (15.3 g, 64 mmol) and a catalytic amount of N,N-dimethylaminopyridine and the solution was stirred at room temperature for 16 h. The solution was concentrated and diluted with 100 mL of EtOAc, washed with 1M HCl (aq) (1×50 mL) and saturated NaHCO₃ (1×50 mL) and dried over Na₂SO₄. The crude product was concentrated and purified by combiflash to yield 13.28 g of the desired carbamic acid ester.

A solution of the above carbamic acid ester (0.300 g, 1.71 mmol), 1,1-dimethyl ethanolamine (0.125 g, 1.40 mmol) and diisopropylethylamine (0.306 mL, 1.76 mmol) in 3 mL of DMF was heated to 60° C. for 24 h. The reaction mixture was cooled to room temperature and concentrated. The residue was diluted with 10 mL of water and 10 mL of EtOAc, and the organic layer was separated. The aqueous layer was extracted with EtOAc (2×50 mL) and the combined organics were washed with saturated NaHCO₃ (1×10 mL) and brine (1×10 mL), then dried over MgSO₄ to yield 0.165 g of the title compound which was taken on without further purification.

### Example 25

### Preparation of 5-(2-methyl-oxazol-5-yl)-thiophene-2-carboxylic acid [1-(2-hydroxy-2-methyl-propyl)-5-pyrrolidin-1-yl-1H-benzoimidazol-2-yl]-amide

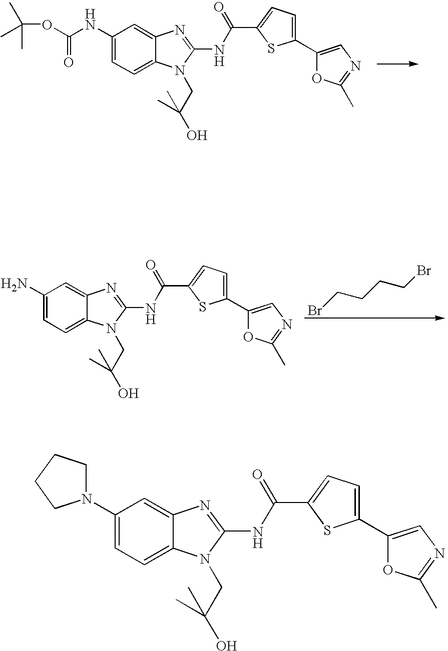

To a stirred solution of (1-(2-hydroxy-2-methyl-propyl)-2-{[5-(2-methyl-oxazol-5-yl)-thiophene-2-carbonyl]-amino}-1H-benzoimidazol-5-yl)-carbamic acid tert-butyl ester (0.100 g, 0.195 mmol) in 5 mL of dichloromethane was added 0.5 ml of TFA. The solution was stirred at room temperature for 16 h and concentrated. The crude product was diluted with 10 mL of dichloromethane, washed with 10 mL of saturated NaHCO₃, back-extracted (2×10 mL) with dichloromethane, and dried over MgSO₄. The solution was concentrated and purified by combiflash to yield 80 mg (99%) of 5-(2-methyl-oxazol-5-yl)-thiophene-2-carboxylic acid [5-amino-1-(2-hydroxy-2-methyl-propyl)-1H-benzoimidazol-2-yl]-amide.

To a solution of the above intermediate (0.080 g, 0.194 mmol) in 2 mL of dimethylacetamide was added diisopropylethylamine (0.007 mL, 0.039 mmol) and 1,4-dibromobutane (0.026 mL, 0.214 mmol). The mixture was heated at 70° C. for 18 h and cooled to room temperature. The crude product was diluted with 5 mL of dichloromethane and washed with 10 mL of saturated NaHCO₃, dried over MgSO₄ and concentrated. The crude product was purified by combiflash to yield 0.044 g (49%) of the title compound.

### Example 26

### Preparation of 1-[4-(2-hydroxy-2-methyl-propylamino)-3-nitro-phenyl]-pyrrolidin-2-one

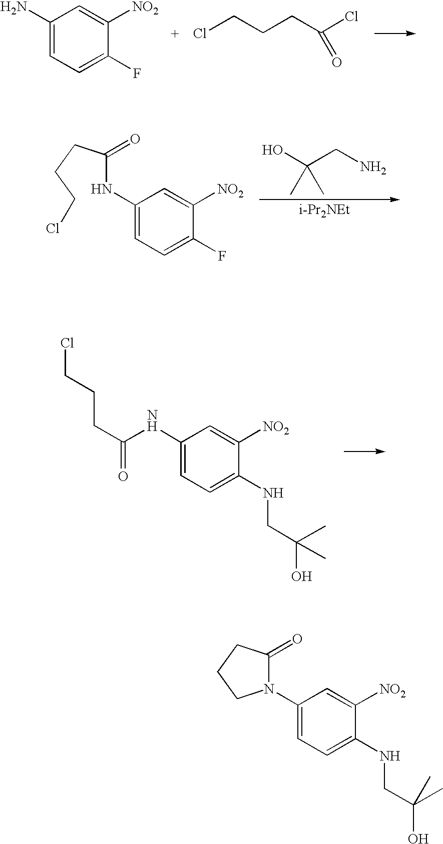

To a solution of 4-fluoro-3-nitroaniline (2.00 g, 12.8 mmol) in 20 mL of toluene was added diisopropylethylamine (4.30 mL, 24.0 mmol) and 4-chlorobutyryl chloride (1.58 mL, 14.1 mmol). The solution was stirred for 16 h and concentrated. The crude product was diluted with 10 mL of water and 20 mL of dichloromethane, and the aqueous layer was extracted (3×10 mL) with dichloromethane. The combined organic layers were dried over MgSO₄ and concentrated to yield 3.0 g of crude amide intermediate which was taken on without further purification.

To a solution of the above amide intermediate (0.320 g, 1.23 mmol) in 5 mL of dimethylformamide was added 1,1-dimethyl ethanolamine (0.140 g, 1.57 mmol). Diisopropylethylamine was added (0.420 mL, 2.39 mmol) and the reaction was stirred at 40° C. for 16 h. The solution was diluted with 10 mL of EtOAc and 10 mL of water, and the organic layer was then washed with saturated NaHCO₃ and dried over Na₂SO₄. The solution was filtered and concentrated to yield 156 mg of 4-chloro-N-[4-(2-hydroxy-2-methyl-propylamino)-3-nitro-phenyl]-butyramide that was taken on without further purification.

To a solution of the above butyramide intermediate (0.156 g, 0.473 mmol) in 1 mL of MeOH was added 1 mL of a 2M sodium methoxide in MeOH solution and stirred overnight. The solution was diluted with 5 mL of dichloromethane and 5 mL of water and the aqueous layer was extracted (2×5 mL) with dichloromethane and dried over MgSO₄, concentrated and purified by combiflash to yield 85 mg (61%) of the title compound.

### Example 27

### Preparation of 5-(2-methyl-oxazol-5-yl)-thiophene-2-carboxylic acid [1-(2-hydroxy-2-methyl-propyl)-5-oxazol-5-yl-1H-benzoimidazol-2-yl]-amide

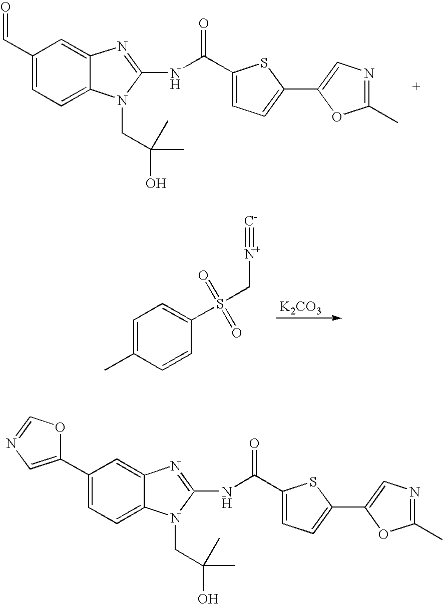

A mixture of 1 (0.200 g, 0.471 mmol), tosylmethyl isocyanide (0.095 g, 0.471 mmol) and K₂CO₃ (0.195 g, 1.41 mmol) in 5 mL of MeOH was heated to 60° C. for 1 h. The mixture was concentrated and the crude product was recrystallized from EtOH as the potassium salt. The salt was diluted with 10 mL of H₂O and 10 mL of dichloromethane. The organic phase was washed with 5 mL of saturated NH₄Cl and 5 mL of saturated NaCl. The organic solution was dried over MgSO₄ and concentrated, and the crude product was purified by combiflash to yield 61 mg (28%) of the title compound.

The following additional compounds in Table II can be made by methods similar to those in the examples listed above and by methods known in the art.

TABLE II

| | | | Mass |
|---|---|---|---|
| | | | (M+H)⁺ |
| Example | Structure | NAME | m/z |
| Example 28 | 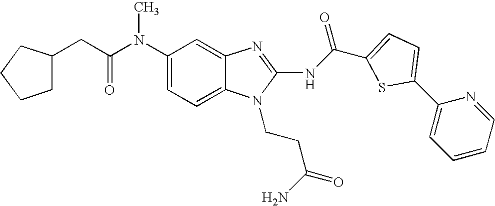 | 5-Pyridin-2-yl-thiophene-2- carboxylic acid {1-(2-carbamoyl- ethyl)-5-[(2-cyclopentyl-acetyl)- methyl-amino]-1H- benzoimidazol-2-yl}-amide | 531 |
| Example 29 | 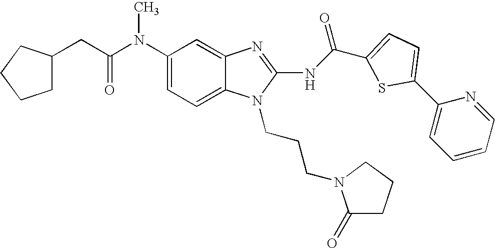 | 5-Pyridin-2-yl-thiophene-2- carboxylic acid {5-[(2- cyclopentyl-acetyl)-methyl- amino]-1-[3-(2-oxo-pyrrolidin-1- yl)-propyl]-1H-benzoimidazol-2- yl}-amide | 585 |
| Example 30 | 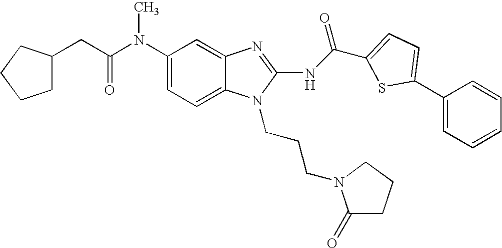 | 5-Phenyl-thiophene-2-carboxylic acid {5-[(2-cyclopentyl-acetyl)- methyl-amino]-1-[3-(2-oxo- pyrrolidin-1-yl)-propyl]-1H- benzoimidazol-2-yl}-amide | 584 |
| Example 31 | 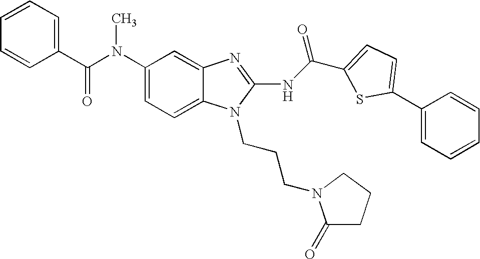 | 5-Phenyl-thiophene-2-carboxylic acid {5-(benzoyl-methyl-amino)- 1-[3-(2-oxo-pyrrolidin-1-yl)- propyl]-1H-benzoimidazol-2-yl}- amide | 578 |
| Example 32 | 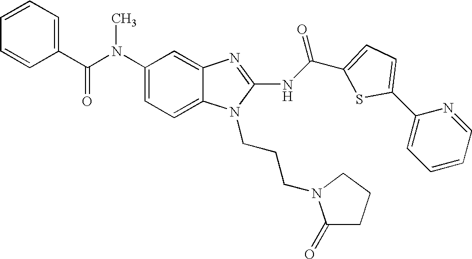 | 5-Pyridin-2-yl-thiophene-2- carboxylic acid {5-(benzoyl- methyl-amino)-1-[3-(2-oxo- pyrrolidin-1-yl)-propyl]-1H- benzoimidazol-2-yl}-amide | 579 |
| Example 33 | 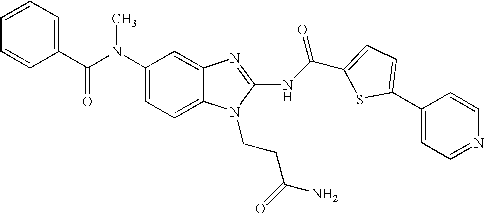 | 5-Pyridin-4-yl-thiophene-2- carboxylic acid [5-(benzoyl- methyl-amino)-1-(2-carbamoyl- ethyl)-1H-benzoimidazol-2-yl]- amide | 525 |
| Example 34 | 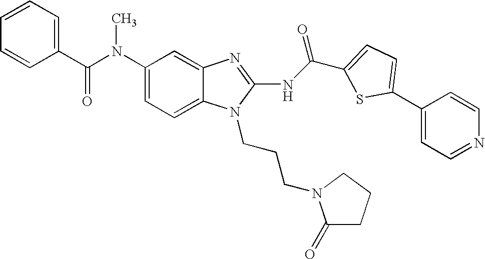 | 5-Pyridin-4-yl-thiophene-2- carboxylic acid {5-(benzoyl- methyl-amino)-1-[3-(2-oxo- pyrrolidin-1-yl)-propyl]-1H- benzoimidazol-2-yl}-amide | 579 |
| Example 35 | 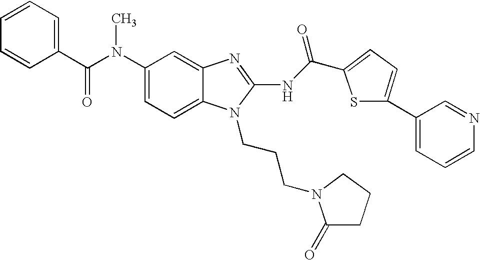 | 5-Pyridin-3-yl-thiophene-2- carboxylic acid {5-(benzoyl- methyl-amino)-1-[3-(2-oxo- pyrrolidin-1-yl)-propyl]-1H- benzoimidazol-2-yl}-amide | 579 |
| Example 36 | 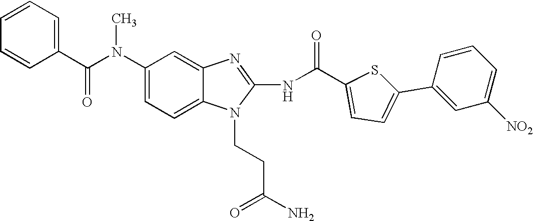 | 5-(3-Nitro-phenyl)-thiophene-2- carboxylic acid [5-(benzoyl- methyl-amino)-1-(2-carbamoyl- ethyl)-1H-benzoimidazol-2-yl]- amide | 569 |
| Example 37 | 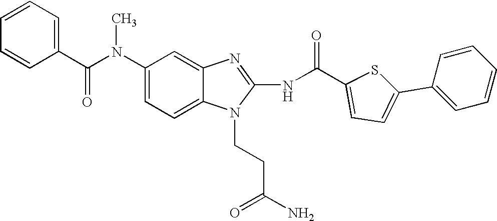 | 5-Phenyl-thiophene-2-carboxylic acid [5-(benzoyl-methyl-amino)- 1-(2-carbamoyl-ethyl)-1H- benzoimidazol-2-yl]-amide | 524 |
| Example 38 | 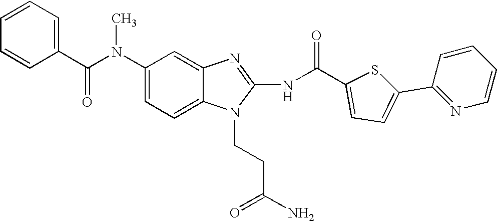 | 5-Pyridin-2-yl-thiophene-2- carboxylic acid [5-(benzoyl- methyl-amino)-1-(2-carbamoyl- ethyl)-1H-benzoimidazol-2-yl]- amide | 525 |
| Example 39 | 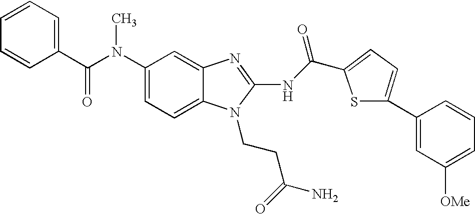 | 5-(3-Methoxy-phenyl)-thiophene- 2-carboxylic acid [5-(benzoyl- methyl-amino)-1-(2-carbamoyl- ethyl)-1H-benzoimidazol-2-yl]- amide | 554 |
| Example 40 | 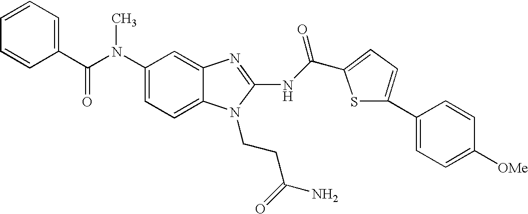 | 5-(4-Methoxy-phenyl)-thiophene- 2-carboxylic acid [5-(benzoyl- methyl-amino)-1-(2-carbamoyl- ethyl)-1H-benzoimidazol-2-yl]- amide | 554 |
| Example 41 | 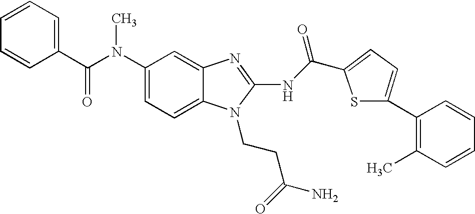 | 5-o-Tolyl-thiophene-2-carboxylic acid [5-(benzoyl-methyl-amino)- 1-(2-carbamoyl-ethyl)-1H- benzoimidazol-2-yl]-amide | 538 |
| Example 42 | 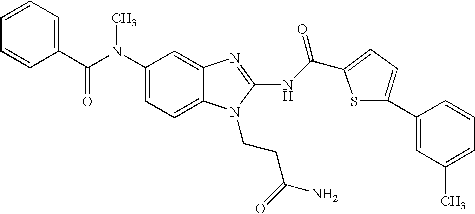 | 5-m-Tolyl-thiophene-2-carboxylic acid [5-(benzoyl-methyl-amino)- 1-(2-carbamoyl-ethyl)-1H- benzoimidazol-2-yl]-amide | 538 |
| Example 43 | 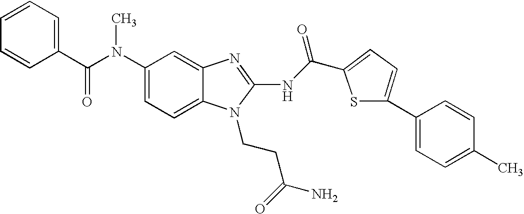 | 5-p-Tolyl-thiophene-2-carboxylic acid [5-(benzoyl-methyl-amino)- 1-(2-carbamoyl-ethyl)-1H- benzoimidazol-2-yl]-amide | 538 |
| Example 44 | 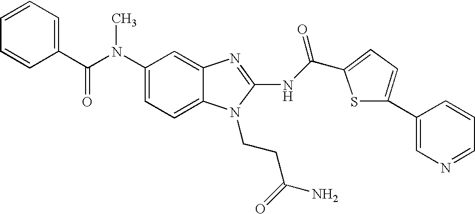 | 5-Pyridin-3-yl-thiophene-2- carboxylic acid [5-(benzoyl- methyl-amino)-1-(2-carbamoyl- ethyl)-1H-benzoimidazol-2-yl]- amide | 525 |
| Example 45 | 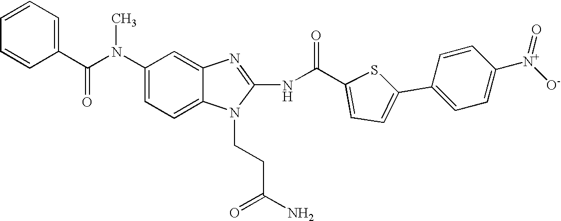 | 5-(4-Nitro-phenyl)-thiophene-2- carboxylic acid [5-(benzoyl- methyl-amino)-1-(2-carbamoyl- ethyl)-1H-benzoimidazol-2-yl]- amide | 569 |
| Example 46 | 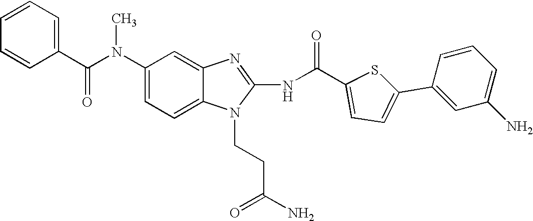 | 5-(3-Amino-phenyl)-thiophene-2- carboxylic acid [5-(benzoyl- methyl-amino)-1-(2-carbamoyl- ethyl)-1H-benzoimidazol-2-yl]- amide | 539 |
| Example 47 | 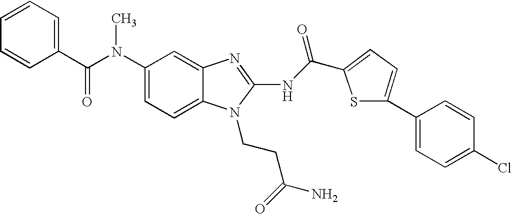 | 5-(4-Chloro-phenyl)-thiophene-2- carboxylic acid [5-(benzoyl- methyl-amino)-1-(2-carbamoyl- ethyl)-1H-benzoimidazol-2-yl]- amide | 558 |
| Example 48 | 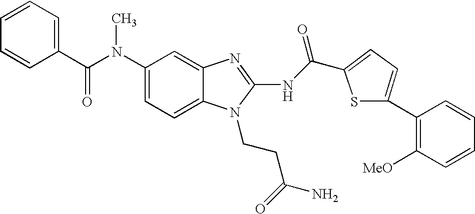 | 5-(2-Methoxy-phenyl)-thiophene- 2-carboxylic acid [5-(benzoyl- methyl-amino)-1-(2-carbamoyl- ethyl)-1H-benzoimidazol-2-yl]- amide | 554 |
| Example 49 | 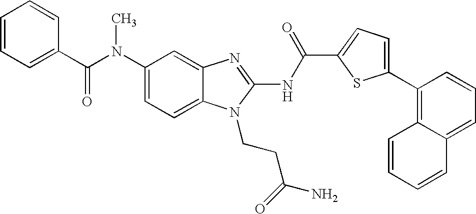 | 5-Naphthalen-1-yl-thiophene-2- carboxylic acid [5-(benzoyl- methyl-amino)-1-(2-carbamoyl- ethyl)-1H-benzoimidazol-2-yl]- amide | 574 |
| Example 50 | 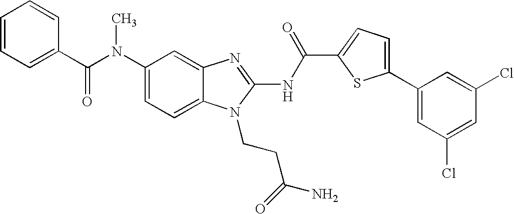 | 5-(3,5-Dichloro-phenyl)- thiophene-2-carboxylic acid [5- (benzoyl-methyl-amino)-1-(2- carbamoyl-ethyl)-1H- benzoimidazol-2-yl]-amide | 593 |
| Example 51 | 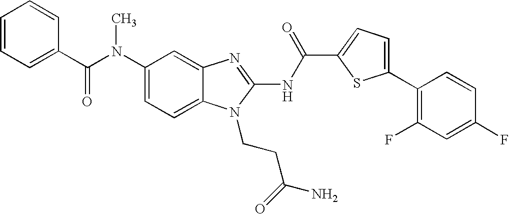 | 5-(2,4-Difluoro-phenyl)- thiophene-2-carboxylic acid [5- (benzoyl-methyl-amino)-1-(2- carbamoyl-ethyl)-1H- benzoimidazol-2-yl]-amide | 560 |
| Example 52 | 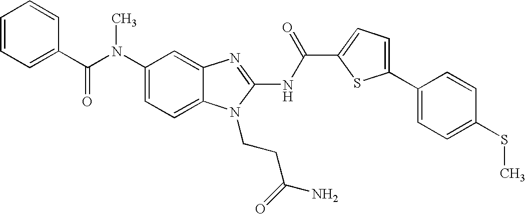 | 5-(4-Methylsulfanyl-phenyl)- thiophene-2-carboxylic acid [5- (benzoyl-methyl-amino)-1-(2- carbamoyl-ethyl)-1H- benzoimidazol-2-yl]-amide | 570 |
| Example 53 | 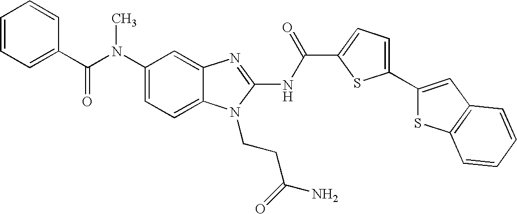 | 5-Benzo[b]thiophen-2-yl- thiophene-2-carboxylic acid [5- (benzoyl-methyl-amino)-1-(2- carbamoyl-ethyl)-1H- benzoimidazol-2-yl]-amide | 580 |
| Example 54 | 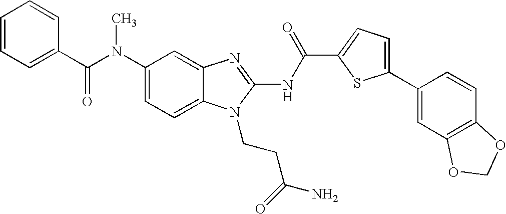 | 5-Benzo[1,3]dioxol-5-yl- thiophene-2-carboxylic acid [5- (benzoyl-methyl-amino)-1-(2- carbamoyl-ethyl)-1H- benzoimidazol-2-yl]-amide | 568 |
| Example 55 | 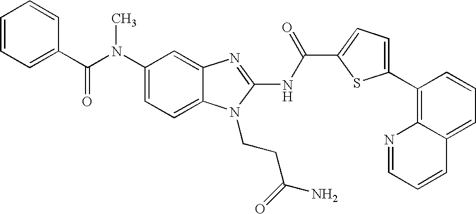 | 5-Quinolin-8-yl-thiophene-2- carboxylic acid [5-(benzoyl- methyl-amino)-1-(2-carbamoyl- ethyl)-1H-benzoimidazol-2-yl]- amide | 575 |
| Example 56 | 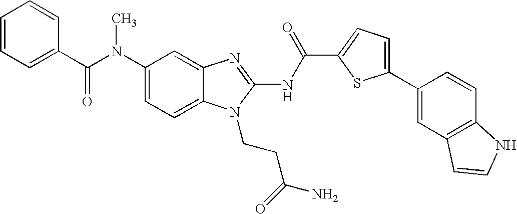 | 5-(1H-Indol-5-yl)-thiophene-2- carboxylic acid [5-(benzoyl- methyl-amino)-1-(2-carbamoyl- ethyl)-1H-benzoimidazol-2-yl]- amide | 563 |
| Example 57 | 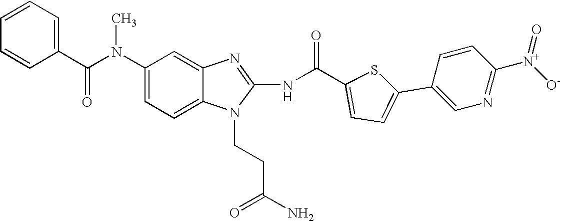 | 5-(6-Nitro-pyridin-3-yl)- thiophene-2-carboxylic acid [5- (benzoyl-methyl-amino)-1-(2- carbamoyl-ethyl)-1H- benzoimidazol-2-yl]-amide | 570 |
| Example 58 | 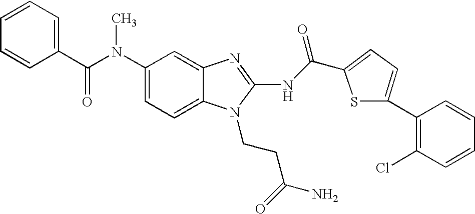 | 5-(2-Chloro-phenyl)-thiophene-2- carboxylic acid [5-(benzoyl- methyl-amino)-1-(2-carbamoyl- ethyl)-1H-benzoimidazol-2-yl]- amide | 558 |
| Example 59 | 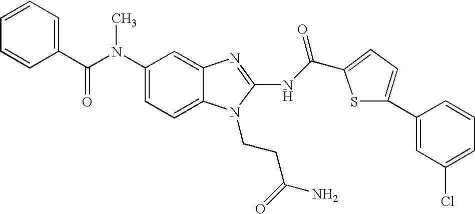 | 5-(3-Chloro-phenyl)-thiophene-2- carboxylic acid [5-(benzoyl- methyl-amino)-1-(2-carbamoyl- ethyl)-1H-benzoimidazol-2-yl]- amide | 558 |
| Example 60 | 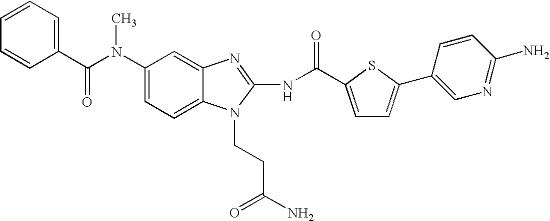 | 5-(6-Amino-pyridin-3-yl)- thiophene-2-carboxylic acid [5- (benzoyl-methyl-amino)-1-(2- carbamoyl-ethyl)-1H- benzoimidazol-2-yl]-amide | 540 |
| Example 61 | 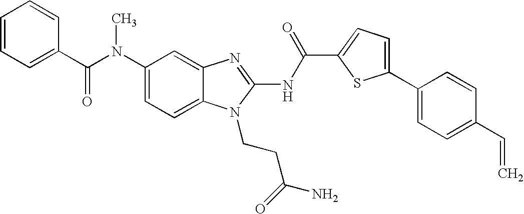 | 5-(4-Vinyl-phenyl)-thiophene-2- carboxylic acid [5-(benzoyl- methyl-amino)-1-(2-carbamoyl- ethyl)-1H-benzoimidazol-2-yl]- amide | 550 |
| Example 62 | 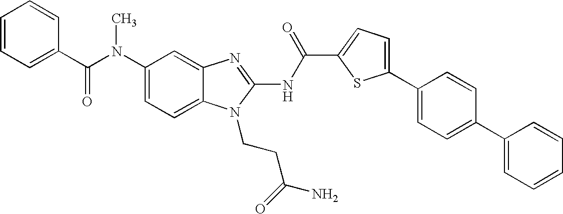 | 5-Biphenyl-4-yl-thiophene-2- carboxylic acid [5-(benzoyl- methyl-amino)-1-(2-carbamoyl- ethyl)-1H-benzoimidazol-2-yl]- amide | 600 |
| Example 63 | 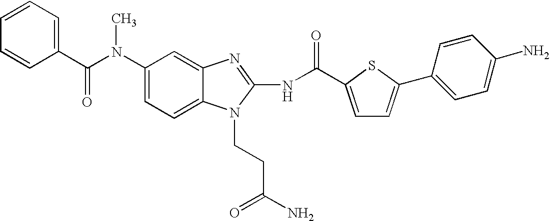 | 5-(4-Amino-phenyl)-thiophene-2- carboxylic acid [5-(benzoyl- methyl-amino)-1-(2-carbamoyl- ethyl)-1H-benzoimidazol-2-yl]- amide | 539 |
| Example 64 | 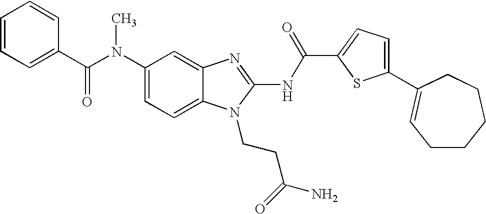 | 5-Cyclohept-1-enyl-thiophene-2- carboxylic acid [5-(benzoyl- methyl-amino)-1-(2-carbamoyl- ethyl)-1H-benzoimidazol-2-yl]- amide | 542 |
| Example 65 | 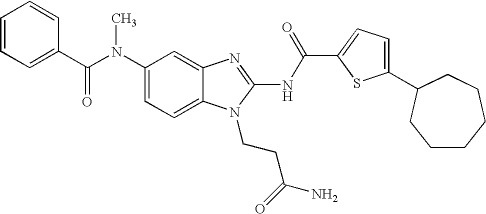 | 5-Cycloheptyl-thiophene-2- carboxylic acid [5-(benzoyl- methyl-amino)-1-(2-carbamoyl- ethyl)-1H-benzoimidazol-2-yl]- amide | 544 |
| Example 66 | 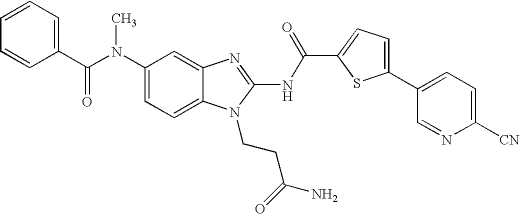 | 5-(6-Cyano-pyridin-3-yl)- thiophene-2-carboxylic acid [5- (benzoyl-methyl-amino)-1-(2- carbamoyl-ethyl)-1H- benzoimidazol-2-yl]-amide | 550 |
| Example 67 | 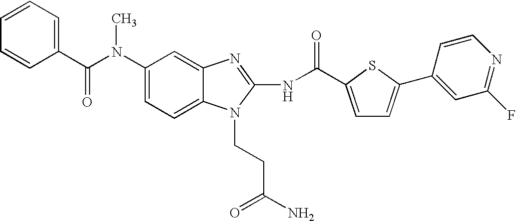 | 5-(2-Fluoro-pyridin-4-yl)- thiophene-2-carboxylic acid [5- (benzoyl-methyl-amino)-1-(2- carbamoyl-ethyl)-1H- benzoimidazol-2-yl]-amide | 543 |
| Example 68 | 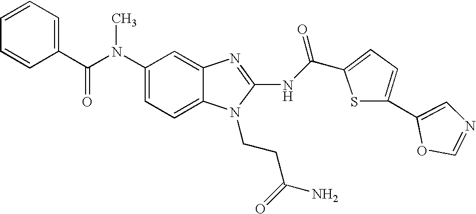 | 5-Oxazol-5-yl-thiophene-2- carboxylic acid [5-(benzoyl- methyl-amino)-1-(2-carbamoyl- ethyl)-1H-benzoimidazol-2-yl]- amide | 515 |
| Example 69 | 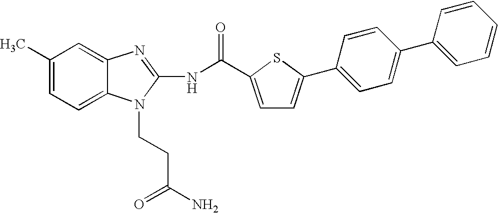 | 5-Biphenyl-4-yl-thiophene-2- carboxylic acid [1-(2-carbamoyl- ethyl)-5-methyl-1H- benzoimidazol-2-yl]-amide | 481 |
| Example 70 | 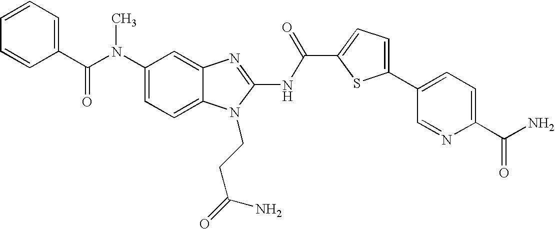 | 5-{5-[5-(Benzoyl-methyl-amino)- 1-(2-carbamoyl-ethyl)-1H- benzoimidazol-2-ylcarbamoyl]- thiophen-2-yl}-pyridine-2- carboxylic acid amide | 568 |
| Example 71 | 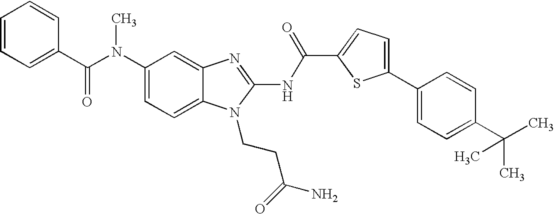 | 5-(4-tert-Butyl-phenyl)-thiophene- 2-carboxylic acid [5-(benzoyl- methyl-amino)-1-(2-carbamoyl- ethyl)-1H-benzoimidazol-2-yl]- amide | 580 |
| Example 72 | 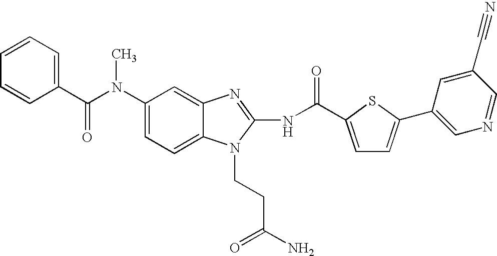 | 5-(5-Cyano-pyridin-3-yl)- thiophene-2-carboxylic acid [5- (benzoyl-methyl-amino)-1-(2- carbamoyl-ethyl)-1H- benzoimidazol-2-yl]-amide | 550 |
| Example 73 | 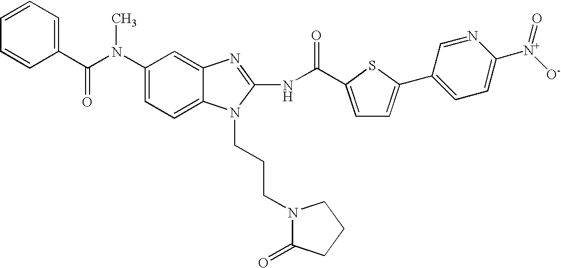 | 5-(6-Nitro-pyridin-3-yl)- thiophene-2-carboxylic acid {5- (benzoyl-methyl-amino)-1-[3-(2- oxo-pyrrolidin-1-yl)-propyl]-1H- benzoimidazol-2-yl}-amide | 624 |
| Example 74 | 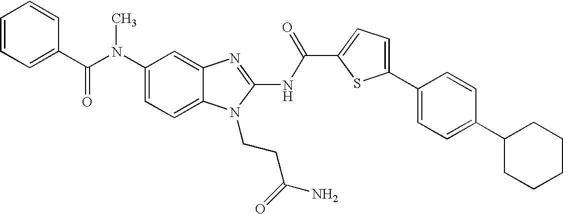 | 5-(4-Cyclohexyl-phenyl)- thiophene-2-carboxylic acid [5- (benzoyl-methyl-amino)-1-(2- carbamoyl-ethyl)-1H- benzoimidazol-2-yl]-amide | 606 |
| Example 75 | 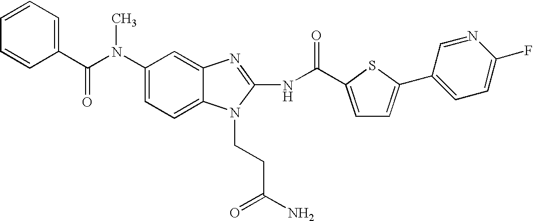 | 5-(6-Fluoro-pyridin-3-yl)- thiophene-2-carboxylic acid [5- (benzoyl-methyl-amino)-1-(2- carbamoyl-ethyl)-1H- benzoimidazol-2-yl]-amide | 543 |
| Example 76 | 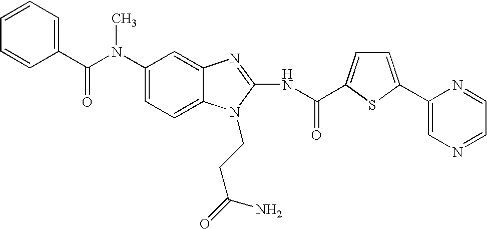 | 5-Pyrazin-2-yl-thiophene-2- carboxylic acid [5-(benzoyl- methyl-amino)-1-(2-carbamoyl- ethyl)-1H-benzoimidazol-2-yl]- amide | 526 |
| Example 77 | 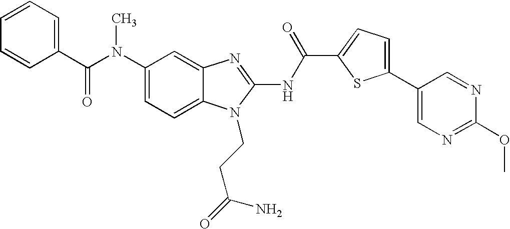 | 5-(2-Methoxy-pyrimidin-5-yl)- thiophene-2-carboxylic acid [5- (benzoyl-methyl-amino)-1-(2- carbamoyl-ethyl)-1H- benzoimidazol-2-yl]-amide | 556 |
| Example 78 | 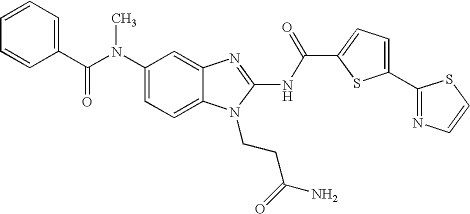 | 5-Thiazol-2-yl-thiophene-2- carboxylic acid [5-(benzoyl- methyl-amino)-1-(2-carbamoyl- ethyl)-1H-benzoimidazol-2-yl]- amide | 531 |
| Example 79 | 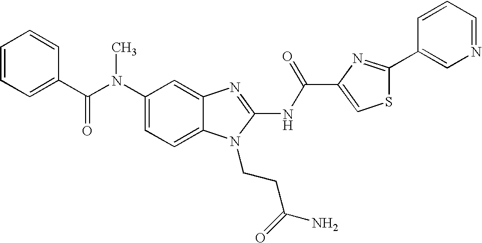 | 2-Pyridin-3-yl-thiazole-4- carboxylic acid [5-(benzoyl- methyl-amino)-1-(2-carbamoyl- ethyl)-1H-benzoimidazol-2-yl]- amide | 526 |
| Example 80 | 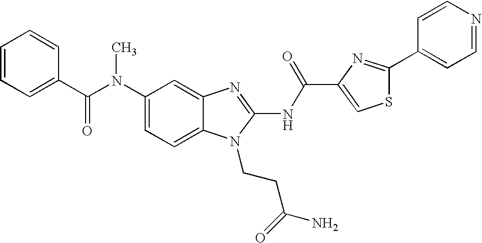 | 2-Pyridin-4-yl-thiazole-4- carboxylic acid [5-(benzoyl- methyl-amino)-1-(2-carbamoyl- ethyl)-1H-benzoimidazol-2-yl]- amide | 526 |
| Example 81 | 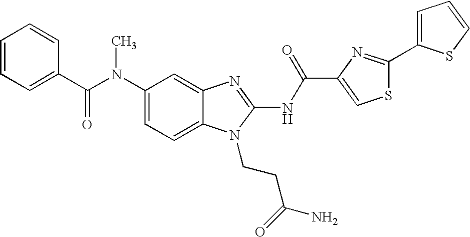 | 2-Thiophen-2-yl-thiazole-4- carboxylic acid [5-(benzoyl- methyl-amino)-1-(2-carbamoyl- ethyl)-1H-benzoimidazol-2-yl]- amide | 531 |
| Example 82 | 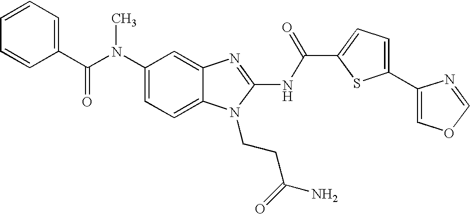 | 5-Oxazol-4-yl-thiophene-2- carboxylic acid [5-(benzoyl- methyl-amino)-1-(2-carbamoyl- ethyl)-1H-benzoimidazol-2-yl]- amide | 515 |
| Example 83 | 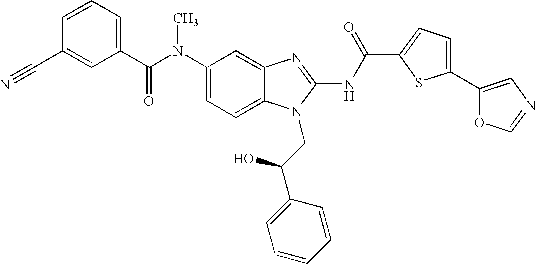 | 5-Oxazol-5-yl-thiophene-2- carboxylic acid [5-(3- cyanobenzoyl-methyl-amino)-1- (2R-hydroxyl-2-phenyl-ethyl)-1H- benzoimidazol-2-yl]-amide | 589 |
| Example 84 | 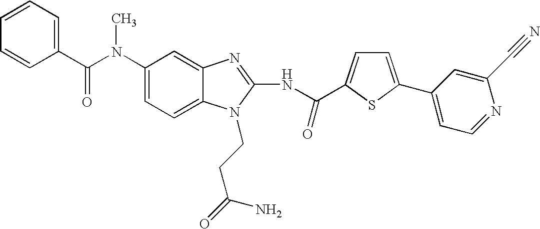 | 5-(2-Cyano-pyridin-4-yl)- thiophene-2-carboxylic acid [5- (benzoyl-methyl-amino)-1-(2- carbamoyl-ethyl)-1H- benzoimidazol-2-yl]-amide | 550 |
| Example 85 | 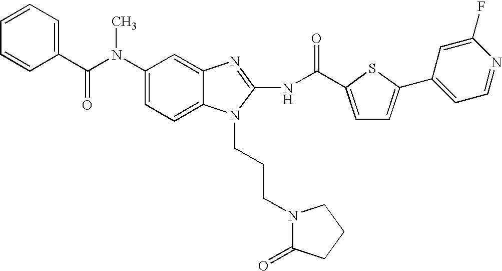 | 5-(2-Fluoro-pyridin-4-yl)- thiophene-2-carboxylic acid {5- (benzoyl-methyl-amino)-1-[3-(2- oxo-pyrrolidin-1-yl)-propyl]-1H- benzoimidazol-2-yl}-amide | 597 |
| Example 86 | 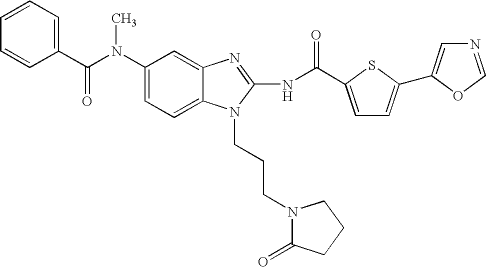 | 5-Oxazol-5-yl-thiophene-2- carboxylic acid {5-(benzoyl- methyl-amino)-1-[3-(2-oxo- pyrrolidin-1-yl)-propyl]-1H- benzoimidazol-2-yl}-amide | 569 |
| Example 87 | 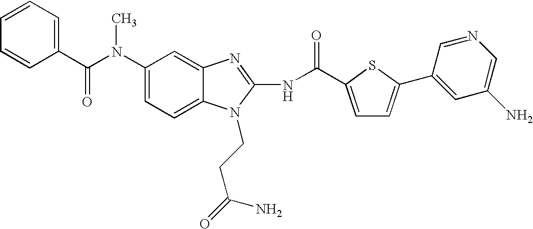 | 5-(5-Amino-pyridin-3-yl)- thiophene-2-carboxylic acid [5- (benzoyl-methyl-amino)-1-(2- carbamoyl-ethyl)-1H- benzoimidazol-2-yl]-amide | 540 |
| Example 88 | 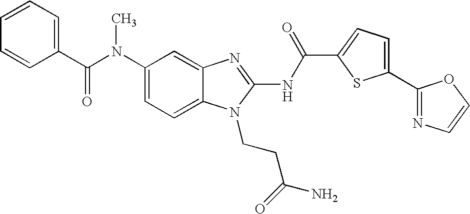 | 5-Oxazol-2-yl-thiophene-2- carboxylic acid [5-(benzoyl- methyl-amino)-1-(2-carbamoyl- ethyl)-1H-benzoimidazol-2-yl]- amide | 515 |
| Example 89 | 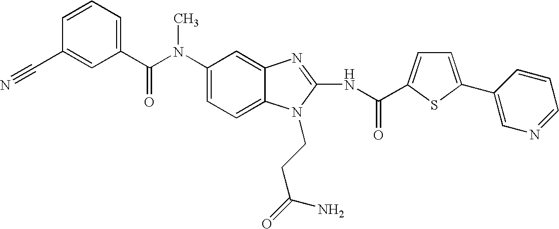 | 5-Pyridin-3-yl-thiophene-2- carboxylic acid {1-(2-carbamoyl- ethyl)-5-[(3-cyano-benzoyl)- methyl-amino]-1H- benzoimidazol-2-yl}-amide | 550 |
| Example 90 | 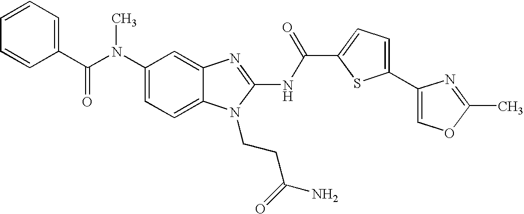 | 5-(2-Methyl-oxazol-4-yl)- thiophene-2-carboxylic acid [5- (benzoyl-methyl-amino)-1-(2- carbamoyl-ethyl)-1H- benzoimidazol-2-yl]-amide | 529 |
| Example 91 | 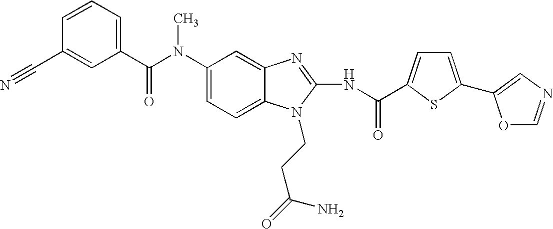 | 5-Oxazol-5-yl-thiophene-2- carboxylic acid {1-(2-carbamoyl- ethyl)-5-[(3-cyano-benzoyl)- methyl-amino]-1H- benzoimidazol-2-yl}-amide | 540 |
| Example 92 | 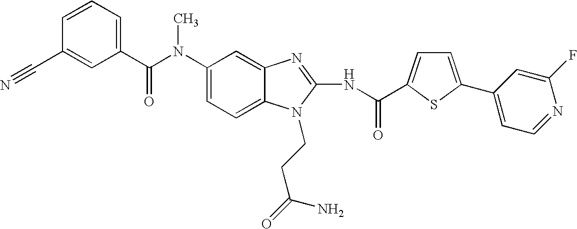 | 5-(2-Fluoro-pyridin-4-yl)- thiophene-2-carboxylic acid {1- (2-carbamoyl-ethyl)-5-[(3-cyano- benzoyl)-methyl-amino]-1H- benzoimidazol-2-yl}-amide | 568 |
| Example 93 | 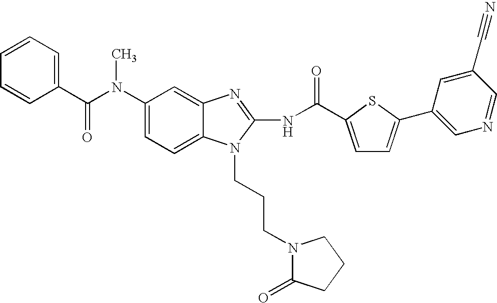 | 5-(5-Cyano-pyridin-3-yl)- thiophene-2-carboxylic acid {5- (benzoyl-methyl-amino)-1-[3-(2- oxo-pyrrolidin-1-yl)-propyl]-1H- benzoimidazol-2-yl}-amide | 604 |
| Example 94 | 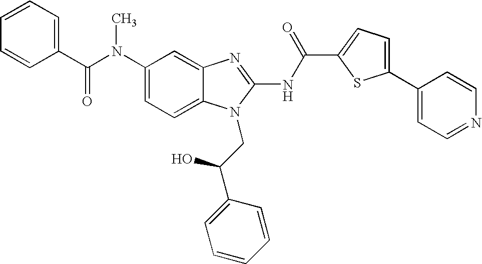 | 5-Pyridin-4-yl-thiophene-2- carboxylic acid [5-(benzoyl- methyl-amino)-1-(2R-hydroxyl- 2phenyl-ethyl)-1H- benzoimidazol-2-yl]-amide | 574 |
| Example 95 | 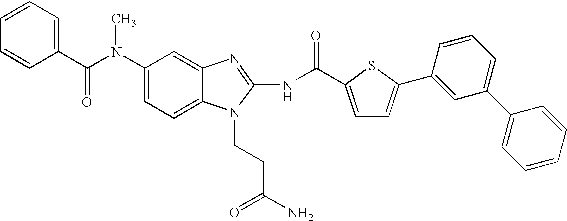 | 5-Biphenyl-3-yl-thiophene-2- carboxylic acid [5-(benzoyl- methyl-amino)-1-(2-carbamoyl- ethyl)-1H-benzoimidazol-2-yl]- amide | 600 |
| Example 96 | 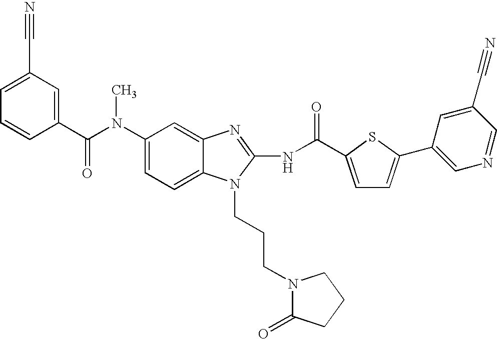 | 5-(5-Cyano-pyridin-3-yl)- thiophene-2-carboxylic acid {5- [(3-cyano-benzoyl)-methyl- amino]-1-[3-(2-oxo-pyrrolidin-1- yl)-propyl]-1H-benzoimidazol-2- yl}-amide | 629 |
| Example 97 | 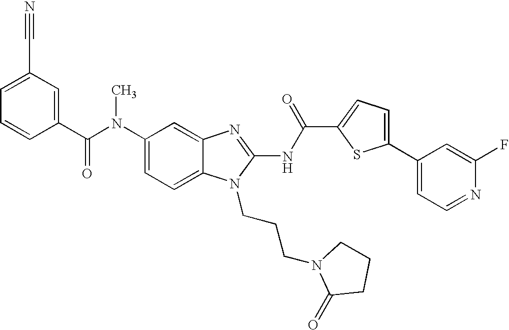 | 5-(2-Fluoro-pyridin-4-yl)- thiophene-2-carboxylic acid {5- [(3-cyano-benzoyl)-methyl- amino]-1-[3-(2-oxo-pyrrolidin-1- yl)-propyl]-1H-benzoimidazol-2- yl}-amide | 622 |
| Example 98 | 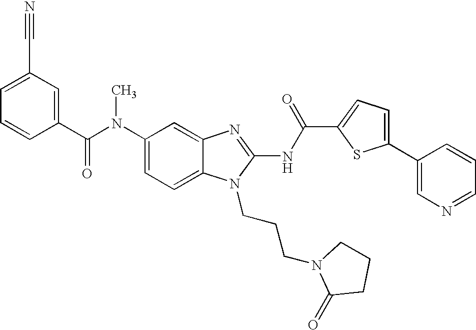 | 5-Pyridin-3-yl-thiophene-2- carboxylic acid {5-[(3-cyano- benzoyl)-methyl-amino]-1-[3-(2- oxo-pyrrolidin-1-yl)-propyl]-1H- benzoimidazol-2-yl}-amide | 604 |
| Example 99 | 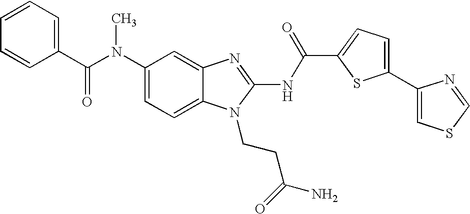 | 5-Thiazol-4-yl-thiophene-2- carboxylic acid [5-(benzoyl- methyl-amino)-1-(2-carbamoyl- ethyl)-1H-benzoimidazol-2-yl]- amide | 531 |
| Example 100 | 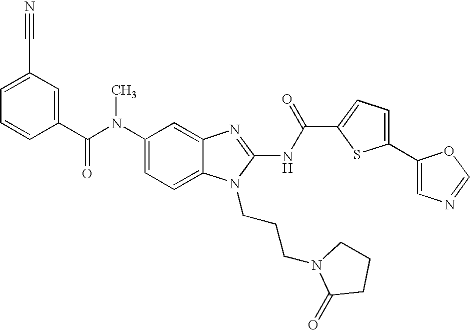 | 5-Oxazol-5-yl-thiophene-2- carboxylic acid {5-[(3-cyano- benzoyl)-methyl-amino]-1-[3-(2- oxo-pyrrolidin-1-yl)-propyl]-1H- benzoimidazol-2-yl}-amide | 594 |
| Example 101 | 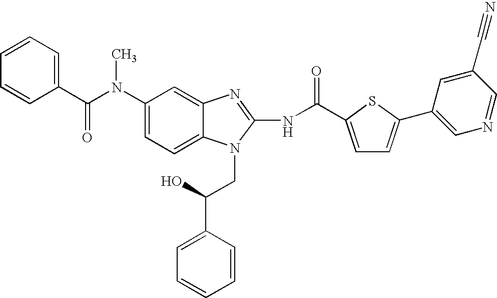 | 5-(5-Cyano-pyridin-3-yl)- thiophene-2-carboxylic acid [5- (benzoyl-methyl-amino)-1-((R)-2- hydroxy-2-phenyl-ethyl)-1H- benzoimidazol-2-yl]-amide | 599 |
| Example 102 | 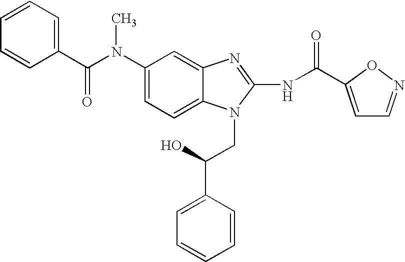 | Isoxazole-5-carboxylic acid [5- (benzoyl-methyl-amino)-1-((R)-2- hydroxy-2-phenyl-ethyl)-1H- benzoimidazol-2-yl]-amide | 482 |
| Example 103 | 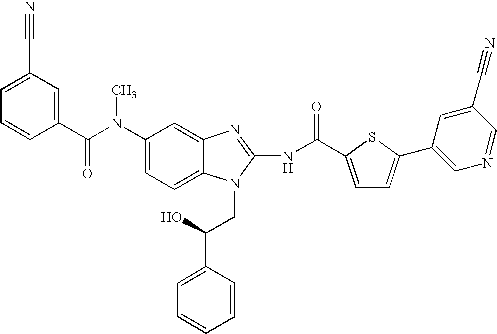 | 5-(5-Cyano-pyridin-3-yl)- thiophene-2-carboxylic acid [5- [(3-cyano-benzoyl)-methyl- amino]-1-((R)-2-hydroxy-2- phenyl-ethyl)-1H-benzoimidazol- 2-yl]-amide | 624 |
| Example 104 | 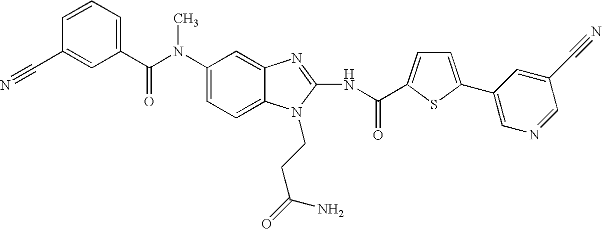 | 5-(5-Cyano-pyridin-3-yl)- thiophene-2-carboxylic acid {1- (2-carbamoyl-ethyl)-5-[(3-cyano- benzoyl)-methyl-amino]-1H- benzoimidazol-2-yl}-amide | 575 |
| Example 105 | 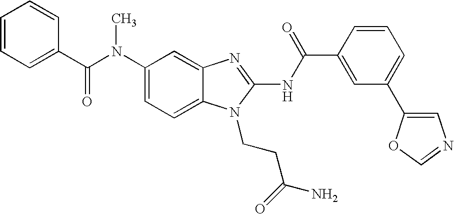 | N-[5-(Benzoyl-methyl-amino)-1- (2-carbamoyl-ethyl)-1H- benzoimidazol-2-yl]-3-oxazol-5- yl-benzamide | 509 |
| Example 106 | 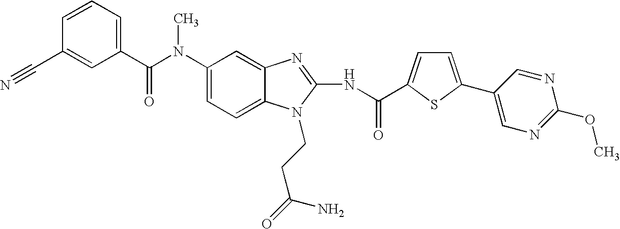 | 5-(2-Methoxy-pyrimidin-5-yl)- thiophene-2-carboxylic acid {1- (2-carbamoyl-ethyl)-5-[(3-cyano- benzoyl)-methyl-amino]-1H- benzoimidazol-2-yl}-amide | 581 |
| Example 107 | 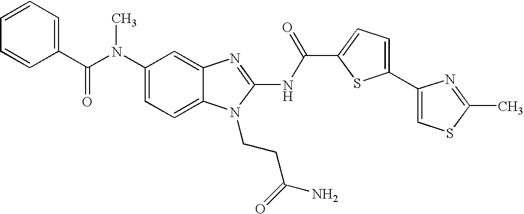 | 5-(2-Methyl-thiazol-4-yl)- thiophene-2-carboxylic acid [5- (benzoyl-methyl-amino)-1-(2- carbamoyl-ethyl)-1H- benzoimidazol-2-yl]-amide | 545 |
| Example 108 | 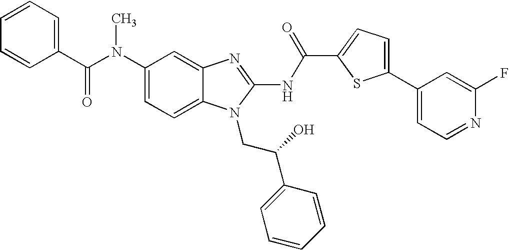 | 5-(2-Fluoro-pyridin-4-yl)- thiophene-2-carboxylic acid [5- (benzoyl-methyl-amino)-1-((R)-2- hydroxy-2-phenyl-ethyl)-1H- benzoimidazol-2-yl]-amide | 592 |
| Example 109 | 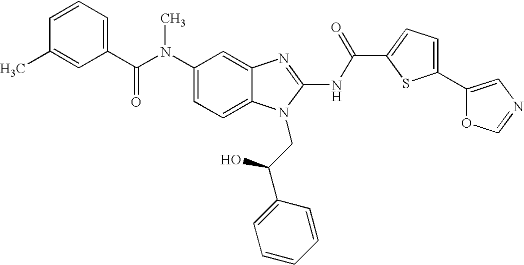 | 5-Oxazol-5-yl-thiophene-2- carboxylic acid {1-((R)-2- hydroxy-2-phenyl-ethyl)-5- [methyl-(3-methyl-benzoyl)- amino]-1H-benzoimidazol-2-yl}- amide | 578 |
| Example 110 | 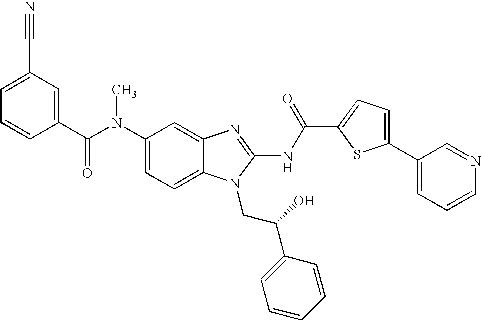 | 5-Pyridin-3-yl-thiophene-2- carboxylic acid [5-[(3-cyano- benzoyl)-methyl-amino]-1-((R)-2- hydroxy-2-phenyl-ethyl)-1H- benzoimidazol-2-yl]-amide | 599 |
| Example 111 | 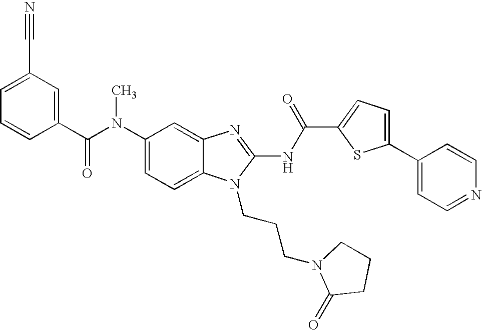 | 5-Pyridin-4-yl-thiophene-2- carboxylic acid {5-[(3-cyano- benzoyl)-methyl-amino]-1-[3-(2- oxo-pyrrolidin-1-yl)-propyl]-1H- benzoimidazol-2-yl}-amide | 604 |
| Example 112 | 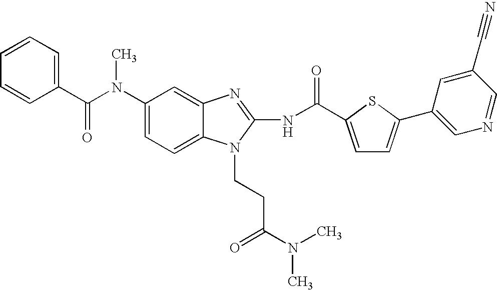 | 5-(5-Cyano-pyridin-3-yl)- thiophene-2-carboxylic acid [5- (benzoyl-methyl-amino)-1-(2- dimethylcarbamoyl-ethyl)-1H- benzoimidazol-2-yl]-amide | 578 |
| Example 113 | 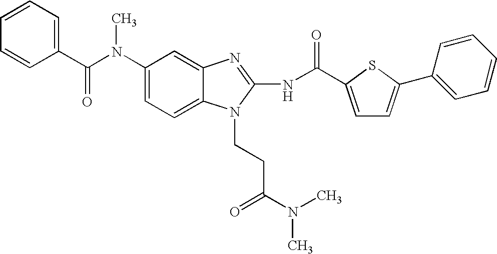 | 5-Phenyl-thiophene-2-carboxylic acid [5-(benzoyl-methyl-amino)- 1-(2-dimethylcarbamoyl-ethyl)- 1H-benzoimidazol-2-yl]-amide | 552 |
| Example 114 | 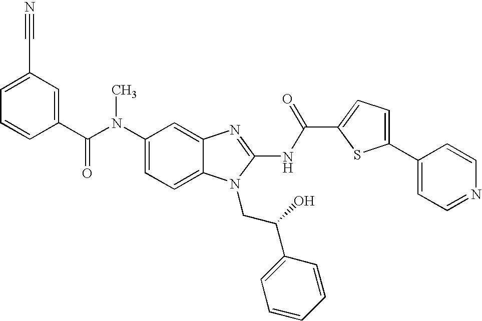 | 5-Pyridin-4-yl-thiophene-2- carboxylic acid [5-[(3-cyano- benzoyl)-methyl-amino]-1-((R)-2- hydroxy-2-phenyl-ethyl)-1H- benzoimidazol-2-yl]-amide | 599 |
| Example 115 | 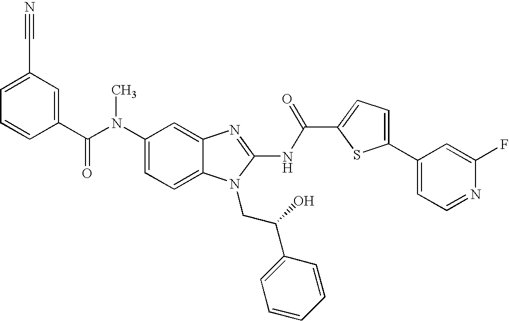 | 5-(2-Fluoro-pyridin-4-yl)- thiophene-2-carboxylic acid [5- [(3-cyano-benzoyl)-methyl- amino]-1-((R)-2-hydroxy-2- phenyl-ethyl)-1H-benzoimidazol- 2-yl]-amide | 617 |
| Example 116 | 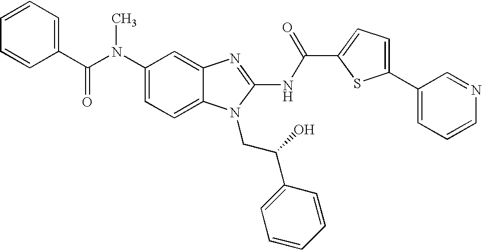 | 5-Pyridin-3-yl-thiophene-2- carboxylic acid [5-(benzoyl- methyl-amino)-1-((R)-2-hydroxy- 2-phenyl-ethyl)-1H- benzoimidazol-2-yl]-amide | 574 |
| Example 117 | 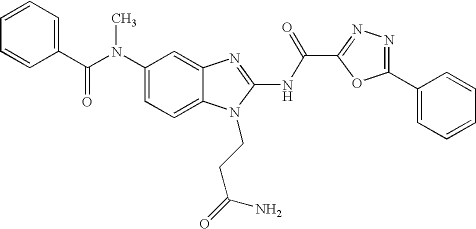 | 5-Phenyl-[1,3,4]oxadiazole-2- carboxylic acid [5-(benzoyl- methyl-amino)-1-(2-carbamoyl- ethyl)-1H-benzoimidazol-2-yl]- amide | 510 |
| Example 118 | 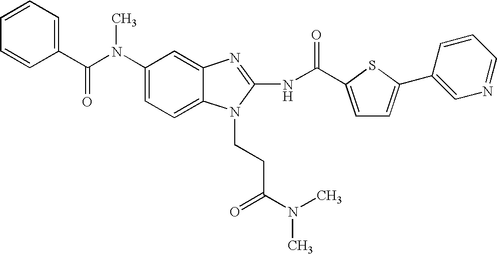 | 5-Pyridin-3-yl-thiophene-2- carboxylic acid [5-(benzoyl- methyl-amino)-1-(2- dimethylcarbamoyl-ethyl)-1H- benzoimidazol-2-yl]-amide | 553 |
| Example 119 | 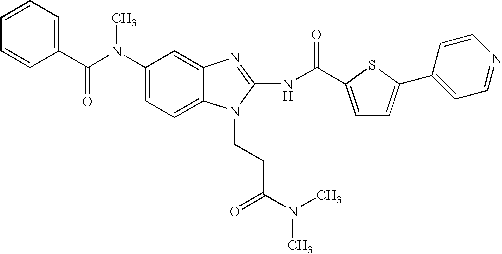 | 5-Pyridin-4-yl-thiophene-2- carboxylic acid [5-(benzoyl- methyl-amino)-1-(2- dimethylcarbamoyl-ethyl)-1H- benzoimidazol-2-yl]-amide | 553 |
| Example 120 | 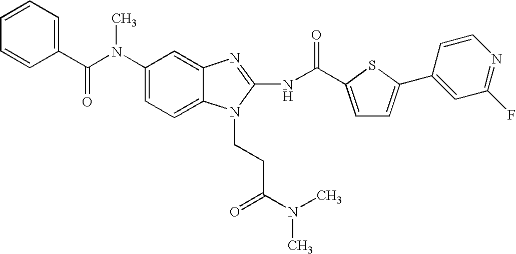 | 5-(2-Fluoro-pyridin-4-yl)- thiophene-2-carboxylic acid [5- (benzoyl-methyl-amino)-1-(2- dimethylcarbamoyl-ethyl)-1H- benzoimidazol-2-yl]-amide | 571 |
| Example 121 | 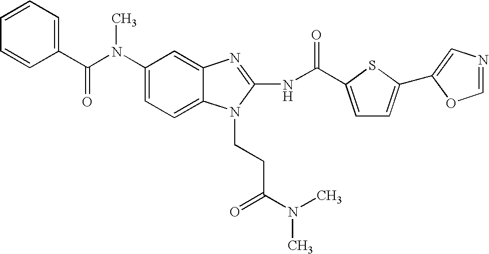 | 5-Oxazol-5-yl-thiophene-2- carboxylic acid [5-(benzoyl- methyl-amino)-1-(2- dimethylcarbamoyl-ethyl)-1H- benzoimidazol-2-yl]-amide | 543 |
| Example 122 | 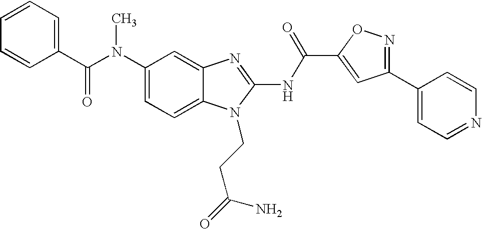 | 3-Phenyl-isoxazole-5-carboxylic acid [5-(benzoyl-methyl-amino)- 1-(2-carbamoyl-ethyl)-1H- benzoimidazol-2-yl]-amide | 510 |
| Example 123 | 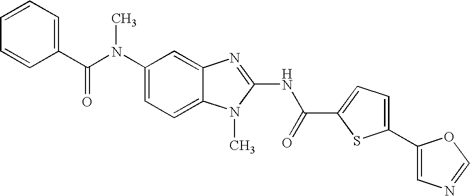 | 5-Oxazol-5-yl-thiophene-2- carboxylic acid [5-(benzoyl- methyl-amino)-1-methyl-1H- benzoimidazol-2-yl]-amide | 458 |
| Example 124 | 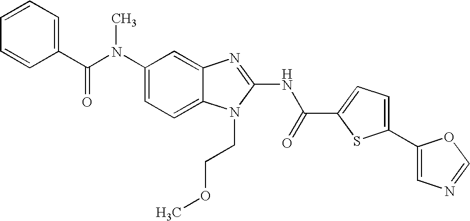 | 5-Oxazol-5-yl-thiophene-2- carboxylic acid [5-(benzoyl- methyl-amino)-1-(2-methoxy- ethyl)-1H-benzoimidazol-2-yl]- amide | 502 |
| Example 125 | 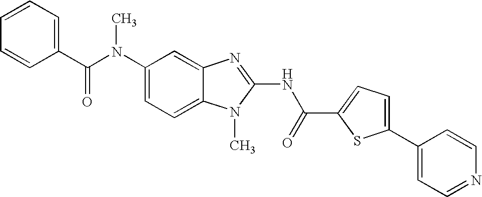 | 5-Pyridin-4-yl-thiophene-2- carboxylic acid [5-(benzoyl- methyl-amino)-1-methyl-1H- benzoimidazol-2-yl]-amide | 468 |
| Example 126 | 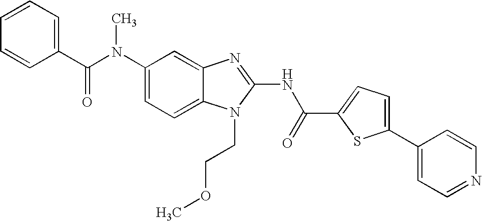 | 5-Pyridin-4-yl-thiophene-2- carboxylic acid [5-(benzoyl- methyl-amino)-1-(2-methoxy- ethyl)-1H-benzoimidazol-2-yl]- amide | 512 |
| Example 127 | 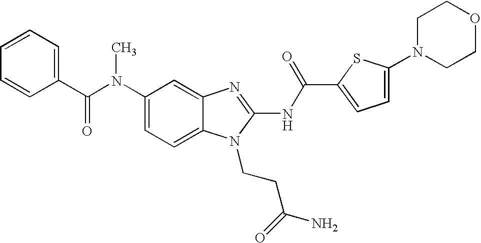 | N-{1-(2-Carbamoyl-ethyl)-2-[2- (5-morpholin-4-yl-thiophen-2-yl)- 2-oxo-ethyl]-1H-benzoimidazol- 5-yl}-N-methyl-benzamide | 533 |
| Example 128 | 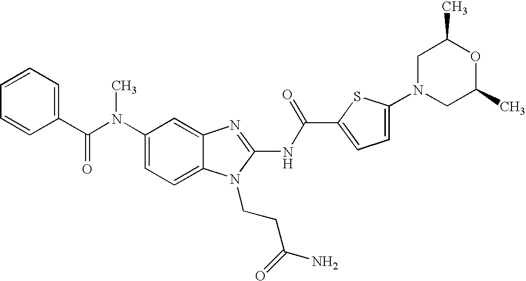 | N-(1-(2-Carbamoyl-ethyl)-2-{2- [5-((2R,6S)-2,6-dimethyl- morpholin-4-yl)-thiophen-2-yl]-2- oxo-ethyl}-1H-benzoimidazol-5- yl)-N-methyl-benzamide | 561 |
| Example 129 | 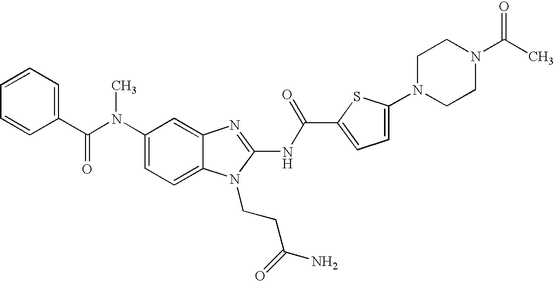 | N-[2-{2-[5-(4-Acetyl-piperazin-1- yl)-thiophen-2-yl]-2-oxo-ethyl}-1- (2-carbamoyl-ethyl)-1H- benzoimidazol-5-yl]-N-methyl- benzamide | 574 |
| Example 130 | 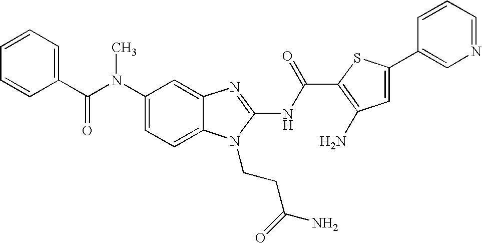 | N-[2-[2-(3-Amino-5-pyridin-3-yl- thiophen-2-yl)-2-oxo-ethyl]-1-(2- carbamoyl-ethyl)-1H- benzoimidazol-5-yl]-N-methyl- benzamide | 540 |
| Example 131 | 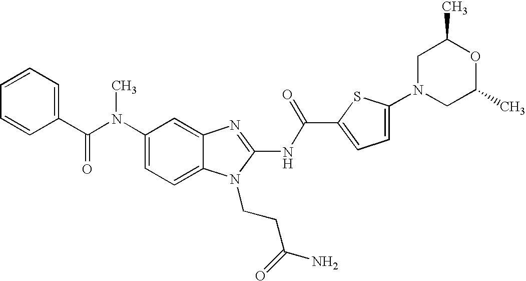 | N-(1-(2-Carbamoyl-ethyl)-2-{2- [5-((2R,6R)-2,6-dimethyl- morpholin-4-yl)-thiophen-2-yl]-2- oxo-ethyl}-1H-benzoimidazol-5- yl)-N-methyl-benzamide | 561 |
| Example 132 | 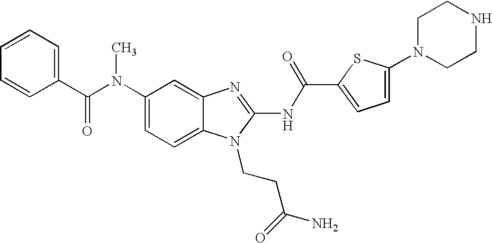 | N-{1-(2-Carbamoyl-ethyl)-2-[2- oxo-2-(5-piperazin-1-yl-thiophen- 2-yl)-ethyl]-1H-benzoimidazol-5- yl}-N-methyl-benzamide | 532 |
| Example 133 | 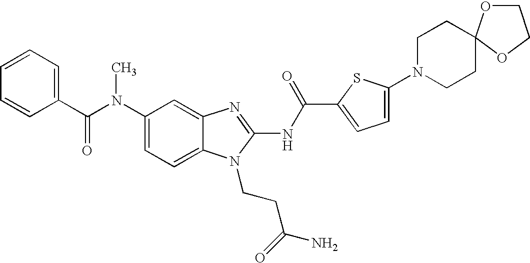 | N-(1-(2-Carbamoyl-ethyl)-2-{2- [5-(1,4-dioxa-8-aza-spiro[4.5]dec- 8-yl)-thiophen-2-yl]-2-oxo-ethyl}- 1H-benzoimidazol-5-yl)-N- methyl-benzamide | 589 |
| Example 134 | 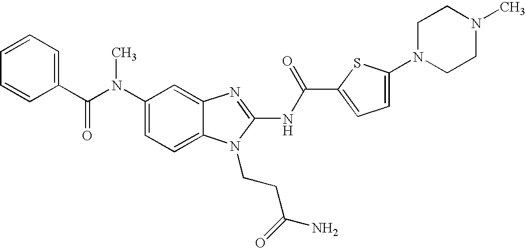 | N-(1-(2-Carbamoyl-ethyl)-2-{2- [5-(4-methyl-piperazin-1-yl)- thiophen-2-yl]-2-oxo-ethyl}-1H- benzoimidazol-5-yl)-N-methyl- benzamide | 546 |
| Example 135 | 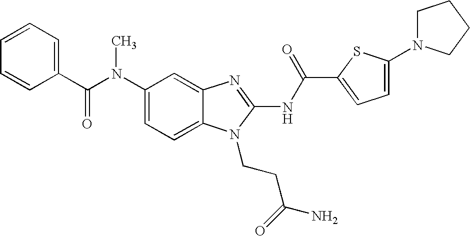 | N-{1-(2-Carbamoyl-ethyl)-2-[2- oxo-2-(5-pyrrolidin-1-yl- thiophen-2-yl)-ethyl]-1H- benzoimidazol-5-yl}-N-methyl- benzamide | 517 |
| Example 136 | 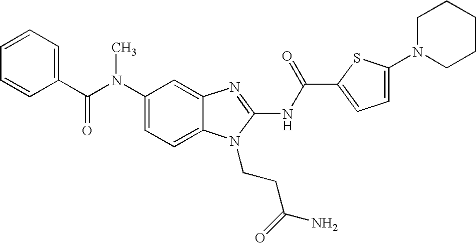 | N-{1-(2-Carbamoyl-ethyl)-2-[2- oxo-2-(5-piperidin-1-yl-thiophen- 2-yl)-ethyl]-1H-benzoimidazol-5- yl}-N-methyl-benzamide | 531 |
| Example 137 | 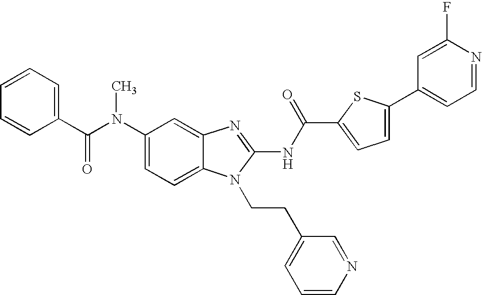 | N-[2-{2-[5-(2-Fluoro-pyridin-4- yl)-thiophen-2-yl]-2-oxo-ethyl}-1- (2-pyridin-3-yl-ethyl)-1H- benzoimidazol-5-yl]-N-methyl- benzamide | 577 |
| Example 138 | 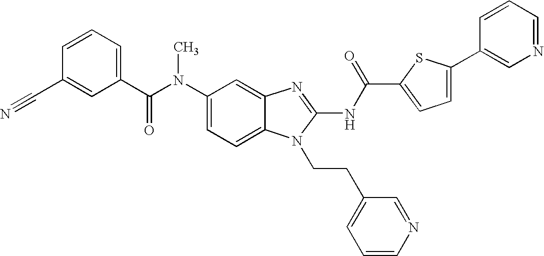 | 3-Cyano-N-methyl-N-[2-[2-oxo- 2-(5-pyridin-3-yl-thiophen-2-yl)- ethyl]-1-(2-pyridin-3-yl-ethyl)- 1H-benzoimidazol-5-yl]- benzamide | 584 |
| Example 139 | 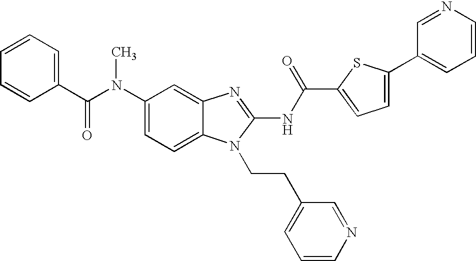 | N-Methyl-N-[2-[2-oxo-2-(5- pyridin-3-yl-thiophen-2-yl)- ethyl]-1-(2-pyridin-3-yl-ethyl)- 1H-benzoimidazol-5-yl]- benzamide | 559 |
| Example 140 | 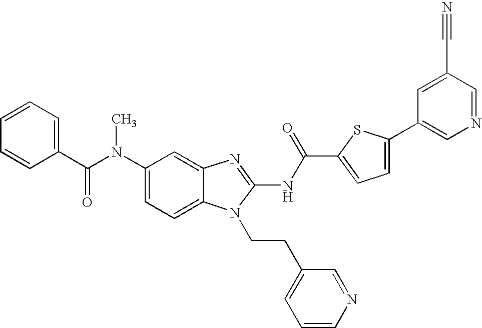 | N-[2-{2-[5-(5-Cyano-pyridin-3- yl)-thiophen-2-yl]-2-oxo-ethyl}-1- (2-pyridin-3-yl-ethyl)-1H- benzoimidazol-5-yl]-N-methyl- benzamide | 584 |
| Example 141 | 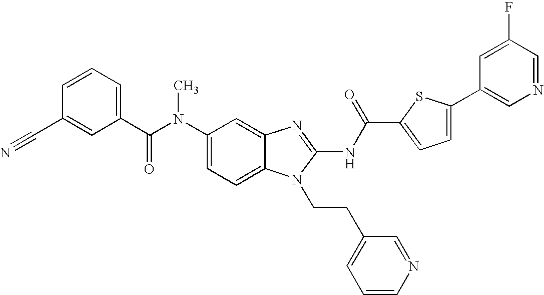 | 5-(2-Fluoro-pyridin-4-yl)- thiophene-2-carboxylic acid [5- [(3-cyano-benzoyl)-methyl- amino]-1-(2-pyridin-3-yl-ethyl)- 1H-benzoimidazol-2-yl]-amide | 602 |
| Example 142 | 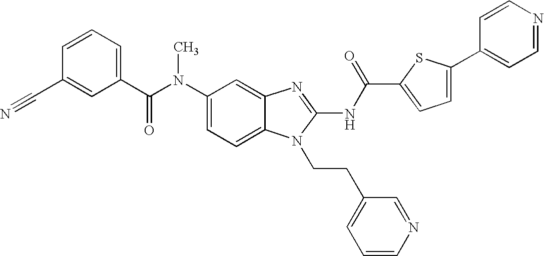 | 5-Pyridin-4-yl-thiophene-2- carboxylic acid [5-[(3-cyano- benzoyl)-methyl-amino]-1-(2- pyridin-3-yl-ethyl)-1H- benzoimidazol-2-yl]-amide | 584 |
| Example 143 | 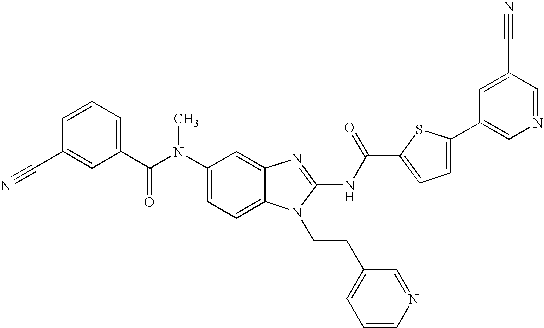 | 5-(5-Cyano-pyridin-3-yl)- thiophene-2-carboxylic acid [5- [(3-cyano-benzoyl)-methyl- amino]-1-(2-pyridin-3-yl-ethyl)- 1H-benzoimidazol-2-yl]-amide | |
| Example 144 | 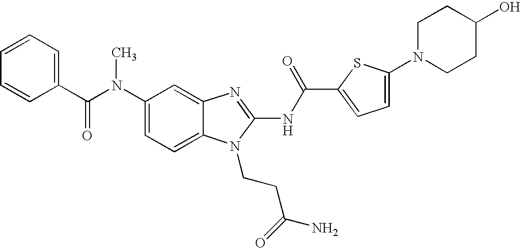 | 5-(4-Hydroxy-piperidin-1-yl)- thiophene-2-carboxylic acid [5- (benzoyl-methyl-amino)-1-(2- carbamoyl-ethyl)-1H- benzoimidazol-2-yl]-amide | 547 |
| Example 145 | 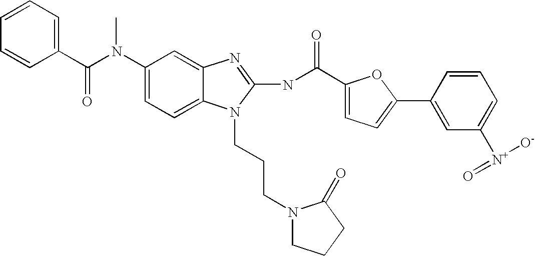 | 5-(3-Nitro-phenyl)-furan-2- carboxylic acid [5-(benzoyl- methyl-amino)-1-(2-carbamoyl- ethyl)-1H-benzoimidazol-2-yl]- amide | 607 |
| Example 146 | 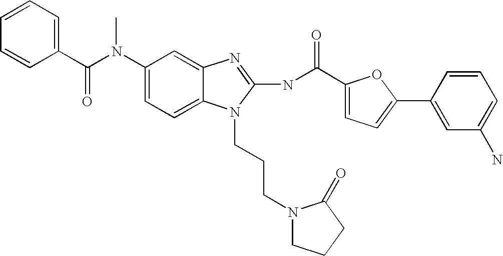 | 5-(3-Amino-phenyl)-furan-2- carboxylic acid [5-(benzoyl- methyl-amino)-1-(2-carbamoyl- ethyl)-1H-benzoimidazol-2-yl]- amide | 576 |
| Example 147 | 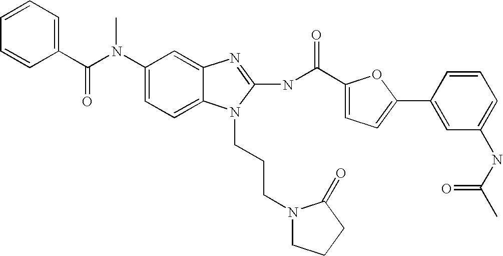 | 5-(3-Acetylamino-phenyl)-furan- 2-carboxylic acid [5-(benzoyl- methyl-amino)-1-(2-carbamoyl- ethyl)-1H-benzoimidazol-2-yl]- amide | 618 |

1. A compound of the formula (I): 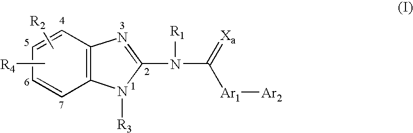
wherein:
R₁ is hydrogen or alkyl;
R₂, covalently attached at the indicated 4-, 5-, 6- or 7-position of the formula (I), is chosen from hydrogen, alkyl, alkoxy and halogen;
Ar₁ is chosen from carbocycle and heteroaryl each optionally substituted with one or more amine, alkyl, alkoxy or halogen;
Ar₂ is chosen from carbocycle, heterocycle and heteroaryl each optionally substituted with one or more Rₐ;
R₃ is C₁₋₁₀ alkyl chain branched or unbranched optionally substituted with one or more R_{b},
or R₃ is the group:
—(CH₂)ₙ-L-R₆, wherein L is chosen from a bond, —NH—C(O)—, —O—C(O)—, —C(O)— and —S(O)ₘ— wherein m is 0, 1 or 2, and wherein said group is optionally substituted by one or more R_{b};
wherein R₆ is independently chosen from hydrogen, hydroxy, alkyl, alkoxy, alkylthio, arylC₀₋₅ alkyl, aryloxyC₀₋₅ alkyl, heteroarylC₀₋₅ alkyl, cycloalkylC₀₋₅ alkyl, heterocyclylC₀₋₅ alkyl and amino said amino is optionally mono-or di-substituted by acyl, alkyl, alkoxycarbonyl, cycloalkylC₀₋₅ alkyl, arylC₀₋₅ alkyl, heteroarylC₀₋₅ alkyl or heterocyclylC₀₋₅ alkyl;
n is 1-10;
R₄ is a group chosen from: 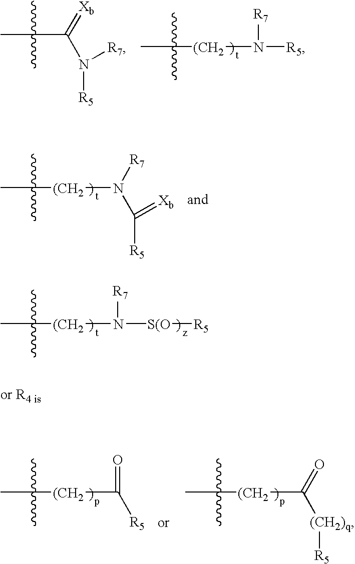
wherein if p or q>0 then a hydrogen atom from their respective —(CH₂)— group(s) may be replaced with a C₁₋₁₀ alkyl wherein one or more —CH₂— groups of said alkyl are optionally replaced by a heteroatom group chosen from O, S and NH,
wherein R₄ is covalently attached at the indicated 5- or 6-position of the formula (I), p, q, t and z are each independently chosen from 0,1 or 2;
R₅ is chosen from arylC₀₋₅ alkyl, alkyl, heteroarylC₀₋₅ alkyl, cycloalkylC₀₋₅ alkyl and heterocyclylC₀₋₅ alkyl, each R₅ optionally substituted with one or more R_{c};
R₇ is hydrogen, alkenyl or alkyl;
or R₅ and R₇ together with the nitrogen atom to which they are attached form: a 4-7-membered monocyclic ring or an 8-14-membered bicyclic ring,
wherein each monocyclic or bicyclic ring optionally contains an additional 1 to 3 heteroatoms chosen from N, O and S and each ring is aromatic or nonaromatic, and wherein each monocyclic or bicyclic ring is optionally substituted by one or more R_{c};
each Rₐ, R_{b} or R_{c} are independently chosen from hydrogen, alkyl, alkenyl, alkynyl, cycloalkyl, cycloalkenyl, aryl, arylalkyl, aryloxy, alkoxy, alkylthio, acyl, alkoxycarbonyl, acyloxy, acylamino, sulphonylamino, aminosulfonyl, alkylsulfonyl, carboxy, carboxamide, oxo, hydroxy, halogen, trifluoromethyl, nitro, nitrile and amino optionally mono-or-di-substituted by alkyl, acyl or alkoxycarbonyl, wherein any of the above R₁, R_{b} or R_{c} are optionally halogenated where possible; and
Xₐ and X_{b} are oxygen or sulfur;
or the pharmaceutically acceptable salts thereof.

2. The compound according to claim 1 and wherein:
R₁ is hydrogen;
R₂ is chosen from hydrogen, C₁₋₃ alkyl, C₁₋₃ alkoxy and halogen;
Ar₁ is phenyl or heteroaryl chosen from thienyl, furanyl, isoxazolyl, oxazolyl, thiazolyl, oxadiazolyl, thiadiazolyl, tetrazolyl, pyrazolyl, pyrrolyl, imidazolyl, pyridinyl, pyrimidinyl, pyrazinyl, pyridazinyl and pyranyl;
Ar₂ is chosen from C₃₋₈ cycloalkyl, C₄₋₈ cycloalkenyl, phenyl, naphthyl and heteroaryl chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothiophenyl, benzodioxolyl, quinolinyl, quinazolinyl and indazolyl each is optionally substituted with one or more Rₐ;
R₃ is C₁₋₁₀ alkyl chain branched or unbranched optionally substituted with one or more R_{b},
or R₃ is:
—(CH₂)ₙ-L-R₆, wherein L is chosen from a bond, —O—C(O)—, —C(O)— and —S(O)ₘ— wherein m is 0, 1 or 2, and wherein said group is optionally substituted by one or more R_{b};
wherein R₆ is independently chosen from hydrogen, hydroxy, C₁₋₅ alkyl, C₁₋₅ alkoxy, C₁₋₅ alkylthio, phenyl, naphthyl, benzyl, phenethyl, heteroarylC₀₋₅ alkyl, C₃₋₇ cycloalkylC₀₋₅ alkyl, heterocyclylC₀₋₅ alkyl and amino said amino is optionally mono- or di-substituted by C₁₋₅ acyl, C₁₋₅ alkyl, C₁₋₅ alkoxycarbonyl, arylC₀₋₅ alkyl, heteroarylC₀₋₅ alkyl or heterocyclylC₀₋₅ alkyl; and wherein each recited heteroaryl in this paragraph is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl and wherein each recited heterocyclyl in this paragraph is chosen from pyrrolidinyl, morpholinyl, thiomorpholinyl, dioxalanyl, piperidinyl and piperazinyl;
R₄ is a group chosen from: 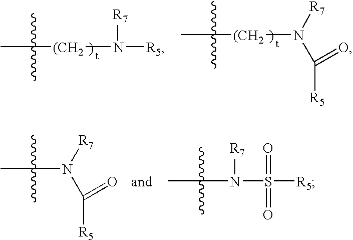
R₅ is chosen from phenyl, naphthyl, benzyl, phenethyl, C₁₋₅ alkyl, heteroarylC₀₋₅ alkyl wherein the heteroaryl is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl, C₃₋₇ cycloalkylC₀₋₅ alkyl and heterocyclylC₀₋₅ alkyl wherein the heterocyclyl is chosen from aziridinyl, pyrrolidinyl, morpholinyl, thiomorpholinyl, tetrahydrofuranyl, dioxalanyl, piperidinyl and piperazinyl, each R₅ is optionally substituted with one or more R_{c};
each Rₐ, R_{b} or R_{c} are independently chosen from hydrogen, C₁₋₅ alkyl, C₂₋₅ alkenyl, C₂₋₅ alkynyl, C₃₋₈ cycloalkyl, C₄₋₈ cycloalkenyl, phenyl, benzyl, phenoxy, C₁₋₅ alkoxy, C₁₋₅ alkylthio, C₁₋₅ acyl, C₁₋₅ alkoxycarbonyl, C₁₋₅ acyloxy, C₁₋₅ acylamino, C₁₋₅ sulphonylamino, aminosulfonyl, C₁₋₅ alkylsulfonyl, carboxy, carboxamide, oxo, hydroxy, halogen, trifluoromethyl, nitro, nitrile and amino optionally mono-or-di-substituted by C₁₋₅ alkyl, C₁₋₅ acyl or C₁₋₅ alkoxycarbonyl, wherein any of the above Rₐ, R_{b} or R_{c} are optionally halogenated where possible;
R₇ is hydrogen, C₃₋₁₀ alkenyl or C₁₋₅ alkyl; and
Xₐ is oxygen.

3. The compound according to claim 2 and wherein:
R₂ is chosen from hydrogen and C₁₋₃ alkyl;
Ar₁ is chosen from phenyl, thienyl, furanyl, isoxazolyl, oxazolyl, imidazolyl, oxadiazolyl, thiadiazolyl, pyrazolyl and pyridinyl;
Ar₂ is chosen from C₄₋₈ cycloalkenyl, C₄₋₈ cycloalkyl, phenyl, naphthyl, benzothiophenyl, benzodioxolyl, quinolinyl, indolyl, thiazolyl, thienyl, furanyl, isoxazolyl, oxazolyl, imidazolyl, thiadiazolyl, pyrazolyl, pyrazinyl and pyridinyl each is optionally substituted with one or more Rₐ;
R₆ is independently chosen from hydroxy, C₁₋₅ alkyl, C₁₋₅ alkoxy, phenyl, benzyl, phenethyl, heteroarylC₀₋₅ alkyl, heterocyclylC₀₋₅ alkyl, C₃₋₇ cycloalkyl and amino said amino is optionally mono-or di-substituted by C₁₋₅ acyl, C₁₋₅ alkyl, C₁₋₅ alkoxycarbonyl, arylC₀₋₅ alkyl or heteroarylC₀₋₅ alkyl;
and wherein each recited heteroaryl in this paragraph is chosen from thienyl, furanyl, isoxazolyl, oxazolyl, thiazolyl, thiadiazolyl, tetrazolyl, pyrazolyl, pyrrolyl and imidazolyl, each optionally substituted by R_{b};
n is 1-6;
R₅ is chosen from phenyl, naphthyl, benzyl, phenethyl, C₁₋₅ alkyl, heteroarylC₀₋₅ alkyl wherein the heteroaryl in this paragraph is chosen from thienyl, furanyl, imidazolyl and pyridinyl, C₃₋₇ cycloalkylC₀₋₅ alkyl and heterocyclylC₀₋₅ alkyl wherein the heterocyclyl is chosen from aziridinyl, pyrrolidinyl, tetrahydrofuranyl, tetrahydropyridinyl, morpholinyl, thiomorpholinyl, piperidinyl and piperazinyl, each R₅ is optionally substituted with one or more R_{c};
R₇ is hydrogen, propenyl or C₁₋₃ alkyl.

4. The compound according to claim 3 and wherein:
R₂ is chosen from hydrogen and methyl;
Ar₂ is chosen from cyclopentyl, cyclohexyl, cycloheptyl, cyclopentenyl, cyclohexenyl, cycloheptenyl,phenyl, naphthyl, benzothiophenyl, benzodioxolyl, quinolinyl, indolyl, thiazolyl, thienyl, furanyl, isoxazolyl, oxazolyl, imidazolyl, thiadiazolyl, oxadiazolyl, pyrazinyl and pyridinyl each is optionally substituted with one or more Rₐ;
R₃ is:
—(CH₂)ₙ—C(O)—R₆ or
—(CH₂)ₙ—R₆;
wherein R₆ is independently chosen from hydroxy, C₁₋₅ alkyl, C₁₋₅ alkoxy, phenyl, morpholinylC₀₋₅ alkyl, piperazinylC₀₋₅ alkyl, imidazolylC₀₋₅ alkyl, pyrrolidinylC₀₋₅ alkyl, pyrrolidinonylC₀₋₅ alkyl, thienylC₀₋₅ alkyl, C₃₋₇ cycloalkyl and amino said amino is optionally mono-or di-substituted by C₁₋₅ alkyl or C₁₋₅ alkoxycarbonyl;
R₅ is chosen from phenyl, furanyl, benzyl, phenethyl, C₁₋₃ alkyl and C₃₋₇ cycloalkylC₀₋₅ alkyl each optionally substituted with one or more R_{c};
each Rₐ, R_{b} or R_{c} are independently chosen from C₁₋₅ alkyl, C₂₋₅ alkenyl, C₃₋₈ cycloalkyl, C₄₋₈ cycloalkenyl, phenyl, C₁₋₅ alkoxy, C₁₋₅ alkylthio, amino optionally mono-or-di-substituted by C₁₋₅ alkyl, C₁₋₅ alkoxycarbonyl, carboxamide, hydroxy, halogen, trifluoromethyl, nitro and nitrile, wherein any of the above Rₐ, R_{b} or R_{c} are optionally halogenated where possible;
R₇ is C₁₋₃ alkyl.

5. The compound according to claim 4 and wherein:
R₂ is hydrogen;
Ar₁ is chosen from phenyl, thienyl, furanyl, isoxazolyl and pyridinyl;
Ar₂ is chosen from cycloheptyl, cycloheptenyl, phenyl, naphthyl, benzothiophenyl, benzodioxolyl, quinolinyl, indolyl, thiazolyl, thienyl, furanyl, isoxazolyl, oxazolyl, thiadiazolyl, pyrazinyl and pyridinyl each is optionally substituted with one or more Rₐ;
R₅ is chosen from methyl, CF₃, cyclopentyl, phenyl and cyclohexyl each optionally substituted with one or more R_{c}; and
n is 2-5.

6. The compound according to claim 5 and wherein:
Ar₁ is chosen from phenyl, thien-2-yl, isoxazol-5-yl and pyridin-3-yl;
Rₐ is chosen from phenyl, C₆₋₈ cycloalkyl, C₆₋₈ cycloalkenyl, C₁₋₃ alkoxy, C₁₋₄ alkyl, C₂₋₃ alkenyl, C₁₋₃ alkylthio, amino, carboxamide, fluoro, chloro, nitro and nitrile;
R₄ is chosen from: 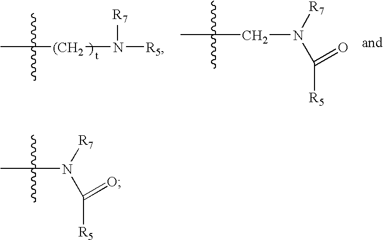
R₆ is independently chosen from hydroxy, methyl, ethyl, C₁₋₃ alkoxy, phenyl, morpholinyl, piperazinyl, imidazolyl, pyrrolidinyl, pyrrolidinonyl, thienylC₀₋₅ alkyl, C₃₋₇ cycloalkyl and amino said amino is optionally mono-or di-substituted by C₁₋₅ alkyl or C₁₋₅ alkoxycarbonyl; and
each R_{b} or R_{c} are independently chosen from C₁₋₃ alkoxy, amino optionally mono-or-di-substituted by C₁₋₃ alkyl, carboxamide, hydroxy, fluoro, chloro, bromo, trifluoromethyl, nitro and nitrile.

7. The compound according to any one of claims 1-6 and wherein:
R₄ is covalently attached at the indicated 5-position of the formula (I) or R₄ is covalently attached at the indicated 6-position of the formula (I).

8. A compound of the formula: 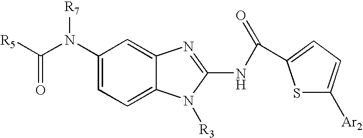
wherein:
Ar₂ is chosen from cycloheptyl, cycloheptenyl, phenyl, naphthyl, benzothiophenyl, benzodioxolyl, quinolinyl, indolyl, thiazolyl, thienyl, furanyl, isoxazolyl, oxazolyl, thiadiazolyl, pyrazinyl and pyridinyl each is optionally substituted with one or more Rₐ chosen from phenyl, C₆₋₈ cycloalkyl, C₆₋₈ cycloalkenyl, C₁₋₃ alkoxy, C₁₋₄ alkyl, C₂₋₃ alkenyl, C₁₋₃ alkylthio, amino, carboxamide, fluoro, chloro, nitro and nitrile;
R₃ is:
—(CH₂)ₙ—C(O)—R₆ or
—(CH₂)ₙ—R₆;
R₆ is independently chosen from hydroxy, methyl, ethyl, C₁₋₃ alkoxy, phenyl, morpholinyl, piperazinyl, imidazolyl, pyrrolidinyl, pyrrolidinonyl, thienylC₀₋₅ alkyl, C₃₋₇ cycloalkyl and amino said amino is optionally mono-or di-substituted by C₁₋₅ alkyl or C₁₋₅ alkoxycarbonyl;
R₅ is chosen from methyl, CF₃, cyclopentyl, phenyl and cyclohexyl each optionally substituted with one or more R_{c} chosen from C₁₋₃ alkoxy, amino optionally mono-or-di-substituted by C₁₋₃ alkyl, carboxamide, hydroxy, fluoro, chloro, bromo, trifluoromethyl, nitro and nitrile and
R₇ is C₁₋₃ alkyl;
or the pharmaceutically acceptable salts thereof.

9. The compound according to claim 8 and wherein:
Ar₂ is chosen from phenyl, indolyl, thiazolyl, thienyl, furanyl, isoxazolyl, oxazolyl, thiadiazolyl, pyrazinyl and pyridinyl each is optionally substituted with one or more Rₐ chosen from phenyl, C₆₋₈ cycloalkyl, C₆₋₈ cycloalkenyl, C₁₋₃ alkoxy, C₁₋₄ alkyl, C₂₋₃ alkenyl, C₁₋₃ alkylthio, amino, carboxamide, fluoro, chloro, nitro and nitrile.

10. The compound according to claim 9 and wherein:
Ar₂ is chosen from 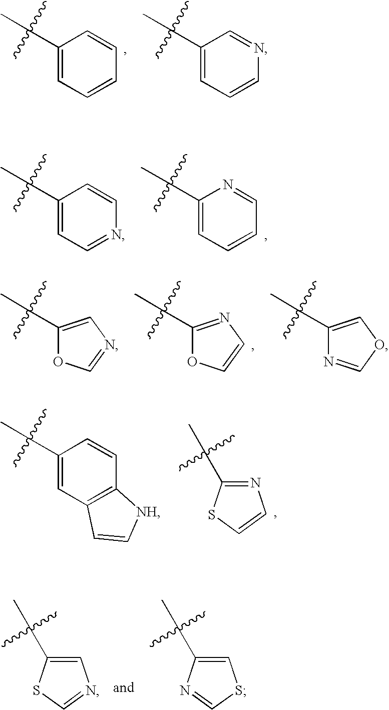
each is optionally substituted with one Rₐ chosen from phenyl, C₁₋₃ alkoxy, C₁₋₄ alkyl, C₂₋₃ alkenyl, C₁₋₃ alkylthio, amino, carboxamide, fluoro, chloro, nitro and nitrile;
with the proviso that if Ar₂ is: 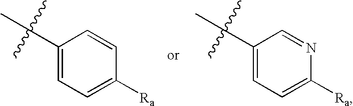
then Rₐ must be —NO₂, —NH₂ or —CN.

11. A pharmaceutical composition comprising a pharmaceutically effective amount of a compound according to claim 1 and one or more pharmaceutically acceptable carriers and/or adjuvants.

12. A method of treating an allergic disorder said method comprising administering to a patient in need thereof a therapeutically effect amount of a compound according to claim 1.

13. A method of treating a disease chosen from chronic inflammation, cancer, contact dermatitis, psoriasis, rheumatoid arthritis, multiple sclerosis, type 1 diabetes, inflammatory bowel disease, Guillain-Barre syndrome, Crohn's disease, ulcerative colitis, graft versus host disease, lupus erythematosus, asthma, chronic obstructive pulmonary disease (COPD), adult respiratory distress syndrome (ARDS), bronchitis, conjunctivitis, dermatitis and allergic rhinitis said method comprising administering to a patient in need thereof a therapeutically effect amount of a compound according to claim 1.
